SEQUENCE LISTING (40 sequences)

SEQ ID NO: 1 - protein, 972 aa, Homo sapiens
  human KIT receptor
MRGARGAWDFLCVLLLLLRVQTGSSQPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKEQIHPHTLFTPLLIGFVIVAGMMCIIVMILTYKYLQKPMYEVQWKVVEEINGNNYVYIDPTQLPYDHKWEFPRNRLSFGKTLGAGAFGKVVEATAYGLIKSDAAMTVAVKMLKPSAHLTEREALMSELKVLSYLGNHMNIVNLLGACTIGGPTLVITEYCCYGDLLNFLRRKRDSFICSKQEDHAEAALYKNLLHSKESSCSDSTNEYMDMKPGVSYVVPTKADKRRSVRIGSYIERDVTPAIMEDDELALDLEDLLSFSYQVAKGMAFLASKNCIHRDLAARNILLTHGRITKICDFGLARDIKNDSNYVVKGNARLPVKWMAPESIFNCVYTFESDVWSYGIFLWELFSLGSSPYPGMPVDSKFYKMIKEGFRMLSPEHAPAEMYDIMKTCWDADPLKRPTFKQIVQLIEKQISESTNHIYSNLANCSPNRQKPVVDHSVRINSVGSTASSSQPLLVHDDV

SEQ ID NO: 2 - protein, 11 aa, Artificial Sequence
  light chain CDR1 of anti-KIT antibody (VL-CDR1 - AbM)
KASQNVRTNVA

SEQ ID NO: 3 - protein, 7 aa, Artificial Sequence
  light chain CDR2 of anti-KIT antibody (VL-CDR2 - AbM)
SASYRYS

SEQ ID NO: 4 - protein, 9 aa, Artificial Sequence
  light chain CDR3 of anti-KIT antibody (VL-CDR3 - AbM)
QQYNSYPRT

SEQ ID NO: 5 - protein, 5 aa, Artificial Sequence
  heavy chain CDR1 of anti-KIT antibody (VH-CDR1)
DYYIN

SEQ ID NO: 6 - protein, 17 aa, Artificial Sequence
  heavy chain CDR2 of anti-KIT antibody (VH-CDR2)
RIYPGSGNTYYNEKFKG

SEQ ID NO: 7 - protein, 7 aa, Artificial Sequence
  heavy chain CDR3 of anti-KIT antibody (VH-CDR3)
GVYYFDY

SEQ ID NO: 8 - protein, 116 aa, Artificial Sequence
  heavy chain variable region of anti-KIT antibody (VH1)
QVQLVQSGAELKKPGASVKLSCKASGYTFTDYYINWVKQAPGKGLEWIARIYPGSGNTYYNEKFKGRATLTAEKSTSTAYMQLSSLRSEDSAVYFCARGVYYFDYWGQGTTVTVSS

SEQ ID NO: 9 - protein, 116 aa, Artificial Sequence
  heavy chain variable region of anti-KIT antibody (VH2)
QVQLVQSGAEVKKPGASVKLSCKASGYTFTDYYINWVKQAPGKGLEWIARIYPGSGNTYYNEKFKGRATLTAEKSTSTAYMQLSSLRSEDTAVYFCARGVYYFDYWGQGTTVTVSS

SEQ ID NO: 10 - protein, 116 aa, Artificial Sequence
  heavy chain variable region of anti-KIT antibody (VH3)
QVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSS

SEQ ID NO: 11 - protein, 116 aa, Artificial Sequence
  heavy chain variable region of anti-KIT antibody (VH4)
QVQLVQSGAEVKKPGASVKVSCKASGYTFTDYYINWVRQAPGKGLEWIARIYPGSGNTYYNEKFKGRATITADKSTSTAYMELSSLRSEDTAVYFCARGVYYFDYWGQGTTVTVSS

SEQ ID NO: 12 - protein, 116 aa, Artificial Sequence
  heavy chain variable region of anti-KIT antibody (VH5)
QVQLVQSGAEVKKPGASVKVSCKASGYTFTDYYINWVRQAPGKGLEWIARIYPGSGNTYYNEKFKGRVTITADKSTSTAYMELSSLRSEDTAVYFCARGVYYFDYWGQGTTVTVSS

SEQ ID NO: 13 - protein, 107 aa, Artificial Sequence
  light chain variable region of anti-KIT antibody (VL1)
DIVMTQSPSFLSASVGDRVTITCKASQNVRTNVAWYQQKPGKAPKALIYSASYRYSGVPDRFTGSGSGTDFTLTISSLQSEDFADYFCQQYNSYPRTFGGGTKVEIK

SEQ ID NO: 14 - protein, 107 aa, Artificial Sequence
  light chain variable region of anti-KIT antibody (VL2)
DIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIK

SEQ ID NO: 15 - protein, 107 aa, Artificial Sequence
  light chain variable region of anti-KIT antibody (VL3)
DIVMTQSPSSLSASVGDRVTITCKASQNVRTNVAWYQQKPGKAPKALIYSASYRYSGVPDRFSGSGSGTDFTLTISSLQPEDFADYFCQQYNSYPRTFGGGTKVEIK

SEQ ID NO: 16 - protein, 107 aa, Artificial Sequence
  light chain variable region of anti-KIT antibody (VL4)
DIVMTQSPSSLSASVGDRVTITCKASQNVRTNVAWYQQKPGKAPKSLIYSASYRYSGVPDRFSGSGSGTDFTLTISSLQPEDFATYYCQQYNSYPRTFGGGTKVEIK

SEQ ID NO: 17 - protein, 107 aa, Artificial Sequence
  light chain variable region consensus sequence
  misc_feature (10)..(10): Xaa can be any amino acid; or in specific enbodiment, it can be amino acid with an aromatic or aliphatic hydroxyl side chain; or in another specific embodiment, Xaa is the amino acid Phe or Ser
  misc_feature (46)..(46): Xaa can be any amino acid; or in specific enbodiment, it can be amino acid with an aliphatic or aliphatic hydroxyl side chain; or in another specific embodiment, Xaa is the amino acid Ala or Ser
  misc_feature (63)..(63): Xaa can be any amino acid; or in specific enbodiment, it can be amino acid with an aliphatic or aliphatic hydroxyl side chain; or in another specific embodiment, Xaa is the amino acid Thr or Ser
  misc_feature (80)..(80): Xaa can be any amino acid; or in specific enbodiment, it can be amino acid with an aliphatic or aliphatic hydroxyl side chain; or in another specific embodiment, Xaa is the amino acid Ser or Pro
  misc_feature (85)..(85): Xaa can be any amino acid; or in specific enbodiment, it can be amino acid with a charged or acidic side chain; or in another specific embodiment, Xaa is the amino acid Asp or Thr
  misc_feature (87)..(87): Xaa can be any amino acid; or in specific enbodiment, it can be amino acid with an aromatic side chain; or in another specific embodiment, Xaa is the amino acid Phe or Tyr
DIVMTQSPSXLSASVGDRVTITCKASQNVRTNVAWYQQKPGKAPKXLIYSASYRYSGVPDRFXGSGSGTDFTLTISSLQXEDFAXYXCQQYNSYPRTFGGGTKVEIK

SEQ ID NO: 18 - protein, 116 aa, Artificial Sequence
  heavy chain variable region consensus sequence
  misc_feature (11)..(11): Xaa can be any amino acid; or in specific enbodiment, it can be amino acid with an aliphatic side chain; or in another specific embodiment, Xaa is the amino acid Leu or Val
  misc_feature (20)..(20): Xaa can be any amino acid; or in specific enbodiment, it can be amino acid with an aliphatic side chain; or in another specific embodiment, Xaa is the amino acid Leu or Val
  misc_feature (38)..(38): Xaa can be any amino acid; or in specific enbodiment, it can be amino acid with a polar or basic side chain; or in another specific embodiment, Xaa is the amino acid Lys or Arg
  misc_feature (68)..(68): Xaa can be any amino acid; or in specific enbodiment, it can be amino acid with an aliphatic side chain; or in another specific embodiment, Xaa is the amino acid Val or Ala
  misc_feature (70)..(70): Xaa can be any amino acid; or in specific enbodiment, it can be amino acid with an aliphatic side chain; or in another specific embodiment, Xaa is the amino acid Leu or Ile
  misc_feature (73)..(73): Xaa can be any amino acid; or in specific enbodiment, it can be amino acid with an acidic side chain; or in another specific embodiment, Xaa is the amino acid Glu or Asp
  misc_feature (82)..(82): Xaa can be any amino acid; or in specific enbodiment, it can be amino acid with an acidic or amide derivative side chain; or in another specific embodiment, Xaa is the amino acid Gln or Glu
  misc_feature (91)..(91): Xaa can be any amino acid; or in specific enbodiment, it can be amino acid with an aliphatic hydroxyl side chain; or in another specific embodiment, Xaa is the amino acid Ser or Thr
QVQLVQSGAEXKKPGASVKXSCKASGYTFTDYYINWVXQAPGKGLEWIARIYPGSGNTYYNEKFKGRXTXTAXKSTSTAYMXLSSLRSEDXAVYFCARGVYYFDYWGQGTTVTVSS

SEQ ID NO: 19 - protein, 464 aa, Artificial Sequence
  heavy chain sequence with mutations
MEWSWVFLFFLSVTTGVHSQVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 234 aa, Artificial Sequence
  light chain sequence
MSVPTQVLGLLLLWLTDARCDIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 445 aa, Artificial Sequence
  mAb1 full-length heavy chain sequence (including Fc)
QVQLVQSGAEVKKPGASVKLSCKASGYTFTDYYINWVRQAPGKGLEWIARIYPGSGNTYYNEKFKGRATLTADKSTSTAYMQLSSLRSEDTAVYFCARGVYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAQGGPSVFLFPPKPKDTLYITREPEVTCVVVDVSHEDPEVKFNWYVDGVEVHNAKTKPREEQYNSTYRVVSVLTVLHQDWLNGKEYKCQVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 214 aa, Artificial Sequence
  mAb1 full-length light chain sequence
DIVMTQSPSSLSASVGDRVTITCKASQNVRTNVAWYQQKPGKAPKALIYSASYRYSGVPDRFTGSGSGTDFTLTISSLQPEDFADYFCQQYNSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - DNA, 1392 bp, Artificial Sequence
  DNA heavy chain sequence of an anti-KIT antibody
atggagtggtcctgggtgttcctgttctttctgtccgtgaccacaggcgtgcacagccaggtgcagctggtgcagtctggagctgaggtgaagaagccaggagcttctgtgaagctgtcctgcaaggccagcggctacaccttcacagactactatatcaactgggtgagacaggctcctggcaagggcctggagtggatcgctcgcatctatccaggctctggcaacacctactataatgagaagtttaagggccgggccaccctgacagctgataagagcacctctacagcttacatgcagctgtccagcctgagatccgaggacaccgccgtgtacttctgcgctcgcggcgtgtactattttgattattggggccagggcaccacagtgaccgtgtcttccgctagcacaaagggcccttccgtgtttccactggctcccagctctaagtccaccagcggaggaacagccgctctgggctgtctggtgaaggactatttcccagagcccgtgaccgtgagctggaactctggcgccctgaccagcggagtgcatacatttcctgctgtgctgcagtccagcggcctgtactctctgtcttccgtggtgaccgtgccaagctcttccctgggcacccagacatatatctgcaacgtgaatcacaagccatccaatacaaaggtggacaagaaggtggagcccaagagctgtgataagacccatacatgccccccttgtcctgctccagaggctcagggaggaccatccgtgttcctgtttccacccaagcctaaggacaccctgtacatcacaagggagccagaggtgacctgcgtggtggtggacgtgagccacgaggatcccgaggtgaagttcaactggtacgtggatggcgtggaggtgcataatgccaagacaaagccaagggaggagcagtacaatagcacctatcgggtggtgtctgtgctgacagtgctgcaccaggactggctgaacggcaaggagtacaagtgccaggtgtctaataaggccctgcccgctcctatcgagaagaccatctccaaggccaagggccagcctagggagccacaggtgtacacactgcctccaagccgggacgagctgaccaagaaccaggtgtctctgacatgtctggtgaagggcttctatccctctgatatcgctgtggagtgggagtccaatggccagcctgagaacaattacaagaccacaccccctgtgctggactccgatggcagcttctttctgtattccaagctgaccgtggataagagcaggtggcagcagggcaacgtgttttcttgttccgtgatgcatgaggctctgcacaatcattacacacagaagagcctgtctctgtcccctggc

SEQ ID NO: 24 - DNA, 702 bp, Artificial Sequence
  DNA light chain sequence of an anti-KIT antibody
atgtccgtgccaacccaggtgctgggactgctgctgctgtggctgaccgacgccaggtgcgatatcgtgatgacacagtccccttccagcctgtctgcttccgtgggcgacagagtgaccatcacctgtaaggccagccagaacgtgcgcaccaatgtggcttggtaccagcagaagccaggcaaggcccccaaggctctgatctatagcgcctcttacaggtatagcggagtgcctgaccggttcaccggatccggaagcggaacagacttcaccctgacaatctcttccctgcagcctgaggacttcgctgattacttttgccagcagtacaactcttatccaaggaccttcggcggcggcacaaaggtggagatcaagcggaccgtggccgctccaagcgtgttcatctttcccccttctgacgagcagctgaagtctggcacagcctccgtggtgtgcctgctgaacaacttctaccccagagaggccaaggtgcagtggaaggtggataacgctctgcagtctggcaattcccaggagagcgtgaccgagcaggactctaaggattccacatatagcctgagctctaccctgacactgtctaaggccgattacgagaagcacaaggtgtatgcttgcgaggtgacccatcagggcctgtccagcccagtgacaaagtccttcaatcgcggcgagtgt

SEQ ID NO: 25 - protein, 7 aa, Artificial Sequence
  heavy chain CDR1 of anti-KIT antibody (VH-CDR1)
GYTFTDY

SEQ ID NO: 26 - protein, 6 aa, Artificial Sequence
  heavy chain CDR2 of anti-KIT antibody (VH-CDR2)
YPGSGN

SEQ ID NO: 27 - protein, 8 aa, Artificial Sequence
  heavy chain CDR3 of anti-KIT antibody (VH-CDR3 - AbM)
GVYYFDYW

SEQ ID NO: 28 - protein, 7 aa, Artificial Sequence
  light chain CDR1 of anti-KIT antibody (VL-CDR1)
SQNVRTN

SEQ ID NO: 29 - protein, 3 aa, Artificial Sequence
  light chain CDR2 of anti-KIT antibody (VL-CDR2)
SAS

SEQ ID NO: 30 - protein, 6 aa, Artificial Sequence
  light chain CDR3 of anti-KIT antibody (VL-CDR3)
YNSYPR

SEQ ID NO: 31 - protein, 4 aa, Artificial Sequence
  heavy chain CDR2 of anti-KIT antibody (VH-CDR2)
PGSG

SEQ ID NO: 32 - protein, 6 aa, Artificial Sequence
  heavy chain CDR3 of anti-KIT antibody (VH-CDR3)
VYYFDY

SEQ ID NO: 33 - protein, 10 aa, Artificial Sequence
  heavy chain CDR1 of anti-KIT antibody (VH-CDR1 - AbM)
GYTFTDYYIN

SEQ ID NO: 34 - protein, 10 aa, Artificial Sequence
  heavy chain CDR2 of anti-KIT antibody (VH-CDR2 - AbM)
RIYPGSGNTY

SEQ ID NO: 35 - protein, 7 aa, Artificial Sequence
  light chain CDR1 of anti-KIT antibody (VL-CDR1 - Contact)
RTNVAWY

SEQ ID NO: 36 - protein, 10 aa, Artificial Sequence
  light chain CDR2 of anti-KIT antibody (VL-CDR2 - Contact)
ALIYSASYRY

SEQ ID NO: 37 - protein, 8 aa, Artificial Sequence
  light chain CDR3 of anti-KIT antibody (VL-CDR3 - Contact)
QQYNSYPR

SEQ ID NO: 38 - protein, 6 aa, Artificial Sequence
  heavy chain CDR1 of anti-KIT antibody (VH-CDR1 - Contact)
TDYYIN

SEQ ID NO: 39 - protein, 13 aa, Artificial Sequence
  heavy chain CDR2 of anti-KIT antibody (VH-CDR2 - Contact)
WIARIYPGSGNTY

SEQ ID NO: 40 - protein, 9 aa, Artificial Sequence
  heavy chain CDR3 of anti-KIT antibody (VH-CDR3 - Contact)
ARGVYYFDY

(19) United States
(12) Patent Application Publication
(10) Pub. No.: US 2024/0117057 A1
(43) Pub. Date: Apr. 11, 2024

(54) ANTI-KIT ANTIBODIES AND USES THEREOF

(71) Applicant: Celldex Therapeutics, Inc., Hampton, NJ (US)

(72) Inventor: Joel Goldstein, Hopewell, NJ (US)

(73) Assignee: Celldex Therapeutics, Inc., Hampton, NJ (US)

(21) Appl. No.: 18/273,680

(22) PCT Filed: Jan. 21, 2022
(86) PCT No.: PCT/US2022/013365

§ 371 Date: Jul. 21, 2023

Related U.S. Application Data
(60) Provisional application No. 63/238,649, filed on Aug. 30, 2021.
(60) Provisional application No. 63/140,642, filed on Jan. 22, 2021.

(51) Int. Cl. C07K 16/28; A61P 17/00

(52) U.S. Cl. CPC C07K 16/2866; A61P 17/00; C07K 2317/52; C07K 2317/76

(57) ABSTRACT

Provided herein are antibodies that immunospecifically bind to KIT, a receptor tyrosine kinase, and uses thereof. Also provided are polynucleotides and vectors encoding such antibodies, cells comprising such polynucleotides or vectors, and methods of making such antibodies.

### CROSS-REFERENCE TO RELATED APPLICATIONS

This application claims the benefit of U.S. Provisional Patent Application Nos. 63/140,642, filed Jan. 22, 2021, and 63/238,649, filed Aug. 30, 2021, which are incorporated by reference herein in their entireties.

### REFERENCE TO SEQUENCE LISTING SUBMITTED ELECTRONICALLY

This application incorporates by reference a Sequence Listing submitted with this application as text file entitled “Seglisting_12638-170-228.txt” created on Jan. 10, 2022 and having a size of 44,171 bytes.

### 1. FIELD

Provided herein are antibodies that immunospecifically bind to KIT, a receptor tyrosine kinase, and uses thereof. Also provided are polynucleotides and vectors encoding such antibodies, cells comprising such polynucleotides or vectors, and methods of making such antibodies.

### 2. BACKGROUND

KIT (or c-Kit) is a type III receptor tyrosine kinase encoded by the c-kit gene. KIT comprises five extracellular immunoglobulin (Ig)-like domains, a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). The human c-kit gene encoding the KIT receptor has been cloned as described by Yarden et al., EMBO J., 1987, 6:3341-3351. KIT is also known as CD117 or stem cell factor receptor (“SCFR”), because it is the receptor for the stem cell factor (“SCF”) ligand (also known as Steel Factor or Kit Ligand). SCF ligand binding to the first three extracellular Ig-like domains of KIT induces receptor dimerization, and thereby activates intrinsic tyrosine kinase activity through the phosphorylation of specific tyrosine residues in the juxtamembrane and kinase domains (see, e.g., Weiss and Schlessinger, Cell, 1998, 94:277-280; Clifford et al., J. Biol. Chem., 2003, 278:31461-31464). Members of the Stat, Src, ERK, and AKT signaling pathways have been shown to be downstream signal transducers of KIT signaling.

The fourth (D4) and fifth (D5) extracellular Ig-like domains of KIT are believed to mediate receptor dimerization (see, e.g., International Patent Application Publication No. WO 2008/153926; Yuzawa et al., Cell, 2007, 130:323-334).

Expression of KIT has been detected in various cell types, such as mast cells, stem cells, brain cells, melanoblasts, ovary cells, and cancer cells (e.g., leukemia cells) (see, e.g., Besmer, P. Curr. Opin. Cell Biol, 1991, 3:939-946; Lyman et al., Blood, 1998, 91: 1101-1134; Ashman, L. K., Int. J. Biochem. Cell Biol, 1999, 31: 1037-1051; Kitamura et al., Mutat. Res., 2001, 477: 165-171; Mol et al., J. Biol. Chem., 2003, 278:31461-31464). Moreover, KIT plays an important role in hematopoiesis, melanogenesis, and gametogenesis (see Ueda et al., Blood, 2002, 99:3342-3349).

Antibodies that immunospecifically bind to human KIT are known, for example from International Patent Publication No WO2014018625A1, which is herein incorporated by reference in its entirety.

There is a need to provide improved antibodies against human KIT.

### 3. SUMMARY

In one aspect, provided herein is an antibody which immunospecifically binds to human KIT, comprising: (i) a light chain variable region (“VL”) comprising VL CDR1, VL CDR2, and VL CDR3 having the amino acid sequences of SEQ ID NO: 2, SEQ ID NO: 3, and SEQ ID NO: 4, respectively; (ii) a heavy chain variable region (“VH”) comprising VH CDR1, VH CDR2, and VH CDR3 having the amino acid sequences of SEQ ID NO: 5, SEQ ID NO: 6, and SEQ ID NO: 7, respectively; and (iii) a modified (e.g., mutated) human IgG1 Fc region or domain which comprises non-naturally occurring amino acids 234A, 235Q and 322Q as numbered by the EU index as set forth in Kabat. In specific embodiments, the modified (e.g., mutated) human IgG1 Fc region or domain further comprises non-naturally occurring amino acids 252Y, 254T and 256E as numbered by the EU index as set forth in Kabat.

In specific embodiments, (i) the VL of the antibody comprises the amino acid sequence:

(SEQ ID NO: 17)
DIVMTQSPSX_{K1}LSASVGDRVTITCKASQNVR
TNVAWYQQKPGKAPKX_{K2}LIYSASYRYSGVPD
RFX_{K3}GSGSGTDFTLTISSLQX_{K4}EDFAXKSY
X_{K6}CQQYNSYPRTFGGGTKVEIK,

wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and (ii) the VH of the antibody comprises the amino acid sequence:

(SEQ ID NO: 18)
QVQLVQSGAEX_{H1}KKPGASVKX_{H2}SCKASGYT
FTDYYINWVX_{H3}QAPGKGLEWIARIYPGSGNT
YYNEKFKGRX_{H4}TXHSTAX_{H6}KSTSTAYMX_{H7}
LSSLRSEDX_{H8}AVYFCARGVYYFDYWGQGTTV
TVSS,
wherein X_{H1} to X_{H8} is any amino acid.

wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7} is an amino acid with an acidic or amide derivative side chain, and X_{H8} is an amino acid with an aliphatic hydroxyl side chain. In a specific embodiment, X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In specific embodiments, (i) the VL of the antibody comprises the amino acid sequence of SEQ ID NO: 13, 14, 15 or 16, and (ii) the VH of the antibody comprises the amino acid sequence of SEQ ID NO: 8, 9, 10, 11 or 12.

In specific embodiments, the antibody comprises: (i) a VL comprising an amino acid sequence of SEQ ID NO: 14; (ii) a VH comprising an amino acid sequence of SEQ ID NO: 10; and (iii) a modified (e.g., mutated) human IgG1 Fc region or domain comprising non-naturally occurring amino acids 234A, 235Q and 322Q as numbered by the EU index as set forth in Kabat.

In specific embodiments, the antibody comprises: (i) a VL comprising an amino acid sequence of SEQ ID NO: 14; (ii) a VH comprising an amino acid sequence of SEQ ID NO: 10; and (iii) a modified (e.g., mutated) human IgG1 Fc region or domain comprising non-naturally occurring amino acids 234A, 235Q, 322Q, 252Y, 254T and 256E as numbered by the EU index as set forth in Kabat.

In specific embodiments, the antibody comprises a heavy chain comprising the amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG

In specific embodiments, the antibody comprises a light chain comprising the amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

In specific embodiments, the antibody comprises (i) a heavy chain comprising the amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG;

and (ii) a light chain comprising the amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In another aspect, provided herein is a conjugate comprising an antibody provided herein linked to an agent.

In another aspect, provided herein is a pharmaceutical composition comprising an antibody or a conjugate provided herein and a pharmaceutically acceptable carrier.

In another aspect, provided herein is a polynucleotide or combination of polynucleotides comprising nucleotide sequences encoding an antibody provided herein, or the VH and VL of said antibody.

In another aspect, provided herein is a vector or combination of vectors comprising a polynucleotide or combination of polynucleotides provided herein.

In another aspect, provided herein is a host cell comprising a vector or combination of vectors provided herein or a polynucleotide or combination of polynucleotides provided herein.

In another aspect, provided herein is a kit comprising an antibody provided herein, a conjugate provided herein, or a pharmaceutical composition provided herein.

In another aspect, provided herein is a method for protecting against, treating or managing a KIT-associated disorder, comprising administering to a subject in need thereof a therapeutically effective amount of an antibody provided herein, a conjugate provided herein, or a pharmaceutical composition provided herein. In specific embodiments, the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis. In a specific embodiment, the KIT-associated disorder is a mast cell related disorder. In a particular embodiment, the mast cell related disorder is chronic urticaria. In one embodiment, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria. In a specific embodiment, the KIT-associated disorder is an eosinophil related disorder such as eosinophilic esophagitis (EoE).

In certain embodiments, a method provide herein further comprises administering a second therapeutic agent to the subject. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

In another aspect, provided herein is a method for inhibiting KIT activity in a cell expressing KIT, comprising contacting the cell with an effective amount of an antibody provided herein, a conjugate provided herein, or a pharmaceutical composition provided herein. In specific embodiments, the method inhibits KIT activity by at least about 10% in the cell expressing KIT.

In another aspect, provided herein is an in vitro method for diagnosing a subject with a KIT-associated disorder, wherein the method comprises contacting cells or a sample obtained from the subject with an antibody provided herein and detecting the expression level of KIT in the cells or the sample.

In another aspect, provided herein is a method of making an antibody, wherein said method comprises culturing, and/or expressing the antibody using, a host cell provided herein. In specific embodiments, the method further comprises purifying the antibody obtained from said host cell.

In another aspect, provided herein is the use of an antibody provided herein, a conjugate provided herein, or a pharmaceutical composition provided herein for the manufacture of a medicament for protecting against, treating or managing a KIT-associated disorder. In specific embodiments, the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis. In a specific embodiment, the KIT-associated disorder is a mast cell related disorder. In a particular embodiment, the mast cell related disorder is chronic urticaria. In one embodiment, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria. In a specific embodiment, the KIT-associated disorder is an eosinophil related disorder such as eosinophilic esophagitis (EoE).

In certain embodiments, the medicament described herein is manufactured to be administered in combination with a second therapeutic agent. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

In another aspect, provided herein is the use of an antibody provided herein, a conjugate provided herein, or a pharmaceutical composition provided herein for the manufacture of a medicament for inhibiting KIT activity in a cell expressing KIT. In specific embodiments, the medicament inhibits KIT activity by at least about 10% in the cell expressing KIT.

In another aspect, provided herein is an antibody described herein, a conjugate described herein, or a pharmaceutical composition described herein, for use in a method for protecting against, treating or managing a KIT-associated disorder. In specific embodiments, the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis. In a specific embodiment, the KIT-associated disorder is a mast cell related disorder. In a particular embodiment, the mast cell related disorder is chronic urticaria. In one embodiment, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria. In a specific embodiment, the KIT-associated disorder is an eosinophil related disorder such as eosinophilic esophagitis (EoE).

In certain embodiments, the method further comprises administering a second therapeutic agent to the subject. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

In another aspect, provided herein is an antibody described herein, a conjugate described herein, or a pharmaceutical composition described herein, for use in a method for inhibiting KIT activity in a cell expressing KIT. In specific embodiments, the method inhibits KIT activity by at least about 10% in the cell expressing KIT.

In another aspect, provided herein is a method for protecting against, treating or managing chronic urticaria in a subject, comprising administering to a subject in need thereof a therapeutically effective amount of (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier. In certain embodiments, the human KIT comprises the amino acid sequence of SEQ ID NO: 1. In certain embodiments, the antibody specifically binds to a D4 or D5 region of human KIT. In certain embodiments, the antibody comprises a modified (e.g., mutated) Fc region or domain (e.g., a modified (e.g., mutated) human Fc region or domain). In certain embodiments, the antibody has reduced Fc receptor binding activity (particularly reduced FcTR binding activity). In certain embodiments, the antibody does not induce significant degranulation of FcgRI-expressing human mast cells. In certain embodiments, the antibody does not show significant Fc receptor-dependent KIT agonist activity. In certain embodiments, the antibody comprises: (A) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 5, SEQ ID NO: 6, and SEQ ID NO: 7, respectively; (B) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 25, SEQ ID NO: 26, and SEQ ID NO: 27, respectively; (C) (i) a VL comprising VL CDR1, VL CDR2, and VL CDR3 having the amino acid sequences of SEQ ID NO: 28, SEQ ID NO: 29, and SEQ ID NO: 30, respectively; and (ii) a VH comprising VH CDR1, VH CDR2, and VH CDR3 having the amino acid sequences of SEQ ID NO: 25, SEQ ID NO: 31, and SEQ ID NO: 32, respectively; (D) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 33, SEQ ID NO: 34, and SEQ ID NO: 27, respectively; or (E) (i) a VL comprising VL CDR1, VL CDR2, and VL CDR3 having the amino acid sequences of SEQ ID NO: 35, SEQ ID NO: 36, and SEQ ID NO: 37, respectively; and (ii) a VH comprising VH CDR1, VH CDR2, and VH CDR3 having the amino acid sequences of SEQ ID NO: 38, SEQ ID NO: 39, and SEQ ID NO: 40, respectively. In one embodiment, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria.

In certain embodiments, a method provide herein further comprises administering a second therapeutic agent to the subject. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

In another aspect, provided herein is the use of (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier, for the manufacture of a medicament for protecting against, treating or managing chronic urticaria in a subject. In certain embodiments, the human KIT comprises the amino acid sequence of SEQ ID NO: 1. In certain embodiments, the antibody specifically binds to a D4 or D5 region of human KIT. In certain embodiments, the antibody comprises a modified (e.g., mutated) Fc region or domain (e.g., a modified (e.g., mutated) human Fc region or domain). In certain embodiments, the antibody has reduced Fc receptor binding activity (particularly reduced FcTR binding activity). In certain embodiments, the antibody does not induce significant degranulation of FcgRI-expressing human mast cells. In certain embodiments, the antibody does not show significant Fc receptor-dependent KIT agonist activity. In certain embodiments, the antibody comprises: (A) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 5, SEQ ID NO: 6, and SEQ ID NO: 7, respectively; (B) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 25, SEQ ID NO: 26, and SEQ ID NO: 27, respectively; (C) (i) a VL comprising VL CDR1, VL CDR2, and VL CDR3 having the amino acid sequences of SEQ ID NO: 28, SEQ ID NO: 29, and SEQ ID NO: 30, respectively; and (ii) a VH comprising VH CDR1, VH CDR2, and VH CDR3 having the amino acid sequences of SEQ ID NO: 25, SEQ ID NO: 31, and SEQ ID NO: 32, respectively; (D) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 33, SEQ ID NO: 34, and SEQ ID NO: 27, respectively; or (E) (i) a VL comprising VL CDR1, VL CDR2, and VL CDR3 having the amino acid sequences of SEQ ID NO: 35, SEQ ID NO: 36, and SEQ ID NO: 37, respectively; and (ii) a VH comprising VH CDR1, VH CDR2, and VH CDR3 having the amino acid sequences of SEQ ID NO: 38, SEQ ID NO: 39, and SEQ ID NO: 40, respectively. In specific embodiments, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria.

In certain embodiments, the medicament described herein is manufactured to be administered in combination with a second therapeutic agent. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

In another aspect, provided herein is (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier, for use in a method for protecting against, treating or managing chronic urticaria. In certain embodiments, the human KIT comprises the amino acid sequence of SEQ ID NO: 1. In certain embodiments, the antibody specifically binds to a D4 or D5 region of human KIT. In certain embodiments, the antibody comprises a modified (e.g., mutated) Fc region or domain (e.g., a modified (e.g., mutated) human Fc region or domain). In certain embodiments, the antibody has reduced Fc receptor binding activity (particularly reduced FcTR binding activity). In certain embodiments, the antibody does not induce significant degranulation of FcgRI-expressing human mast cells. In certain embodiments, the antibody does not show significant Fc receptor-dependent KIT agonist activity. In certain embodiments, the antibody comprises: (A) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 5, SEQ ID NO: 6, and SEQ ID NO: 7, respectively; (B) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 25, SEQ ID NO: 26, and SEQ ID NO: 27, respectively; (C) (i) a VL comprising VL CDR1, VL CDR2, and VL CDR3 having the amino acid sequences of SEQ ID NO: 28, SEQ ID NO: 29, and SEQ ID NO: 30, respectively; and (ii) a VH comprising VH CDR1, VH CDR2, and VH CDR3 having the amino acid sequences of SEQ ID NO: 25, SEQ ID NO: 31, and SEQ ID NO: 32, respectively; (D) (i) a VL comprising VL CDR1, VL CDR2, and VL CDR3 having the amino acid sequences of SEQ ID NO: 2, SEQ ID NO: 3, and SEQ ID NO: 4, respectively; and (ii) a VH comprising VH CDR1, VH CDR2, and VH CDR3 having the amino acid sequences of SEQ ID NO: 33, SEQ ID NO: 34, and SEQ ID NO: 27, respectively; or (E) (i) a VL comprising VL CDR1, VL CDR2, and VL CDR3 having the amino acid sequences of SEQ ID NO: 35, SEQ ID NO: 36, and SEQ ID NO: 37, respectively; and (ii) a VH comprising VH CDR1, VH CDR2, and VH CDR3 having the amino acid sequences of SEQ ID NO: 38, SEQ ID NO: 39, and SEQ ID NO: 40, respectively. In specific embodiments, the chronic urticaria is chronic inducible urticaria. In one embodiment, the chronic inducible urticaria is cold urticaria. In one embodiment, the chronic inducible urticaria is symptomatic dermographism. In one embodiment, the chronic inducible urticaria is cholinergic urticaria. In another embodiment, the chronic urticaria is chronic spontaneous urticaria.

In certain embodiments, the method further comprises administering a second therapeutic agent to the subject. In specific embodiments, the second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

### 3.1 Illustrative Embodiments

1. An antibody which imrnmunospecifically binds to human KIT, comprising:
(i) a light chain variable region (“VL”) comprising VL CDR1, VL CDR2, and VL CDR3 having the amino acid sequences of SEQ ID NO: 2, SEQ ID NO: 3, and SEQ ID NO: 4, respectively;
(ii) a heavy chain variable region (“VH”) comprising VH CDR1, VH CDR2, and VH CDR3 having the amino acid sequences of SEQ ID NO: 5, SEQ ID NO: 6, and SEQ ID NO: 7, respectively; and
(iii) a modified (e.g., mutated) human IgG1 Fc region or domain which comprises non-naturally occurring amino acids 234A, 235Q and 322Q as numbered by the EU index as set forth in Kabat.
   2. The antibody of embodiment 1, wherein the modified (e.g., mutated) human IgG1 Fc region or domain further comprises non-naturally occurring amino acids 252Y, 254T and 256E as numbered by the EU index as set forth in Kabat.
   3. The antibody of embodiment 1 or 2, wherein:
(i) the VL comprises the amino acid sequence:

(SEQ ID NO: 17)
DIVMTQSPSX_{K1}LSASVGDRVTITCKASQNVR
TNVAWYQQKPGKAPKX_{K2}LIYSASYRYSGVPD
RFX_{K3}GSGSGTDFTLTISSLQX_{K4}EDFAXKSY
X_{K6}CQQYNSYPRTFGGGTKVEIK,

wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) the VH comprises the amino acid sequence:

(SEQ ID NO: 18)
QVQLVQSGAEX_{H1}KKPGASVKX_{H2}SCKASGYT
FTDYYINWVX_{H3}QAPGKGLEWIARIYPGSGNT
YYNEKFKGRX_{H4}TXHSTAX_{H6}KSTSTAYMX_{H7}
LSSLRSEDX_{H8}AVYFCARGVYYFDYWGQGTTV
TVSS,
wherein X_{H1} to X_{H8} is any amino acid.

wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H5} is an amino acid with an aliphatic side chain, X_{H6} is an amino acid with an acidic side chain, X_{H7}; is an amino acid with an acidic or amide derivative side chain, and X_{H8} is an amino acid with an aliphatic hydroxyl side chain.
4. The antibody of embodiment 3, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} iS the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.
5. The antibody of any one of embodiments 1 to 4, wherein:
i) the VL comprises the amino acid sequence of SEQ ID NO: 13, 14, 15 or 16, and
ii) the VH comprises the amino acid sequence of SEQ ID NO: 8, 9, 10, 11 or 12.
   6 The antibody of any one of embodiments 1 to 5, comprising:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q and 322Q as numbered by the EU index as set forth in Kabat.
   7. The antibody of any one of embodiments 1 to 5, comprising:
(i) a VL comprising an amino acid sequence of SEQ ID NO: 14;
(ii) a VH comprising an amino acid sequence of SEQ ID NO: 10; and
(iii) a modified (e.g., mutated) human IgG1 Fc region or domain comprising non-naturally occurring amino acids 234A, 235Q, 322Q, 252Y, 254T and 256E as numbered by the EU index as set forth in Kabat.
   8. The antibody of any one of embodiments 1 to 5, which comprises a heavy chain comprising the amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG.

9. The antibody of any one of embodiments 1 to 5, which comprises a light chain comprising the amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSENRGEC.

10. The antibody of any one of embodiments 1 to 5, which comprises a heavy chain comprising the amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG;

and a light chain comprising the amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

11. A conjugate comprising the antibody of any one of embodiments 1 to 10 linked to an agent.
12. A pharmaceutical composition comprising the antibody of any one of embodiments 1 to 10, or the conjugate of embodiment 11, and a pharmaceutically acceptable carrier.
13. A polynucleotide or combination of polynucleotides comprising nucleotide sequences encoding the antibody of any one of embodiments 1 to 10, or the VH and VL of said antibody.
14. A vector or combination of vectors comprising the polynucleotide or combination of polynucleotides of embodiment 13.
15. A host cell comprising the vector or combination of vectors of embodiment 14 or the polynucleotide or combination of polynucleotides of embodiment 13.
16. A kit comprising the antibody of any one of embodiments 1 to 10, the conjugate of embodiment 11, or the pharmaceutical composition of embodiment 12.
17. A method for protecting against, treating or managing a KIT-associated disorder, comprising administering to a subject in need thereof a therapeutically effective amount of the antibody of any one of embodiments 1 to 10, the conjugate of embodiment 11, or the pharmaceutical composition of embodiment 12.
18. The method of embodiment 17, wherein the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis.
19. The method of embodiment 18, wherein the KIT-associated disorder is a mast cell related disorder.
20. The method of embodiment 19, wherein the mast cell related disorder is chronic urticaria.
21. The method of embodiment 20, wherein the chronic urticaria is chronic inducible urticaria.
22. The method of embodiment 21, wherein the chronic inducible urticaria is cold urticaria.
23. The method of embodiment 21, wherein the chronic inducible urticaria is symptomatic dermographisn.
24. The method of embodiment 21, wherein the chronic inducible urticaria is cholinergic urticaria.
25. The method of embodiment 20, wherein the chronic urticaria is chronic spontaneous urticaria.
26. The method of embodiment 18, wherein the KIT-associated disorder is an eosinophil related disorder.
27. The method of any one of embodiments 17 to 26, further comprising administering a second therapeutic agent to the subject.
28. The method of embodiment 27, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
29. A method for inhibiting KIT activity in a cell expressing KIT, comprising contacting the cell with an effective amount of the antibody of any one of embodiments 1 to 10, the conjugate of embodiment 11, or the pharmaceutical composition of embodiment 12.
30. The method of embodiment 29, wherein the method inhibits KIT activity by at least about 10% in the cell expressing KIT.
31. An in vitro method for diagnosing a subject with a KIT-associated disorder, wherein the method comprises contacting cells or a sample obtained from the subject with the antibody of any one of embodiments 1 to 10 and detecting the expression level of KIT in the cells or the sample.
32. A method of making an antibody, wherein said method comprises culturing, and/or expressing the antibody using, the host cell of embodiment 15.
33. The method of embodiment 32, further comprising purifying the antibody obtained from said host cell.
34. Use of the antibody of any one of embodiments 1 to 10, the conjugate of embodiment 11, or the pharmaceutical composition of embodiment 12, for the manufacture of a medicament for protecting against, treating or managing a KIT-associated disorder.
35. The use of embodiment 34, wherein the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis.
36. The use of embodiment 35, wherein the KIT-associated disorder is a mast cell related disorder.
37. The use of embodiment 36, wherein the mast cell related disorder is chronic urticaria.
38. The use of embodiment 37, wherein the chronic urticaria is chronic inducible urticaria.
39. The use of embodiment 38, wherein the chronic inducible urticaria is cold urticaria.
40. The use of embodiment 38, wherein the chronic inducible urticaria is symptomatic dermographism.
41. The use of embodiment 38, wherein the chronic inducible urticaria is cholinergic urticaria.
42. The use of embodiment 37, wherein the chronic urticaria is chronic spontaneous urticaria.
43. The use of embodiment 35, wherein the KIT-associated disorder is an eosinophil related disorder.
44. The use of any one of embodiments 34 to 43, wherein the medicament is manufactured to be administered in combination with a second therapeutic agent.
45. The use of embodiment 44, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
46. Use of the antibody of any one of embodiments 1 to 10, the conjugate of embodiment 11, or the pharmaceutical composition of embodiment 12, for the manufacture of a medicament for inhibiting KIT activity in a cell expressing KIT.
47. The use of embodiment 46, wherein the medicament inhibits KIT activity by at least about 10% in the cell expressing KIT.
48. The antibody of any one of embodiments 1 to 10, the conjugate of embodiment 11, or the pharmaceutical composition of embodiment 12, for use in a method for protecting against, treating or managing a KIT-associated disorder.
49. The antibody, conjugate, or pharmaceutical composition for use of embodiment 48, wherein the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis.
50. The antibody, conjugate, or pharmaceutical composition for use of embodiment 49, wherein the KIT-associated disorder is a mast cell related disorder.
51. The antibody, conjugate, or pharmaceutical composition for use of embodiment 50, wherein the mast cell related disorder is chronic urticaria.
52. The antibody, conjugate, or pharmaceutical composition for use of embodiment 51, wherein the chronic urticaria is chronic inducible urticaria.
53. The antibody, conjugate, or pharmaceutical composition for use of embodiment 52, wherein the chronic inducible urticaria is cold urticaria.
54. The antibody, conjugate, or pharmaceutical composition for use of embodiment 52, wherein the chronic inducible urticaria is symptomatic dermographism.
55. The antibody, conjugate, or pharmaceutical composition for use of embodiment 52, wherein the chronic inducible urticaria is cholinergic urticaria.
56. The antibody, conjugate, or pharmaceutical composition for use of embodiment 51, wherein the chronic urticaria is chronic spontaneous urticaria.
57. The antibody, conjugate, or pharmaceutical composition for use of embodiment 49, wherein the KIT-associated disorder is an eosinophil related disorder.
58. The antibody, conjugate, or pharmaceutical composition for use of any one of embodiments 48 to 57, wherein the method further comprises administering a second therapeutic agent to the subject.
59. The antibody, conjugate, or pharmaceutical composition for use of embodiment 58, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
60. The antibody of any one of embodiments 1 to 10, the conjugate of embodiment 11, or the pharmaceutical composition of embodiment 12, for use in a method for inhibiting KIT activity in a cell expressing KIT.
61. The antibody, conjugate, or pharmaceutical composition for use of embodiment 60, wherein the method inhibits KIT activity by at least about 10% in the cell expressing KIT.
62. A method for protecting against, treating or managing chronic urticaria in a subject, comprising administering to a subject in need thereof a therapeutically effective amount of (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier.
63. The method of embodiment 62, wherein the human KIT comprises the amino acid sequence of SEQ ID NO: 1.
64. The method of embodiment 62 or 63, wherein the antibody specifically binds to a D4 or D5 region of human KIT.
65. The method of any one of embodiments 62 to 64, wherein the antibody comprises a modified (e.g., mutated) Fc region or domain.
66. The method of any one of embodiments 62 to 65, wherein the antibody comprises a modified (e.g., mutated) human Fc region or domain.
67. The method of any one of embodiments 62 to 66, wherein the antibody has reduced Fc receptor binding activity (particularly reduced FcTR binding activity).
68. The method of any one of embodiments 62 to 67, wherein the antibody does not induce significant degranulation of FcgRI-expressing human mast cells.
69. The method of any one of embodiments 62 to 68, wherein the antibody does not show significant Fc receptor-dependent KIT agonist activity.
70. The method of any one of embodiments 62 to 69, wherein the antibody comprises:
(A) (i) a VL comprising VL CDR1, VL CDR2, and VL CDR3 having the amino acid sequences of SEQ ID NO: 2, SEQ ID NO: 3, and SEQ ID NO: 4, respectively; and
   (ii) a VH comprising VH CDR1, V H CDR2, and VH CDR3 having the amino acid sequences of SEQ ID NO: 5, SEQ ID NO: 6, and SEQ ID NO: 7, respectively;
(B) (i) a VL comprising VL CDR1, VL CDR2, and VL CDR3 having the amino acid sequences of SEQ ID NO: 2, SEQ ID NO: 3, and SEQ ID NO: 4, respectively; and
   (ii) a VH comprising VH CDR1, VH CDR2, and VH CDR3 having the amino acid sequences of SEQ ID NO: 25, SEQ ID NO: 26, and SEQ ID NO: 27, respectively;
(C) (i) a VL comprising VL CDR1, VL CDR2, and ML CDR3 having the amino acid sequences of SEQ ID NO: 28, SEQ ID NO: 29, and SEQ ID NO: 30, respectively; and
   (ii) a MH comprising VH CDR1, MH CDR2, and VH CDR3 having the amino acid sequences of SEQ ID NO: 25, SEQ ID NO: 31, and SEQ ID NO: 32, respectively;
(D) (i) a VL comprising VL CDR1, VL CDR2, and VL CDR3 having the amino acid sequences of SEQ ID NO: 21 SEQ ID NO: 3, and SEQ ID NO: 4, respectively; and
   (ii) a VH comprising VH CDR1, VH CDR2, and VH CDR3 having the amino acid sequences of SEQ ID NO: 33, SEQ ID NO: 34, and SEQ ID NO: 27, respectively; or
(E) (i) a VL comprising VL CDR1, VL CDR2, and VL CDR3 having the amino acid sequences of SEQ ID NO: 35, SEQ ID NO: 36, and SEQ ID NO: 37, respectively; and
   (ii) a VH comprising VH CDR1, VH CDR2, and VH CDR3 having the amino acid sequences of SEQ ID NO: 38, SEQ ID NO: 39, and SEQ ID NO: 40, respectively.
      71. The method of embodiment 70, wherein the chronic urticaria is chronic inducible urticaria.
      72. The method of embodiment 71, wherein the chronic inducible urticaria is cold urticaria.
      73. The method of embodiment 71, wherein the chronic inducible urticaria is symptomatic dermographism.
      74. The method of embodiment 71, wherein the chronic inducible urticaria is cholinergic urticaria.
      75. The method of embodiment 70, wherein the chronic urticaria is chronic spontaneous urticaria.
      76. The method of any one of embodiments 70 to 75, further comprising administering a second therapeutic agent to the subject.
      77. The method of embodiment 76, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
      78. Use of (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier, for the manufacture of a medicament for protecting against, treating or managing chronic urticaria in a subject.
      79. The use of embodiment 78, wherein the human KIT comprises the amino acid sequence of SEQ ID NO: 1.
      80. The use of embodiment 78 or 79, wherein the antibody specifically binds to a D4 or D5 region of human KIT.
      81. The use of any one of embodiments 78 to 80, wherein the antibody comprises a modified (e.g., mutated) Fe region or domain.
      82. The use of any one of embodiments 78 to 81, wherein the antibody comprises a modified (e.g., mutated) human Fe region or domain.
      83. The use of any one of embodiments 78 to 82, wherein the antibody has reduced Fc receptor binding activity (particularly reduced FcTR binding activity).
      84. The use of any one of embodiments 78 to 83, wherein the antibody does not induce significant degranulation ofFcgRI-expressing human mast cells.
      85. The use of any one of embodiments 78 to 84, wherein the antibody does not show significant Fc receptor-dependent KIT agonist activity.
      86. The use of any one of embodiments 78 to 85, wherein the antibody comprises:
(A) (i) a VL comprising VL CDR1, VL CDR2, and VL CDR3 having the amino acid sequences of SEQ ID NO: 2, SEQ ID NO: 3, and SEQ ID NO: 4, respectively; and
   (ii) a VH comprising VH CDR1, VH CDR2, and VH CDR3 having the amino acid sequences of SEQ ID NO: 5, SEQ ID NO: 6, and SEQ ID NO: 7, respectively;
(B) (i) a VL comprising VL CDR1, VL CDR2, and VL CDR3 having the amino acid sequences of SEQ ID NO: 21 SEQ ID NO: 3, and SEQ ID NO: 4, respectively; and
   (ii) a VH comprising VH CDR1, VH CDR2, and VH CDR3 having the amino acid sequences of SEQ ID NO: 25, SEQ ID NO: 26, and SEQ ID NO: 27, respectively;
(C) (i) a VL comprising VL CDR1, VL CDR2, and VL CDR3 having the amino acid sequences of SEQ ID NO: 28, SEQ ID NO: 29, and SEQ ID NO: 30, respectively; and
   (ii) a VH comprising VH CDR1, VHI CDR2, and VH CDR3 having the amino acid sequences of SEQ ID NO: 25, SEQ ID NO: 31, and SEQ ID NO: 32, respectively;
(D) (i) a VL comprising VL CDR1, VL CDR2, and VL CDR3 having the amino acid sequences of SEQ ID NO: 21 SEQ ID NO: 3, and SEQ ID NO: 4, respectively; and
   (ii) a VH comprising VH CDR1, VH CDR2, and VH CDR3 having the amino acid sequences of SEQ ID NO: 33, SEQ ID NO: 34, and SEQ ID NO: 27, respectively; or
(E) (i) a VL comprising VL CDR1, VL CDR2, and VL CDR3 having the amino acid sequences of SEQ ID NO: 35, SEQ ID NO: 36, and SEQ ID NO: 37, respectively; and
   (ii) a VH comprising VH CDR1, VH CDR2, and VH CDR3 having the amino acid sequences of SEQ ID NO: 38, SEQ ID NO: 39, and SEQ ID NO: 40, respectively.
      87. The use of embodiment 86, wherein the chronic urticaria is chronic inducible urticaria.
      88. The use of embodiment 87, wherein the chronic inducible urticaria is cold urticaria.
      89. The use of embodiment 87, wherein the chronic inducible urticaria is symptomatic dermographism.
      90. The use of embodiment 87, wherein the chronic inducible urticaria is cholinergic urticaria.
      91. The use of embodiment 86, wherein the chronic urticaria is chronic spontaneous urticaria.
      92. The use of any one of embodiments 86 to 91, wherein the medicament is manufactured to be administered in combination with a second therapeutic agent.
      93. The use of embodiment 92, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
      94. An antibody which immunospecifically binds to human KIT or an antigen binding fragment thereof, a conjugate comprising the antibody or antigen binding fragment thereof linked to an agent, or a pharmaceutical composition comprising the antibody or antigen binding fragment thereof or the conjugate, and a pharmaceutically acceptable carrier, for use in a method for protecting against, treating or managing chronic urticaria in a subject.
      95. The antibody, conjugate, or pharmaceutical composition for use of embodiment 94, wherein the human KIT comprises the amino acid sequence of SEQ ID NO: 1.
      96. The antibody, conjugate, or pharmaceutical composition for use of embodiment 94 or 95, wherein the antibody specifically binds to a D4 or D5 region of human KIT.
      97. The antibody, conjugate, or pharmaceutical composition for use of any one of embodiments 94 to 96, wherein the antibody comprises a modified (e.g., mutated) Fe region or domain.
      98. The antibody, conjugate, or pharmaceutical composition for use of any one of embodiments 94 to 97, wherein the antibody comprises a modified (e.g., mutated) human Fc region or domain.
      99. The antibody, conjugate, or pharmaceutical composition for use of any one of embodiments 94 to 98, wherein the antibody has reduced Fc receptor binding activity (particularly reduced FcTR binding activity).
      100. The antibody, conjugate, or pharmaceutical composition for use of any one of embodiments 94 to 99, wherein the antibody does not induce significant degranulation of FcgRI-expressing human mast cells.
      101. The antibody, conjugate, or pharmaceutical composition for use of any one of embodiments 94 to 100, wherein the antibody does not show significant Fe receptor-dependent KIT agonist activity.
      102. The antibody, conjugate, or pharmaceutical composition for use of any one of embodiments 94 to 101, wherein the antibody comprises:
(A) (i) a VL comprising VL CDR1, VL CDR2, and VL CDR3 having the amino acid sequences of SEQ ID NO: 21 SEQ ID NO: 3, and SEQ ID NO: 4, respectively; and
   (ii) a VI comprising VH CDR1, VH CDR2, and VH CDR3 having the amino acid sequences of SEQ ID NO: 5, SEQ ID NO: 6, and SEQ ID NO: 7, respectively;
(B) (i) a VL comprising VL CDR1, VL CDR2, and VH CDR3 having the amino acid sequences of SEQ ID NO: 2, SEQ ID NO: 3, and SEQ ID NO: 4, respectively; and
   (ii) a VH comprising VH CDR1, VH CDR2, and VH CDR3 having the amino acid sequences of SEQ ID NO: 25, SEQ ID NO: 26, and SEQ ID NO: 27, respectively;
(C) (i) a VL comprising VL CDR1, L CDR2, and VL CDR3 having the amino acid sequences of SEQ ID NO: 28, SEQ ID NO: 29, and SEQ ID NO: 30, respectively; and
   (ii) a VH comprising VH CDR1, VH CDR2, and VH CDR3 having the amino acid sequences of SEQ ID NO: 25, SEQ ID NO: 31, and SEQ ID NO: 32, respectively;
(D) (i) a VL comprising VL CDR 1, VL CDR-2, and VL CDR3 having the amino acid sequences of SEQ ID NO: 2, SEQ ID NO: 3, and SEQ ID NO: 4, respectively; and
   (ii) a VH comprising VH CDR1, VH CDR2, and VH CDR3 having the amino acid sequences of SEQ ID NO: 33, SEQ ID NO: 34, and SEQ ID NO: 27, respectively; or
(E) (i) a VL comprising VL CDR1, VL CDR2, and VL CDR3 having the amino acid sequences of SEQ ID NO: 35, SEQ ID NO: 36, and SEQ ID NO: 37, respectively; and
   (ii) a VH comprising VH CDR1, VH CDR2, and VH CDR3 having the amino acid sequences of SEQ ID NO: 38, SEQ ID NO: 39, and SEQ ID NO: 40, respectively.
      103. The antibody, conjugate, or pharmaceutical composition for use of embodiment 102, wherein the chronic urticaria is chronic inducible urticaria.
      104. The antibody, conjugate, or pharmaceutical composition for use of embodiment 103, wherein the chronic inducible urticaria is cold urticaria.
      105. The antibody, conjugate, or pharmaceutical composition for use of embodiment 103, wherein the chronic inducible urticaria is symptomatic dermographism.
      106. The antibody, conjugate, or pharmaceutical composition for use of embodiment 103, wherein the chronic inducible urticaria is cholinergic urticaria.
      107. The antibody, conjugate, or pharmaceutical composition for use of embodiment 102, wherein the chronic urticaria is chronic spontaneous urticaria.
      108. The antibody, conjugate, or pharmaceutical composition for use of any one of embodiments 102 to 107, wherein the method further comprises administering a second therapeutic agent to the subject.
      109. The antibody, conjugate, or pharmaceutical composition for use of embodiment 108, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

### 4. BRIEF DESCRIPTION OF THE FIGURES

FIG. 1 depicts the amino acid sequence of full length human KIT (SEQ ID NO: 1), GenBank™ accession number AAC50969. The first through fifth extracellular Ig-like domains (i.e., D1, D2, D3, D4, and D5) are indicated; “{” depicts the amino-terminal residue of each domain and “}” depicts the carboxyl-terminal residue of each domain. The D1 domain is depicted at P34 to R112, the D2 domain is depicted at D113 to P206, the D3 domain is depicted at A207 to D309, the D4 domain is depicted at K310 to N410, the hinge region between D4 and D5 is located at V409 to N410, and the D5 domain is depicted at T411 to K509. Also, the D1/D2 hinge region is located at D113 to L117; the D2/D3 hinge region is located at P206 to A210; and the D3/D4 hinge region is located at D309 to G311. The D4/D5 region comprises K310 to K509. The transmembrane domain comprises residues F525 to Q545, and the kinase domain comprises residues K589 to S933.

FIGS. 2A-2E depict effects of a particular anti-KIT antibody according to the present invention, mAb1, on plasma tryptase levels.

FIGS. 3A and 3B depict further effects of a particular anti-KIT antibody according to the present invention, mAb1, on plasma tryptase levels.

FIG. 4 depicts effects of a particular anti-KIT antibody according to the present invention, mAb1, on plasma Stem Cell Factor (SCF) levels.

FIG. 5 depicts effects of a particular anti-KIT antibody according to the present invention, mAb1, and a corresponding antibody with the same variable region sequences but an unmutated (wild type) human IgG1 sequence, mAbc, on SCF-induced activation of wild-type KIT and downstream intracellular signaling pathways.

FIG. 6 depicts effects of a particular anti-KIT antibody according to the present invention, mAb1, and a corresponding antibody with the same variable region sequences but an unmutated (wild type) human IgG1 sequence, mAbc, on SCF-dependent cell proliferation.

FIG. 7 shows the binding affinities of a particular anti-KIT antibody according to the present invention, mAb1, and a corresponding antibody with the same variable region sequences but an unmutated (wild type) human IgG1 sequence, mAbc, for recombinant human Fc-gamma receptors (FcTRs) and human neonatal Fc Receptor (FcRn).

FIGS. 8A-8N depict the binding curves of a particular anti-KIT antibody according to the present invention, mAb1, and a corresponding antibody with the same variable region sequences but an unmutated (wild type) human IgG1 sequence, mAbc, for recombinant human Fc-gamma receptors (FcTRs) and human neonatal Fc Receptor (FcRn). FIG. 8A depicts the binding curves of mAb1 for FcγRI. FIG. 8B depicts the binding curves of mAb1 for FcγRIIa. FIG. 8C depicts the binding curves of mAb1 for FcγRIIb. FIG. 8D depicts the binding curves of mAb1 for FcγRIIIa. FIG. 8E depicts the binding curves of mAb1 for FcγRIIIb. FIG. 8F depicts the binding curves of mAb1 for FcRn (pH 6.0). FIG. 8G depicts the binding curves of mAb1 for FcRn (pH 7.2). FIG. 8H depicts the binding curves of mAbc for FcγRI. FIG. 8I depicts the binding curves of mAbc for FcγRIIa. FIG. 8J depicts the binding curves of mAbc for FcγRIIb. FIG. 8K depicts the binding curves of mAbc for FcγRIIIa. FIG. 8L depicts the binding curves of mAbc for FcγRIIIb. FIG. 8M depicts the binding curves of mAbc for FcRn (pH 6.0). FIG. 8N depicts the binding curves of mAbc for FcRn (pH 7.2).

FIG. 9 depicts effects of a particular anti-KIT antibody according to the present invention, mAb1, and a corresponding antibody with the same variable region sequences but an unmutated (wild type) human IgG1 sequence, mAbc, on antibody-dependent cellular cytotoxicity (ADCC) activity.

FIG. 10 depicts effects of a particular anti-KIT antibody according to the present invention, mAb1, on specific cytokine production. The conditions shown for each bar graph are, from left to right: PHA, LPS, huIgG1 (soluble), mAb1 0.02 nM (soluble), mAb1 0.2 nM (soluble), mAb1 40 nM (soluble), mAb1 0.02 nM (dry coated), mAb1 0.2 nM (dry coated), and mAb1 40 nM (dry coated).

FIG. 11 depicts a schematic illustrating the roles of KIT signaling in mast cells and the action of mAb1 on the KIT receptor.

FIGS. 12A-12D show that a single dose of mAb1 resulted in a rapid and durable response with a 95% complete response (CR) rate in patients with chronic inducible urticaria (CIndU). 10/10 cold urticaria (ColdU) patients achieved CR (FIG. 12A). 8/9 symptomatic dermographism (SD) patients achieved CR and 1/9 SD patients achieved partial response (PR) (FIG. 12B). CR=negative provocation test at ≤4° C. or 0 pins; PR=improvement by 4° C. or ≥2 pins; maximum response for each patient is shown. TempTest® results over time in ColdU patients are shown in FIG. 12C. Among completed ColdU patients (n=8), CR was sustained for a median duration of 77 days (FIG. 12C). FricTest® results over time in SD patients are shown in FIG. 12D. Among completed SD patients (n=6), CR was sustained for a median duration of 57 days (FIG. 12D).

FIGS. 13A-13B show an overall disease improvement as evidenced by physician's global assessment (Phys-GA) and patient's global assessment (Pat-GA). Phys-GA and Pat-GA assess disease severity using a Likert scale of 0-3, where 0 is none and 3 is severe.

FIGS. 14A-14D show that mAb1 treatment markedly depleted skin mast cells and serum tryptase. FIG. 14A shows that mAb1 reduced skin mast cell number (n=14, * means p<0.05, ** means p<0.01, * ** means p<0.001, and **** means p<0.0001). FIG. 14B shows that mAb1 reduced serum tryptase below detection in all patients (tryptase values below assay limit of quantitation (LLoQ=1 ng/mL) was normalized to 0). FIG. 14C shows the mast cell and tryptase kinetics. FIG. 14D shows that skin mast cell numbers correlated with serum tryptase levels (p<0.0001; R²=0.45)).

FIGS. 15A-15D show that the kinetics for skin mast cell and tryptase depletion mirrored decreases in provocation thresholds. FIG. 15A shows the mast cell kinetics and the TempTest® results over time in ColdU patients. FIG. 15B shows the mast cell kinetics and the FricTest® results over time in SD patients. FIG. 15C shows the tryptase kinetics and the TempTest® results over time in ColdU patients (tryptase values below LLoQ were normalized to 0; critical temperature threshold values below 4° C. (negative test) was assigned a value of 3° C.). FIG. 15D shows the tryptase kinetics and the FricTest® results over time in SD patients.

FIGS. 16A-16D show that hematology parameters generally remained within the normal ranges and that mild, transient, and asymptomatic decreases in hemoglobin and white blood cell (WBC) parameters were noted. FIG. 16A shows the level of hemoglobin (HgB) over time. FIG. 16B shows the WBC count over time. FIG. 16C shows the platelet count over time. FIG. 16D shows the absolute neutrophil count (ANC) over time. In each graph, shaded area represents the corresponding normal range.

FIGS. 17A-17B show that a single 3 mg/kg dose of mAb1 resulted in rapid and sustained improvement in urticaria control in cold urticaria (ColdU) patients (n=10, see FIG. 17A) and symptomatic dermographism (SD) patients (n=10, see FIG. 17B). An urticaria control test (UCT) score=16 means complete control of urticaria, a UCT score≥12 means well controlled status of urticaria, and a UCT score<12 means poorly controlled status of urticaria. In each graph, mean UCT scores±SEM are displayed.

FIGS. 18A-18B show that a single 3 mg/kg dose of mAb1 resulted in rapid and sustained improvement in urticaria control in ColdU and SD patients. FIG. 18A shows that 100% patients achieved “well controlled” status (UCT score≥12) by week 8. FIG. 18B shows that 63% patients achieved “complete control” status (UCT score=16) by week 8.

FIGS. 19A-19B show that mAb1 greatly reduced disease impact on the quality of life of patients with cold urticaria (ColdU, n=10, see FIG. 19A) and symptomatic dermographism (SD, n=10, see FIG. 19B). Mean DLQI scores±SEM are displayed.

FIGS. 20A-20B show that mAbIgreatly reduced disease impact on the quality of life of patients with cold urticaria (ColdU) and symptomatic dermographism (SD). FIG. 20A shows that 93% patients achieved clinically significant improvement in quality of life by week 4. †: a reduction of DLQI≥4 point is minimal clinically important difference (MCID). *: only patients whose baseline DLQI scores were ≥4 were included. FIG. 20B shows that 58% patients reported no disease impact on quality of life by week 4. †̅: all responses provided for each week were included.

FIGS. 21A-21B show that a single 3 mg/kg dose of mAb1 resulted in rapid and durable improvement in provocation tests with a 95% complete response (CR) and profound tryptase reduction. FIG. 21 A shows that mAb1 resulted in rapid and durable improvement in provocation tests with a 95% complete response. Disease activity was assessed by critical temperature threshold (CTT) per TempTest® for cold urticaria (ColdU) and critical friction threshold (CFT) per FricTest® for symptomatic dermographism (SD). *: Critical temperature threshold values below 4° C. (negative test) were assigned a value of 3° C. 10/10 ColdU and 9/10 SD patients experienced CR on study. CR=negative provocation test at ≤4° C. (for ColdU) or 0 pins (for SD). In the graph, mean values±SEM are displayed. FIG. 21B shows that mAb1 resulted in rapid, durable and profound tryptase reduction. Tryptase values below lower limit of quantitation (1 ng/mL) were normalized to 0. In the graph, mean values±SEM are displayed.

### 5. DETAILED DESCRIPTION

Provided herein are antibodies, and antigen binding fragments thereof, that immunospecifically bind to human KIT (e.g., a KIT polypeptide containing a human KIT D4 or D5 domain), and conjugates thereof. In preferred embodiments, provided herein are antibodies that immunospecifically bind to human KIT, in particular antibodies having reduced Fc receptor binding activity (particularly reduced FcγR binding activity) and improved pharmacokinetics, reduced degranulation of FcgRI-expressing human mast cells and/or reduced Fc receptor-dependent KIT agonist activity. In certain embodiments, provided herein are antigen binding fragments that immunospecifically bind to human KIT. Also provided are isolated nucleic acids (polynucleotides) encoding such antibodies or antigen-binding fragments thereof. Further provided are vectors (e.g., expression vectors) and cells (e.g., host cells) comprising nucleic acids (polynucleotides) encoding such antibodies or antigen-binding fragments thereof. Also provided are methods of making such antibodies, cells, e.g., host cells. Also provided herein are methods and uses for protecting against, treating or managing a KIT-associated disorder or disease comprising administering an antibody described herein, or an antigen-binding fragment thereof or a conjugate thereof. Also provided herein are methods for diagnosing a KIT-associated disorder or disease comprising contacting a sample with an antibody or antigen binding fragment thereof described herein and determining the expression level of KIT in the sample relative to a reference sample (e.g., a control sample). Further provided herein are methods and uses for inhibiting KIT activity in a cell expressing KIT comprising contacting the cell with an effective amount of an antibody described herein or an antigen-binding fragment thereof. Also further provided herein are methods for inducing or enhancing cell differentiation or apoptosis in a cell expressing KIT comprising contacting the cell with an effective amount of an antibody described herein or an antigen-binding fragment thereof.

As used herein, “administer” or “administration” refers to the act of injecting or otherwise physically delivering a substance (e.g., a humanized anti-KIT antibody provided herein or an antigen-binding fragment thereof) to a subject or a patient (e.g., human), such as by mucosal, topical, intradermal, parenteral, intravenous, intramuscular delivery and/or any other method of physical delivery described herein or known in the art.

As used herein, the terms “effective amount” or “therapeutically effective amount” refer to an amount of a therapy (e.g., an antibody or pharmaceutical composition provided herein) which is sufficient to reduce and/or ameliorate the severity and/or duration of a given disease and/or a symptom related thereto. These terms also encompass an amount necessary for the reduction, slowing, or amelioration of the advancement or progression of a given disease, reduction, slowing, or amelioration of the recurrence, development or onset of a given disease, and/or to improve or enhance the prophylactic or therapeutic effect(s) of another therapy (e.g., a therapy other than an anti-KIT antibody provided herein). In some embodiments, “effective amount” as used herein also refers to the amount of an antibody described herein to achieve a specified result, for example, reduction in the number and/or activity of mast cells, reduction in the number and/or activity of eosinophils, inhibition (e.g., partial inhibition) of a KIT biological activity of a cell, such as inhibition of cell proliferation or cell survival, or enhancement or induction of apoptosis or cell differentiation, and the like.

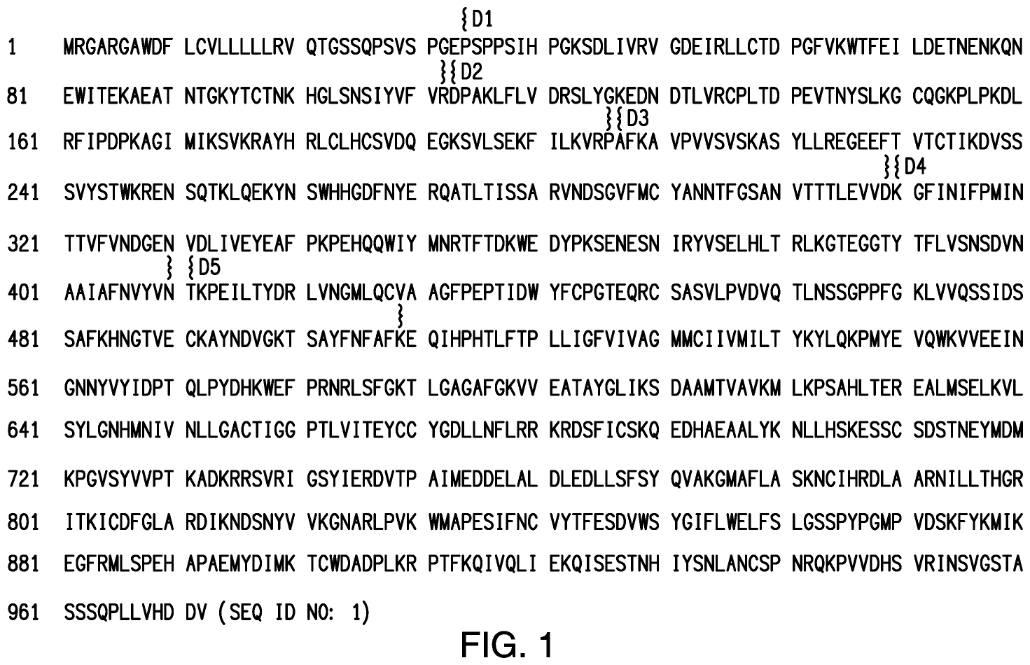

As used herein, the terms “D4 or D5 region” or “D4/D5 domain” refer to a region within a KIT polypeptide spanning the fourth Ig-like extracellular (“D4”) domain, the fifth Ig-like extracellular (“D5”) domain, and the hinge region in between the D4 and D5 domains (“D4-D5 hinge region”), of KIT, in the following order from the amino terminus to the carboxyl terminus: D4, D4-D5 hinge region, and D5. As used herein, amino acids V308 to H515 of FIG. 1 are considered an example of a D4/D5 region or domain.

As used herein, the terms “KIT” or “KIT receptor” or “KIT polypeptide” refer to any form of full-length KIT including, but not limited to, native KIT, an isoform of KIT, an interspecies KIT homolog, or a KIT variant, e.g., naturally occurring (for example, allelic or splice variant, or mutant, e.g., somatic mutant) or artificially constructed variant (for example, a recombinant or chemically modified variant). KIT is a type III receptor tyrosine kinase encoded by the c-kit gene (see, e.g., Yarden et ah, Nature, 1986, 323:226-232; Ullrich and Schlessinger, Cell, 1990, 61:203-212; Clifford et al., J. Biol. Chem., 2003, 278:31461-31464; Yarden et al., EMBO J., 1987, 6:3341-3351; Mol et al., J. Biol. Chem., 2003, 278:31461-31464). GenBank™ accession number NM 000222 provides an exemplary human KIT nucleic acid sequence. GenBank™ accession numbers NP 001087241, PI 0721, and AAC50969 provide exemplary human KIT amino acid sequences. GenBank™ accession number AAH75716 provides an exemplary murine KIT amino acid sequence. Native KIT comprises five extracellular immunoglobulin (Ig)-like domains (D1, D2, D3, D4, D5), a single transmembrane region, an inhibitory cytoplasmic juxtamembrane domain, and a split cytoplasmic kinase domain separated by a kinase insert segment (see, e.g., Yarden et al., Nature, 1986, 323:226-232; Ullrich and Schlessinger, Cell, 1990, 61:203-212; Clifford et al., J. Biol. Chem., 2003, 278:31461-31464). An exemplary amino acid sequence of the D4/D5 region of human KIT is provided in FIG. 1, at amino acid residues V308 to H515. In a specific embodiment, KIT is human KIT. In a particular embodiment, KIT can exist as a monomer, dimer, multimer, native form, or denatured form.

As used herein, the term “in combination” in the context of the administration of other therapies refers to the use of more than one therapy. The use of the term “in combination” does not restrict the order in which therapies are administered. The therapies may be administered, e.g., serially, sequentially, concurrently, or concomitantly.

As used herein, the terms “KIT-associated disorder” or “KIT-associated disease” are used interchangeably and refer to any disease that is completely or partially caused by, associated with, or is the result of, KIT expression and/or activity or lack thereof. In one aspect, a KIT-associated disorder or disease can be known to one of skill in the art or can be ascertained by one of skill in the art. In a certain embodiment, a KIT-associated disease or disorder is associated with KIT expression and/or activity. For example, KIT expression and/or activity may contribute, in combination with one or more other factors (e.g., mutation or expression and/or activity of another gene), to development and/or progression of a KIT-associated disease or disorder. In a certain embodiment, a KIT-associated disease or disorder is associated with one or more mutations of KIT.

In certain embodiments, a KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis. In certain embodiments, a KIT-associated disorder is fibrosis or an inflammatory disorder, e.g., inflammatory bowel disease (IBD), such as Crohn's disease (CD) or ulcerative colitis (UC). In other embodiments, a KIT-associated disease is cancer, such as lung cancer (e.g., small cell lung cancer), leukemia, neuroblastoma, melanoma, sarcoma (e.g., Ewing's sarcoma) or gastrointestinal stromal tumor (GIST). In a specific embodiment, the KIT-associated disorder is a mast cell related disorder. In a specific embodiment, the KIT-associated disorder is an eosinophil related disorder such as eosinophilic esophagitis (EoE).

As used herein, the terms “treat,” “treatment” and “treating” refer to the reduction or amelioration of the progression, severity, and/or duration of a KIT-associated disease (e.g., cancer, inflammatory disorder, or fibrosis) resulting from the administration of one or more therapies (including, but not limited to, the administration of one or more prophylactic or therapeutic agents, such as an antibody provided herein).

As used herein, the terms “manage”, “managing,” and “management” refer to the beneficial effects that a subject derives from a therapy (e.g., a prophylactic or therapeutic agent), which does not result in a cure. In certain embodiments, a subject is administered one or more therapies (e.g., prophylactic or therapeutic agents, such as an antibody described herein) to “manage” a disorder, or one or more symptoms thereof, so as to prevent the progression or worsening of the disorder.

As used herein, the terms “protect against,” “impede,” or “impeding” in the context of a disorder, refer to the total or partial inhibition (e.g., less than 100%, 95%, 90%, 80%, 70%, 60%, 50%, 40%, 30%, 20%, 10%, or 5%) or blockage of the development, recurrence, onset or spread of the disorder, and/or symptom related thereto, resulting from the administration of a therapy or combination of therapies provided herein (e.g., a combination of prophylactic or therapeutic agents, such as an antibody described herein).

As used herein, the term “prophylactic agent” refers to any agent that can totally or partially inhibit the development, recurrence, onset or spread of a disorder, and/or symptom related thereto in a subject. In certain embodiments, the term “prophylactic agent” refers to an antibody described herein. In certain other embodiments, the term “prophylactic agent” refers to an agent other than an antibody described herein. Generally, a prophylactic agent is an agent which is known to be useful to or has been or is currently being used to prevent the disorder, and/or a symptom related thereto or impede the onset, development, progression and/or severity of the disorder, and/or a symptom related thereto. In specific embodiments, the prophylactic agent is a human anti-KIT antibody, such as a humanized or a fully human anti-KIT monoclonal antibody.

As used herein, the term “side effects” or “adverse effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) can be harmful or uncomfortable or risky. Examples of side effects include, diarrhea, cough, gastroenteritis, wheezing, nausea, vomiting, anorexia, abdominal cramping, fever, pain, loss of body weight, dehydration, alopecia, dyspnea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (71^{st} ed., 2017).

As used herein, the terms “subject” and “patient” are used interchangeably. As used herein, a subject is a mammal such as a non-primate (e.g., cows, pigs, horses, cats, dogs, goats, rabbits, rats, mice, etc.) or a primate (e.g., monkey and human), for example a human. In one embodiment, the subject is a mammal, e.g., a human, diagnosed with a disorder. In another embodiment, the subject is a mammal, e.g., a human, at risk of developing a KIT-associated disorder. In another embodiment, the subject is a non-human primate. In a specific embodiment, the subject is a human adult. In a specific embodiment, the subject is an adult human subject at least 18 years old. In a specific embodiment, the subject is a human child. In a specific embodiment, the subject is a human child between 1 year old to 18 years old. In a specific embodiment, the subject is a human between 1 year to 3 years old. In a specific embodiment, the subject is a human between 3 years to 12 years old or between 12 years to 18 years old.

As used herein, the terms “therapies” and “therapy” can refer to any protocol(s), method(s), compositions, formulations, and/or agent(s) that can be used in the prevention, protection against, treatment, management, or amelioration of a condition or disorder or symptom thereof or one or more symptoms or condition associated therewith. In certain embodiments, the terms “therapies” and “therapy” refer to drug therapy, adjuvant therapy, radiation, surgery, biological therapy, supportive therapy, and/or other therapies useful in protection against, treatment, management, prevention, or amelioration of a condition or disorder or one or more symptoms thereof or one or more symptoms or condition associated therewith. In certain embodiments, the term “therapy” refers to a therapy other than an anti-KIT antibody described herein or pharmaceutical composition thereof. In specific embodiments, an “additional therapy” and “additional therapies” refer to a therapy other than a treatment using an anti-KIT antibody described herein or pharmaceutical composition thereof. In a specific embodiment, a therapy includes the use of an anti-KIT antibody described herein as an adjuvant therapy. For example, using an anti-KIT antibody described herein in conjunction with a drug therapy, biological therapy, surgery, and/or supportive therapy.

As used herein, the term “therapeutic agent” refers to any agent that can be used in the protection against, treatment, management or amelioration of a disorder and/or a symptom related thereto. In certain embodiments, the term “therapeutic agent” refers to an anti-KIT antibody described herein or an antigen-binding fragment thereof. In certain other embodiments, the term “therapeutic agent” refers to an agent other than an antibody described herein. In specific embodiments, a therapeutic agent is an agent which is known to be useful for, or has been or is currently being used for the protection against, treatment, management or amelioration of a disorder or one or more symptoms related thereto.

As used in this specification and the appended claims, the singular forms “a”, “an” and “the” include plural referents unless the context clearly dictates otherwise. The terms “a” (or “an”), as well as the terms “one or more,” and “at least one” can be used interchangeably herein.

It is understood that wherever aspects are described herein with the language “comprising,” otherwise analogous aspects described in terms of “consisting of” and/or “consisting essentially of” are also provided.

As used herein and unless otherwise specified, the terms “about” and “approximately” shall be construed so as to allow normal variation as judged by a person of skill in the art, such as, for example, a variation within 20% or 10% or 5%. In specific embodiments, the terms “about” and “approximately” encompass the exact value recited.

### 5.1 Antibodies

Provided herein are antibodies (e.g., anti-KIT antibodies) that specifically bind to a KIT receptor (e.g., extracellular domain of a human KIT receptor for example as set forth in SEQ ID NO: 1 or FIG. 1), or an antigen binding fragment thereof.

As used herein, the terms “antibody” and “immunoglobulin” and “Ig” are terms of art and can be used interchangeably herein and refer to a molecule with an antigen binding site that immunospecifically binds an antigen.

Antibodies include, for example, monoclonal antibodies, recombinantly produced antibodies, monospecific antibodies, multispecific antibodies (including bispecific antibodies), human antibodies, humanized antibodies and chimeric antibodies. In certain embodiments, antibodies described herein refer to polyclonal antibody populations. Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In certain embodiments, antibodies described herein are IgG antibodies, or a class (e.g., human IgG1 or IgG4) or subclass thereof.

As used herein, an “antigen” is a moiety or molecule that contains an epitope, and, as such, also is specifically bound by an antibody. In a specific embodiment, the antigen, to which an antibody described herein binds, is KIT (e.g., human KIT), or a fragment thereof, for example, an extracellular domain of KIT (e.g., human KIT) or a D4 region of KIT (e.g., human KIT).

As used herein, the terms “antigen binding domain,” “antigen binding region,” “antigen binding fragment,” and similar terms refer to a portion of an antibody molecule which comprises the amino acid residues that interact with an antigen and confer on the antibody molecule its specificity for the antigen (e.g., the complementarity determining regions (CDR)). The antigen binding region can be derived from any animal species, such as rodents (e.g., mouse, rat or hamster) and humans. The CDRs of an antibody molecule can be determined by any method well known to one of skill in the art. In particular, the CDRs can be determined according to the Kabat numbering system (see Kabat et al. (1991) Sequences of Proteins of Immunological Interest. (U.S. Department of Health and Human Services, Washington, D.C.) 5^{th} ed.). In certain aspects, the CDRs of an antibody can be determined according to (i) the Chothia numbering scheme, which will be referred to herein as the “Chothia CDRs” (see, e.g., Chothia and Lesk, 1987, J. Mol. Biol, 196:901-917; Al-Lazikani et al., 1997, J. Mol. Biol, 273:927-948; and U.S. Pat. No. 7,709,226); (ii) the IMGT numbering system, for example, as described in Lefranc, M.-P., 1999, The Immunologist, 7: 132-136 and Lefranc, M.-P. et al., 1999, Nucleic Acids Res., 27:209-212; (iii) the AbM numbering system, for example, as described in MacCallum et al., 1996, J. Mol. Biol., 262:732-745 and Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001); or (iv) the Contact numbering system, which is based on analysis of the available complex crystal structures (bioinf.org.uk/abs) (see, e.g., MacCallum et al., (1996) J Mol Biol 5:732-745). In preferred embodiments, the antigen binding fragment described herein comprise a full-length heavy chain Fc region or domain (e.g., a full-length human IgG1, human IgG2, human IgG3, or human IgG4 Fc region or domain) or a partial heavy chain Fc region or domain (e.g., a partial human IgG1, human IgG2, human IgG3, or human IgG4 Fc region or domain).

As used herein, the term “constant region” or “constant domain” refers to an antibody portion, e.g., a carboxyl terminal portion of a light and/or heavy chain which is not directly involved in binding of an antibody to antigen but which exhibits or contributes to various effector functions, such as interaction with the Fc receptor. The terms refer to a portion of an immunoglobulin molecule having a generally more conserved amino acid sequence relative to an immunoglobulin variable domain.

As used herein, an “epitope” is a term in the art and refers to a localized region of an antigen to which an antibody can specifically bind. A region or a polypeptide contributing to an epitope can be contiguous amino acids of the polypeptide or an epitope can come together from two or more non-contiguous regions of the polypeptide.

As used herein, the term “heavy chain” when used in reference to an antibody refers to any distinct types, e.g., alpha (a), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG₂, IgG₃ and IgG₄. In a specific embodiment, the heavy chain is a human heavy chain.

As used herein, the terms “immunospecifically binds,” “immunospecifically recognizes,” “specifically binds,” and “specifically recognizes” are analogous terms in the context of antibodies and refer to molecules that bind to an antigen (e.g., epitope or immune complex) as such binding is understood by one skilled in the art. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In a specific embodiment, molecules that immunospecifically bind to an antigen bind to the antigen with a Kₐ that is at least 2 logs, 2.5 logs, 3 logs, 4 logs or greater than the Kₐ when the molecules bind to another antigen. In another specific embodiment, molecules that immunospecifically bind to an antigen do not cross react with other proteins. In another specific embodiment, molecules that immunospecifically bind to an antigen do not cross react with other non-KIT proteins.

As used herein, an “isolated” or “purified” antibody is substantially free of cellular material or other contaminating proteins from the cell or tissue source from which the antibody is derived, or substantially free of chemical precursors or other chemicals when chemically synthesized. In a specific embodiment, the antibody or antigen binding fragment described herein is isolated.

The terms “Kabat numbering,” and like terms are recognized in the art and refer to a system of numbering amino acid residues in the heavy and light chain variable regions of an antibody, or an antigen binding portion thereof (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35 (“CDR1”), amino acid positions 50 to 65 (“CDR2”), and amino acid positions 95 to 102 (“CDR3”). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

As used herein, the term “light chain” when used in reference to an antibody refers to any distinct types, e.g., kappa (x) of lambda (k) based on the amino acid sequence of the constant domains. Light chain amino acid sequences are well known in the art. In specific embodiments, the light chain is a human light chain.

As used herein, the term “monoclonal antibody” refers to an antibody obtained from a population of homogenous or substantially homogeneous antibodies, and each monoclonal antibody will typically recognize a single epitope on the antigen. The term “monoclonal” is not limited to any particular method for making the antibody. Generally, a population of monoclonal antibodies can be generated by cells, a population of cells, or a cell line. In specific embodiments, a “monoclonal antibody,” as used herein, is an antibody produced by a single hybridoma or other cell (e.g., host cell producing a recombinant antibody), wherein the antibody immunospecifically binds to a KIT epitope (e.g., an epitope of a D4 of human KIT) as determined, e.g., by ELISA or other antigen-binding or competitive binding assay known in the art or in the Examples provided herein. Monoclonal antibodies described herein can, for example, be made by the hybridoma method as described in Kohler et ah; Nature, 256:495 (1975) or can be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel et ah, eds., John Wiley and Sons, New York). In specific embodiments, a monoclonal antibody is a monospecific antibody in that its antigen binding regions are specific for the same epitope. In further specific embodiments, a monoclonal monospecific antibody can be monovalent (having one antigen binding region) or multivalent (having more than one antigen binding regions), for example, bivalent (having two antigen binding regions).

As used herein, the term “naked antibody” refers to an antibody which is not linked, fused or conjugated to another agent or molecule (e.g., label or drug), peptide or polypeptide. In specific embodiments, a naked antibody expressed by a mammalian host cell can be glycosylated by the host cell's glycosylation machinery, for example glycosylation enzymes. In certain embodiment, a naked antibody is not glycosylated when it is expressed by a host cell which does not have its own glycosylation machinery, for example glycosylation enzymes. In certain embodiments, a naked antibody is a whole antibody, and in other embodiments, a naked antibody is an antigen binding fragment of a whole antibody, such as a Fab antibody.

As used herein, the term “polyclonal antibodies” refers to an antibody population that includes a variety of different antibodies directed to the same and to different epitopes within an antigen or antigens. Methods for producing polyclonal antibodies are known in the art (See, e.g., see, for example, Chapter 11 in: Short Protocols in Molecular Biology, (2002) 5th Ed., Ausubel et ah, eds., John Wiley and Sons, New York).

As used herein, the term “recombinant human antibody” includes human antibodies that are isolated, prepared, expressed, or created by recombinant means, such as antibodies expressed using a recombinant expression vector transfected into a host cell, antibodies isolated from a recombinant, combinatorial human antibody library, antibodies isolated from an animal (e.g., a mouse, rabbit, goat, or cow) that is transgenic and/or transchromosomal for human immunoglobulin genes (see e.g., Taylor, L. D. et al. (1992) Nucl. Acids Res. 20:6287-6295) or antibodies prepared, expressed, created or isolated by any other means that involves creation, e.g., via synthesis, genetic engineering of DNA sequences that encode human immunoglobulin sequences, or splicing of sequences that encode human immunoglobulins, e.g., human immunoglobulin gene sequences, to other such sequences. Such recombinant human antibodies can have variable and constant regions derived from human germline immunoglobulin sequences. In certain embodiments, the amino acid sequences of such recombinant human antibodies have been modified such thus the amino acid sequences of the VH and/or VL regions of the recombinant antibodies are sequences that, while derived from and related to human germline VH and VL sequences, do not naturally exist within the human antibody germline repertoire in vivo. As a non-limiting example, a recombinant human antibody can be obtained by assembling several human sequence fragments into a composite human sequence of a recombinant human antibody.

As used herein, the terms “variable region” or “variable domain” refer to a portion of an antibody, generally, a portion of a light or heavy chain, typically about the amino-terminal 110 to 120 amino acids in the mature heavy chain and about 90 to 100 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction of the antibody with antigen. In a specific embodiment, numbering of amino acid positions of antibodies described herein is according to the EU Index, as in Kabat et al. (1991) Sequences of Proteins of Immunological Interest, Fifth Edition, U.S. Department of Health and Human Services, NIH Publication No. 91-3242 (“Kabat et al.”). In certain aspects, the CDRs of an antibody can be determined according to (i) the Chothia numbering scheme, which will be referred to herein as the “Chothia CDRs” (see, e.g., Chothia and Lesk, 1987, J. Mol. Biol, 196:901-917; Al-Lazikani et al., 1997, J. Mol. Biol, 273:927-948; and U.S. Pat. No. 7,709,226); (ii) the IMGT numbering system, for example, as described in Lefranc, M.-P., 1999, The Immunologist, 7:132-136 and Lefranc, M.-P. et al., 1999, Nucleic Acids Res., 27:209-212; (iii) the AbM numbering system, for example, as described in MacCallum et al., 1996, J. Mol. Biol., 262:732-745 and Martin, A., “Protein Sequence and Structure Analysis of Antibody Variable Domains,” in Antibody Engineering, Kontermann and Dubel, eds., Chapter 31, pp. 422-439, Springer-Verlag, Berlin (2001); or (iv) the Contact numbering system, which is based on analysis of the available complex crystal structures (bioinf.org.uk/abs) (see, e.g., MacCallum et al., (1996) J Mol Biol 5:732-745). In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs). As a non-limiting example, a variable region described herein is obtained from assembling two or more fragments of human sequences into a composite human sequence.

In a specific aspect, anti-KIT antibodies (e.g., humanized antibodies) provided herein comprise a light chain variable region (“VL”) comprising VL CDRs 1-3 and a heavy chain variable region (“VH”) comprising VH CDRs 1-3 as set forth in Table 1. In a specific aspect, anti-KIT antibodies (e.g., humanized antibodies) provided herein comprise a light chain variable region (“VL”) comprising VL CDRs 1-3 and a heavy chain variable region (“VH”) comprising VH CDRs 1-3 as set forth in Table 2 (set 1 or set 2). In a specific aspect, anti-KIT antibodies (e.g., humanized antibodies) provided herein comprise a light chain variable region (“VL”) comprising VL CDRs 1-3 and a heavy chain variable region (“VH”) comprising VH CDRs 1-3 as set forth in Table 3 (AbM CDRs or Contact CDRs).

In a specific aspect, anti-KIT antibodies (e.g., humanized antibodies) provided herein comprise a VL comprising VL CDRs 1-3 as set forth in Table 1 (SEQ ID NOs: 2-4) and a VH comprising VH CDRs 1-3 as set forth in Table 1 (SEQ ID NOs: 5-7). In a particular embodiment, such anti-KIT antibody is a naked antibody. In a specific embodiment, such anti-KIT antibody is a bivalent monospecific antibody. In a specific embodiment, such anti-KIT antibody is a bispecific antibody. In a certain embodiment, such anti-KIT antibody is not a bispecific antibody.

TABLE 1
CDR Amino Acid Sequences

| | amino acid sequence | SEQ ID NO: |
|---|---|---|
| VL CDR1 | KASQNVRTNVA | 2 |
| VL CDR2 | SASYRYS | 3 |
| VL CDR3 | QQYNSYPRT | 4 |
| VH CDR1 | DYYIN | 5 |
| VH CDR2 | RIYPGSGNTYYNEKFKG | 6 |
| VH CDR3 | GVYYFDY | 7 |

TABLE 2
CDR Amino Acid Sequences

| | Set 1 | | Set 2 | |
|---|---|---|---|---|
| | amino acid | SEQ ID | amino acid | SEQ ID |
| | sequence | NO: | sequence | NO: |
| VL CDR1 | KASQNVRTNVA | 2 | SQNVRTN | 28 |
| VL CDR2 | SASYRYS | 3 | SAS | 29 |
| VL CDR3 | QQYNSYPRT | 4 | YNSYPR | 30 |
| VH CDR1 | GYTFTDY | 25 | GYTFTDY | 25 |
| VH CDR2 | YPGSGN | 26 | PGSG | 31 |
| VH CDR3 | GVYYFDYW | 27 | VYYFDY | 32 |

TABLE 3
CDR Amino Acid Sequences

| | AbM | | Contact | |
|---|---|---|---|---|
| | amino acid | SEQ ID | amino acid | SEQ ID |
| | sequence | NO: | sequence | NO: |
| VL CDR1 | KASQNVRTNVA | 2 | RTNVAWY | 35 |
| VL CDR2 | SASYRYS | 3 | ALIYSASYRY | 36 |
| VL CDR3 | QQYNSYPRT | 4 | QQYNSYPR | 37 |
| VH CDR1 | GYTFTDYYIN | 33 | TDYYIN | 38 |
| VH CDR2 | RIYPGSGNTY | 34 | WIARIYPGSGNTY | 39 |
| VH CDR3 | GVYYFDYW | 27 | ARGVYYFDY | 40 |

In a particular aspect, an anti-KIT antibody (e.g., humanized antibody) provided herein comprises:
(i) a VL comprising the amino acid sequence:

(SEQ ID NO: 17)
DIVMTQSPSX_{K1}LSASVGDRVTITCKASQN
VRTNVAWYQQKPGKAPKX_{K2}LIYSASYRYS
GVPDRFX_{K3}GSGSGTDFTLTISSLQX_{K4}ED
FAX_{K5}YX_{K6}CQQYNSYPRTFGGGTKVEIK,
wherein X_{K1} to X_{K6} is any amino acid;

and
(ii) a VH comprising the amino acid sequence:

(SEQ ID NO: 18)
QVQLVQSGAEX_{H1}KKPGASVKX_{H2}SCKASGYT
FTDYYINWVX_{H3}QAPGKGLEWIARIYPGSGNT
YYNEKFKGRX_{H4}TX_{H5}TAX_{H6}KSTSTAYMX_{H7}
LSSLRSEDX_{H8}AVYFCARGVYYFDYWGQGTT
VTVSS,
wherein X_{H1} to X_{H8} is any amino acid.

In a particular embodiment, Xxi is an amino acid with an aromatic or aliphatic hydroxyl side chain, X_{K2} is an amino acid with an aliphatic or aliphatic hydroxyl side chain X_{K3} is an amino acid with an aliphatic hydroxyl side chain X_{K4} is an amino acid with an aliphatic hydroxyl side chain or is P, X_{K5} is an amino acid with a charged or acidic side chain, X_{K6} is an amino acid with an aromatic side chain, X_{H1} is an amino acid with an aliphatic side chain, X_{H2} is an amino acid with an aliphatic side chain X_{H3} is an amino acid with a polar or basic side chain X_{H4} is an amino acid with an aliphatic side chain X_{H5} is an amino acid with an aliphatic side chain X_{H6} is an amino acid with an acidic side chain, X_{H7} is an amino acid with an acidic or amide derivative side chain, and X_{H8} is an amino acid with an aliphatic hydroxyl side chain.

In a specific embodiment, X_{K1} is the amino acid F or S, X_{K2} is the amino acid A or S, X_{K3} is the amino acid T or S, X_{K4} is the amino acid S or P, X_{K5} is the amino acid D or T X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In a particular aspect, an anti-KIT antibody (e.g., humanized antibody) provided herein comprises:
(i) a VL comprising the amino acid sequence:

(SEQ ID NO: 17)
DIVMTQSPSX_{K1}LSASVGDRVTITCKASQNVR
TNVAWYQQKPGKAPKX_{K2}LIYSASYRYSGVPD
RFX_{K3}GSGSGTDFTLTISSLQX_{K4}EDFAX_{K5}Y
X_{K6}CQQYNSYPRTFGGGTKVEIK,
wherein X_{K1} to X_{K6} is any amino acid;

and
(ii) a VH comprising a VH CDR1, VH CDR2, and VH CDR3 comprising the amino acid sequences of SEQ ID NO: 5, SEQ ID NO: 6, and SEQ ID NO: 7, respectively.

In a particular embodiment, Xxi is an amino acid with an aromatic or aliphatic hydroxyl side chain, X_{K2} is an amino acid with an aliphatic or aliphatic hydroxyl side chain X_{K3} is an amino acid with an aliphatic hydroxyl side chain X_{K4} is an amino acid with an aliphatic hydroxyl side chain or is P, X_{K5} is an amino acid with a charged or acidic side chain, and X_{K6} is an amino acid with an aromatic side chain.

In a specific embodiment, X_{K1} is the amino acid F or S, X_{K2} is the amino acid A or S, X_{K3} is the amino acid T or S, X_{K4} is the amino acid S or P, X_{K5} is the amino acid D or T, and X_{K6} is the amino acid F or Y.

In a particular aspect, an anti-KIT antibody (e.g., humanized antibody) provided herein comprises:
(i) a VL comprising a VL CDR1, VL CDR2, and VL CDR3 having the amino acid sequences of SEQ ID NO: 2, SEQ ID NO: 3, and SEQ ID NO: 4, respectively; and
(ii) a VH comprising the amino acid sequence:

(SEQ ID NO: 18)
QVQLVQSGAEX_{H1}KKPGASVKX_{H2}SCKASGYT
FTDYYINWVX_{H3}QAPGKGLEWIARIYPGSGNT
YYNEKFKGRX_{H4}TXHSTAX_{H6}KSTSTAYMX_{H7}
LSSLRSEDX_{H8}AVYFCARGVYYFDYWGQGTTV
TVSS,
wherein X_{H1} to X_{H8} is any amino acid.

In a particular embodiment, X_{H1} is an amino acid with an aliphatic side chain, X_{H2} is an amino acid with an aliphatic side chain X_{H3} is an amino acid with a polar or basic side chain X_{H4} is an amino acid with an aliphatic side chain X_{H5} is an amino acid with an aliphatic side chain X_{H6} is an amino acid with an acidic side chain, X_{H7} is an amino acid with an acidic or amide derivative side chain, and X_{H8} is an amino acid with an aliphatic hydroxyl side chain.

In a specific embodiment, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

In a specific aspect, anti-KIT antibodies (e.g., humanized antibodies) provided herein comprise a heavy chain variable region (“VH”) comprising an amino acid sequence selected from Table 4 (SEQ ID NOs: 8-12) and/or a light chain variable region (“VL”) comprising an amino acid sequence selected from Table 5 (SEQ ID NOs: 13-16). In a particular embodiment, such anti-KIT antibody is a naked antibody. In a specific embodiment, such anti-KIT antibody is a bivalent monospecific antibody. In a specific embodiment, such anti-KIT antibody is a bispecific antibody. In a certain embodiment, such anti-KIT antibody is not a bispecific antibody.

TABLE 4
VH amino acid sequence

| | Amino Acid Sequence | SEQ ID NO: |
|---|---|---|
| VH1 | QVQLVQSGAELKKPGASVKLSCKASGYTF | 8 |
| | TDYYINWVKQAPGKGLEWIARIYPGSGNTY | |
| | YNEKFKGRATLTAEKSTSTAYMQLSSLRSE | |
| | DSAVYFCARGVYYFDYWGQGTTVTVSS | |
| VH2 | QVQLVQSGAEVKKPGASVKLSCKASGYTF | 9 |
| | TDYYINWVKQAPGKGLEWIARIYPGSGNTY | |
| | YNEKFKGRATLTAEKSTSTAYMQLSSLRSE | |
| | DTAVYFCARGVYYFDYWGQGTTVTVSS | |
| VH3 | QVQLVQSGAEVKKPGASVKLSCKASGYTF | 10 |
| | TDYYINWVRQAPGKGLEWIARIYPGSGNTY | |
| | YNEKFKGRATLTADKSTSTAYMQLSSLRSE | |
| | DTAVYFCARGVYYFDYWGQGTTVTVSS | |
| VH4 | QVQLVQSGAEVKKPGASVKVSCKASGYTF | 11 |
| | TDYYINWVRQAPGKGLEWIARIYPGSGNTY | |
| | YNEKFKGRATITADKSTSTAYMELSSLRSE | |
| | DTAVYFCARGVYYFDYWGQGTTVTVSS | |
| VH5 | QVQLVQSGAEVKKPGASVKVSCKASGYTF | 12 |
| | TDYYINWVRQAPGKGLEWIARIYPGSGNTY | |
| | YNEKFKGRVTITADKSTSTAYMELSSLRSE | |
| | DTAVYFCARGVYYFDYWGQGTTVTVSS | |

TABLE 5
VL Amino Acid Sequence

| | Amino Acid Sequence | SEQ ID NO: |
|---|---|---|
| VL1 | DIVMTQSPSFLSASVGDRVTITCKASQNVR | 13 |
| | TNVAWYQQKPGKAPKALIYSASYRYSGVP | |
| | DRFTGSGSGTDFTLTISSLQSEDFADYFCQQ | |
| | YNSYPRTFGGGTKVEIK | |
| VL2 | DIVMTQSPSSLSASVGDRVTITCKASQNVR | 14 |
| | TNVAWYQQKPGKAPKALIYSASYRYSGVP | |
| | DRFTGSGSGTDFTLTISSLQPEDFADYFCQQ | |
| | YNSYPRTFGGGTKVEIK | |
| VL3 | DIVMTQSPSSLSASVGDRVTITCKASQNVR | 15 |
| | TNVAWYQQKPGKAPKALIYSASYRYSGVP | |
| | DRFSGSGSGTDFTLTISSLQPEDFADYFCQQ | |
| | YNSYPRTFGGGTKVEIK | |
| VL4 | DIVMTQSPSSLSASVGDRVTITCKASQNVR | 16 |
| | TNVAWYQQKPGKAPKSLIYSASYRYSGVP | |
| | DRFSGSGSGTDFTLTISSLQPEDFATYYCQQ | |
| | YNSYPRTFGGGTKVEIK | |

In a specific aspect, anti-KIT antibodies (e.g., humanized antibodies) provided herein comprise a VH comprising the amino acid sequence of SEQ ID NO: 8, and/or a VL comprising the amino acid sequence of SEQ ID NO: 13. In one embodiment, the anti-KIT antibody provided herein comprises a VH comprising the amino acid sequence of SEQ ID NO: 8, and/or a VL comprising the amino acid sequence of SEQ ID NO: 14. In one embodiment, the anti-KIT antibody provided herein comprises a VH comprising the amino acid sequence of SEQ ID NO: 8, and/or a VL comprising the amino acid sequence of SEQ ID NO: 15. In one embodiment, the anti-KIT antibody provided herein comprises a VH comprising the amino acid sequence of SEQ ID NO: 8, and/or a VL comprising the amino acid sequence of SEQ ID NO: 16.

In one embodiment, the anti-KIT antibody provided herein comprises a VH comprising the amino acid sequence of SEQ ID NO: 9, and/or a VL comprising the amino acid sequence of SEQ ID NO: 13. In one embodiment, the anti-KIT antibody provided herein comprises a VH comprising the amino acid sequence of SEQ ID NO: 9, and/or a VL comprising the amino acid sequence of SEQ ID NO: 14. In one embodiment, the anti-KIT antibody provided herein comprises a VH comprising the amino acid sequence of SEQ ID NO: 9, and/or a VL comprising the amino acid sequence of SEQ ID NO: 15. In one embodiment, the anti-KIT antibody provided herein comprises a VH comprising the amino acid sequence of SEQ ID NO: 9, and/or a VL comprising the amino acid sequence of SEQ ID NO: 16.

In one embodiment, the anti-KIT antibody provided herein comprises a VH comprising the amino acid sequence of SEQ ID NO: 10, and/or a VL comprising the amino acid sequence of SEQ ID NO: 13. In one embodiment, the anti-KIT antibody provided herein comprises a VH comprising the amino acid sequence of SEQ ID NO: 10, and/or a VL comprising the amino acid sequence of SEQ ID NO: 14. In one embodiment, the anti-KIT antibody provided herein comprises a VH comprising the amino acid sequence of SEQ ID NO: 10, and a VL comprising the amino acid sequence of SEQ ID NO: 14. In one embodiment, the anti-KIT antibody provided herein comprises a VH comprising the amino acid sequence of SEQ ID NO: 10, and/or a VL comprising the amino acid sequence of SEQ ID NO: 15. In one embodiment, the anti-KIT antibody provided herein comprises a VH comprising the amino acid sequence of SEQ ID NO: 10, and/or a VL comprising the amino acid sequence of SEQ ID NO: 16.

In one embodiment, the anti-KIT antibody provided herein comprises a VH comprising the amino acid sequence of SEQ ID NO: 11, and/or a VL comprising the amino acid sequence of SEQ ID NO: 13. In one embodiment, the anti-KIT antibody provided herein comprises a VH comprising the amino acid sequence of SEQ ID NO: 11, and/or a VL comprising the amino acid sequence of SEQ ID NO: 14. In one embodiment, the anti-KIT antibody provided herein comprises a VH comprising the amino acid sequence of SEQ ID NO: 11, and/or a VL comprising the amino acid sequence of SEQ ID NO: 15. In one embodiment, the anti-KIT antibody provided herein comprises a VH comprising the amino acid sequence of SEQ ID NO: 11, and/or a VL comprising the amino acid sequence of SEQ ID NO: 16.

In one embodiment, the anti-KIT antibody provided herein comprises a VH comprising the amino acid sequence of SEQ ID NO: 12, and/or a VL comprising the amino acid sequence of SEQ ID NO: 13. In one embodiment, the anti-KIT antibody provided herein comprises a VH comprising the amino acid sequence of SEQ ID NO: 12, and/or a VL comprising the amino acid sequence of SEQ ID NO: 14. In one embodiment, the anti-KIT antibody provided herein comprises a VH comprising the amino acid sequence of SEQ ID NO: 12, and/or a VL comprising the amino acid sequence of SEQ ID NO: 15. In one embodiment, the anti-KIT antibody provided herein comprises a VH comprising the amino acid sequence of SEQ ID NO: 12, and/or a VL comprising the amino acid sequence of SEQ ID NO: 16.

In a specific aspect, anti-KIT antibodies (e.g., humanized antibodies) provided herein comprise:
(i) a VL comprising an amino acid sequence that is: at least 90% identical to SEQ ID NO: 13, at least 88% identical to SEQ ID NO: 14, at least 87% identical to SEQ ID NO: 15, or at least 84% identical to SEQ ID NO: 16; and
(ii) a VH comprising an amino acid sequence that is: at least 93% identical to SEQ ID NO: 8, at least 92% identical to SEQ ID NO: 9, at least 90% identical to SEQ ID NO: 10, at least 87% identical to SEQ ID NO: 11, or at least 86% identical to SEQ ID NO: 12.

Prior anti-KIT antibodies have been found to induce degranulation of FcgRI-expressing human mast cells and/or to show Fc receptor-dependent KIT agonist activity, which may give rise to undesirable infusion-related reactions (IRRs) among other adverse effects.

In various embodiments, an anti-KIT antibody or antigen binding fragment described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain). Preferably, an anti-KIT antibody or antigen binding fragment described herein has reduced Fc receptor binding activity (particularly reduced FcTR binding activity), does not induce degranulation of FcgRI-expressing human mast cells, and/or show Fc receptor-dependent KIT agonist activity. In certain embodiments, one or more of these properties of the anti-KIT antibody or antigen binding fragment result from the modified (e.g., mutated) Fc region or domain.

In specific embodiments, an anti-KIT antibody or antigen binding fragment described herein has reduced Fc receptor binding activity (particularly reduced FcTR binding activity). In specific embodiments, an anti-KIT antibody or antigen binding fragment described herein does not have significant Fc receptor (particularly FcTR) binding activity. In specific embodiments, an anti-KIT antibody or antigen binding fragment described herein has no detectable Fc receptor (particularly FcTR) binding activity. In particular embodiments, an anti-KIT antibody or antigen binding fragment described herein has at least 10% less, at least 20% less, at least 30% less, at least 40% less, at least 50% less, at least 60% less, at least 70% less, at least 80% less, at least 90% less, at least 95% less, or at least 99% less Fc receptor (particularly FcTR) binding activity compared to an appropriate control antibody or antigen binding fragment. When an anti-KIT antibody or antigen binding fragment described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain, in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment described herein comprises a modified (e.g., mutated) human IgG1 Fc region or domain and has at least 10% less, at least 20% less, at least 30% less, at least 40% less, at least 50% less, at least 60% less, at least 70% less, at least 80% less, at least 90% less, at least 95% less, or at least 99% less Fc receptor (particularly FcTR) binding activity compared to a corresponding antibody or antigen binding fragment having the same VH and VL but with a wild-type (unmodified) human IgG1 Fc region or domain.

In specific embodiments, an anti-KIT antibody or antigen binding fragment described herein does not induce significant degranulation of FcgRI-expressing human mast cells (e.g., as determined, for example, by % release of beta-hexosaminidase from human mast cells in culture (e.g., in presence of IFN gamma)). In specific embodiments, an anti-KIT antibody or antigen binding fragment described herein does not induce detectable degranulation of FcgRI-expressing human mast cells (e.g., as determined, for example, by % release of beta-hexosaminidase from human mast cells in culture (e.g., in presence of IFN gamma)). In particular embodiments, an anti-KIT antibody or antigen binding fragment described herein induces at least 10% less, at least 20% less, at least 30% less, at least 40% less, at least 50% less, at least 60% less, at least 70% less, at least 80% less, at least 90% less, at least 95% less, or at least 99% less degranulation of FcgRI-expressing human mast cells (e.g., as determined, for example, by % release of beta-hexosaminidase from human mast cells in culture (e.g., in presence of IFN gamma)) compared to an appropriate control antibody or antigen binding fragment. In particular embodiments, release of beta-hexosaminidase from human mast cells in culture in presence of IFN gamma is reduced by more than 50% with an anti-KIT antibody or antigen binding fragment described herein compared to an appropriate control antibody or antigen binding fragment. In particular embodiments, release of beta-hexosaminidase from human mast cells in culture in presence of IFN gamma is reduced by more than 60%, more than 70%, or more than 80% with an anti-KIT antibody or antigen binding fragment described herein compared to an appropriate control antibody or antigen binding fragment. When an anti-KIT antibody or antigen binding fragment described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain), in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment described herein comprises a modified (e.g., mutated) human IgG1 Fc region or domain and induces at least 10% less, at least 20% less, at least 30% less, at least 40% less, at least 50% less, at least 60% less, at least 70% less, at least 80% less, at least 90% less, at least 95% less, or at least 99% less degranulation of FcgRI-expressing human mast cells (e.g., as determined, for example, by % release of beta-hexosaminidase from human mast cells in culture (e.g., in presence of IFN gamma)) compared to a corresponding antibody or antigen binding fragment having the same VH and VL but with a wild-type (unmodified) human IgG1 Fc region or domain. In particular embodiments, release of beta-hexosaminidase from human mast cells in culture in presence of IFN gamma is reduced by more than 50% with an anti-KIT antibody or antigen binding fragment described herein that comprises a modified (e.g., mutated) human IgG1 Fc region or domain, compared to a corresponding antibody or antigen binding fragment having the same VH and VL but with a wild-type (unmodified) human IgG1 Fc region or domain. In particular embodiments, release of beta-hexosaminidase from human mast cells in culture in presence of IFN gamma is reduced by more than 60%, more than 70%, or more than 80% with an anti-KIT antibody or antigen binding fragment described herein that comprises a modified (e.g., mutated) human IgG1 Fc region or domain, compared to a corresponding antibody or antigen binding fragment having the same VH and VL but with a wild-type (unmodified) human IgG1 Fc region or domain.

In specific embodiments, an anti-KIT antibody or antigen binding fragment described herein does not show significant Fc receptor-dependent KIT agonistic activity (e.g., as determined, for example, by KIT phosphorylation). In specific embodiments, an anti-KIT antibody or antigen binding fragment described herein does not show detectable Fc receptor-dependent KIT agonistic activity (e.g., as determined, for example, by KIT phosphorylation). In particular embodiments, an anti-KIT antibody or antigen binding fragment described herein induces at least 10% less, at least 20% less, at least 30% less, at least 40% less, at least 50% less, at least 60% less, at least 70% less, at least 80% less, at least 90% less, at least 95% less, or at least 99% less Fc receptor-dependent KIT activity (e.g., as determined, for example, by KIT phosphorylation) compared to an appropriate control antibody or antigen binding fragment. In particular embodiments, Fc receptor-dependent KIT agonist activity (as determined by KIT phosphorylation with Fc receptors crosslinked) is reduced by more than 50% with an anti-KIT antibody or antigen binding fragment described herein compared to an appropriate control antibody or antigen binding fragment. In particular embodiments, Fc receptor-dependent KIT agonist activity (as determined by KIT phosphorylation with Fc receptors crosslinked) is reduced by more than 60%, more than 70%, or more than 80% with an anti-KIT antibody or antigen binding fragment described herein compared to an appropriate control antibody or antigen binding fragment. When an anti-KIT antibody or antigen binding fragment described herein comprises a modified (e.g., mutated) Fc region or domain (e.g., a modified (e.g., mutated) human IgG Fc region or domain, such as a modified (e.g., mutated) human IgG1, IgG2, IgG3, or IgG4 Fc region or domain), in preferred embodiments the appropriate control antibody or antigen binding fragment is an antibody or antigen binding fragment having the same VH and VL but with a wild-type (unmodified) Fc region or domain of the same isotype. In particular embodiments, an anti-KIT antibody or antigen binding fragment described herein comprises a modified (e.g., mutated) human IgG1 Fc region or domain and induces at least 10% less, at least 20% less, at least 30% less, at least 40% less, at least 50% less, at least 60% less, at least 70% less, at least 80% less, at least 90% less, at least 95% less, or at least 99% less Fc receptor-dependent KIT activity (e.g., as determined, for example, by KIT phosphorylation) compared to a corresponding antibody or antigen binding fragment having the same VH and VL but with a wild-type (unmodified) human IgG1 Fc region or domain. In specific embodiments, an anti-KIT antibody or antigen binding fragment described herein does not show significant or detectable Fc receptor-dependent KIT agonistic activity as described herein even when cross-linked on THP-1 cells. In particular embodiments, Fc receptor-dependent KIT agonist activity (as determined by KIT phosphorylation with Fc receptors crosslinked) is reduced by more than 50% with an anti-KIT antibody or antigen binding fragment described herein that comprises a modified (e.g., mutated) human IgG1 Fc region or domain, compared to a corresponding antibody or antigen binding fragment having the same VH and VL but with a wild-type (unmodified) human IgG1 Fc region or domain. In particular embodiments, Fc receptor-dependent KIT agonist activity (as determined by KIT phosphorylation with Fc receptors crosslinked) is reduced by more than 60%, more than 70%, or more than 80% with an anti-KIT antibody or antigen binding fragment described herein that comprises a modified (e.g., mutated) human IgG1 Fc region or domain, compared to a corresponding antibody or antigen binding fragment having the same VH and VL but with a wild-type (unmodified) human IgG1 Fc region or domain.

In various embodiments, an anti-KIT antibody or antigen binding fragment described herein (1) reduces disease activity in a CIndU patient (e.g., a CIndU patient whose CIndU is refractory to antihistamine treatment), (2) reduces skin mast cell number in a CIndU patient (e.g., a CIndU patient whose CIndU is refractory to antihistamine treatment), (3) reduces tryptase level in a CIndU patient (e.g., a CIndU patient whose CIndU is refractory to antihistamine treatment), (4) improves urticaria control in a CIndU patient (e.g., a CIndU patient whose CIndU is refractory to antihistamine treatment), (5) improves quality of life in a CIndU patient (e.g., a CIndU patient whose CIndU is refractory to antihistamine treatment), and/or (δ) maintains hematology parameters (such as hemoglobin (HgB) level, white blood cell (WBC) count, platelet count, and/or absolute neutrophil count (ANC)) in a patient such as a CIndU patient (e.g., a CIndU patient whose CIndU is refractory to antihistamine treatment) within the normal ranges.

In certain embodiments, an anti-KIT antibody or antigen binding fragment described herein can significantly decrease the critical temperature threshold value in a TempTest® for a CIndU patient (e.g., a CIndU patient whose CIndU is refractory to antihistamine treatment), relative to the value before treatment. In specific embodiments, an anti-KIT antibody or antigen binding fragment described herein can decrease the critical temperature threshold value in a TempTest® for a CIndU patient (e.g., a CIndU patient whose CIndU is refractory to antihistamine treatment) by at least 5° C., at least 6° C., at least 7° C., at least 8° C., at least 9° C., at least 10° C., at least 11° C., at least 12° C., at least 13° C., at least 14° C., at least 15° C., at least 16° C., at least 17° C., at least 18° C., at least 19° C., or at least 20° C. (e.g., in a week, in 2 weeks, in 4 weeks, in 6 weeks, in 8 weeks, in 10 weeks, or in 12 weeks after treatment with the anti-KIT antibody or antigen binding fragment), relative to the value before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly decrease the pin number in a FricTest® for a CIndU patient (e.g., a CIndU patient whose CIndU is refractory to antihistamine treatment), relative to the pin number before treatment. In specific embodiments, an anti-KIT antibody or antigen binding fragment described herein can decrease the pin number in a FricTest® for a CIndU patient (e.g., a CIndU patient whose CIndU is refractory to antihistamine treatment) by at least 1, at least 2, at least 3, or at least 4 (e.g., in a week, in 2 weeks, in 4 weeks, in 6 weeks, in 8 weeks, in 10 weeks, or in 12 weeks after treatment with the anti-KIT antibody or antigen binding fragment), relative to the pin number before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly improve physician's global assessment (Phys-GA) and/or patient's global assessment (Pat-GA), relative to the level before treatment. In specific embodiments, an anti-KIT antibody or antigen binding fragment described herein can improve physician's global assessment (Phys-GA) and/or patient's global assessment (Pat-GA) by reducing the Likert scale (of 0-3, where 0 is none and 3 is severe) by at least 0.2, at least 0.3, at least 0.4, at least 0.5, at least 0.6, at least 0.7, at least 0.8, at least 0.9, at least 1.0, at least 1.1, at least 1.2, or at least 1.3 (e.g., in a week, in 2 weeks, in 4 weeks, in 6 weeks, in 8 weeks, in 10 weeks, or in 12 weeks after treatment with the anti-KIT antibody or antigen binding fragment), relative to the level before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly reduce skin mast cell number in a CIndU patient (e.g., a CIndU patient whose CIndU is refractory to antihistamine treatment), relative to the number before treatment. In specific embodiments, an anti-KIT antibody or antigen binding fragment described herein can reduce skin mast cell number in a CIndU patient (e.g., a CIndU patient whose CIndU is refractory to antihistamine treatment) by at least 20%, at least 40%, at least 60%, or at least 80% (e.g., in a week, in 2 weeks, in 4 weeks, in 6 weeks, in 8 weeks, in 10 weeks, or in 12 weeks after treatment with the anti-KIT antibody or antigen binding fragment), relative to the number before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly reduce serum tryptase in a CIndU patient (e.g., a CIndU patient whose CIndU is refractory to antihistamine treatment), relative to the level before treatment. In specific embodiments, an anti-KIT antibody or antigen binding fragment described herein can reduce serum tryptase in a CIndU patient (e.g., a CIndU patient whose CIndU is refractory to antihistamine treatment) by at least 50%, at least 70%, or at least 90% (e.g., in a week, in 2 weeks, in 4 weeks, in 6 weeks, in 8 weeks, in 10 weeks, or in 12 weeks after treatment with the anti-KIT antibody or antigen binding fragment), relative to the level before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly improve urticaria control in a CIndU patient (e.g., a CIndU patient whose CIndU is refractory to antihistamine treatment), relative to the level before treatment. In specific embodiments, an anti-KIT antibody or antigen binding fragment described herein can improve urticaria control in a CIndU patient (e.g., a CIndU patient whose CIndU is refractory to antihistamine treatment) by increasing the urticaria control test (UCT) score by at least 2, at least 3, at least 4, at least 5, at least 6, at least 7, at least 8, at least 9, at least 10, at least 11, at least 12, at least 13, at least 14, at least 15, or 16, or by increasing the UCT score to at least 12, at least 13, at least 14, at least 15, or 16 (e.g., in a week, in 2 weeks, in 4 weeks, in 6 weeks, in 8 weeks, in 10 weeks, or in 12 weeks after treatment with the anti-KIT antibody or antigen binding fragment), relative to the level before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein can significantly improve quality of life in a CIndU patient (e.g., a CIndU patient whose CIndU is refractory to antihistamine treatment), relative to the level before treatment. In specific embodiments, an anti-KIT antibody or antigen binding fragment described herein can improve quality of life in a CIndU patient (e.g., a CIndU patient whose CIndU is refractory to antihistamine treatment) by decreasing the dermatology life quality index (DLQI) by at least 2, at least 3, at least 4, at least 5, at least 6, at least 7, at least 8, at least 9, at least 10, at least 12, at least 14, at least 16, at least 18, at least 20, or at least 25, or by decreasing the DLQI to at most 5, at most 4, at most 3, at most 2, at most 1, or 0 (e.g., in a week, in 2 weeks, in 4 weeks, in 6 weeks, in 8 weeks, in 10 weeks, or in 12 weeks after treatment with the anti-KIT antibody or antigen binding fragment), relative to the level before treatment. In specific embodiments, the effect of the anti-KIT antibody or antigen binding fragment is sustained for at least 2 weeks, at least 4 weeks, at least 6 weeks, at least 8 weeks, at least 10 weeks, or at least 12 weeks.

In certain embodiments, an anti-KIT antibody or antigen binding fragment described herein maintains hematology parameters (such as hemoglobin (HgB) level, white blood cell (WBC) count, platelet count, and/or absolute neutrophil count (ANC)) in a patient within the normal ranges. In certain embodiments, an anti-KIT antibody or antigen binding fragment described herein maintains hematology parameters (such as hemoglobin (HgB) level, white blood cell (WBC) count, platelet count, and/or absolute neutrophil count (ANC)) in a CIndU patient (e.g., a CIndU patient whose CIndU is refractory to antihistamine treatment) within the normal ranges. In specific embodiments, the hematology parameters are maintained for at least 2 weeks, at least 4 weeks, at least 6 weeks, at least 8 weeks, at least 10 weeks, or at least 12 weeks.

In various embodiments, an anti-KIT antibody described herein has one or more of the properties described herein. In various embodiments, an antigen binding fragment of an anti-KIT antibody described herein has one or more of the properties described herein.

In specific embodiments, an antibody described herein comprises a modified Fc region or domain, wherein the Fc region or domain comprises at least one (e.g., one, two, three, four, five or six) amino acid modifications (e.g. substitution, deletion or addition) or at least one (e.g., one, two, three, four, five or six) non-naturally occurring amino acid residues.

In a specific embodiment, an antibody described herein comprises a modified Fc region or domain, wherein the Fc region or domain is an Fc region or domain of human IgG1 and comprises at least one (e.g., one, two, three, four, five or six) amino acid modifications (e.g. substitution, deletion or addition) or at least one (e.g., one, two, three, four, five or six) non-naturally occurring amino acid residues selected from the group consisting of 234A, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241L, 241Y, 241E, 241R. 243W, 243L 243Y, 243R, 243Q, 244H, 245A, 247V, 247G, 252Y, 254T, 256E, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 269H, 269Y, 269F, 269R, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 313F, 322Q, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 332H, 332Y, and 332A as numbered by the EU index as set forth in Kabat. Optionally, the Fc region or domain may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 04/016750; WO 04/029207; WO 04/035752 and WO 05/040217). In a specific embodiment, an antibody described herein comprises a modified Fc region or domain, wherein the Fc region or domain is an Fc region or domain of human IgG2 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified Fc region or domain, wherein the Fc region or domain is an Fc region or domain of human IgG3 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified Fc region or domain, wherein the Fc region or domain is an Fc region or domain of human IgG4 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art.

In a specific embodiment, an antibody described herein comprises a modified Fc region or domain, wherein the Fc region or domain is an Fc region or domain of human IgG1 and comprises at least one (e.g., one, two, three, four, five or six) amino acid modifications (e.g. substitution, deletion or addition) or at least one non-naturally occurring amino acid residue (e.g., one, two, three, four, five or six) selected from the group consisting of 234A, 234D, 234E, 234N, 234Q, 234T, 234H, 234Y, 2341, 234V, 234F, 235A, 235D, 235R, 235W, 235P, 235S, 235N, 235Q, 235T, 235H, 235Y, 2351, 235V, 235F, 236E, 239D, 239E, 239N, 239Q, 239F, 239T, 239H, 239Y, 2401, 240A, 240T, 240M, 241W, 241L, 241Y, 241E, 241R. 243W, 243L 243Y, 243R, 243Q, 244H, 245A, 247V, 247G, 252Y, 254T, 256E, 2621, 262A, 262T, 262E, 2631, 263A, 263T, 263M, 264L, 2641, 264W, 264T, 264R, 264F, 264M, 264Y, 264E, 265G, 265N, 265Q, 265Y, 265F, 265V, 2651, 265L, 265H, 265T, 2661, 266A, 266T, 266M, 267Q, 267L, 269H, 269Y, 269F, 269R, 296E, 296Q, 296D, 296N, 296S, 296T, 296L, 2961, 296H, 269G, 297S, 297D, 297E, 298H, 298I, 298T, 298F, 2991, 299L, 299A, 299S, 299V, 299H, 299F, 299E, 313F, 322Q, 325Q, 325L, 3251, 325D, 325E, 325A, 325T, 325V, 325H, 327G, 327W, 327N, 327L, 328S, 328M, 328D, 328E, 328N, 328Q, 328F, 328I, 328V, 328T, 328H, 328A, 329F, 329H, 329Q, 330K, 330G, 330T, 330C, 330L, 330Y, 330V, 3301, 330F, 330R, 330H, 332D, 332S, 332W, 332F, 332E, 332N, 332Q, 332T, 332H, 332Y, and 332A as numbered by the EU index as set forth in Kabat. Optionally, the Fc region or domain may comprise additional and/or alternative non-naturally occurring amino acid residues known to one skilled in the art (see, e.g., U.S. Pat. Nos. 5,624,821; 6,277,375; 6,737,056; PCT Patent Publications WO 01/58957; WO 04/016750; WO 04/029207; WO 04/035752 and WO 05/040217). In a specific embodiment, an antibody described herein comprises a modified Fc region or domain, wherein the Fc region or domain is an Fc region or domain of human IgG2 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified Fc region or domain, wherein the Fc region or domain is an Fc region or domain of human IgG3 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art. In a specific embodiment, an antibody described herein comprises a modified Fc region or domain, wherein the Fc region or domain is an Fc region or domain of human IgG4 and comprises at least one (e.g., one, two, three, four, five or six) amino acid modifications (e.g. substitution, deletion or addition) or at least one (e.g., one, two, three, four, five or six) non-naturally occurring amino acid residues, which are equivalents to the amino acid residue(s) described herein for a human IgG1 Fc region or domain, as can be determined by one of skill in the art.

In a certain aspect, provided herein is an antibody comprising an Fc region or domain, wherein the Fc region or domain is an Fc region or domain of human IgG1 and comprises at least a non-naturally occurring amino acid at one or more positions selected from the group consisting of 239, 330 and 332, as numbered by the EU index as set forth in Kabat. In a specific embodiment, provided herein is an antibody comprising an Fc region or domain, wherein the Fc region or domain is an Fc region or domain of human IgG1 and comprises at least one non-naturally occurring amino acid selected from the group consisting of 239D, 330L and 332E, as numbered by the EU index as set forth in Kabat. Optionally, the Fc region or domain may further comprise additional non-naturally occurring amino acid at one or more positions selected from the group consisting of 252, 254, and 256, as numbered by the EU index as set forth in Kabat. In a specific embodiment, provided herein is an antibody comprising an Fc region or domain, wherein the Fc region or domain is an Fc region or domain of human IgG1 and comprises at least one non-naturally occurring amino acid selected from the group consisting of 239D, 330L and 332E, as numbered by the EU index as set forth in Kabat and at least one non-naturally occurring amino acid at one or more positions are selected from the group consisting of 252Y, 254T and 256E, as numbered by the EU index as set forth in Kabat. In a specific embodiment, provided herein is an antibody comprising an Fc region or domain, wherein the Fc region or domain is an Fc region or domain of human IgG2, IgG3, or IgG4, and comprises at least one non-naturally occurring amino acid residue that is an equivalent(s) to the amino acid residue(s) described herein for a human IgG1 Fc region or domain, as can be determined by one of skill in the art. In a specific embodiment, provided herein is an antibody comprising an Fc region or domain, wherein the Fc region or domain is an Fc region or domain of human IgG2, IgG3, or IgG4, and comprises at least one non-naturally occurring amino acid residue at one or more positions that are equivalent(s) to the positions described herein for a human IgG1 Fc region or domain, as can be determined by one of skill in the art. In one embodiment, an Fc region or domain comprising such sequence exhibits one or more Fc activity, for example, binding affinity to an Fc receptor or effector function, such as ADCC or CDC. In a specific embodiment, an Fc region or domain comprising such sequence exhibits reduced Fc activity, for example, reduced binding affinity to an Fc receptor or reduced effector function, such as ADCC or CDC. In a particular embodiment, an Fc region or domain comprising such sequence exhibits enhanced FcRn activity, for example, enhanced half-life.

Additional non-limiting examples of Fc region or domain modifications are provided in Ghetie et al., 1997, Nat Biotech. 15:637-40; Duncan et al., 1988, Nature 332:563-564; Lund et al., 1991, J. Immunol 147:2657-2662; Lund et al., 1992, Mol Immunol 29:53-59; Alegre et al., 1994, Transplantation 57: 1537-1543; Hutchins et al., 1995, Proc Natl. Acad Sci USA 92: 11980-11984; Jefferis et al., 1995, Immunol Lett. 44: 111-117; Lund et al., 1995, Faseb J 9: 115-119; Jefferis et al., 1996, Immunol Lett 54: 101-104; Lund et al., 1996, J Immunol 157:4963-4969; Armour et al., 1999, Eur J Immunol 29:2613-2624; Idusogie et al., 2000, J Immunol 164:4178-4184; Reddy et al., 2000, J Immunol 164: 1925-1933; Xu et al., 2000, Cell Immunol 200: 16-26; Idusogie et al., 2001, J Immunol 166:2571-2575; Shields et al., 2001, J Biol Chem 276:6591-6604; Jefferis et al., 2002, Immunol Lett 82:57-65; Presta et al., 2002, Biochem Soc Trans 30:487-490); U.S. Pat. Nos. 5,624,821; 5,885,573; 5,677,425; 6,165,745; 6,277,375; 5,869,046; 6,121,022; 5,624,821; 5,648,260; 6,528,624; 6,194,551; 6,737,056; 6,821,505; 6,277,375; 8,163,882; 7,355,008; 7,960,512; 8,039,592; 8,039,359; 8,101,720; 7,214,775; 7,682,610; 7,741,442; U.S. Patent Publication Nos. 2004/0002587 and PCT Publications WO 94/29351; WO 99/58572; WO 00/42072; WO 04/029207; WO 04/099249; WO 04/063351.

In specific embodiments, the antibody described herein comprises a modified (e.g., mutated) human IgG1 Fc region or domain, which comprises non-naturally occurring amino acids 234A, 235Q and 322Q as numbered by the EU index as set forth in Kabat. In a particular embodiment, the modified (e.g., mutated) human IgG1 Fc region or domain further comprises non-naturally occurring amino acids 252Y, 254T and 256E as numbered by the EU index as set forth in Kabat.

In certain embodiments, the antibody described herein comprises a modified (e.g., mutated) human IgG2 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG2 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In certain embodiments, the antibody described herein comprises a modified (e.g., mutated) human IgG3 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG3 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In certain embodiments, the antibody described herein comprises a modified (e.g., mutated) human IgG4 Fc region or domain, which comprises non-naturally occurring amino acids that are equivalents to 234A, 235Q and 322Q as numbered by the EU index as set forth in Kabat for human IgG1 Fc region or domain, as can be determined by one of skill in the art. In certain embodiments, the antibody described herein comprises a modified (e.g., mutated) human IgG4 Fc region or domain, which comprises non-naturally occurring amino acids that are equivalents to 234A, 235Q, 322Q, 252Y, 254T and 256E as numbered by the EU index as set forth in Kabat for human IgG1 Fc region or domain, as can be determined by one of skill in the art.

In a specific embodiment, the antibody described herein comprises the VL and VH CDR sequences set forth in Table 1 and a modified (e.g., mutated) human IgG1 Fc region or domain, wherein the modified (e.g., mutated) human IgG1 Fc region or domain comprises non-naturally occurring amino acids 234A, 235Q, and 322Q as numbered by the EU index as set forth in Kabat.

In a preferred embodiment, the antibody described herein comprises the VL and VH CDR sequences set forth in Table 1 and a modified (e.g., mutated) human IgG1 Fc region or domain, wherein the modified (e.g., mutated) human IgG1 Fc region or domain comprises non-naturally occurring amino acids 234A, 235Q, 322Q, 252Y, 254T and 256E as numbered by the EU index as set forth in Kabat.

Thus, in one aspect, provided herein is an antibody, which immunospecifically binds to human KIT, comprising:

(i) a VL comprising a VL CDR1, VL CDR2, and VL CDR3 having the amino acid sequences of SEQ ID NO: 2, SEQ ID NO: 3, and SEQ ID NO: 4, respectively; (ii) a VH comprising VH CDR1, VH CDR2, and VH CDR3 having the amino acid sequences of SEQ ID NO: 5, SEQ ID NO: 6, and SEQ ID NO: 7, respectively; and (iii) a modified (e.g., mutated) human IgG1 Fc region or domain comprising non-naturally occurring amino acids 234A, 235Q, and 322Q as numbered by the EU index as set forth in Kabat.

Thus, in a further aspect, provided herein is an antibody, which immunospecifically binds to human KIT, comprising:

(i) a VL comprising a VL CDR1, VL CDR2, and VL CDR3 having the amino acid sequences of SEQ ID NO: 2, SEQ ID NO: 3, and SEQ ID NO: 4, respectively; (ii) a VH comprising VH CDR1, VH CDR2, and VH CDR3 having the amino acid sequences of SEQ ID NO: 5, SEQ ID NO: 6, and SEQ ID NO: 7, respectively; and (iii) a modified (e.g., mutated) human IgG1 Fc region or domain comprising non-naturally occurring amino acids 234A, 235Q, 322Q, 252Y, 254T and 256E as numbered by the EU index as set forth in Kabat.

In a further aspect, provided herein is an antibody, which immunospecifically binds to human KIT, comprising: (i) a VL comprising the amino acid sequence:DIVMTQSPSXKiLSASVGDRVTITCKASQNVRTNVAWYQQKPGKAPKX_{K2}LIYS ASYRYSGVPDRFX_{K3}GSGSGTDFTLTISSLQX_{K4}EDFAX_{K5} YX_{K6}CQQYNSYPRTFGGGTKV EIK (SEQ ID NO: 17), wherein XKJ is an amino acid with an aromatic or aliphatic hydroxyl side chain, X_{K2} is an amino acid with an aliphatic or aliphatic hydroxyl side chain, X₃ is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and (ii) a VH comprising the amino acid sequence: QVQLVQSGAEXHiKKPGASVKX_{H2}SCKASGYTFTDYYINWVX_{H3}QAPGKGLEWIARIYPG SGNTYYNEKFKGRX_{H4}TX_{H5}TAX_{H6}KSTSTAYMXi7LSSLRSE)X_{H8}AVYFCARGVYYFDY WGQGTTVTVSS (SEQ ID NO: 18) wherein X_{H1} is an amino acid with an aliphatic side chain, X_{H2} is an amino acid with an aliphatic side chain, X_{H3} is an amino acid with a polar or basic side chain, XH₄ is an amino acid with an aliphatic side chain, XH is an amino acid with an aliphatic side chain, X_{H6} is an amino acid with an acidic side chain, XH₇ is an amino acid with an acidic or amide derivative side chain, and X_{H8} is an amino acid with an aliphatic hydroxyl side chain; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat.

In a further aspect, provided herein is an antibody, which immunospecifically binds to human KIT, comprising: i) a VL which comprises the amino acid sequence of SEQ ID NO: 13, 14, 15, or 16, and ii) a VH comprising the amino acid sequence of SEQ ID NO: 8, 9, 10, 11, or 12; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat.

In a further aspect, provided herein is an antibody, which immunospecifically binds to human KIT, comprising: i) a VL which comprises the amino acid sequence of SEQ ID NO: 14 and ii) a VH which comprises the amino acid sequence of SEQ ID NO: 10; and (iii) a modified (e.g., mutated) human IgG1 Fc region or domain comprising non-naturally occurring amino acids 234A, 235Q, 322Q and preferably also 252Y, 254T and 256E as numbered by the EU index as set forth in Kabat.

In specific embodiments, the antibody provided herein comprises a heavy chain comprising the following amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG.

In specific embodiments, the antibody provided herein comprises a light chain comprising the following amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In specific embodiments, the antibody provided herein comprises a heavy chain comprising the following amino acid sequence:

(SEQ ID NO: 21)
QVQLVQSGAEVKKPGASVKLSCKASGYTFTDYYINWVRQAPGKGLEWIA
RIYPGSGNTYYNEKFKGRATLTADKSTSTAYMQLSSLRSEDTAVYFCAR
GVYYFDYWGQGTTVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSCDKTHTCPPCPAPEAQGGPSVFLFPPK
PKDTLYITREPEVTCVVVDVSHEDPEVKFNWYVDGVEVHNAKTKPREEQ
YNSTYRVVSVLTVLHQDWLNGKEYKCQVSNKALPAPIEKTISKAKGQPR
EPQVYTLPPSRDELTKNQVSLTCLVKGFYPSDIAVEWESNGQPENNYKT
TPPVLDSDGSFFLYSKLTVDKSRWQQGNVFSCSVMHEALHNHYTQKSLS
LSPG;

and a light chain comprising the following amino acid sequence:

(SEQ ID NO: 22)
DIVMTQSPSSLSASVGDRVTITCKASQNVRTNVAWYQQKPGKAPKALIY
SASYRYSGVPDRFTGSGSGTDFTLTISSLQPEDFADYFCQQYNSYPRTF
GGGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

In a specific embodiment, provided herein is an antibody comprising: (i) a heavy chain comprising the amino acid sequence:

(SEQ ID NO: 19)
MEWSWVFLFFLSVTTGVHSQVQLVQSGAEVKKPGASVKLSCKASGYTFTDYYINWVRQAP
GKGLEWIARIYPGSGNTYYNEKFKGRATLTADKSTSTAYMQLSSLRSEDTAVYFCARGVYYFDY
WGQGTTVTVSSASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKVEPKSCDKTHTC
PPCPAPE [symbol] GGPSVFLFPPKPKDTL [symbol] I [symbol] R [symbol] PEVTCVVDVSHEDPEVKFNWYVDGVEVH
NAKTKPREEQYNSTYRVVSVLTVLHQDWLNGKEYKC [symbol] VSNKALPAPIEKTISKAKGQP
REPQVYTLPPSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG,

wherein the leader sequence is shown in bold italic type, the variable region (VH) is shown in italic type and the constant region is shown underlined. In addition the mutations in the constant region (compared to wild type human IgG1) are shown double underlined; and (ii) a light chain comprising the amino acid sequence:

(SEQ ID NO: 20)
MSVPTQVLGLLLLWLTDARCDIVMTQSPSSLSASVGDRVTITCKASQNVRTNVAWYQQKPG
KAPKALIYSASYRYSGVPDRFTGSGSGTDFTLTISSLQPEDFADYFCQQYNSYPRTFGGGTKVE
IKRTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC,

wherein the leader sequence is shown in bold italic type, the variable region (VL) is shown in italic type and the constant region is shown underlined.

In specific embodiments, an anti-KIT antibody described herein does not bind to (e.g., has no detectable binding to) any human Fc-gamma receptor (FcTR receptor). In a specific embodiment, an anti-KIT antibody described herein does not bind to (e.g., has no detectable binding to) human FcγRI. In a specific embodiment, an anti-KIT antibody described herein does not bind to (e.g., has no detectable binding to) human FcγRIIa. In a specific embodiment, an anti-KIT antibody described herein does not bind to (e.g., has no detectable binding to) human FcγRIIb. In a specific embodiment, an anti-KIT antibody described herein does not bind to (e.g., has no detectable binding to) human FcγRIIIa. In a specific embodiment, an anti-KIT antibody described herein does not bind to (e.g., has no detectable binding to) human FcγRIIIb.

In specific embodiments, an anti-KIT antibody described herein comprises a modified (e.g., mutated) human IgG constant region (e.g., a modified (e.g., mutated) human IgG1, IgG2, IgG3, or IgG4 constant region) and has an enhanced binding (e.g., an at least 2-fold, 5-fold, 10-fold, 50-fold, 100-fold, 500-fold, 1000-fold, 5000-fold, or 10000-fold higher binding affinity) to human neonatal Fc Receptor (FcRn) relative to a corresponding antibody with the same variable region sequences but an unmodified (wild type) human IgG constant region. In a specific embodiment, an anti-KIT antibody described herein binds to FcRn at pH 6.0 with a KD of less than 20 nM. In a specific embodiment, an anti-KIT antibody described herein binds to FcRn at pH 6.0 with a KD of less than 2 nM. In a specific embodiment, an anti-KIT antibody described herein binds to FcRn at pH 6.0 with a KD of less than 1 nM. In a specific embodiment, an anti-KIT antibody described herein binds to FcRn at pH 6.0 with a KD of less than 500 nM. In a specific embodiment, an anti-KIT antibody described herein binds to FcRn at pH 6.0 with a KD of less than 400 pM. In a specific embodiment, an anti-KIT antibody described herein binds to FcRn at pH 7.2 with a KD of less than 200 nM. In a specific embodiment, an anti-KIT antibody described herein binds to FcRn at pH 7.2 with a KD of less than 150 nM. In a specific embodiment, an anti-KIT antibody described herein binds to FcRn at pH 7.2 with a KD of less than 100 nM. In a specific embodiment, an anti-KIT antibody described herein binds to FcRn at pH 7.2 with a KD of less than 80 nM.

In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits no antibody-dependent cellular cytotoxicity (ADCC). In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits reduced (e.g., at least 10% less, at least 20% less, at least 30% less, at least 40% less, at least 50% less, at least 60% less, at least 70% less, at least 80% less, at least 90% less, at least 95% less, or at least 99% less) ADCC, relative to a corresponding antibody with the same variable region sequences but an unmodified (wild type) human IgG constant region.

In specific embodiments, an anti-KIT antibody described herein comprises a modified (e.g., mutated) human IgG constant region (e.g., a modified (e.g., mutated) human IgG1, IgG2, IgG3, or IgG4 constant region) and exhibits reduced (e.g., at least 10% less, at least 20% less, at least 30% less, at least 40% less, at least 50% less, at least 60% less, at least 70% less, at least 80% less, at least 90% less, at least 95% less, or at least 99% less) production of cytokines (e.g., IFN-7, IL-1β, IL-2, IL-6, IL-8, IL-10, and/or TNF-α), relative to a corresponding antibody with the same variable region sequences but an unmodified (wild type) human IgG constant region.

In specific aspects, also provided are antigen binding fragments of the antibodies described herein, preferably antigen binding fragments that comprise a full-length heavy chain Fc region or domain (e.g., a full-length human IgG1, human IgG2, human IgG3, or human IgG4 Fc region or domain). In specific aspects, also provided are antigen binding fragments of the antibodies described herein that comprise a partial heavy chain Fc region or domain (e.g., a partial human IgG1, human IgG2, human IgG3, or human IgG4 Fc region or domain).

In certain aspects, anti-KIT antibodies or antigen binding fragments thereof can be obtained using methods known in the art, for example, see Section 5.4 below.

In a particular aspect, an anti-KIT antibody or an antigen binding fragment thereof provided herein specifically binds to a D4 domain of human KIT and a D5 region of KIT, e.g., human KIT. In another specific embodiment, an anti-KIT antibody or an antigen binding fragment thereof provided herein specifically binds to a D5 domain of KIT, e.g., human KIT, with lower affinity than to a D4 domain of KIT, e.g., human KIT. In a particular embodiment, an anti-KIT antibody or an antigen binding fragment thereof provided herein specifically binds to a D4 domain of KIT, e.g., human KIT, with higher affinity than to a D5 domain of KIT, e.g., human KIT; for example, the higher affinity is at least 1 fold, 2 fold, 3 fold, 4 fold, 5 fold, 10 fold, 20 fold, 50 fold, 100 fold, 500 fold, or 1000 fold as determined by methods known in the art, e.g., ELISA or Biacore assays.

In a particular embodiment, an anti-KIT antibody or an antigen binding fragment thereof provided herein specifically binds to a D4 or D4/D5 region of KIT, e.g., human KIT, and has at least 1 fold, 2 fold, 3 fold, 4 fold, 5 fold, or 10 fold higher affinity for a KIT antigen consisting essentially of a D4 domain only than a KIT antigen consisting essentially of a D5 domain only.

In a particular embodiment, an anti-KIT antibody or an antigen binding fragment thereof provided herein specifically binds to a KIT polypeptide (e.g., the D4 region of human KIT) with an EC₅₀ (half maximal effective concentration) value of about 50 nM, 10 nM, 500 pM, 300 pM, 200 pM, 100 pM or 50 pM or less as determined by an assay described in the art, such as ELISA.

In a particular embodiment, an anti-KIT antibody or an antigen binding fragment thereof provided herein specifically binds to a KIT polypeptide (e.g., the D4 region of human KIT) with an EC₅₀ value of about 200 pM or 150 pM or less as determined by an assay described in the art, such as ELISA or FACs with CHO-WT-KIT cells (CHO cells engineered to recombinantly express wild-type human KIT).

In a particular embodiment, an anti-KIT antibody or an antigen binding fragment thereof provided herein is capable of blocking KIT phosphorylation with IC₅₀ (50% inhibition concentration) value of about 600 pM or less.

In a particular embodiment, an anti-KIT antibody or an antigen binding fragment thereof provided herein is capable of inducing or enhancing KIT receptor internalization, e.g., by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, relative to internalization in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor internalization, e.g., by at least about 25% or 35%, optionally to about 75%, as assessed by methods described herein or known to one of skill in the art, relative to internalization in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor internalization, e.g., by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art, relative to internalization in the presence of an unrelated antibody (e.g., an antibody that does not immunospecifically bind to KIT). Techniques for the quantitation or visualization of cell surface receptors are well known in the art and include a variety of fluorescent and radioactive techniques. For example, one method involves incubating the cells with a radiolabeled anti-receptor antibody. Alternatively, the natural ligand of the receptor can be conjugated to a fluorescent molecule or radioactive-label and incubated with the cells. Additional receptor internalization assays are well known in the art and are described in, for example, Jimenez et al., Biochemical Pharmacology, 1999, 57:1125-1131; Bernhagen et al., Nature Medicine, 2007, 13:587-596; and Conway et al., J. Cell Physiol., 2001, 189:341-55.

In a particular embodiment, an anti-KIT antibody or an antigen binding fragment thereof provided herein is capable of inducing or enhancing KIT receptor turnover, e.g., by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assay), relative to turnover in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor turnover, by at least about 25% or 35%, optionally to about 75%, as assessed by methods described herein or known to one of skill in the art (e.g., pulse-chase assay), relative to turnover in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor turnover, by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assay), relative to turnover in the presence of an unrelated antibody (e.g., an antibody that does not immunospecifically bind to KIT). Methods for the determining receptor turnover are well known in the art. For example, cells expressing KIT can be pulse-labeled using ³⁵S-EXPRESS Protein Labeling mix (NEG772, NEN Life Science Products), washed and chased with unlabeled medium for a period of time before protein lysates from the labeled cells are immunoprecipitated using an anti-KIT antibody and resolved by SDS-PAGE and visualized (e.g., exposed to a PhosphoImager screen (Molecular Dynamics), scanned using the Typhoon8600 scanner (Amersham), and analyzed using ImageQuant software (Molecular Dynamics)) (see, e.g., Chan et al., Development, 2004, 131:5551-5560).

In a particular embodiment, an anti-KIT antibody or an antigen binding fragment thereof provided herein is capable of inducing or enhancing KIT receptor degradation, by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., pulse-chase assays), relative to degradation in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor degradation, by at least about 25% or 35%, optionally to about 75%, as assessed by methods described herein or known to one of skill in the art (e.g., pulse-chase assays), relative to degradation in the presence of an unrelated antibody (e.g., an antibody that does not immunospecifically bind to KIT). In a particular embodiment, an anti-KIT antibody or an antigen binding fragment thereof provided herein is capable of inducing or enhancing KIT receptor degradation, by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., pulse-chase assays), relative to degradation in the presence of an unrelated antibody (e.g., an antibody that does not immunospecifically bind to KIT). Techniques for quantitating or monitoring ubiquitination and/or degradation (e.g., kinetics or rate of degradation) of cell surface receptors are well known in the art and involve a variety of fluorescent and radioactive techniques (see, e.g., International Patent Application Publication No. WO 2008/153926 A2). For example, pulse chase experiments or experiments using radiolabeled ligands such as ¹²⁵I-SCF can be carried out to quantitatively measure degradation of KIT.

In particular embodiments, an anti-KIT antibody or an antigen binding fragment thereof provided herein does not bind the extracellular ligand binding site of KIT, e.g., the SCF binding site of KIT. In particular embodiments, an anti-KIT antibody or an antigen binding fragment thereof provided herein does not inhibit ligand binding to KIT, e.g., does not inhibit KIT ligand (e.g., SCF) binding to KIT, as determined by a method described in the art, for example, ELISA. In certain embodiments, an anti-KIT antibody or an antigen binding fragment thereof provided herein does not fully inhibit, or partially inhibits, ligand binding to KIT, e.g., does not fully inhibit, or partially inhibits, KIT ligand (e.g., SCF) binding to KIT, as determined by a method described in the art, for example, ELISA or FACS (fluorescence-activated cell sorting).

In specific aspects, anti-KIT antibodies (e.g., human or humanized antibodies) provided herein are inhibitory antibodies, that is, antibodies that inhibit (e.g., partially inhibit) KIT activity, i.e., one or more KIT activities. In a specific embodiment, partial inhibition of a KIT activity results in, for example, about 25% to about 65% or 75% inhibition. In a specific embodiment, partial inhibition of a KIT activity results in, for example, about 35% to about 85% or 95% inhibition. Non-limiting examples of KIT activities include KIT dimerization, KIT phosphorylation (e.g., tyrosine phosphorylation), signaling downstream of KIT (e.g. Stat, AKT, MAPK, or Ras signaling), induction or enhancement of gene transcription (e.g., c-Myc), induction or enhancement of cell proliferation or cell survival. In a particular embodiment, an antibody described herein inhibits KIT phosphorylation (e.g., ligand-induced phosphorylation).

In a specific embodiment, an anti-KIT antibody or an antigen binding fragment thereof provided herein inhibits KIT tyrosine phosphorylation in the KIT cytoplasmic domain.

In another particular embodiment, an anti-KIT antibody or an antigen binding fragment thereof provided herein inhibits cell proliferation, for example, mast cell proliferation or eosinophil proliferation. In yet another particular embodiment, an anti-KIT antibody or an antigen binding fragment thereof provided herein inhibits cell survival, for example mast cell survival or eosinophil cell survival. In certain aspects, inhibition of cell proliferation, for example, mast cell proliferation or eosinophil proliferation, is at least 10%, 20%, 30%, 40%, 50%, 60%, 70%, 80%, or 90%.

In another particular embodiment, an anti-KIT antibody or an antigen binding fragment thereof provided herein inhibits mast cell activation or eosinophil activation. In certain aspects, inhibition of mast cell activation or activity or eosinophil activation or activity, is at least 10%, 20%, 30%, 40%, 50%, 60%, 70%, 80%, or 90%.

In a specific embodiment, an anti-KIT antibody or an antigen binding fragment thereof provided herein inhibits eosinophil or mast cell degranulation (see, e.g., Staats et al., 2012, Med. Chem. Commun., 2013, 4:88-94; and Ochkur et al., 2012, J. Immunol. Methods, 384: 10-20). In certain aspects, inhibition of eosinophil or mast cell degranulation is at least 10%, 20%, 30%, 40%, 50%, 60%, 70%, 80%, or 90%.

In another particular embodiment, an anti-KIT antibody or an antigen binding fragment thereof provided herein inhibits mast cell mediator release. In certain aspects, mast cell mediator release is at least 10%, 20%, 30%, 40%, 50%, 60%, 70%, 80%, or 90%. Assays to measure mast cell activity, such as release of mediators, from mast cell cultures, such as rodent and human mast cell cultures, have been described (see, e.g., Kuehn et al., “Measuring Mast Cell Mediator Release,” in Current Protocols in Immunology, Unite 7.38.1-7.38.9, November 2010 (John Wiley & Sons, Inc.). In certain aspects, CD34 peripheral blood progenitor cells or a mast cell line, such as HMC-1 or human LAD2 mast cell line can be used in these assays to ascertain the effects of an anti-KIT antibody on mast cells.

In a specific embodiment, an anti-KIT antibody or an antigen binding fragment thereof provided herein induces apoptosis, for example mast cell apoptosis or eosinophil apoptosis. In another specific embodiment, an anti-KIT antibody or an antigen binding fragment thereof provided herein induces cell differentiation, e.g., mast cell differentiation.

In a particular embodiment, an anti-KIT antibody or an antigen binding fragment thereof provided herein can achieve any one of the following: reduction in the number and/or activity of eosinophils, reduction in mast cell proliferation, reduction in plasma tryptase levels, reduction in plasma SCF levels, reduction in mast cell number or amount, inhibition or reduction in mast cell activity, reduction in mast cell induced production or release of inflammatory factors, reduction in release of inflammatory factors, restoration of mast cell homeostasis, reduced mast cell migration, reduced mast cell adhesion, inhibition or reduction in mast cell recruitment of eosinophils, and inhibition or reduction in antigen-mediated degranulation of mast cells.

In a particular embodiment, an anti-KIT antibody or an antigen binding fragment thereof provided herein inhibits KIT activity but does not inhibit KIT dimerization. In another particular embodiment, an anti-KIT antibody or an antigen binding fragment thereof provided herein inhibits KIT activity and does not inhibit ligand binding to KIT, e.g., does not inhibit KIT ligand (e.g., SCF) binding to KIT, but does inhibit KIT dimerization.

In a particular embodiment, an anti-KIT antibody or an antigen binding fragment thereof provided herein inhibits a KIT activity, such as ligand-induced tyrosine phosphorylation of a KIT cytoplasmic domain, by about 25% to about 65% or 75%, as determined by a cell-based phosphorylation assay well known in the art, for example, the cell-based phosphorylation assay described herein. In a certain embodiment, an anti-KIT antibody or an antigen binding fragment thereof provided herein inhibits a KIT activity, such as ligand-induced tyrosine phosphorylation of a KIT cytoplasmic domain, by about 35% to about 85% or 95%, as determined by a cell-based phosphorylation assay well known in the art, for example, the cell-based phosphorylation assay described herein.

In a particular embodiment, an anti-KIT antibody or an antigen binding fragment thereof provided herein inhibits a KIT activity, such as ligand-induced tyrosine phosphorylation of a KIT cytoplasmic domain, with a 50% inhibition concentration (IC₅₀) of less than about 600 pM, or less than about 500 pM, or less than about 250 pM, as determined by a cell-based phosphorylation assay well known in the art, for example, the cell-based phosphorylation assay described herein. In a specific embodiment, the IC₅₀ is less than about 550 pM or 200 pM. In a specific embodiment, the IC₅₀ is in the range of about 50 pM to about 225 pM, or in the range of 100 pM to about 600 pM. In a specific embodiment, the IC₅₀ is in the range of about 50 pM to about 550 pM, or about 50 pM to about 600 pM, or about 150 pM to about 550 pM.

In a specific embodiment, an anti-KIT antibody or an antigen binding fragment thereof provided herein, (i) immunospecifically binds to a KIT polypeptide comprising the D4 and/or D5 region of human KIT, (ii) inhibits KIT phosphorylation (e.g., tyrosine phosphorylation), and (iii) does not fully inhibit, or partially inhibits, KIT ligand (e.g., SCF) binding to KIT. In yet another specific embodiment, such an antibody does not inhibit KIT dimerization. In yet another specific embodiment, such an antibody can be recombinantly expressed by CHO cells at an average titer of at least 0.5 μg/mL, for example at least 1.0 μg/mL. In a further specific embodiment, such an antibody comprises a VH domain and a VL domain that are non-immunogenic, for example, the VH domain and VL domain do not contain T cell epitopes.

In other specific embodiments, an anti-KIT antibody or an antigen binding fragment thereof provided herein immunospecifically binds to a monomeric form of KIT (e.g., human KIT). In particular embodiments, an anti-KIT antibody or an antigen binding fragment thereof provided herein specifically bind to a monomeric form of KIT (e.g., human KIT). In specific embodiments, an anti-KIT antibody or an antigen binding fragment thereof provided herein specifically binds to a dimeric form of KIT (e.g., human KIT).

In specific embodiments, an anti-KIT antibody or an antigen binding fragment thereof provided herein does not bind to a monomeric form of KIT and specifically binds to a dimeric form of KIT or multimeric form of KIT. In certain embodiments, an antibody has higher affinity for a KIT monomer than a KIT dimer. In certain embodiments, an antibody has higher affinity for a KIT monomer than a KIT multimer.

In specific embodiments, an anti-KIT antibody or an antigen binding fragment thereof provided herein specifically binds to a native isoform or native variant of KIT (that is a naturally occurring isoform or variant of KIT in an animal (e.g., monkey, mouse, goat, donkey, dog, cat, rabbit, pig, rat, human, frog, or bird) that can be isolated from an animal, preferably a human). In particular embodiments, an anti-KIT antibody or an antigen binding fragment thereof provided herein specifically binds to human KIT or a fragment thereof. In specific embodiments, an anti-KIT antibody or an antigen binding fragment thereof provided herein specifically binds to human KIT or a fragment thereof and does not specifically bind to a non-human KIT (e.g. monkey, mouse, goat, donkey, dog, cat, rabbit, pig, rat, or bird) or a fragment thereof. In specific embodiments, an anti-KIT antibody or an antigen binding fragment thereof provided herein specifically binds to human KIT or a fragment thereof and does not specifically bind to murine KIT. In certain embodiments, an anti-KIT antibody or an antigen binding fragment thereof provided herein specifically binds to human KIT or a fragment thereof (e.g., a D4 region of human KIT) and to canine (dog) and non-human primate (e.g., monkey) KIT. In certain embodiments, an anti-KIT antibody or an antigen binding fragment thereof provided herein specifically binds to human KIT or a fragment thereof (e.g., a D4 region of human KIT) and to canine (dog) and non-human primate (e.g., monkey) KIT, but does not specifically bind to murine or rat KIT or a fragment thereof (e.g., a D4 region of murine KIT).

In certain embodiments, an anti-KIT antibody or an antigen binding fragment thereof provided herein specifically binds to human KIT or a fragment thereof (e.g. a D4 region of human KIT) and to canine (dog), feline (cat) and cynomologous KIT, but does not specifically bind to murine or rat KIT or a fragment thereof (e.g., a D4 region of murine KIT).

In specific embodiments, an anti-KIT antibody or an antigen binding fragment thereof provided herein specifically binds to human KIT or a fragment thereof (e.g. a D4 region of human KIT), and to canine (dog), feline (cat) and cynomologous KIT, with high affinity (e.g., at least 0.5 fold, 1 fold, 2 fold, 3 fold, 4 fold, 5 fold, or 10 fold) than to murine or rat KIT or a fragment thereof (e.g., a D4 region of murine KIT).

In certain embodiments, an anti-KIT antibody or an antigen binding fragment thereof provided herein specifically binds to an extracellular domain of human KIT comprising a mutation, for example a somatic mutation, such as a mutation in exon 9 of human KIT wherein the Ala and Tyr residues at positions 502 and 503 are duplicated (see, e.g., Marcia et al., (2000) Am. J. Pathol. 156(3):791-795; and Debiec-Rychter et al., (2004) European Journal of Cancer. 40:689-695, which are both incorporated herein by reference in their entireties, describing KIT mutations).

In certain embodiments, an anti-KIT antibody or an antigen binding fragment thereof provided herein specifically binds to an extracellular domain of human KIT which is glycosylated. In certain embodiments, an antibody described herein or antigen-binding fragment thereof binds to two different glycosylated forms of an extracellular domain of human KIT. For example, two forms of human KIT with different molecular weights, indicating different glycosylation patterns, have been observed by immunoblotting.

In certain embodiments, an antibody described herein may specifically bind to both of these forms of human KIT which have different glycosylation patterns, e.g., one form is more glycosylated than the other. In certain embodiments, an antibody described herein or antigen-binding fragment thereof binds to an extracellular domain of human KIT which is not glycosylated.

In a specific embodiment, an anti-KIT antibody or antigen binding fragment thereof provided herein is a bivalent monospecific antibody, in that it has two antigen binding regions (e.g., two identical antigen binding regions) and both antigen binding regions specifically bind the same antigen, KIT (e.g., human KIT). In certain embodiments, the antigen binding region comprises the VH and VL CDRs as set forth in Table 1. In particular embodiments, the antigen binding region comprises a VH comprising the amino acid sequence of any one of SEQ ID NOs: 8-12, and/or a VL comprising the amino acid sequence of any one of SEQ ID NOs: 13-16. In certain aspects, an anti-KIT antibody or antigen binding fragment thereof provided herein is not a bispecific antibody.

In a specific embodiment, antibodies described herein are monoclonal antibodies or isolated monoclonal antibodies. In another specific embodiment, an antibody described herein is a humanized monoclonal antibody. In a particular embodiment, an antibody described herein is a recombinant antibody, for example, a recombinant human antibody, recombinant humanized antibody or a recombinant monoclonal antibody. In certain embodiments, an antibody described herein contains non-human amino acid sequences, e.g., non-human CDRs or non-human (e.g., non-human primate) framework residues.

In particular embodiments provided herein, recombinant antibodies can be isolated, prepared, expressed, or created by recombinant means, such as antibodies expressed using a recombinant expression vector transfected into a host cell, antibodies isolated from a recombinant, combinatorial antibody library, or antibodies prepared, expressed, created or isolated by any other means that involves creation, e.g., via synthesis, genetic engineering of DNA sequences that encode human immunoglobulin sequences, or splicing of sequences that encode human immunoglobulins, e.g., human immunoglobulin gene sequences, to other such sequences. In certain embodiments, the amino acid sequences of such recombinant antibodies have been modified such thus the amino acid sequences of such antibodies, e.g., VH and/or VL regions, are sequences that do not naturally exist within an organism's antibody germline repertoire in vivo, for example a murine or human germline repertoire. In a particular embodiment, a recombinant antibody can be obtained by assembling several sequence fragments that naturally exist in an organism (e.g., primate, such as human) into a composite sequence of a recombinant antibody, wherein the composite sequence does not naturally exist within an organism (e.g., primate such as human).

Antibodies provided herein include immunoglobulin molecules of any type (e.g., IgG, IgE, IgM, IgD, IgA and IgY), class (e.g., IgG1, IgG2, IgG3, IgG4, IgA1 and IgA2) or subclass of immunoglobulin molecule. In a specific embodiment, an antibody provided herein is an IgG antibody (e.g., human IgG antibody), or a class (e.g., human IgG1 or IgG4) or subclass thereof. In another specific embodiment, an antibody described herein is an IgG1 (e.g., human IgG1 (isotype a, z, or f)) or IgG4 antibody. In certain embodiments, an antibody described herein is a whole or entire antibody, e.g., a whole or entire humanized, human, or composite human antibody.

In specific aspects, the antibody provided herein comprises an antibody light chain and heavy chain, e.g., a separate light chain and heavy chain. With respect to the light chain, in a specific embodiment, the light chain of an antibody described herein is a kappa light chain. In another specific embodiment, the light chain of an antibody described herein is a lambda light chain. In yet another specific embodiment, the light chain of an antibody described herein is a human kappa light chain or a human lambda light chain. In a particular embodiment, an antibody described herein comprises a human light chain constant region. Non-limiting examples of human light chain constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242.

With respect to the heavy chain, in a specific embodiment, the heavy chain of an antibody described herein can be an alpha (a), delta (δ), epsilon (ε), gamma (γ) or mu (p) heavy chain. In another specific embodiment, the heavy chain of an antibody described can comprise a human alpha (a), delta (δ), epsilon (ε), gamma (γ) or mu (p) heavy chain. In a particular embodiment, an antibody described herein comprises a human heavy chain constant region (e.g., a human IgG constant region, for example, a human IgG1, IgG2, IgG3, or IgG4 constant region). Non-limiting examples of human heavy chain constant region sequences have been described in the art, e.g., see U.S. Pat. No. 5,693,780 and Kabat et al. (1991) Sequences of Proteins of Immunological Interest, Fifth Edition, U.S. Department of Health and Human Services, NIH Publication No. 91-3242. In a specific embodiment, the antibody described herein comprises a modified (e.g., mutated) human Fc region or domain (e.g., a modified (e.g., mutated) human IgG1 Fc region or domain, a modified (e.g., mutated) human IgG2 Fc region or domain, a modified (e.g., mutated) human IgG3 Fc region or domain, or a modified (e.g., mutated) human IgG4 Fc region or domain).

In certain embodiments, anti-KIT antibodies described herein are human, composite human, or humanized monoclonal antibodies. In a particular embodiment, an antibody described herein is an engineered antibody, for example, antibody produced by recombinant methods. In a specific embodiment, an antibody described herein is a humanized antibody comprising one or more non-human (e.g. rodent or murine) CDRs and one or more human framework regions (FR), and optionally human heavy chain constant region and/or light chain constant region. In a specific embodiment, an antibody described herein comprises one or more primate (or non-human primate) framework regions. In a specific embodiment, an antibody described herein does not comprise non-human primate framework regions.

Antibodies provided herein can include antibodies comprising chemical modifications, for example, antibodies which have been chemically modified, e.g., by covalent attachment of any type of molecule to the antibody. For example, but not by way of limitation, an anti-KIT antibody can be glycosylated, acetylated, pegylated, phosphorylated, or amidated, can be derivitized via protective/blocking groups, or can further comprise a cellular ligand and or other protein or peptide (e.g., a heterologous protein or peptide), etc. For example, an antibody provided herein can be chemically modified, e.g., by glycosylation, acetylation, pegylation, phosphorylation, amidation, derivatization by known protecting/blocking groups, proteolytic cleavage, linkage to a cellular ligand or other protein, etc. Further, an anti-KIT antibody described herein can contain one or more non-classical amino acids.

In one embodiment, an anti-KIT antibody provided herein is a naked antibody which is not linked, fused or conjugated (e.g. artificially linked, fused or conjugated) to another molecule, peptide or polypeptide (for example, a heterologous polypeptide). In a particular embodiment, an anti-KIT antibody provided herein is not an antibody-drug conjugate. In a particular embodiment, an anti-KIT antibody provided herein is not a fusion protein. In particular embodiments, an anti-KIT antibody described herein does not comprise any non-classical amino acids.

### 5.1.1 Antibody Conjugates

In some embodiments, provided herein are antibodies (e.g., human or humanized antibodies), or antigen-binding fragments thereof, conjugated or recombinantly fused to a diagnostic, detectable or therapeutic agent or any other molecule. The conjugated or recombinantly fused antibodies can be useful, e.g., for monitoring or prognosing the onset, development, progression and/or severity of a KIT-associated disorder or disease, for example, as part of a clinical testing procedure, such as determining the efficacy of a particular therapy. The conjugated or recombinantly fused antibodies can be useful, e.g., for protecting against, treating or managing a KIT-associated disorder, or for protecting against, treating or managing effects of a KIT-associated disorder. Antibodies described herein can also be conjugated to a molecule (e.g., polyethylene glycol) which can affect one or more biological and/or molecular properties of the antibodies, for example, stability (e.g., in serum), half-life, solubility, and antigenicity.

In a particular aspect, provided herein is a conjugate comprising an agent (e.g., therapeutic agent) linked to an antibody described herein (or an antigen-binding fragment thereof). In specific embodiments, a conjugate comprises an antibody described herein and a molecule (e.g., therapeutic or drug moiety), wherein the antibody is linked directly to the molecule, or by way of one or more linkers. In certain embodiments, an antibody is covalently conjugated to a molecule. In a particular embodiment, an antibody is noncovalently conjugated to a molecule. In specific embodiments, an antibody described herein, e.g., an antibody conjugated to an agent, binds to wild-type human KIT. In certain embodiments, an antibody described herein, e.g., antibody conjugated to an agent, binds to an extracellular domain of human KIT comprising a mutation, for example a somatic mutation associated with cancer (e.g., GIST), such as a mutation in exon 9 of human KIT wherein the Ala and Tyr residues at positions 502 and 503 are duplicated.

Such diagnosis and detection can be accomplished, for example, by coupling the antibody to detectable molecules or substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I,), carbon (14C), sulfur (³⁵S), tritium (³H), indium (¹¹⁵In, ¹¹³In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F), ¹⁵³Sm, ¹⁷⁷Lu, ¹⁵⁹Gd, ¹⁴⁹Pm, ¹⁴⁰La, ¹⁷⁵Yb, 166Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge ⁵⁷Co, ⁶⁵Zn, ⁸⁵Sr, ³²P, ¹⁵³Gd, ¹⁶⁹Yb, ⁵¹Cr, ⁵⁴Mn, ⁷⁵Se, ¹¹³Sn, and ¹¹⁷Sn; and positron emitting metals using various positron emission tomographies, and non-radioactive paramagnetic metal ions.

Provided are antibodies described herein, or antigen-binding fragments thereof, conjugated or recombinantly fused to a therapeutic moiety (or one or more therapeutic moieties) and uses of such antibodies. The antibody can be conjugated or recombinantly fused to a therapeutic moiety, such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells. Therapeutic moieties include, but are not limited to, auristatin or a derivative thereof, e.g., monomethyl auristatin E (MMAE), monomethyl auristatin F (MMAF), auristatin PYE, and auristatin E (AE) (see, e.g., U.S. Pat. No. 7,662,387 and U.S. Pat. Application Publication Nos. 2008/0300192 and 2008/0025989, each of which is incorporated herein by reference); a microtubule-disrupting agent, e.g., maytansine or a derivative thereof, e.g., maytansinoid DM1 (see, e.g., U.S. Pat. Nos. 7,851,432, 7,575,748, and 5,416,064, each of which is incorporated herein by reference); a prodrug, e.g., a prodrug of a CC-1065 (rachelmycin) analogue; antimetabolites (e.g., methotrexate, 6-mercaptopurine, 6-thioguanine, cytarabine, 5-fluorouracil decarbazine); alkylating agents (e.g., mechlorethamine, thioepa chlorambucil, melphalan, carmustine (BCNU) and lomustine (CCNU), cyclothosphamide, busulfan, dibromomannitol, streptozotocin, mitomycin C, and cisdichlorodiamine platinum (II) (DDP), and cisplatin); minor-groove-binding alkylating agent; anthracyclines (e.g., daunorubicin (formerly daunomycin) and doxorubicin); antibiotics (e.g., d actinomycin (formerly actinomycin), bleomycin, mithramycin, and anthramycin (AMC)); Auristatin molecules (e.g., auristatin PHE, bryostatin 1, and solastatin 10; see Woyke et al., Antimicrob. Agents Chemother. 46:3802-8 (2002), Woyke et al., Antimicrob. Agents Chemother. 45:3580-4 (2001), Mohammad et al., Anticancer Drugs 12:735-40 (2001), Wall et al., Biochem. Biophys. Res. Commun. 266:76-80 (1999), Mohammad et al., Int. J. Oncol. 15:367-72 (1999), all of which are incorporated herein by reference); hormones (e.g., glucocorticoids, progestins, androgens, and estrogens), DNA-repair enzyme inhibitors (e.g., etoposide or topotecan), kinase inhibitors (e.g., compound ST1571, imatinib mesylate (Kantarjian et al., Clin Cancer Res. 8(7):2167-76 (2002)); cytotoxic agents (e.g., paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucorticoids, procaine, tetracaine, lidocaine, propranolol, and puromycin and analogs or homologs thereof and those compounds disclosed in U.S. Pat. Nos. 6,245,759, 6,399,633, 6,383,790, 6,335,156, 6,271,242, 6,242,196, 6,218,410, 6,218,372, 6,057,300, 6,034,053, 5,985,877, 5,958,769, 5,925,376, 5,922,844, 5,911,995, 5,872,223, 5,863,904, 5,840,745, 5,728,868, 5,648,239, 5,587,459, each of which is incorporated herein by reference with respect to such compound disclosure); farnesyl transferase inhibitors (e.g., R115777, BMS-214662, and those disclosed by, for example, U.S. Pat. Nos. 6,458,935, 6,451,812, 6,440,974, 6,436,960, 6,432,959, 6,420,387, 6,414,145, 6,410,541, 6,410,539, 6,403,581, 6,399,615, 6,387,905, 6,372,747, 6,369,034, 6,362,188, 6,342,765, 6,342,487, 6,300,501, 6,268,363, 6,265,422, 6,248,756, 6,239,140, 6,232,338, 6,228,865, 6,228,856, 6,225,322, 6,218,406, 6,211,193, 6,187,786, 6,169,096, 6,159,984, 6,143,766, 6,133,303, 6,127,366, 6,124,465, 6,124,295, 6,103,723, 6,093,737, 6,090,948, 6,080,870, 6,077,853, 6,071,935, 6,066,738, 6,063,930, 6,054,466, 6,051,582, 6,051,574, and 6,040,305, each of which is incorporated herein by reference with respect to such inhibitor disclosure); topoisomerase inhibitors (e.g., camptothecin; irinotecan; SN-38; topotecan; 9-aminocamptothecin; GG-211 (GI 147211); DX-8951f, IST-622; rubitecan; pyrazoloacridine; XR-5000; saintopin; UCE6; UCE1022; TAN-1518A; TAN 1518B; KT6006; KT6528; ED-110; NB-506; ED-110; NB-506; and rebeccamycin); bulgarein; DNA minor groove binders such as Hoescht dye 33342 and Hoechst dye 33258; nitidine; fagaronine; epiberberine; coralyne; beta-lapachone; BC-4-1; bisphosphonates (e.g., alendronate, cimadronte, clodronate, tiludronate, etidronate, ibandronate, neridronate, olpandronate, risedronate, piridronate, pamidronate, zolendronate) HMG-CoA reductase inhibitors, (e.g., lovastatin, simvastatin, atorvastatin, pravastatin, fluvastatin, statin, cerivastatin, lescol, lupitor, rosuvastatin and atorvastatin); antisense oligonucleotides (e.g., those disclosed in the U.S. Pat. Nos. 6,277,832, 5,998,596, 5,885,834, 5,734,033, and 5,618,709, each of which is incorporated herein by reference with respect to such oligonucleotides); adenosine deaminase inhibitors (e.g., Fludarabine phosphate and 2-Chlorodeoxyadenosine); ibritumomab tiuxetan (Zevalin®); tositumomab (Bexxar®)) and pharmaceutically acceptable salts, solvates, clathrates, and prodrugs thereof.

In particular embodiments, a therapeutic moiety or drug moiety is an antitubulin drug, such as an auristatin or a derivative thereof. Non-limiting examples of auristatins include monomethyl auristatin E (MMAE), monomethyl auristatin F (MMAF), auristatin PYE, and auristatin E (AE) (see, e.g., U.S. Pat. No. 7,662,387 and U.S. Pat. Application Publication Nos. 2008/0300192 and 2008/0025989, each of which is incorporated herein by reference). In certain embodiments, a therapeutic moiety or drug moiety is a microtubule-disrupting agent such as maytansine or a derivative thereof, e.g., maytansinoid DM1 or DM4 (see, e.g., U.S. Pat. Nos. 7,851,432, 7,575,748, and 5,416,064, each of which is incorporated herein by reference). In certain embodiments, a therapeutic moiety or drug moiety is a prodrug, e.g., a prodrug of a CC-1065 (rachelmycin) analogue (see, e.g., U.S. Patent Application Publication No. 2008/0279868, and PCT International Patent Application Publication Nos. WO 2009/017394, WO 2010/062171, and WO 2007/089149, each of which is incorporated herein by reference).

In a specific embodiment, the antibody and therapeutic/drug agent are conjugated by way of one or more linkers. In another specific embodiment, the antibody and therapeutic/drug agent are conjugated directly.

In specific embodiments, non-limiting examples of therapeutic moieties or drug moieties for conjugation to an antibody described herein include calicheamicins (e.g., LL-E33288 complex, for example, gamma-calicheamicin, see, e.g., U.S. Pat. No. 4,970,198) and derivatives thereof (e.g., gamma calicheamicin hydrazide derivatives), ozogamicins, duocarmycins and derivatives thereof (e.g., CC-1065 (NSC 298223), or an achiral analogue of duocarmycin (for example AS-1-145 or centanamycin)), taxanes and derivatives thereof, and enediynes and derivatives thereof (See, e.g., PCT International Patent Application Publication Nos. WO 2009/017394, WO 2010/062171, WO 2007/089149, WO 2011/021146, WO 2008/150261, WO 2006/031653, WO 2005/089809, WO 2005/089807, and WO 2005/089808, each of which is incorporated by reference herein in its entirety).

Non-limiting examples of calicheamicins suitable for conjugation to an antibody described herein are disclosed, for example, in U.S. Pat. Nos. 4,671,958; 5,053,394; 5,037,651; 5,079,233; and 5,108,912; and PCT International Patent Application Publication Nos. WO 2011/021146, WO 2008/150261, WO 2006/031653, WO 2005/089809, WO 2005/089807, and WO 2005/089808; each of which is incorporated herein by reference for such calicheamcin disclosure. In particular embodiments, these compounds may contain a methyltrisulfide that reacts with appropriate thiols to form disulfides, and at the same time introduces a functional group such as a hydrazide or other functional group that may be useful for conjugating calicheamicin to an antibody described herein. In certain embodiments, stabilizing the disulfide bond that is present in calicheamicin conjugates by adding dimethyl substituents may yield an improved antibody/drug conjugate. In specific embodiments, the calicheamicin derivative is N-acetyl gamma calicheamicin dimethyl hydrazide, or NAc-gamma DMH (CL-184,538), as one of the optimized derivatives for conjugation. Disulfide analogs of calicheamicin which can be conjugated to an antibody described herein are described, for example, in U.S. Pat. Nos. 5,606,040 and 5,770,710, each of which is incorporated herein by reference for such compound disclosure. In a certain embodiment, a moiety (e.g., calicheamicin or a derivative thereof) is conjugated to an antibody by a linker. In a particular embodiment, a moiety (e.g., calicheamicin or a derivative thereof) is hydrolyzed from the antibody-drug conjugate at the linker. In one embodiment, a moiety (e.g., calicheamicin or a derivative thereof) is hydrolyzed from an antibody conjugate at the linker between about a pH of 3.0 and pH 4.0 for 1-24 hours at a temperature from 20 to 50° C., preferably 37° C.

In specific embodiments, non-limiting examples of therapeutic moieties or drug moieties for conjugation to an antibody described herein include pyrrolobenzodiazepines (PBDs) and derivatives thereof, for example, PBD dimers (e.g., SJG-136 or SG2000), C2-unsaturated PBD dimers, pyrrolobenzodiazepine dimers bearing C2 aryl substitutions (e.g., SG2285), PBD dimer pro-drug that is activated by hydrolysis (e.g., SG2285), and polypyrrole-PBD (e.g., SG2274) (see, e.g., PCT International Patent Application Publication Nos. WO 2000/012507, WO 2007/039752, WO 2005/110423, WO 2005/085251, and WO 2005/040170, and U.S. Pat. No. 7,612,062, each of which is incorporated herein by reference for such compound disclosure).

In addition, an antibody described herein can be conjugated to therapeutic moieties such as a radioactive metal ion, such as alpha-emitters such as ²¹³Bi or macrocyclic chelators useful for conjugating radiometal ions, including but not limited to, ¹³¹In, ¹³¹LU, ¹³¹Y, ¹³¹Ho, ¹³¹Sm, to polypeptides. In certain embodiments, the macrocyclic chelator is 1,4,7,10-tetraazacyclododecane-N,N′,N″,N″′-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

In certain embodiments, an antibody described herein, or an antigen-binding fragment thereof, is conjugated to one or more molecules (e.g., therapeutic or drug moiety) directly or indirectly via one or more linker molecules. In particular embodiments, a linker is an enzyme-cleavable linker or a disulfide linker. In a specific embodiment, the cleavable linker is cleavable via an enzyme such an aminopeptidase, an aminoesterase, a dipeptidyl carboxy peptidase, or a protease of the blood clotting cascade. In particular embodiments, a linker comprises 1, 2, 3, 4, 5, 6, 7, 8, 9, 10, 11, 12, 13, 14, 15, or 20 amino acid residues. In certain embodiments, a linker consists of 1 to 10 amino acid residues, 1 to 15 amino acid residues, 5 to 20 amino acid residues, 10 to 25 amino acid residues, 10 to 30 amino acid residues, or 10 to 50 amino acid residues.

In certain embodiments, a moiety is conjugated to an antibody by one or more linkers. In a particular embodiment, a moiety is hydrolyzed from the antibody-drug conjugate at the linker. In one embodiment, a moiety is hydrolyzed from the antibody conjugate at the linker between about a pH of 3.0 and pH 4.0 for about 1-24 hours, and at a temperature from about 20 to 50° C., preferably 37° C. In a specific embodiment, a linker is stable in the blood stream but releases the conjugated moiety once it is inside the targeted cells. In certain embodiments, a moiety is conjugated to an antibody described herein via one or more triazole-containing linkers (see, e.g., International Patent Application Publication No. WO 2007/018431, which is incorporated herein by reference). Non-limiting examples of linkers and spacers for incorporation into antibody-drug conjugates described herein are disclosed in PCT International Patent Application Publication Nos. WO 2007/018431, WO 2004/043493, and WO 2002/083180.

Moreover, antibodies described herein can be fused to marker sequences, such as a peptide to facilitate purification. In preferred embodiments, the marker amino acid sequence is a hexa-histidine peptide, such as the tag provided in a pQE vector (QIAGEN, Inc.), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “FLAG” tag.

Methods for fusing or conjugating therapeutic moieties (including polypeptides) to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), Thorpe et al., 1982, Immunol. Rev. 62:119-58; U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., Proc. Natl. Acad. Sci. USA, 88: 10535-10539, 1991; Traunecker et al., Nature, 331:84-86, 1988; Zheng et al., J. Immunol., 154:5590-5600, 1995; Vil et al., Proc. Natl. Acad. Sci. USA, 89:11337-11341, 1992, which are incorporated herein by reference in their entireties.

Antibodies described herein can also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

In a certain aspect, an antibody described herein or an antigen-binding fragment thereof is an extracellular drug conjugate (ECD) comprising an antibody linked to a drug, optionally by a linker (see, e.g., PCT International Patent Application Publication No. WO 2011/031870). The drug can act outside of the cell, and thus internalization of the conjugate is not required. After an ECD binds a target cell, the drug sends a signal into the cell.

In one embodiment, the linker of the ECD is a non-cleavable linker. Examples of non-cleavable linkers include linkers that contain polyethylene glycol chains or polyethylene chains that are not acid or base sensitive (such as hydrazone containing linkers), are not sensitive to reducing or oxidizing agents (such as those containing disulfide linkages), and are not sensitive to enzymes that may be found in cells or circulatory system. Specific examples of non-cleavable linkers include SMCC linker (US Patent Application 20090202536). For illustrative purposes, examples of cleavable linkers include linkers that contain non-hindered glutathione sensitive disulfides, esters, peptide sequences sensitive to the peptidases such as cathepsin or plasmin, pH sensitive hydrazones (see Bioconjugate Chem., 2010, 21 (1), pp 5-13) and non-hindered disulfide linker SPP (US Patent Application 20090202536).

In certain aspects, an ECD comprises a drug or agent that is a cardiac glycoside, for example, proscillaridin or a sugar-enhanced proscillaridin. In one embodiment, the agent is composed from a cardiac glycoside which is void a sugar. In various embodiments, the cardiac glycoside is a compound identified in PCT Pub. No. WO 2010/017480 (PCT/US2009/053159).

### 5.2 Polynucleotides

In certain aspects, provided herein are polynucleotides and combination of polynucleotides comprising a nucleotide sequence(s) encoding an antibody (e.g., human or humanized antibody) described herein or a fragment thereof (e.g., a variable light chain region and/or variable heavy chain region) that immunospecifically binds to a KIT antigen. Also provided herein are polynucleotides encoding KIT antigens for generating anti-KIT antibodies described herein.

As used herein, an “isolated” polynucleotide or nucleic acid molecule is one which is separated from other nucleic acid molecules which are present in the natural source (e.g., in a human) of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1% (in particular less than about 10%) of other material, e.g., cellular material, culture medium, other nucleic acid molecules, chemical precursors and/or other chemicals. In a specific embodiment, a nucleic acid molecule(s) encoding an antibody described herein is isolated or purified.

In particular aspects, provided herein is a polynucleotide or a combination of polynucleotides comprising nucleotide sequences encoding an antibody or antigen binding fragment thereof described herein, or the VH and VL of said antibody or antigen binding fragment thereof. In a specific embodiment, provided herein is a polynucleotide comprising a nucleotide sequence encoding the VH of an antibody or antigen binding fragment thereof described herein. In a specific embodiment, provided herein is a polynucleotide comprising a nucleotide sequence encoding the VL of an antibody or antigen binding fragment thereof described herein. In a specific embodiment, provided herein is a polynucleotide comprising a first nucleotide sequence encoding the VH of an antibody or antigen binding fragment thereof described herein and a second nucleotide sequence encoding the VL of said antibody or antigen binding fragment. In a specific embodiment, provided herein is a combination of two polynucleotides, wherein the first polynucleotide of the combination comprises a first nucleotide sequence encoding the VH of an antibody or antigen binding fragment thereof described herein and the second polynucleotide of the combination comprises a second nucleotide sequence encoding the VL of said antibody or antigen binding fragment. In a specific embodiment, provided herein is a polynucleotide comprising a nucleotide sequence encoding the heavy chain of an antibody described herein. In a specific embodiment, provided herein is a polynucleotide comprising a nucleotide sequence encoding the light chain of an antibody described herein. In a specific embodiment, provided herein is a polynucleotide comprising a first nucleotide sequence encoding the heavy chain of an antibody described herein and a second nucleotide sequence encoding the light chain of said antibody. In a specific embodiment, provided herein is a combination of two polynucleotides, wherein the first polynucleotide of the combination comprises a first nucleotide sequence encoding the heavy chain of an antibody described herein and the second polynucleotide of the combination comprises a second nucleotide sequence encoding the light chain of said antibody.

In a specific embodiment, the polynucleotide provided herein comprises the nucleotide sequence of SEQ ID NO: 23. In a specific embodiment, the polynucleotide provided herein comprises the nucleotide sequence of SEQ ID NO: 24. In a specific embodiment, the polynucleotide provided herein comprises a nucleotide sequence of SEQ ID NO: 23 and a nucleotide sequence of SEQ ID NO: 24. In a specific embodiment, the combination of polynucleotides provided herein comprises a first polynucleotide comprising a nucleotide sequence of SEQ ID NO: 23 and a second polynucleotide comprising a nucleotide sequence of SEQ ID NO: 24.

Also provided herein are polynucleotides encoding an anti-KIT antibody or a fragment thereof that are optimized, e.g., by codon/RNA optimization, replacement with heterologous signal sequences, and elimination of mRNA instability elements. Methods to generate optimized nucleic acids encoding an anti-KIT antibody or a fragment thereof (e.g., light chain, heavy chain, VH domain, or VL domain) for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In some embodiments, it can be desirable to alter one or more codons to encode a conservative mutation, e.g., a similar amino acid with similar chemical structure and properties and/or function as the original amino acid. Such methods can increase expression of an anti-KIT antibody or fragment thereof by at least 1 fold, 2 fold, 3 fold, 4 fold, 5 fold, 10 fold, 20 fold, 30 fold, 40 fold, 50 fold, 60 fold, 70 fold, 80 fold, 90 fold, or 100 fold or more relative to the expression of an anti-KIT antibody encoded by polynucleotides that have not been optimized.

In certain embodiments, an optimized polynucleotide sequence encoding an anti-KIT antibody described herein or a fragment thereof (e.g., VL domain and/or VH domain) can hybridize to an antisense (e.g., complementary) polynucleotide of an unoptimized polynucleotide sequence encoding an anti-KIT antibody described herein or a fragment thereof (e.g., VL domain and/or VH domain). In specific embodiments, an optimized nucleotide sequence encoding an anti-KIT antibody described herein or a fragment hybridizes under high stringency conditions to antisense polynucleotide of an unoptimized polynucleotide sequence encoding an anti-KIT antibody described herein or a fragment thereof. In a specific embodiment, an optimized nucleotide sequence encoding an anti-KIT antibody described herein or a fragment thereof hybridizes under high stringency, intermediate or lower stringency hybridization conditions to an antisense polynucleotide of an unoptimized nucleotide sequence encoding an anti-KIT antibody described herein or a fragment thereof. Information regarding hybridization conditions have been described, see, e.g., U.S. Patent Application Publication No. US 2005/0048549 (e.g., paragraphs 72-73), which is incorporated herein by reference.

In certain embodiments, an optimized polynucleotide sequence encoding a VL region of an antibody described herein is at least 75%, at least 80%, at least 85%, at least 90%, at least 95%, at least 98% identical to the nucleotide sequence of SEQ ID NO: 23. In certain embodiments, an optimized polynucleotide sequence encoding a VH region of an antibody described herein is at least 75%, at least 80%, at least 85%, at least 90%, at least 95%, at least 98% identical to the nucleotide sequence of SEQ ID NO: 24.

The polynucleotides can be obtained, and the nucleotide sequence of the polynucleotides determined, by any method known in the art. Nucleotide sequences encoding antibodies described herein and modified versions of these antibodies can be determined using methods well known in the art, i.e., nucleotide codons known to encode particular amino acids are assembled in such a way to generate a nucleic acid that encodes the antibody. Such a polynucleotide encoding the antibody can be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the antibody of interest. Such PCR amplification methods can be used to obtain nucleic acids comprising the sequence encoding the light chain and/or heavy chain of an antibody. Such PCR amplification methods can be used to obtain nucleic acids comprising the sequence encoding the variable light chain region and/or the variable heavy chain region of an antibody. The amplified nucleic acids can be cloned into vectors for expression in host cells and for further cloning, for example, to generate chimeric and humanized antibodies.

If a clone containing a nucleic acid encoding a particular antibody is not available, but the sequence of the antibody molecule is known, a nucleic acid encoding the immunoglobulin can be chemically synthesized or obtained from a suitable source (e.g., an antibody cDNA library or a cDNA library generated from, or nucleic acid, preferably poly A+RNA, isolated from, any tissue or cells expressing the antibody, such as hybridoma cells selected to express an antibody described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR can then be cloned into replicable cloning vectors using any method well known in the art.

DNA encoding anti-KIT antibodies described herein can be readily isolated and sequenced using conventional procedures (e.g., by using oligonucleotide probes that are capable of binding specifically to genes encoding the heavy and light chains of the anti-KIT antibodies). Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce immunoglobulin protein, to obtain the synthesis of anti-KIT antibodies in the recombinant host cells.

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a heavy chain constant region, e.g., the human gamma 1 or gamma 4 constant region, and the PCR amplified VL domains can be cloned into vectors expressing a light chain constant region, e.g., human kappa or lambda constant regions. In certain embodiments, the vectors for expressing the VH or VL domains comprise an EF-1α promoter, a secretion signal, a cloning site for the variable domain, constant domains, and a selection marker such as neomycin. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

The DNA also can be modified, for example, by substituting the coding sequence for human heavy and light chain constant domains in place of the murine sequences, or by covalently joining to the immunoglobulin coding sequence all or part of the coding sequence for a non-immunoglobulin polypeptide.

### 5.3 Host Cells and Recombinant Expression of Antibodies

In certain aspects, provided herein are host cells recombinantly expressing the antibodies described herein (or an antigen-binding fragment thereof) and related expression vectors. Provided herein are vectors and combination of vectors (e.g., expression vectors) comprising polynucleotides comprising nucleotide sequences encoding anti-KIT antibodies or an antigen-binding fragment for recombinant expression in host cells, preferably in mammalian cells. Also provided herein are host cells comprising such vectors or combination of vectors for recombinantly expressing anti-KIT antibodies described herein (e.g., human or humanized antibody).

Recombinant expression of an antibody described herein (e.g., a full-length antibody, heavy and/or light chain of an antibody, or a single chain antibody described herein) that immunospecifically binds to a KIT antigen involves construction of an expression vector(s) containing a polynucleotide(s) that encode the antibody. Once a polynucleotide encoding an antibody molecule, heavy and/or light chain of an antibody, or fragment thereof (preferably, but not necessarily, containing the heavy and/or light chain variable domain) described herein has been obtained, the vector(s) for the production of the antibody molecule can be produced by recombinant DNA technology using techniques well-known in the art. Thus, methods for preparing a protein by expressing a polynucleotide containing an antibody (or VH/VL or heavy/light chain) encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see, e.g., International Publication Nos. WO 86/05807 and WO 89/01036; and U.S. Pat. No. 5,122,464) and the variable domain of the antibody can be cloned into such a vector for expression of the entire heavy, the entire light chain, or both the entire heavy and light chains.

In a specific embodiment, provided herein is a vector comprising a polynucleotide encoding a VH of an antibody described herein or its antigen binding fragment. In a specific embodiment, provided herein is a vector comprising a polynucleotide encoding a VL of an antibody described herein or its antigen binding fragment. In a specific embodiment, provided herein is a vector comprising a polynucleotide encoding a VH and a VL of an antibody described herein or its antigen binding fragment. In a specific embodiment, provided herein is a vector comprising a first polynucleotide encoding a VH of an antibody described herein or its antigen binding fragment and a second polynucleotide encoding the VL of the antibody or antigen binding fragment. In a specific embodiment, provided herein is a combination of two vectors, wherein the first vector of the combination comprises a first polynucleotide encoding the VH of an antibody described herein or its antigen binding fragment, and the second vector of the combination comprises a second polynucleotide encoding the VL of the antibody or antigen binding fragment. In a specific embodiment, provided herein is a vector comprising a polynucleotide encoding a heavy chain of an antibody described herein. In a specific embodiment, provided herein is a vector comprising a polynucleotide encoding a light chain of an antibody described herein. In a specific embodiment, provided herein is a vector comprising a polynucleotide encoding a heavy chain and a light chain of an antibody described herein. In a specific embodiment, provided herein is a vector comprising a first polynucleotide encoding a VH of an antibody described herein and a second polynucleotide encoding the VL of the antibody. In a specific embodiment, provided herein is a combination of two vectors, wherein the first vector of the combination comprises a first polynucleotide encoding the VH of an antibody described herein, and the second vector of the combination comprises a second polynucleotide encoding the VL of the antibody.

An expression vector or a combination of expression vectors can be transferred to a cell (e.g., host cell) by conventional techniques and the resulting cells can then be cultured by conventional techniques to produce an antibody described herein or a fragment thereof. Thus, provided herein are host cells containing a polynucleotide or a combination of polynucleotides encoding an antibody described herein or fragments thereof, or a heavy or light chain thereof, or fragment thereof, or a single chain antibody described herein, operably linked to a promoter for expression of such sequences in the host cell. In certain embodiments, for the expression of double-chained antibodies, vectors encoding both the heavy and light chains, individually, can be co-expressed in the host cell for expression of the entire immunoglobulin molecule, as detailed below. In certain embodiments, a host cell contains a vector comprising a polynucleotide encoding both the heavy chain and light chain (or both the VH and VL) of an antibody described herein, or a fragment thereof. In specific embodiments, a host cell contains two different vectors, a first vector comprising a polynucleotide encoding a heavy chain (or VH) of an antibody described herein, or a fragment thereof, and a second vector comprising a polynucleotide encoding a light chain (or VL) of an antibody described herein, or a fragment thereof. In other embodiments, a first host cell comprises a first vector comprising a polynucleotide encoding a heavy chain (or VH) of an antibody described herein, or a fragment thereof, and a second host cell comprises a second vector comprising a polynucleotide encoding a light chain (or VL) of an antibody described herein, or a fragment thereof.

A variety of host-expression vector systems can be utilized to express antibody molecules described herein (see, e.g., U.S. Pat. No. 5,807,715). Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, MDCK, HEK 293, NSO, PER.C6, VERO, CRL7030, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific embodiment, cells for expressing antibodies described herein or an antigen-binding fragment thereof are CHO cells, for example CHO cells from the CHO GS System™ (Lonza). In a specific embodiment, a mammalian expression vector is pOptiVEC™ or pcDNA3.3. Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In certain embodiments, antibodies described herein are produced by CHO cells or NSO cells. In a specific embodiment, the expression of nucleotide sequences encoding antibodies described herein which immunospecifically bind to a KIT antigen is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruther et al., 1983, EMBO 12:1791), in which the antibody coding sequence can be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodopterafrugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al., 1987, Methods in Enzymol. 153:51-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT20 and T47D, NSO (a murine myeloma cell line that does not endogenously produce any immunoglobulin chains), CRL7030 and HsS78Bst cells. In certain embodiments, humanized monoclonal anti-KIT antibodies described herein are produced in mammalian cells, such as CHO cells.

For long-term, high-yield production of recombinant proteins, stable expression is preferred. For example, cell lines which stably express the antibody molecule can be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express the antibody molecule. Such engineered cell lines can be particularly useful in screening and evaluation of compositions that interact directly or indirectly with the antibody molecule.

A number of selection systems can be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIB TECH 11(5):155-2 15); and hygro, which confers resistance to hygromycin (Santerre et al., 1984, Gene 30:147). Methods commonly known in the art of recombinant DNA technology can be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific embodiments, an antibody described herein is isolated or purified. Generally, an isolated antibody is one that is substantially free of other antibodies with different antigenic specificities than the isolated antibody. For example, in a particular embodiment, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (e.g., antibody fragments). When the antibody is recombinantly produced, it is also generally substantially free of culture medium, i.e., culture medium represents less than about 20%, 10%, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In a specific embodiment, antibodies described herein are isolated or purified.

### 5.4 Antibody Production

Antibodies (e.g., human or humanized antibodies) described herein (or an antigen-binding fragment thereof) that immunospecifically bind to a KIT antigen can be produced by any method known in the art for the synthesis of antibodies, for example, by chemical synthesis or by recombinant expression techniques. In a particular aspect, provided herein are methods for making an antibody described herein, comprising culturing, and/or expressing such antibody using, a host cell described herein, which methods optionally further comprise purifying the antibody obtained from the host cell. The methods described herein employs, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual, Cold Spring Harbor Laboratory Press; Sambrook et al. (1989), Molecular Cloning: A Laboratory Manual, Second Edition, Cold Spring Harbor Laboratory Press; Sambrook et al. (2001) Molecular Cloning: A Laboratory Manual, Cold Spring Harbor Laboratory Press, Cold Spring Harbor, NY; Ausubel et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, TRL Press; Eckstein (ed.) (1991) Oligonucleotides and Analogues: A Practical Approach, IRL Press; Birren et al. (eds.) (1999) Genome Analysis: A Laboratory Manual, Cold Spring Harbor Laboratory Press.

For example, humanized antibodies can be produced using a variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(δ):805-814; and Roguska et al., 1994, PNAS 91:969-973), chain shuffling (U.S. Pat. No. 5,565,332), and techniques disclosed in, e.g., U.S. Pat. Nos. 6,407,213, 5,766,886, WO 9317105, Tan et al., J. Immunol. 169: 1119 25 (2002), Caldas et al., Protein Eng. 13(5):353-60 (2000), Morea et al., Methods 20(3):267 79 (2000), Baca et al., J. Biol. Chem.272(16): 10678-84 (1997), Roguska et al., Protein Eng. 9(10):895 904 (1996), Couto et al., Cancer Res. 55 (23 Supp):5973s-5977s (1995), Couto et al., Cancer Res. 55(8): 1717-22 (1995), Sandhu J S, Gene 150(2):409-10 (1994), and Pedersen et al., J. Mol. Biol. 235(3):959-73 (1994). See also U.S. Patent Pub. No. US 2005/0042664 A1 (Feb. 24, 2005), which is incorporated herein by reference.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling et al., in: Monoclonal Antibodies and T-Cell Hybridomas 563 681 (Elsevier, N.Y., 1981). The term “monoclonal antibody” as used herein is not limited to antibodies produced through hybridoma technology. For example, monoclonal antibodies can be produced by recombinant technology, e.g. recombinant monoclonal antibodies expressed by a host cell, such as a mammalian host cell.

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., extracellular domain of human KIT) used for immunization. Alternatively, lymphocytes may be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). Additionally, a RIMMS (repetitive immunization multiple sites) technique can be used to immunize an animal (Kilptrack et al., 1997 Hybridoma 16:381-9, which is incorporated herein by reference).

Non-limiting examples of myeloma cell lines include murine myeloma lines, such as those derived from MOPC-21 and MPC-11 mouse tumors available from the Salk Institute Cell Distribution Center, San Diego, CA, USA, and SP-2 or X63-Ag8.653 cells available from the American Type Culture Collection, Rockville, MD, USA. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, pp. 51-63 (Marcel Dekker, Inc., New York, 1987)).

Antibodies described herein include antibody fragments which recognize specific KIT antigens and can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of an antibody molecule and contains the complete light chain paired with the VH and CHI domains of the heavy chain. A F(ab′)₂ fragment contains the two antigen-binding arms of an antibody molecule linked by disulfide bonds in the hinge region.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

Single domain antibodies, for example, antibodies lacking the light chains, can be produced by methods well-known in the art. See Riechmann et al., 1999, J. Immunol. 231:25-38; Nuttall et al., 2000, Curr. Pharm. Biotechnol. 1 (3):253-263; Muylderman, 2001, J. iotechnol. 74(4):277302; U.S. Pat. No. 6,005,079; and International Publication Nos. WO 94/04678, WO 94/25591, and WO 01/44301.

### 5.5 Methods of Treatment and Medical Uses

Provided herein are methods for impeding, preventing, protecting against, treating and/or managing a KIT-associated disorder or disease. Such methods comprise administering to a subject in need thereof a therapeutically effective amount of an anti-KIT antibody described herein (e.g., humanized antibodies), antigen-binding fragments thereof, conjugates thereof, or a pharmaceutical composition described herein. In certain aspects, also provided herein are methods for preventing, impeding, protecting against, treating or managing one or more symptoms of a KIT-associated disorder or disease.

In specific embodiments, methods described herein for treating a KIT-associated disorder or disease provide for the reduction or amelioration of the progression, severity, and/or duration of a KIT-associated disorder or disease resulting from the administration of one or more therapies (including, but not limited to, the administration of one or more prophylactic or therapeutic agents, such as an anti-KIT antibody described herein). In further specific embodiments, methods described herein for treating a KIT-associated disorder or disease relate to reducing one or more symptoms of a KIT-associated disorder or disease. In specific embodiments, an antibody described herein, or an antigen-binding fragment thereof, or a conjugate thereof, or a pharmaceutical composition described herein, is for use in protecting against, treating or managing a KIT-associated disorder. In a particular embodiment, a KIT-associated disease or disorder being treated or managed or against which is protected with an anti-KIT antibody described herein, or an antigen-binding fragment thereof, or a conjugate thereof, or a pharmaceutical composition described herein, is associated with KIT expression and/or activity, e.g., involves cells expressing KIT and/or exhibiting KIT activity, but is not caused by or the result of KIT expression or activity.

In a specific embodiment, the antibody used in the methods described herein is internalized by the cell to which it binds. In a particular embodiment, a conjugate is used in the methods described herein, wherein the conjugate comprises an antibody described herein (e.g., a humanized anti-KIT antibody), or a KIT-binding fragment thereof. In a specific embodiment, the conjugate comprises an antibody described herein (e.g., a humanized anti-KIT antibody), or a KIT-binding fragment thereof, linked, covalently or non-covalently, to a therapeutic agent, such as a toxin. In a certain embodiment, the conjugate used in the methods described herein is internalized into a cell to which it binds.

In certain embodiments, KIT is aberrantly (e.g., highly) expressed by cells, for example, KIT is overexpressed. In particular embodiments, KIT expression (e.g., on the cell surface) is at least about 10%, 20%, 30%, 40%, 50%, 60%, 70%, 80%, 90%, or 100% higher than KIT expression on the surface of a control cell (e.g., a cell expressing normal levels of KIT, for example, a normal, e.g., human, mast cell, stem cell, brain cell, melanoblast, or ovary cell). In particular embodiments, KIT expression yields at least about 10%, 20%, 30%, 40%, 50%, 60%, 70%, 80%, 90%, or 100% higher cell surface KIT expression than the average KIT expression on the surface of a control cell population (e.g., a cell population expressing normal levels of KIT, for example, a normal, e.g., human, mast cell population, stem cell population, brain cell population, melanoblast population, or ovary cell population). In specific embodiments, such control cells can be obtained or derived from a healthy individual (e.g., healthy human). In some embodiments, KIT can be aberrantly upregulated in a particular cell type, whether or not KIT is aberrantly expressed on the cell surface. In particular embodiments, KIT signaling or activity can be aberrantly upregulated in a particular cell type, whether or not KIT is aberrantly expressed on the cell surface. In particular embodiments, KIT signaling is at least about 10%, 20%, 30%, 40%, 50%, 60%, 70%, 80%, 90%, or 100% higher than KIT signaling of a control cell (e.g., a cell containing normal KIT signaling, for example, a mast cell, stem cell, brain cell, melanoblast, or ovary cell). In particular embodiments, KIT signaling is at least about 10%, 20%, 30%, 40%, 50%, 60%, 70%, 80%, 90%, or 100% higher than average KIT signaling of a control cell population (e.g., a cell population exhibiting normal KIT signaling, for example, a normal, e.g., human, mast cell population, stem cell population, brain cell population, melanoblast population, or ovary cell population). In certain embodiments, normal, aberrant or excessive cell signaling is caused by binding of KIT to a KIT ligand. In other embodiments, aberrant or excessive cell signaling occurs independent of binding of KIT to a KIT ligand.

In certain aspects, a KIT-associated disorder or disease can be characterized by gain-of-function KIT activity, increase in KIT activity, or overexpression of KIT. In one embodiment, a KIT-associated disorder or disease is completely or partially caused by or is the result of gain-of-function KIT activity or expression, e.g., overexpression, of KIT. In certain embodiments, the gain-of-function KIT activity can occur independent of KIT ligand (e.g., SCF) binding KIT receptor. In particular aspects, high or overexpression of KIT in a cell refers to an expression level which is at least about 35%, 45%, 55%, or 65% more than the expression level of a reference cell known to have normal KIT expression or KIT activity or more than the average expression level of KIT in a population of cells or samples known to have normal KIT expression or KIT activity. Expression levels of KIT can be assessed by methods described herein or known to one of skill in the art (e.g., Western blotting or immunohistorychemistry). In particular embodiments, a KIT-associated disorder or disease is characterized by KIT activity which is higher than normal KIT activity and contributes to cellular transformation, neoplasia, and tumorogenesis. In particular aspects, high or increase of KIT activity in a cell refers to a KIT activity level which is at least about 35%, 45%, 55%, or 65% more than the expression level of a reference cell known to have normal KIT activity or more than the average level of KIT activity in a population of cells or samples known to have normal KIT activity. Non-limiting examples of a KIT activity includes tyrosine phosphorylation of the cytoplasmic domain of KIT, and signaling downstream of KIT, such as Stat or Akt signaling.

In certain embodiments, a KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis. In certain embodiments, a KIT-associated disorder is fibrosis or an inflammatory disorder, e.g., inflammatory bowel disease (IBD), such as Crohn's disease (CD) or ulcerative colitis (UC). In other embodiments, a KIT-associated disease is cancer, such as lung cancer (e.g., small cell lung cancer), leukemia, neuroblastoma, melanoma, sarcoma (e.g., Ewing's sarcoma) or gastrointestinal stromal tumor (GIST). In other embodiments, a KIT-associated disease is a systemic mast cell disorder (e.g., mastocytosis), hematologic disorder, fibrosis (e.g., idiopathic pulmonary fibrosis (TPF), scleroderma, or myelofibrosis), or an inflammatory condition such as asthma, rheumatoid arthritis, inflammatory bowel disease, or allergic inflammation.

In a specific embodiment, the KIT-associated disorder is a mast cell related disorder. In a specific embodiment, the KIT-associated disorder is an eosinophil related disorder such as eosinophilic esophagitis (EoE).

Mast cells, derived from bone marrow progenitors, are large cells found in connective tissues throughout the body, most abundantly in the submucosal tissues and the dermis. They contain large granules that store a variety of mediator molecules including the vasoactive amine histamine, and have high-affinity Fes receptors (FcsRI) that allow them to bind IgE monomers. Antigen-binding to IgE bound to mast cells triggers mast-cell degranulation and mast-cell activation, producing a local or systemic immediate hypersensitivity reaction. Therefore, mast cells play important roles in inflammatory and allergic reactions. However, without proper balance and regulation, mast cells can also be responsible for detrimental exaggerated reactions to antigen observed in disorders such as anaphylaxis, atopy, and rhinitis.

KIT signaling is important for mast cell development and homeostasis, for example, expansion of mast cells from their progenitor cells and their subsequent maturation and survival in their resident tissues, homing of mast cells to their sites of residence in vivo, and promoting adhesion of mast cells to extracellular matrix proteins. Activation mutations of KIT, such as at amino acid residue 816 or 560 of KIT, have been associated with mastocytosis, characterized by overproduction of mast cells, and gastrointestinal stromal cell tumors (GIST).

In a particular embodiment, a method described herein for protecting against, treating or managing a KIT-mediated disorder, e.g., fibrosis or an inflammatory condition (e.g., asthma, rheumatoid arthritis, inflammatory bowel disease, and allergic inflammation), in a subject in need thereof, can achieve at least one, two, three, four or more of the following effects due to administration of a therapeutically effective amount of an anti-KIT antibody described herein: (i) the reduction or amelioration of the severity of fibrosis or an inflammatory condition (e.g., asthma, rheumatoid arthritis, inflammatory bowel disease, and allergic inflammation) and/or one or more symptoms associated therewith; (ii) the reduction in the duration of one or more symptoms associated with fibrosis or an inflammatory condition (e.g., asthma, rheumatoid arthritis, inflammatory bowel disease, and allergic inflammation); (iii) the prevention in the recurrence of fibrosis or an inflammatory condition (e.g., asthma, rheumatoid arthritis, inflammatory bowel disease, and allergic inflammation); (iv) the reduction in hospitalization of a subject; (v) the reduction in hospitalization length; (vi) the inhibition (e.g., partial inhibition) of the progression of fibrosis or an inflammatory condition (e.g., asthma, rheumatoid arthritis, inflammatory bowel disease, and allergic inflammation) and/or one or more symptoms associated therewith; (vii) the enhancement or improvement of the therapeutic effect of another therapy (e.g., anti-inflammatory therapy such as steriods); (viii) an increase in the number of patients in remission (i.e., a time period characterized by no or minimal symptoms associated with the inflammatory condition); (ix) an increase in the length of remission in patients; (x) a decrease in hospitalization rate; (xi) the reduction in the number of symptoms associated with fibrosis or an inflammatory condition (e.g., asthma, rheumatoid arthritis, inflammatory bowel disease, and allergic inflammation); (xii) a decrease in the concentration of one or more inflammatory mediators (e.g., cytokines or interleukins) in biological specimens (e.g., plasma, serum, cerebral spinal fluid, urine, or any other biofluids) of a subject with fibrosis or an inflammatory condition (e.g., asthma, rheumatoid arthritis, inflammatory bowel disease, and allergic inflammation); and (xiii) improvement in the quality of life as assessed by methods well known in the art, e.g., questionnaires.

Other non-limiting examples of KIT-associated disorders or diseases include systemic mast cell disorders (e.g., mastocytosis), hematologic disorders, fibrosis (e.g., idiopathic pulmonary fibrosis (TPF), scleroderma, or myelofibrosis).

As used herein, the term “mast cell related disorder” or “mast cell related disorders” refers to disorders where mast cell activity contributes to the pathology and/or mast cells are found in abnormal amounts, such as above-normal amounts or below-normal amounts, in various parts of the body. For example, mast cell related disorders can exhibit accumulation of pathological mast cells in potentially any or all organs and tissues and/or aberrant release of one or more mast cell mediators such as inflammatory mediators. Non-limiting examples of inflammatory mediators released by mast cells include any of. (i) granule-associated mediators, including histamine, serotonin (5-hydroxytryptamine), and a variety of proteases and peptidases; (ii) eicosanoids such as prostaglandin D2 (PGD2) and leukotriene C4 (LTC4); and (iii) cytokines including interleukin-2 (IL-2), IL-3, IL-4, IL-5, IL-6, IL-10, IL-13, granulocyte-macrophage colony-stimulating factor (GM-CSF), and tumor necrosis factor a (TNFa), and chemokines including CCL-2, CCL-3, CCL-5, and CXCL8.

In a specific aspect, a mast cell related disorder is a mast cell related disorder of the nervous system, e.g., central nervous system, such as NMO, NMOSD, MS, or NF (e.g., NF type 1 (NF1), NF type 2 (NF2), or Schwannomatosis).

MS is a chronic inflammatory demyelinating disorder of the central nervous system (brain and spinal cord) involving episodes where white matter within the brain or spinal cord becomes inflamed and then damaged by the individual's own immune system. These inflamed areas become scarred within the brain and spinal cord. The damage disrupts the ability of parts of the nervous system to communicate, resulting in a variety of symptoms, including physical, mental, and/or psychiatric problems. Forms of MS include, but are not limited to, relapsing forms, with symptoms either occurring in isolated attacks, and progressive forms, with symptoms building up over time. Guidelines for diagnosing MS have been described, see, for example, National Collaborating Centre for Chronic Conditions (UK), “Multiple Sclerosis: National Clinical Guideline for Diagnosis and Management in Primary and Secondary Care,” London: Royal College of Physicians (UK), 2004, (NICE Clinical Guidelines, No. 8.), available from: http://www.ncbi.nlm.nih.gov/books/NBK48919/. Symptoms of MS can manifest as any neurological symptom or sign such as autonomic, visual, motor, and sensory problems. Non-limiting examples of symptoms of MS include loss of sensitivity or changes in sensation such as tingling, pins and needles or numbness, muscle weakness, very pronounced reflexes, muscle spasms, or difficulty in moving, difficulties with coordination and balance (ataxia), problems with speech or swallowing, visual problems (nystagmus, optic neuritis or double vision), fatigue, acute or chronic pain, bladder and bowel difficulties, emotional problems such as depression or unstable mood, Uhthoff s phenomenon (a worsening of symptoms due to exposure to higher than usual temperatures), and Lhermitte's sign (an electrical sensation that runs down the back when bending the neck). In specific aspects, provided herein are methods for protecting against, treating, alleviating, or managing one or more of these symptoms of MS by administering to a subject in need thereof a therapeutically effective amount of an antibody which specifically binds to KIT (e.g., human KIT) or an antigen binding fragment thereof.

Other non-limiting examples of mast cell related disorders include, for example, anaphylaxis, atopic disease, mast cell activation syndrome, allergic rhinitis, food and venom-related allergies (e.g., tree nut, shellfish, fish, hymenoptera venom or bee sting allergies), psoriasis, atopic dermatitis, rosacea, eczema, tubulointerstitial nephritis, glomerulonephritis, diabetic nephropathy, allograft rejection, amyloidosis, renovascular ischemia, reflux nephropathy, polycystic kidney disease, drug-induced nephropathy, post transplant ion fibrosis, and liver fibrosis (e.g., due to alcohol consumption, viral hepatitis B and C, and non-alcoholic steatohepatits (NASH)), parasite infection (e.g., schistosomiasis, amebiasis, echinococcosis), and non-IgE mast cell mediated activation such as angioedema and anaphylaxis.

Alternatively the mast cell related disorder may be urticaria, particularly chronic urticaria, including chronic spontaneous urticaria, chronic idiopathic urticaria and chronic induced urticaria (i.e., chronic inducible urticaria (CIndU). In specific embodiments, the mast cell related disorder is chronic inducible urticaria. Chronic inducible urticarias are forms of urticaria that have an attributable trigger associated with them, typically resulting in wheals (hives) or angioedema. In a specific embodiment, the chronic inducible urticaria is cold urticaria (ColdU). People afflicted with cold urticaria experience symptoms like itching, burning wheals and angioedema when their skin is exposed to temperatures below skin temperature. In another specific embodiment, the chronic inducible urticaria is symptomatic dermographism (SD). Symptomatic dermographism is characterized by the development of a wheal and flare reaction in response to stroking, scratching or rubbing of the skin and usually occurs within minutes of the inciting stimulus. In another specific embodiment, the chronic inducible urticaria is cholinergic urticaria. Cholinergic urticaria is triggered by the body's sweating response to active or passive body warming, and is characterized by small (1-4 mm) wheals surrounded by bright red flares. Common triggers include exercise, hot baths/showers, fever, occlusive dressings, eating spicy foods and emotional stress. In another specific embodiment, the chronic inducible urticaria is heat urticaria. In another specific embodiment, the chronic inducible urticaria is delayed pressure urticaria. In another specific embodiment, the chronic inducible urticaria is solar urticaria. In another specific embodiment, the chronic inducible urticaria is vibratory urticaria. In another specific embodiment, the chronic inducible urticaria is contact urticaria. In another specific embodiment, the chronic inducible urticaria is aquagenic urticaria. Antihistamines are approved therapies for chronic inducible urticarias.

In various embodiments, the patient having a mast cell related disorder such as urticaria (e.g., chronic inducible urticaria) or an eosinophil related disorder such as eosinophilic esophagitis has failed one or more prior treatments for the disorder. In certain embodiments, the one or more prior treatments comprise at least one standard of care therapy for the disorder. In certain embodiments, the one or more prior treatments are all standard of care therapies for the disorder. In certain embodiments, the patient having the mast cell related disorder such as urticaria (e.g., chronic inducible urticaria) or the eosinophil related disorder such as eosinophilic esophagitis has failed an antihistamine treatment(s) for the disorder. In specific embodiments, the patient having the mast cell related disorder such as urticaria (e.g., chronic inducible urticaria) or the eosinophil related disorder such as eosinophilic esophagitis has failed an H1-antihistamine treatment(s) for the disorder. In specific embodiments, the patient having the mast cell related disorder such as urticaria (e.g., chronic inducible urticaria) or the eosinophil related disorder such as eosinophilic esophagitis has failed an H2-antihistamine treatment(s) for the disorder. In specific embodiments, the patient having the mast cell related disorder such as urticaria (e.g., chronic inducible urticaria) or the eosinophil related disorder such as eosinophilic esophagitis has failed both H1- and H2-antihistamine treatments for the disorder. In certain embodiments, the patient having the mast cell related disorder such as urticaria (e.g., chronic inducible urticaria) or the eosinophil related disorder such as eosinophilic esophagitis has failed a treatment(s) with one or more leukotriene receptor antagonists for the disorder. In certain embodiments, the patient having the mast cell related disorder such as urticaria (e.g., chronic inducible urticaria) or the eosinophil related disorder such as eosinophilic esophagitis has failed a treatment(s) with one or more immunomodulators or anti-inflammatory agents for the disorder. In specific embodiments, the patient having the mast cell related disorder such as urticaria (e.g., chronic inducible urticaria) or the eosinophil related disorder such as eosinophilic esophagitis has failed a treatment with an IgE inhibitor such as an anti-IgE antibody, e.g., omalizumab (Xolair®) or ligelizumab, for the disorder. In specific embodiments, the patient having the mast cell related disorder such as urticaria (e.g., chronic inducible urticaria) or the eosinophil related disorder such as eosinophilic esophagitis has failed a treatment with an IL-4R inhibitor such as an anti-IL-4R antibody, e.g., dupilumab (Dupixent®), for the disorder. In specific embodiments, the patient having the mast cell related disorder such as urticaria (e.g., chronic inducible urticaria) or the eosinophil related disorder such as eosinophilic esophagitis has failed a treatment with an IL-5R inhibitor such as an anti-IL-5R antibody, e.g., benralizumab (Fasenra®), for the disorder. In specific embodiments, the patient having the mast cell related disorder such as urticaria (e.g., chronic inducible urticaria) or the eosinophil related disorder such as eosinophilic esophagitis has failed a treatment with an IL-5 inhibitor such as an anti-IL-5 antibody, e.g., mepolizumab, for the disorder. In specific embodiments, the patient having the mast cell related disorder such as urticaria (e.g., chronic inducible urticaria) or the eosinophil related disorder such as eosinophilic esophagitis has failed a treatment with a Siglec 8 inhibitor such as an anti-Siglec 8 antibody, e.g., lirentelimab, for the disorder. In specific embodiments, the patient having the mast cell related disorder such as urticaria (e.g., chronic inducible urticaria) or the eosinophil related disorder such as eosinophilic esophagitis has failed a treatment with a TSLP or TSLPR inhibitor such as an anti-TSLP or anti-TSLPR antibody, e.g., tezepelumab (Tezspire^{T}M), for the disorder. In specific embodiments, the patient having the mast cell related disorder such as urticaria (e.g., chronic inducible urticaria) or the eosinophil related disorder such as eosinophilic esophagitis has failed a treatment with a C5aR inhibitor such as an anti-C5aR antibody, e.g., avdoralimab, for the disorder. In specific embodiments, the patient having the mast cell related disorder such as urticaria (e.g., chronic inducible urticaria) or the eosinophil related disorder such as eosinophilic esophagitis has failed a treatment with a CD200R inhibitor such as an anti-CD200R antibody, e.g., LY3454738, for the disorder. In certain embodiments, the patient having the mast cell related disorder such as urticaria (e.g., chronic inducible urticaria) or the eosinophil related disorder such as eosinophilic esophagitis has failed a treatment(s) with one or more Bruton's Tyrosine Kinase (BTK) inhibitors, e.g., remibrutinib and/or rilzabrutinib, for the disorder. In certain embodiments, the patient having the mast cell related disorder such as urticaria (e.g., chronic inducible urticaria) or the eosinophil related disorder such as eosinophilic esophagitis has failed: (1) an antihistamine treatment(s) (e.g., H1- and/or 112-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib, for the disorder. In certain embodiments, the patient having the mast cell related disorder such as urticaria (e.g., chronic inducible urticaria) or the eosinophil related disorder such as eosinophilic esophagitis has failed: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder. In certain embodiments, the patient having the mast cell related disorder such as urticaria (e.g., chronic inducible urticaria) or the eosinophil related disorder such as eosinophilic esophagitis has failed: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®), for the disorder. In certain embodiments, the patient having the mast cell related disorder such as urticaria (e.g., chronic inducible urticaria) or the eosinophil related disorder such as eosinophilic esophagitis has failed: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab, for the disorder. In certain embodiments, the patient having the mast cell related disorder such as urticaria (e.g., chronic inducible urticaria) or the eosinophil related disorder such as eosinophilic esophagitis has failed: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®), for the disorder.

A patient is considered to have failed a treatment for a disorder if the disorder is refractory to the treatment, resistant to the treatment, relapses after the treatment, and/or if the patient has discontinued the treatment due to intolerance of the treatment.

In various embodiments, the mast cell related disorder such as urticaria (e.g., chronic inducible urticaria) or the eosinophil related disorder such as eosinophilic esophagitis is refractory to one or more prior treatments for the disorder. In certain embodiments, the one or more prior treatments comprise at least one standard of care therapy for the disorder. In certain embodiments, the one or more prior treatments are all standard of care therapies for the disorder. In certain embodiments, the mast cell related disorder such as urticaria (e.g., chronic inducible urticaria) or the eosinophil related disorder such as eosinophilic esophagitis is refractory to an antihistamine treatment(s). In specific embodiments, the mast cell related disorder such as urticaria (e.g., chronic inducible urticaria) or the eosinophil related disorder such as eosinophilic esophagitis is refractory to an H1-antihistamine treatment(s). In specific embodiments, the mast cell related disorder such as urticaria (e.g., chronic inducible urticaria) or the eosinophil related disorder such as eosinophilic esophagitis is refractory to an H2-antihistamine treatment(s). In specific embodiments, the mast cell related disorder such as urticaria (e.g., chronic inducible urticaria) or the eosinophil related disorder such as eosinophilic esophagitis is refractory to both H1- and H2-antihistamine treatments. In certain embodiments, the mast cell related disorder such as urticaria (e.g., chronic inducible urticaria) or the eosinophil related disorder such as eosinophilic esophagitis is refractory to a treatment(s) with one or more leukotriene receptor antagonists. In certain embodiments, the mast cell related disorder such as urticaria (e.g., chronic inducible urticaria) or the eosinophil related disorder such as eosinophilic esophagitis is refractory to a treatment(s) with one or more immunomodulators or anti-inflammatory agents. In specific embodiments, the mast cell related disorder such as urticaria (e.g., chronic inducible urticaria) or the eosinophil related disorder such as eosinophilic esophagitis is refractory to a treatment with an IgE inhibitor such as an anti-IgE antibody, e.g., omalizumab (Xolair®) or ligelizumab. In specific embodiments, the mast cell related disorder such as urticaria (e.g., chronic inducible urticaria) or the eosinophil related disorder such as eosinophilic esophagitis is refractory to a treatment with an IL-4R inhibitor such as an anti-IL-4R antibody, e.g., dupilumab (Dupixent®). In specific embodiments, the mast cell related disorder such as urticaria (e.g., chronic inducible urticaria) or the eosinophil related disorder such as eosinophilic esophagitis is refractory to a treatment with an IL-5R inhibitor such as an anti-IL-5R antibody, e.g., benralizumab (Fasenra®). In specific embodiments, the mast cell related disorder such as urticaria (e.g., chronic inducible urticaria) or the eosinophil related disorder such as eosinophilic esophagitis is refractory to a treatment with an IL-5 inhibitor such as an anti-IL-5 antibody, e.g., mepolizumab. In specific embodiments, the mast cell related disorder such as urticaria (e.g., chronic inducible urticaria) or the eosinophil related disorder such as eosinophilic esophagitis is refractory to a treatment with a Siglec 8 inhibitor such as an anti-Siglec 8 antibody, e.g., lirentelimab. In specific embodiments, the mast cell related disorder such as urticaria (e.g., chronic inducible urticaria) or the eosinophil related disorder such as eosinophilic esophagitis is refractory to a treatment with a TSLP or TSLPR inhibitor such as an anti-TSLP or anti-TSLPR antibody, e.g., tezepelumab (Tezspire™). In specific embodiments, the mast cell related disorder such as urticaria (e.g., chronic inducible urticaria) or the eosinophil related disorder such as eosinophilic esophagitis is refractory to a treatment with a C5aR inhibitor such as an anti-C5aR antibody, e.g., avdoralimab. In specific embodiments, the mast cell related disorder such as urticaria (e.g., chronic inducible urticaria) or the eosinophil related disorder such as eosinophilic esophagitis is refractory to a treatment with a CD200R inhibitor such as an anti-CD200R antibody, e.g., LY3454738. In certain embodiments, the mast cell related disorder such as urticaria (e.g., chronic inducible urticaria) or the eosinophil related disorder such as eosinophilic esophagitis is refractory to a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is refractory to: (1) an antihistamine treatment(s) (e.g., H1- and/or H2-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire^{T}M), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is refractory to: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire^{T}M), a C5aR inhibitor such as an anti-C5aR antibody, e.g., avdoralimab, and/or a CD200R inhibitor such as an anti-CD200R antibody, e.g., LY3454738). In certain embodiments, the mast cell related disorder such as urticaria (e.g., chronic inducible urticaria) or the eosinophil related disorder such as eosinophilic esophagitis is refractory to: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®). In certain embodiments, the mast cell related disorder such as urticaria (e.g., chronic inducible urticaria) or the eosinophil related disorder such as eosinophilic esophagitis is refractory to: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab. In certain embodiments, the mast cell related disorder such as urticaria (e.g., chronic inducible urticaria) or the eosinophil related disorder such as eosinophilic esophagitis is refractory to: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®).

In various embodiments, the mast cell related disorder such as urticaria (e.g., chronic inducible urticaria) or the eosinophil related disorder such as eosinophilic esophagitis is resistant to one or more prior treatments for the disorder. In certain embodiments, the one or more prior treatments comprise at least one standard of care therapy for the disorder. In certain embodiments, the one or more prior treatments are all standard of care therapies for the disorder. In certain embodiments, the mast cell related disorder such as urticaria (e.g., chronic inducible urticaria) or the eosinophil related disorder such as eosinophilic esophagitis is resistant to an antihistamine treatment(s). In specific embodiments, the mast cell related disorder such as urticaria (e.g., chronic inducible urticaria) or the eosinophil related disorder such as eosinophilic esophagitis is resistant to an H1-antihistamine treatment(s). In specific embodiments, the mast cell related disorder such as urticaria (e.g., chronic inducible urticaria) or the eosinophil related disorder such as eosinophilic esophagitis is resistant to an H2-antihistamine treatment(s). In specific embodiments, the mast cell related disorder such as urticaria (e.g., chronic inducible urticaria) or the eosinophil related disorder such as eosinophilic esophagitis is resistant to both H1- and H2-antihistamine treatments. In certain embodiments, the mast cell related disorder such as urticaria (e.g., chronic inducible urticaria) or the eosinophil related disorder such as eosinophilic esophagitis is resistant to a treatment(s) with one or more leukotriene receptor antagonists. In certain embodiments, the mast cell related disorder such as urticaria (e.g., chronic inducible urticaria) or the eosinophil related disorder such as eosinophilic esophagitis is resistant to a treatment(s) with one or more immunomodulators or anti-inflammatory agents. In specific embodiments, the mast cell related disorder such as urticaria (e.g., chronic inducible urticaria) or the eosinophil related disorder such as eosinophilic esophagitis is resistant to a treatment with an IgE inhibitor such as an anti-IgE antibody, e.g., omalizumab (Xolair®) or ligelizumab. In specific embodiments, the mast cell related disorder such as urticaria (e.g., chronic inducible urticaria) or the eosinophil related disorder such as eosinophilic esophagitis is resistant to a treatment with an IL-4R inhibitor such as an anti-IL-4R antibody, e.g., dupilumab (Dupixent®). In specific embodiments, the mast cell related disorder such as urticaria (e.g., chronic inducible urticaria) or the eosinophil related disorder such as eosinophilic esophagitis is resistant to a treatment with an IL-5R inhibitor such as an anti-IL-5R antibody, e.g., benralizumab (Fasenra®). In specific embodiments, the mast cell related disorder such as urticaria (e.g., chronic inducible urticaria) or the eosinophil related disorder such as eosinophilic esophagitis is resistant to a treatment with an IL-5 inhibitor such as an anti-IL-5 antibody, e.g., mepolizumab. In specific embodiments, the mast cell related disorder such as urticaria (e.g., chronic inducible urticaria) or the eosinophil related disorder such as eosinophilic esophagitis is resistant to a treatment with a Siglec 8 inhibitor such as an anti-Siglec 8 antibody, e.g., lirentelimab. In specific embodiments, the mast cell related disorder such as urticaria (e.g., chronic inducible urticaria) or the eosinophil related disorder such as eosinophilic esophagitis is resistant to a treatment with a TSLP or TSLPR inhibitor such as an anti-TSLP or anti-TSLPR antibody, e.g., tezepelumab (Tezspire^{T}M). In specific embodiments, the mast cell related disorder such as urticaria (e.g., chronic inducible urticaria) or the eosinophil related disorder such as eosinophilic esophagitis is resistant to a treatment with a C5aR inhibitor such as an anti-C5aR antibody, e.g., avdoralimab. In specific embodiments, the mast cell related disorder such as urticaria (e.g., chronic inducible urticaria) or the eosinophil related disorder such as eosinophilic esophagitis is resistant to a treatment with a CD200R inhibitor such as an anti-CD200R antibody, e.g., LY3454738. In certain embodiments, the mast cell related disorder such as urticaria (e.g., chronic inducible urticaria) or the eosinophil related disorder such as eosinophilic esophagitis is resistant to a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is resistant to: (1) an antihistamine treatment(s) (e.g., H1- and/or 112-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is resistant to: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738). In certain embodiments, the mast cell related disorder such as urticaria (e.g., chronic inducible urticaria) or the eosinophil related disorder such as eosinophilic esophagitis is resistant to: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®). In certain embodiments, the mast cell related disorder such as urticaria (e.g., chronic inducible urticaria) or the eosinophil related disorder such as eosinophilic esophagitis is resistant to: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab. In certain embodiments, the mast cell related disorder such as urticaria (e.g., chronic inducible urticaria) or the eosinophil related disorder such as eosinophilic esophagitis is resistant to: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®).

In various embodiments, the mast cell related disorder such as urticaria (e.g., chronic inducible urticaria) or the eosinophil related disorder such as eosinophilic esophagitis is both refractory and resistant to one or more prior treatments for the disorder. In certain embodiments, the one or more prior treatments comprise at least one standard of care therapy for the disorder. In certain embodiments, the one or more prior treatments are all standard of care therapies for the disorder. In certain embodiments, the mast cell related disorder such as urticaria (e.g., chronic inducible urticaria) or the eosinophil related disorder such as eosinophilic esophagitis is both refractory and resistant to an antihistamine treatment(s). In specific embodiments, the mast cell related disorder such as urticaria (e.g., chronic inducible urticaria) or the eosinophil related disorder such as eosinophilic esophagitis is both refractory and resistant to an H1-antihistamine treatment(s). In specific embodiments, the mast cell related disorder such as urticaria (e.g., chronic inducible urticaria) or the eosinophil related disorder such as eosinophilic esophagitis is both refractory and resistant to an H2-antihistamine treatment(s). In specific embodiments, the mast cell related disorder such as urticaria (e.g., chronic inducible urticaria) or the eosinophil related disorder such as eosinophilic esophagitis is both refractory and resistant to both H1- and H2-antihistamine treatments. In certain embodiments, the mast cell related disorder such as urticaria (e.g., chronic inducible urticaria) or the eosinophil related disorder such as eosinophilic esophagitis is both refractory and resistant to a treatment(s) with one or more leukotriene receptor antagonists. In certain embodiments, the mast cell related disorder such as urticaria (e.g., chronic inducible urticaria) or the eosinophil related disorder such as eosinophilic esophagitis is both refractory and resistant to a treatment(s) with one or more immunomodulators or anti-inflammatory agents. In specific embodiments, the mast cell related disorder such as urticaria (e.g., chronic inducible urticaria) or the eosinophil related disorder such as eosinophilic esophagitis is both refractory and resistant to a treatment with an IgE inhibitor such as an anti-IgE antibody, e.g., omalizumab (Xolair®) or ligelizumab. In specific embodiments, the mast cell related disorder such as urticaria (e.g., chronic inducible urticaria) or the eosinophil related disorder such as eosinophilic esophagitis is both refractory and resistant to a treatment with an IL-4R inhibitor such as an anti-IL-4R antibody, e.g., dupilumab (Dupixent®). In specific embodiments, the mast cell related disorder such as urticaria (e.g., chronic inducible urticaria) or the eosinophil related disorder such as eosinophilic esophagitis is both refractory and resistant to a treatment with an IL-5R inhibitor such as an anti-IL-5R antibody, e.g., benralizumab (Fasenra®). In specific embodiments, the mast cell related disorder such as urticaria (e.g., chronic inducible urticaria) or the eosinophil related disorder such as eosinophilic esophagitis is both refractory and resistant to a treatment with an IL-5 inhibitor such as an anti-IL-5 antibody, e.g., mepolizumab. In specific embodiments, the mast cell related disorder such as urticaria (e.g., chronic inducible urticaria) or the eosinophil related disorder such as eosinophilic esophagitis is both refractory and resistant to a treatment with a Siglec 8 inhibitor such as an anti-Siglec 8 antibody, e.g., lirentelimab. In specific embodiments, the mast cell related disorder such as urticaria (e.g., chronic inducible urticaria) or the eosinophil related disorder such as eosinophilic esophagitis is both refractory and resistant to a treatment with a TSLP or TSLPR inhibitor such as an anti-TSLP or anti-TSLPR antibody, e.g., tezepelumab (Tezspire™). In specific embodiments, the mast cell related disorder such as urticaria (e.g., chronic inducible urticaria) or the eosinophil related disorder such as eosinophilic esophagitis is both refractory and resistant to a treatment with a C5aR inhibitor such as an anti-C5aR antibody, e.g., avdoralimab. In specific embodiments, the mast cell related disorder such as urticaria (e.g., chronic inducible urticaria) or the eosinophil related disorder such as eosinophilic esophagitis is both refractory and resistant to a treatment with a CD200R inhibitor such as an anti-CD200R antibody, e.g., LY3454738. In certain embodiments, the mast cell related disorder such as urticaria (e.g., chronic inducible urticaria) or the eosinophil related disorder such as eosinophilic esophagitis is both refractory and resistant to a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is both refractory and resistant to: (1) an antihistamine treatment(s) (e.g., H1- and/or H2-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is both refractory and resistant to: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738). In certain embodiments, the mast cell related disorder such as urticaria (e.g., chronic inducible urticaria) or the eosinophil related disorder such as eosinophilic esophagitis is both refractory and resistant to: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®). In certain embodiments, the mast cell related disorder such as urticaria (e.g., chronic inducible urticaria) or the eosinophil related disorder such as eosinophilic esophagitis is both refractory and resistant to: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab. In certain embodiments, the mast cell related disorder such as urticaria (e.g., chronic inducible urticaria) or the eosinophil related disorder such as eosinophilic esophagitis is both refractory and resistant to: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®).

In various embodiments, the mast cell related disorder such as urticaria (e.g., chronic inducible urticaria) or the eosinophil related disorder such as eosinophilic esophagitis is a relapsed disorder that has relapsed after one or more prior treatments for the disorder. In certain embodiments, the one or more prior treatments comprise at least one standard of care therapy for the disorder. In certain embodiments, the one or more prior treatments are all standard of care therapies for the disorder. In certain embodiments, the mast cell related disorder such as urticaria (e.g., chronic inducible urticaria) or the eosinophil related disorder such as eosinophilic esophagitis is a relapsed disorder that has relapsed after an antihistamine treatment(s). In specific embodiments, the mast cell related disorder such as urticaria (e.g., chronic inducible urticaria) or the eosinophil related disorder such as eosinophilic esophagitis is a relapsed disorder that has relapsed after an H1-antihistamine treatment(s). In specific embodiments, the mast cell related disorder such as urticaria (e.g., chronic inducible urticaria) or the eosinophil related disorder such as eosinophilic esophagitis is a relapsed disorder that has relapsed after an H2-antihistamine treatment(s). In specific embodiments, the mast cell related disorder such as urticaria (e.g., chronic inducible urticaria) or the eosinophil related disorder such as eosinophilic esophagitis is a relapsed disorder that has relapsed after both H1- and H2-antihistamine treatments. In certain embodiments, the mast cell related disorder such as urticaria (e.g., chronic inducible urticaria) or the eosinophil related disorder such as eosinophilic esophagitis is a relapsed disorder that has relapsed after a treatment(s) with one or more leukotriene receptor antagonists. In certain embodiments, the mast cell related disorder such as urticaria (e.g., chronic inducible urticaria) or the eosinophil related disorder such as eosinophilic esophagitis is a relapsed disorder that has relapsed after a treatment(s) with one or more immunomodulators or anti-inflammatory agents. In specific embodiments, the mast cell related disorder such as urticaria (e.g., chronic inducible urticaria) or the eosinophil related disorder such as eosinophilic esophagitis is a relapsed disorder that has relapsed after a treatment with an IgE inhibitor such as an anti-IgE antibody, e.g., omalizumab (Xolair®) or ligelizumab. In specific embodiments, the mast cell related disorder such as urticaria (e.g., chronic inducible urticaria) or the eosinophil related disorder such as eosinophilic esophagitis is a relapsed disorder that has relapsed after a treatment with an IL-4R inhibitor such as an anti-IL-4R antibody, e.g., dupilumab (Dupixent®). In specific embodiments, the mast cell related disorder such as urticaria (e.g., chronic inducible urticaria) or the eosinophil related disorder such as eosinophilic esophagitis is a relapsed disorder that has relapsed after a treatment with an IL-5R inhibitor such as an anti-IL-5R antibody, e.g., benralizumab (Fasenra®). In specific embodiments, the mast cell related disorder such as urticaria (e.g., chronic inducible urticaria) or the eosinophil related disorder such as eosinophilic esophagitis is a relapsed disorder that has relapsed after a treatment with an IL-5 inhibitor such as an anti-TL-5 antibody, e.g., mepolizumab. In specific embodiments, the mast cell related disorder such as urticaria (e.g., chronic inducible urticaria) or the eosinophil related disorder such as eosinophilic esophagitis is a relapsed disorder that has relapsed after a treatment with a Siglec 8 inhibitor such as an anti-Siglec 8 antibody, e.g., lirentelimab. In specific embodiments, the mast cell related disorder such as urticaria (e.g., chronic inducible urticaria) or the eosinophil related disorder such as eosinophilic esophagitis is a relapsed disorder that has relapsed after a treatment with a TSLP or TSLPR inhibitor such as an anti-TSLP or anti-TSLPR antibody, e.g., tezepelumab (Tezspire™). In specific embodiments, the mast cell related disorder such as urticaria (e.g., chronic inducible urticaria) or the eosinophil related disorder such as eosinophilic esophagitis is a relapsed disorder that has relapsed after a treatment with a C5aR inhibitor such as an anti-C5aR antibody, e.g., avdoralimab. In specific embodiments, the mast cell related disorder such as urticaria (e.g., chronic inducible urticaria) or the eosinophil related disorder such as eosinophilic esophagitis is a relapsed disorder that has relapsed after a treatment with a CD200R inhibitor such as an anti-CD200R antibody, e.g., LY3454738. In certain embodiments, the mast cell related disorder such as urticaria (e.g., chronic inducible urticaria) or the eosinophil related disorder such as eosinophilic esophagitis is a relapsed disorder that has relapsed after a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is a relapsed disorder that has relapsed after: (1) an antihistamine treatment(s) (e.g., H1- and/or H2-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib. In certain embodiments, the mast cell related disorder such as urticaria (e.g., chronic inducible urticaria) or the eosinophil related disorder such as eosinophilic esophagitis is a relapsed disorder that has relapsed after: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738). In certain embodiments, the mast cell related disorder such as urticaria (e.g., chronic inducible urticaria) or the eosinophil related disorder such as eosinophilic esophagitis is a relapsed disorder that has relapsed after: (1) an antihistamine treatment(s) (e.g., H1- and/or H2-antihistamine treatment(s)), (2) a treatment with an IgE inhibitor such as an anti-IgE antibody, e.g., omalizumab (Xolair®) or ligelizumab, and (3) a treatment with an IL-4R inhibitor such as an anti-IL-4R antibody, e.g., dupilumab (Dupixent®). In certain embodiments, the mast cell related disorder such as urticaria (e.g., chronic inducible urticaria) or the eosinophil related disorder such as eosinophilic esophagitis is a relapsed disorder that has relapsed after: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab. In certain embodiments, the mast cell related disorder such as urticaria (e.g., chronic inducible urticaria) or the eosinophil related disorder such as eosinophilic esophagitis is a relapsed disorder that has relapsed after: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®).

In various embodiments, the patient having the mast cell related disorder such as urticaria (e.g., chronic inducible urticaria) or the eosinophil related disorder such as eosinophilic esophagitis has discontinued one or more prior treatments for the disorder due to intolerance of the treatment(s). In certain embodiments, the one or more prior treatments comprise at least one standard of care therapy for the disorder. In certain embodiments, the one or more prior treatments are all standard of care therapies for the disorder. In certain embodiments, the patient having the mast cell related disorder such as urticaria (e.g., chronic inducible urticaria) or the eosinophil related disorder such as eosinophilic esophagitis has discountinued an antihistamine treatment(s) for the disorder due to intolerance of the treatment(s). In specific embodiments, the patient having the mast cell related disorder such as urticaria (e.g., chronic inducible urticaria) or the eosinophil related disorder such as eosinophilic esophagitis has discountinued an H1-antihistamine treatment(s) for the disorder due to intolerance of the treatment(s). In specific embodiments, the patient having the mast cell related disorder such as urticaria (e.g., chronic inducible urticaria) or the eosinophil related disorder such as eosinophilic esophagitis has discontinued an 112-antihistamine treatment(s) for the disorder due to intolerance of the treatment(s). In specific embodiments, the patient having the mast cell related disorder such as urticaria (e.g., chronic inducible urticaria) or the eosinophil related disorder such as eosinophilic esophagitis has discontinued both H1- and 112-antihistamine treatments for the disorder due to intolerance of the treatments. In certain embodiments, the patient having the mast cell related disorder such as urticaria (e.g., chronic inducible urticaria) or the eosinophil related disorder such as eosinophilic esophagitis has discountinued a treatment(s) with one or more leukotriene receptor antagonists for the disorder due to intolerance of the treatment(s). In certain embodiments, the patient having the mast cell related disorder such as urticaria (e.g., chronic inducible urticaria) or the eosinophil related disorder such as eosinophilic esophagitis has discontinued a treatment(s) with one or more immunomodulators or anti-inflammatory agents for the disorder due to intolerance of the treatment(s). In specific embodiments, the patient having the mast cell related disorder such as urticaria (e.g., chronic inducible urticaria) or the eosinophil related disorder such as eosinophilic esophagitis has discontinued a treatment with an IgE inhibitor such as an anti-IgE antibody, e.g., omalizumab (Xolair®) or ligelizumab, for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with an IL-4R inhibitor such as an anti-IL-4R antibody, e.g., dupilumab (Dupixent®), for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with an IL-5R inhibitor such as an anti-IL-5R antibody, e.g., benralizumab (Fasenra®), for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with an IL-5 inhibitor such as an anti-IL-5 antibody, e.g., mepolizumab, for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with a Siglec 8 inhibitor such as an anti-Siglec 8 antibody, e.g., lirentelimab, for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with a TSLP or TSLPR inhibitor such as an anti-TSLP or anti-TSLPR antibody, e.g., tezepelumab (Tezspire™), for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with a C5aR inhibitor such as an anti-C5aR antibody, e.g., avdoralimab, for the disorder due to intolerance of the treatment. In specific embodiments, the patient having the mast cell related disorder such as urticaria (e.g., chronic inducible urticaria) or the eosinophil related disorder such as eosinophilic esophagitis has discontinued a treatment with a CD200R inhibitor such as an anti-CD200R antibody, e.g., LY3454738, for the disorder due to intolerance of the treatment. In certain embodiments, the patient having the mast cell related disorder such as urticaria (e.g., chronic inducible urticaria) or the eosinophil related disorder such as eosinophilic esophagitis has discontinued a treatment(s) with one or more BTK inhibitors, e.g., remibrutinib and/or rilzabrutinib, for the disorder due to intolerance of the treatment. In certain embodiments, the patient having the mast cell related disorder such as urticaria (e.g., chronic inducible urticaria) or the eosinophil related disorder such as eosinophilic esophagitis has discontinued: (1) an antihistamine treatment(s) (e.g., H1- and/or H2-antihistamine treatment(s)), (2) a treatment(s) with one or more leukotriene receptor antagonists, (3) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), and/or (4) a treatment(s) with one or more BTK inhibitors, e.g., remibrutinib and/or rilzabrutinib, for the disorder due to intolerance of the treatment(s). In certain embodiments, the patient having the mast cell related disorder such as urticaria (e.g., chronic inducible urticaria) or the eosinophil related disorder such as eosinophilic esophagitis has discontinued: (1) an antihistamine treatment(s) (e.g., H1- and/or H2-antihistamine treatment(s)), and (2) a treatment(s) with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738), for the disorder due to intolerance of the treatments. In certain embodiments, the patient having the mast cell related disorder such as urticaria (e.g., chronic inducible urticaria) or the eosinophil related disorder such as eosinophilic esophagitis has discontinued: (1) an antihistamine treatment(s) (e.g., H1- and/or H2-antihistamine treatment(s)), (2) a treatment with a IgE inhibitor such as an anti-IgE antibody, e.g., omalizumab (Xolair®) or ligelizumab, and (3) a treatment with a IL-4R inhibitor such as an anti-IL-4R antibody, e.g., dupilumab (Dupixent®), for the disorder due to intolerance of the treatments. In certain embodiments, the patient having the mast cell related disorder such as urticaria (e.g., chronic inducible urticaria) or the eosinophil related disorder such as eosinophilic esophagitis has discontinued: (1) an antihistamine treatment(s) (e.g., H1- and/or H2-antihistamine treatment(s)), and (2) a treatment with an IgE inhibitor such as an anti-IgE antibody, e.g., omalizumab (Xolair®) or ligelizumab, for the disorder due to intolerance of the treatments. In certain embodiments, the patient having the mast cell related disorder such as urticaria (e.g., chronic inducible urticaria) or the eosinophil related disorder such as eosinophilic esophagitis has discontinued: (1) a treatment with an IgE inhibitor such as an anti-IgE antibody, e.g., omalizumab (Xolair®) or ligelizumab, and (2) a treatment with an IL-4R inhibitor such as an anti-IL-4R antibody, e.g., dupilumab (Dupixent®), for the disorder due to intolerance of the treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with one or more antihistamines (e.g., H1- and/or H2-antihistamine treatment(s)) for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite the use of an H1-antihistamine alone or in combination with a H2-antihistamine and/or leukotriene receptor antagonist for the disorder.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with one or more leukotriene receptor antagonists for the disorder.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with an IL-5R inhibitor such as an anti-IL-5R antibody, e.g., benralizumab (Fasenra®), for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with an IL-5 inhibitor such as an anti-IL-5 antibody, e.g., mepolizumab, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with a Siglec 8 inhibitor such as an anti-Siglec 8 antibody, e.g., lirentelimab, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with a TSLP or TSLPR inhibitor such as an anti-TSLP or anti-TSLPR antibody, e.g., tezepelumab (Tezspire™), for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with a C5aR inhibitor such as an anti-C5aR antibody, e.g., avdoralimab, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with a CD200R inhibitor such as an anti-CD200R antibody, e.g., LY3454738, for the disorder.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment with one or more BTK inhibitors, e.g., remibrutinib and/or rilzabrutinib, for the disorder.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatments with (1) one or more antihistamines, as described above, and (2) one or more immunomodulators or anti-inflammatory agents, as described above, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatments with (1) one or more antihistamines, as described above, and (2) an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis who remains symptomatic despite treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the patient has failed said treatment with one or more antihistamines (e.g., H1- and/or H2-antihistamine treatment(s)).

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the patient has failed said treatment with one or more leukotriene receptor antagonists.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire^{T}M), a C5aR inhibitor such as an anti-C5aR antibody, e.g., avdoralimab, and/or a CD200R inhibitor such as an anti-CD200R antibody, e.g., LY3454738) for the disorder, wherein the patient has failed said treatment with one or more immunomodulators or anti-inflammatory agents. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the patient has failed said treatment with an IgE inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the patient has failed said treatment with an IL-4R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the patient has failed said treatment with an IL-5R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-IL-5 antibody, e.g., mepolizumab, for the disorder, wherein the patient has failed said treatment with an IL-5 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the patient has failed said treatment with a Siglec 8 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the patient has failed said treatment with a TSLP or TSLPR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the patient has failed said treatment with a C5aR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the patient has failed said treatment with a CD200R inhibitor.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the patient has failed said treatment with one or more BTK inhibitors.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the patient has failed said treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the patient has failed said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the patient has failed said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the patient has failed said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the patient has failed said treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the disorder is refractory to said treatment with one or more antihistamines (e.g., H1- and/or H2-antihistamine treatment(s)).

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the disorder is refractory to said treatment with one or more leukotriene receptor antagonists.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder, wherein the disorder is refractory to said treatment with one or more immunomodulators or anti-inflammatory agents. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the disorder is refractory to said treatment with an IgE inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the disorder is refractory to said treatment with an IL-4R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the disorder is refractory to said treatment with an IL-5R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-TL-5 antibody, e.g., mepolizumab, for the disorder, wherein the disorder is refractory to said treatment with an IL-5 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the disorder is refractory to said treatment with a Siglec 8 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the disorder is refractory to said treatment with a TSLP or TSLPR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the disorder is refractory to said treatment with a C5aR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the disorder is refractory to said treatment with a CD200R inhibitor.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the disorder is refractory to said treatment with one or more BTK inhibitors.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the disorder is refractory to said treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the disorder is refractory to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the disorder is refractory to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the disorder is refractory to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the disorder is refractory to said treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the disorder is resistant to said treatment with one or more antihistamines (e.g., H1- and/or H2-antihistamine treatment(s)).

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the disorder is resistant to said treatment with one or more leukotriene receptor antagonists.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire^{T}M), a C5aR inhibitor such as an anti-C5aR antibody, e.g., avdoralimab, and/or a CD200R inhibitor such as an anti-CD200R antibody, e.g., LY3454738) for the disorder, wherein the disorder is resistant to said treatment with one or more immunomodulators or anti-inflammatory agents. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the disorder is resistant to said treatment with an IgE inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the disorder is resistant to said treatment with an IL-4R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the disorder is resistant to said treatment with an IL-5R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-IL-5 antibody, e.g., mepolizumab, for the disorder, wherein the disorder is resistant to said treatment with an IL-5 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the disorder is resistant to said treatment with a Siglec 8 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the disorder is resistant to said treatment with a TSLP or TSLPR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the disorder is resistant to said treatment with a C5aR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the disorder is resistant to said treatment with a CD200R inhibitor.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the disorder is resistant to said treatment with one or more BTK inhibitors.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the disorder is resistant to said treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the disorder is resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the disorder is resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the disorder is resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the disorder is resistant to said treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the disorder is both refractory and resistant to said treatment with one or more antihistamines (e.g., H1- and/or H2-antihistamine treatment(s)).

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the disorder is both refractory and resistant to said treatment with one or more leukotriene receptor antagonists.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder, wherein the disorder is both refractory and resistant to said treatment with one or more immunomodulators or anti-inflammatory agents. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the disorder is both refractory and resistant to said treatment with an IgE inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the disorder is both refractory and resistant to said treatment with an IL-4R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the disorder is both refractory and resistant to said treatment with an IL-5R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-IL-5 antibody, e.g., mepolizumab, for the disorder, wherein the disorder is both refractory and resistant to said treatment with an IL-5 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the disorder is both refractory and resistant to said treatment with a Siglec 8 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the disorder is both refractory and resistant to said treatment with a TSLP or TSLPR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the disorder is both refractory and resistant to said treatment with a C5aR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the disorder is both refractory and resistant to said treatment with a CD200R inhibitor.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the disorder is both refractory and resistant to said treatment with one or more BTK inhibitors.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the disorder is both refractory and resistant to said treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the disorder is both refractory and resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the disorder is both refractory and resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the disorder is both refractory and resistant to said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the disorder is both refractory and resistant to said treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the disorder is a relapsed disorder that has relapsed after said treatment with one or more antihistamines (e.g., H1- and/or H2-antihistamine treatment(s)).

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the disorder is a relapsed disorder that has relapsed after said treatment with one or more leukotriene receptor antagonists.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-TL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder, wherein the disorder is a relapsed disorder that has relapsed after said treatment with one or more immunomodulators or anti-inflammatory agents. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the disorder is a relapsed disorder that has relapsed after said treatment with an IgE inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the disorder is a relapsed disorder that has relapsed after said treatment with an IL-4R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the disorder is a relapsed disorder that has relapsed after said treatment with an IL-5R inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-IL-5 antibody, e.g., mepolizumab, for the disorder, wherein the disorder is a relapsed disorder that has relapsed after said treatment with an IL-5 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the disorder is a relapsed disorder that has relapsed after said treatment with a Siglec 8 inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the disorder is a relapsed disorder that has relapsed after said treatment with a TSLP or TSLPR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the disorder is a relapsed disorder that has relapsed after said treatment with a C5aR inhibitor. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the disorder is a relapsed disorder that has relapsed after said treatment with a CD200R inhibitor.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the disorder is a relapsed disorder that has relapsed after said treatment with one or more BTK inhibitors.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the disorder is a relapsed disorder that has relapsed after said treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the disorder is a relapsed disorder that has relapsed after said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the disorder is a relapsed disorder that has relapsed after said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the disorder is a relapsed disorder that has relapsed after said treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the disorder is a relapsed disorder that has relapsed after said treatments.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more antihistamines (e.g., H1- and/or H2-antihistamine treatment(s)) for the disorder, wherein the patient has discontinued said treatment with one or more antihistamines (e.g., H1- and/or H2-antihistamine treatment(s)) due to intolerance of the treatment.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more leukotriene receptor antagonists for the disorder, wherein the patient has discontinued said treatment with one or more leukotriene receptor antagonists due to intolerance of the treatment.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder, wherein the patient has discontinued said treatment with one or more immunomodulators or anti-inflammatory agents due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IgE inhibitor such as an anti-IgE antibody, e.g., omalizumab (Xolair®) or ligelizumab, for the disorder, wherein the patient has discontinued said treatment with an IgE inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-4R inhibitor such as an anti-IL-4R antibody, e.g., dupilumab (Dupixent®), for the disorder, wherein the patient has discontinued said treatment with an IL-4R inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5R inhibitor such as an anti-IL-5R antibody, e.g., benralizumab (Fasenra®), for the disorder, wherein the patient has discontinued said treatment with an IL-5R inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with an IL-5 inhibitor such as an anti-IL-5 antibody, e.g., mepolizumab, for the disorder, wherein the patient has discontinued said treatment with an IL-5 inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a Siglec 8 inhibitor such as an anti-Siglec 8 antibody, e.g., lirentelimab, for the disorder, wherein the patient has discontinued said treatment with a Siglec 8 inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a TSLP or TSLPR inhibitor such as an anti-TSLP or anti-TSLPR antibody, e.g., tezepelumab (Tezspire™), for the disorder, wherein the patient has discontinued said treatment with a TSLP or TSLPR inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a C5aR inhibitor such as an anti-C5aR antibody, e.g., avdoralimab, for the disorder, wherein the patient has discontinued said treatment with a C5aR inhibitor due to intolerance of the treatment. In a specific embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with a CD200R inhibitor such as an anti-CD200R antibody, e.g., LY3454738, for the disorder, wherein the patient has discontinued said treatment with a CD200R inhibitor due to intolerance of the treatment.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment with one or more BTK inhibitors, e.g., remibrutinib and/or rilzabrutinib, for the disorder, wherein the patient has discontinued said treatment with one or more BTK inhibitors due to intolerance of the treatment.

In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatment(s) with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder; wherein the patient has discontinued said treatment(s) due to intolerance of the treatment(s). In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) one or more immunomodulators or anti-inflammatory agents, as described above, for the disorder; wherein the patient has discontinued said treatments due to intolerance of the treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder; wherein the patient has discontinued said treatments due to intolerance of the treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) one or more antihistamines, as described above, and (2) an IgE inhibitor such as an anti-IgE antibody, e.g., omalizumab (Xolair®) or ligelizumab, for the disorder; wherein the patient has discontinued said treatments due to intolerance of the treatments. In one embodiment, an anti-KIT antibody, antigen binding fragment thereof, or conjugate described herein is administered to a patient having a mast cell related disorder such as urticaria (e.g., chronic inducible urticaria) or an eosinophil related disorder such as eosinophilic esophagitis following treatments with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder; wherein the patient has discontinued said treatments due to intolerance of the treatments.

Eosinophils are white blood cells activated by the lymphocytes of the adaptive immune response and are important in defense against parasitic infections. The level of eosinophils in the blood is normally low, and it can increase markedly in certain situations, such as atopy, which can result in eosinophilia, an abnormally large number of eosinophils in the blood.

As used herein, the term “eosinophil related disorder” or “eosinophil related disorders” refers to disorders that arise when eosinophils are found in abnormal amounts, such as above-normal amounts or below-normal amounts, in various parts of the body. For example, when the body produces too many eosinophils, they can cause chronic inflammation resulting in tissue damage. In certain aspects, an eosinophil disorder may be associated with an abnormal amount of eosinophil in a tissue for a prolonged period of time in response to a trigger. For example, higher amounts of eosinophils may be produced in response to a trigger, such as an infection or allergen, but the high amounts of eosinophils do not decrease at a normal rate and thus are maintained at a high amount for a longer period of time than expected.

Eosinophil related disorders can be diagnosed according to the location where the levels of eosinophils are elevated. Non-limiting examples of eosinophil related disorders include allergic disorders, infectious diseases, blood disorders, immunologic disorders and reactions, endocrine disorders, pulmonary conditions, gastrointestinal disorders, neurologic disorders, rheumatologic disorders, cardiac conditions, and renal diseases. In a specific embodiment, the eosinophil related disorder described herein is eosinophilic esophagitis (EoE). In certain aspects, eosinophilia is an eosinophil related disorder characterized by a peripheral blood eosinophil count greater than a normal level, for example, greater than 450/μL. Eosinophilia can be induced or triggered by a variety of conditions, such as allergy or infection. In particular aspects, elevated levels of eosinophils are observed locally, for example, in the lung, heart, spinal cord, or brain.

Non-limiting examples of neurologic disorders involving eosinophils include central nervous system infections, ventriculoperitoneal shunts, and drug-induced adverse reactions. In certain aspects, an increase in eosinophil count or activity can be detected in cerebrospinal fluid (CSF) or in other samples obtained from tissue of fluid of the central nervous system.

Non-limiting examples of eosinophil or mast cell related indications include upper airway diseases such as allergic rhinitis and sinusitis, foreign body aspiration, glottic stenosis, tracheal stenosis, laryngotracheomalacia, vascular rings, chronic obstructive pulmonary disease (COPD), and congestive heart failure, eosinophilic bronchitis, polychondritis, sarcoidosis, papillomatosis, arthritis (e.g., rheumatoid arthritis) and Wegener's granulomatosis.

In certain embodiments, “eosinophil related disorder” or “eosinophil related disorders” can involve disorders where eosinophil activity contributes to the disorder, e.g., disorders that arise when eosinophils are found in abnormal amounts, such as above-normal amounts or below-normal amounts, in various parts of the body.

In a certain aspect, an anti-KIT antibody or antigen binding fragment thereof described herein or a pharmaceutical composition thereof is administered to a subject in need thereof in accordance with the methods provided herein for treating an eosinophil or mast cell related disorder (e.g., an eosinophil or mast cell related disorder in the nervous system, e.g., central nervous system), at a dosage and a frequency of administration that achieves one or more of the following in a subject diagnosed with an eosinophil or mast cell related disorder: reduction in the number and/or activity of eosinophils, reduction in mast cell proliferation, reduction in mast cell number or amount, inhibition or reduction in mast cell activity, reduction in mast cell induced production of inflammatory factors, reduction in production of inflammatory factors, restoration of mast cell homeostasis, reduced mast cell migration, reduced mast cell adhesion, inhibition or reduction in mast cell recruitment of eosinophils, and inhibition or reduction in antigen-mediated degranulation of mast cells.

In other embodiments, a KIT-associated disease is a cancer such as breast cancer, leukemia (e.g., chronic myelogenous leukemia, acute myeloid leukemia, mast cell leukemia), lung cancer (e.g., small cell lung cancer), neuroblastoma, gastrointestinal stromal tumors (GIST), melanoma, colorectal cancer, sarcoma (e.g., Ewing's sarcoma), and germ cell tumors (e.g., seminoma). In a particular embodiment, a cancer which is treated or managed or against which is protected by the methods provided herein is characterized by a gain-of-function KIT mutation or overexpression of KIT.

In specific embodiments, a cancer treated in accordance with the methods described herein can be any type of cancer which comprises cancer or tumor cells expressing cell surface KIT or a mutated form thereof, which can be confirmed by any histologically or cytologically method known to one of skill in the art.

In certain embodiments, a cancer is metastatic. In certain embodiments, a cancer is an advanced cancer which has spread outside the site or organ of origin, either by local invasion or metastasis.

In particular embodiments, a cancer is a recurrent cancer which has regrown, either at the initial site or at a distant site, after a response to initial therapy (e.g., after surgery to remove the tumor and adjuvant therapy following surgery). In some embodiments, a cancer is a refractory cancer which progresses even though an anti-tumor agent, such as a chemotherapeutic agent, is being administered, or has been administered, to the cancer patient. A non-limiting example of a refractory cancer is one which is refractory to a tyrosine kinase inhibitor, such as GLEEVEC® (imatinib mesylate), SUTENT® (SU11248 or sunitinib), TRESSA™ (gefitinib), TARCEVA® (erlotinib), NEXAVAR® (sorafenib), or VOTRIENT™ (pazopanib). In some embodiments, a cancer is a refractory cancer which progresses even though radiation or chemotherapy is being administered, or has been administered, to the cancer patient.

In specific embodiments, provided herein are methods for treating a refractory cancer in a patient in need thereof comprising administering to the patient a therapeutically effective amount of an antibody described herein, wherein the refractory cancer is refractory or resistant to an anti-cancer agent such as a tyrosine kinase inhibitor (e.g., GLEEVEC® (imatinib mesylate) or SUTENT® (SU11248 or Sunitinib)). Other non-limiting examples of tyrosine kinse inhibitors include 706 and AMNI07 (nilotinib). RAD00I, PKC412, gefitinib (TRESSA™), erlotinib (TARCEVA®), sorafenib (NEXAVAR®), pazopanib (VOTRIENT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™), vandetanib (ZACTIMA™), and vatalanib. In certain embodiments, the refractory cancer was initially responsive to an anti-cancer agent, such as a tyrosine kinase inhibitor (e.g., GLEEVEC® or SU11248 (i.e., sunitinib)), but has developed resistance the anti-cancer agent. In certain embodiments, a subject has one or more mutations in KIT that confers resistance to an anti-cancer agent such as a tyrosine kinase inhibitor.

In particular embodiments, an antibody described herein is administered to a patient who has previously received, or is currently receiving, one or more anti-cancer therapies, for example, a chemotherapeutic agent, or a tyrosine kinase inhibitor (e.g., GLEEVEC® (imatinib mesylate), SUTENT® (SU11248 or sunitinib), TRESSA™ (gefitinib), TARCEVA® (erlotinib), NEXAVAR® (sorafenib), or VOTRIENT™ (pazopanib)) or a histone deacetylase inhibitor (e.g., vorinostat or suberoylanilide hydroxamic acid (SAHA)). In other particular embodiments, an antibody described herein is administered to a patient who is, or is suspected of being, resistant or refractory to an anti-cancer therapy, for example, a tyrosine kinase inhibitor, e.g., GLEEVEC® (imatinib mesylate), SUTENT® (SU11248 or sunitinib), IRESSA™ (gefitinib), TARCEVA® (erlotinib), NEXAVAR® (sorafenib), or VOTRIENT™ (pazopanib).

In particular embodiments, an antibody described herein, or an antigen binding fragment thereof (e.g., KIT-binding fragment thereof), or a conjugate thereof) is administered to a patient who has previously received, or is currently receiving, one or more anti-cancer therapies, for example, an anti-growth factor receptor antibody (e.g., anti-HER2 antibody, anti-EGFR antibody, anti-VEGFR antibody, or anti-KIT antibody), or anti-growth factor antibody (e.g., anti-EGF antibody, anti-VEGF antibody). In other particular embodiments, an antibody described herein is administered to a patient who is, or is suspected of being, resistant or refractory to an anti-cancer therapy, for example, an anti-growth factor receptor antibody (e.g., anti-HER2 antibody, anti-EGFR antibody, anti-VEGFR antibody, or anti-KIT antibody) or anti-growth factor antibody (e.g., anti-EGF antibody, anti-VEGF antibody).

In a particular embodiment, a method described herein for protecting against, treating or managing cancer in a subject in need thereof, can achieve at least one, two, three, four or more of the following effects due to administration of a therapeutically effective amount of an anti-KIT antibody described herein: (i) the reduction or amelioration of the severity of cancer (e.g., leukemia, lung cancer, or gastrointestinal stromal cancer) and/or one or more symptoms associated therewith; (ii) the reduction in the duration of one or more symptoms associated with a cancer (e.g., leukemia, lung cancer, or gastrointestinal stromal cancer); (iii) the prevention in the recurrence of a tumor (e.g., lung tumor or gastrointestinal stromal tumor); (iv) the regression of a cancer (e.g., leukemia, lung cancer, or gastrointestinal stromal tumor) and/or one or more symptoms associated therewith; (v) the reduction in hospitalization of a subject; (vi) the reduction in hospitalization length; (vii) the increase in the survival of a subject; (viii) the inhibition of the progression of a cancer (e.g., leukemia, lung cancer, or gastrointestinal stromal tumor) and/or one or more symptoms associated therewith; (ix) the enhancement or improvement of the therapeutic effect of another therapy (e.g., surgery, radiation, chemotherapy, or another tyrosine kinase inhibitor); (x) a reduction or elimination in the cancer cell population (e.g., leukemia cell population, lung cancer cell population, gastrointestinal stromal tumor cell population); (xi) a reduction in the growth of a tumor or neoplasm; (xii) a decrease in tumor size (e.g., volume or diameter); (xiii) a reduction in the formation of a newly formed tumors; (xiv) eradication, removal, or control of primary, regional and/or metastatic cancer; (xv) ease in removal of a tumor by reducing tumor and/or edema-related vascularization prior to surgery; (xvi) a decrease in the number or size of metastases; (xvii) a reduction in mortality; (xviii) an increase in tumor-free survival rate of patients; (xvix) an increase in relapse-free survival; (xx) an increase in the number of patients in remission; (xxi) a decrease in hospitalization rate; (xxii) the size of the tumor is maintained and does not increase or increases by less than the increase of a tumor after administration of a standard therapy as measured by conventional methods available to one of skill in the art, such as computed tomography (CT) scan, magnetic resonance imaging (MRI), dynamic contrast-enhanced MRI (DCE-MRI), or a positron emission tomography (PET) scan; (xxiii) the prevention of the development or onset of one or more symptoms associated cancer; (xxiv) an increase in the length of remission in patients; (xxv) the reduction in the number of symptoms associated with cancer; (xxvi) an increase in symptom-free survival of cancer patients; (xxvii) a decrease in the concentration of one or more inflammatory mediators (e.g., cytokines or interleukins) in biological specimens (e.g., plasma, serum, cerebral spinal fluid, urine, or any other biofluids) of a subject with a cancer (e.g., leukemia, lung cancer, or gastrointestinal stromal cancer); (xxviii) a decrease in circulating tumor cells (CTCs) in the blood of a subject with cancer (e.g., leukemia, lung cancer, or gastrointestinal stromal cancer); (xxix) inhibition (e.g., partial inhibition) or decrease in tumor metabolism or perfusion; and (xxx) improvement in the quality of life as assessed by methods well known in the art, e.g., questionnaires.

In certain aspects, provided herein are methods for killing cancer cells in an individual, wherein said method comprises administering to an individual in need thereof an effective amount of an antibody described herein (e.g., a humanized anti-KIT antibody), or an antigen-binding fragment thereof, or a conjugate thereof. In certain aspects, provided herein are methods for inhibiting growth or proliferation of cancer cells in an individual, wherein said method comprises administering to an individual in need thereof an effective amount of an antibody described herein (e.g., a humanized anti-KIT antibody), or an antigen-binding fragment thereof, or a conjugate thereof. In certain embodiments, partial inhibition of growth or proliferation of cancer cells is achieved, for example, inhibition of at least about 20% to about 55% of growth or proliferation of cancer cells.

In certain aspects, provided herein are methods for reducing tumor size or load in an individual in need thereof, wherein said method comprises administering to said individual an effective amount of an antibody described herein (e.g., a humanized anti-KIT antibody), or an antigen-binding fragment thereof, or a conjugate thereof.

In certain embodiments, an anti-KIT antibody described herein may be administered by any suitable method to a subject in need thereof. Non-limiting examples of administration methods include mucosal, intradermal, intravenous, intratumoral, subcutaneous, intramuscular delivery and/or any other method of physical delivery described herein or known in the art. In one embodiment, an anti-KIT antibody or a pharmaceutical composition thereof is administered systemically (e.g., parenterally) to a subject in need thereof. In another embodiment, an anti-KIT antibody or a pharmaceutical composition thereof is administered locally (e.g., intratumorally) to a subject in need thereof. In a specific embodiment, an anti-KIT antibody or a pharmaceutical composition thereof is administered intravenously. In a specific embodiment, an anti-KIT antibody or a pharmaceutical composition thereof is administered subcutaneously. Each dose may or may not be administered by an identical route of administration. In some embodiments, an anti-KIT antibody described herein can be administered via multiple routes of administration simultaneously or subsequently to other doses of the same or a different an anti-KIT antibody described herein.

When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof. In certain embodiments, an anti-KIT antibody described herein is administered prophylactically or therapeutically to a subject. An anti-KIT antibody described herein can be prophylactically or therapeutically administered to a subject so as to prevent, lessen or ameliorate a KIT-associated disorder or disease (e.g., cancer, inflammatory condition, fibrosis) or symptom thereof.

The dosage and frequency of administration of an anti-KIT antibody described herein or a pharmaceutical composition thereof is administered to a subject in need thereof (e.g., mammal, such as human, dog or cat) in accordance with the methods for treating a KIT-associated disorder, eosinophil related disorder or mast cell related disorder provided herein will be efficacious while minimizing side effects. The exact dosage of an anti-KIT antibody described herein to be administered to a particular subject or a pharmaceutical composition thereof can be determined in light of factors related to the subject that requires treatment. Factors which can be taken into account include the severity of the disease state, general health of the subject, age, and weight of the subject, diet, time and frequency of administration, combination(s) with other therapeutic agents or drugs, reaction sensitivities, and tolerance/response to therapy. The dosage and frequency of administration of an anti-KIT antibody described herein or a pharmaceutical composition thereof can be adjusted over time to provide sufficient levels of the anti-KIT antibody or to maintain the desired effect.

The precise dose to be employed in the formulation will also depend on the route of administration, and the seriousness of the disorder or disease and should be decided according to the judgment of the practitioner and each patient's circumstances.

In certain aspects, for the anti-KIT antibodies described herein, the dosage administered to a patient, to prevent, protect against, manage, or treat a KIT-associated disorder, eosinophil related disorder or mast cell related disorder is typically 0.1 mg/kg to 100 mg/kg of the patient's body weight. In a specific embodiment, the dosage administered to a patient, to prevent, protect against, manage, or treat a KIT-associated disorder, eosinophil related disorder (such as eosinophilic esophagitis (EoE)) or mast cell related disorder (e.g., chronic urticaria, such as chronic inducible urticaria) is about 3 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of the antibodies described herein can be reduced by enhancing uptake and tissue penetration of the antibodies by modifications such as, for example, lipidation.

In one embodiment, approximately 0.001 mg/kg (mg of antibody per kg weight of a subject) to approximately 500 mg/kg of an anti-KIT antibody described herein is administered to prevent, protect against, manage, or treat a KIT-associated disorder, a mast cell associated disorder or an eosinophil-related disorder such as eosinophilic esophagitis (EoE).

In some embodiments, an effective amount of an antibody provided herein is from about 0.01 mg to about 1,000 mg. In specific embodiments, an “effective amount” or “therapeutically effective amount” of an anti-KIT antibody described herein refers to an amount of an anti-KIT antibody described herein which is sufficient to achieve at least one, two, three, four or more of the following effects: the reduction or amelioration of the severity of a KIT-associated disorder, a mast cell associated disorder or an eosinophil-related disorder, and/or one or more symptoms associated therewith; the reduction in the duration of one or more symptoms associated with a KIT-associated disorder, a mast cell associated disorder or an eosinophil-related disorder; the prevention in the recurrence of one or more symptoms of a KIT-associated disorder, a mast cell associated disorder or an eosinophil-related disorder, and/or one or more symptoms associated therewith; the reduction in hospitalization of a subject; the reduction in hospitalization length; the inhibition (e.g., partial inhibition) of the progression of a KJT-associated disorder, a mast cell associated disorder or an eosinophil-related disorder, and/or one or more symptoms associated therewith; the prevention of the development or onset of one or more symptoms associated with a KIT-associated disorder, a mast cell associated disorder or an eosinophil-related disorder; a decrease in the concentration of one or more inflammatory mediators (e.g., cytokines or interleukins) in biological specimens (e.g., plasma, serum, cerebral spinal fluid, urine, or any other bio fluids) of a subject with a KIT-associated disorder, a mast cell associated disorder or an eosinophil-related disorder; and improvement in the quality of life as assessed by methods well known in the art, e.g., questionnaires. In some embodiments, “effective amount” as used herein also refers to the amount of an antibody described herein to achieve a specified result (e.g., inhibition of one or more KIT biological activities of a cell, such as inhibition of cell proliferation).

In some embodiments, an anti-KIT antibody described herein is administered as necessary, e.g., weekly, biweekly (i.e., once every two weeks), monthly, bimonthly, trimonthly, etc.

In some embodiments, a single dose of an anti-KIT antibody described herein is administered one or more times to a patient to impede, prevent, protect against, manage, treat and/or ameliorate a KIT-associated disorder, a mast cell associated disorder or an eosinophil-related disorder such as eosinophilic esophagitis (EoE).

In particular embodiments, an anti-KIT antibody or pharmaceutical composition thereof is administered to a subject in accordance with the methods for treating a KIT-associated disorder, a mast cell associated disorder or an eosinophil-related disorder, provided herein in cycles, wherein the anti-KIT antibody or pharmaceutical composition is administered for a period of time, followed by a period of rest (i.e., the anti-KIT antibody or pharmaceutical composition is not administered for a period of time).

The methods provided herein involve administering an anti-KIT antibody by any suitable routes. Non-limiting examples of routes of administration include, parenteral administration for example subcutaneous, intramuscular or intravenous administration, epidural administration, enteric administration, intracerebral administration, nasal administration, intraarterial administration, intracardiac administration, intraosseous infusion, intrathecal administration, and intraperitoneal administration. Methods provided herein include routes of administration targeting the brain, an ocular tissue or organ, spinal cord, or ear or auricular tissue. In a particular aspect, methods provided herein include routes of administration targeting the nervous system, e.g., central nervous system.

In specific embodiments, methods provided herein involve administering an anti-KIT antibody via a route suitable for crossing the blood-brain barrier.

Also, presented herein are combination therapies for the treatment of a KIT-associated disorder or disease (e.g., mast cell related disorder such as urticaria (e.g., chronic inducible urticaria), eosinophil related disorder such as eosinophilic esophagitis, cancer, inflammatory condition, fibrosis) which involve the administration of an anti-KIT antibody described herein (e.g., a humanized anti-KIT antibody), or an antigen-binding fragment thereof (e.g., KIT-binding fragment thereof), or an antibody conjugate thereof in combination with one or more additional therapies (e.g., a second therapeutic agent, such as a chemotherapeutic agent, tyrosine kinase inhibitor, PGP inhibitors, HSP-90 inhibitors, proteosome inhibitors, histone deacetylase inhibitor, antibody, or cytokine) to a subject in need thereof. In a specific embodiment, presented herein are combination therapies for the treatment of a KIT-associated disorder or disease (e.g., mast cell related disorder such as urticaria (e.g., chronic inducible urticaria), eosinophil related disorder such as eosinophilic esophagitis, cancer, inflammatory condition, fibrosis) which involve the administration of an amount (e.g., a therapeutically effective amount or a sub-optimal amount) of an anti-KIT antibody described herein in combination with an amount (e.g., a therapeutically effective amount or a sub-optimal amount) of another therapy (e.g., chemotherapeutic agent, tyrosine kinase inhibitor, antibody, cytokine, antihistamine, leukotriene receptor antagonist, immunomodulator, anti-inflammatory agent, or histone deacetylase inhibitor) to a subject in need thereof. In various embodiments, the combination therapies are administered to the subject at about the same time, the same day, the same week, or the same treatment cycle, or on similar or overlapping dosing schedules. In some embodiments, the combination therapies are administered to the subject simultaneously, concurrently or concomitantly. In other embodiments, the combination therapies are administered to the subject sequentially. In one embodiment, the anti-KIT antibody, antigen binding fragment thereof, or conjugate described herein is administered to the subject prior to the other therapy(ies) in the combination. In another embodiment, the anti-KIT antibody, antigen binding fragment thereof, or conjugate described herein is administered to the subject subsequent to the other therapy(ies) in the combination.

In combination therapies, one or more anti-KIT antibodies provided herein (e.g., a humanized anti-KIT antibody), or an antigen-binding fragment thereof (e.g., KIT-binding fragment thereof), or an antibody conjugate thereof can be administered prior to, concurrently with, or subsequent to the administration of one or more additional therapies (e.g., agents, surgery, or radiation) for use in protecting against, treating, managing, and/or ameoliorating a KIT-associated disorder or disease (e.g., mast cell related disorder such as urticaria (e.g., chronic inducible urticaria), eosinophil related disorder such as eosinophilic esophagitis, cancer, inflammatory condition, fibrosis). The use of the term “in combination” does not restrict the order in which one or more anti-KIT antibodies and one or more additional therapies are administered to a subject. In specific embodiments, the therapies can be administered serially or sequentially.

In specific embodiments, one or more anti-KIT antibodies provided herein (e.g., a humanized anti-KIT antibody), or an antigen-binding fragment thereof (e.g., KIT-binding fragment thereof), or an antibody conjugate thereof can be administered prior to, concurrently with, or subsequent to the administration of one or more additional therapies such as anticancer agents, for example, tyrosine kinase inhibitors (e.g., imatinib myselyate (Gleevec®) or sunitinib (SUTENT), or histone deacetylase inhibitors (e.g., vorinostat or suberoylanilide hydroxamic acid (SAHA)), for protecting against, treating, managing, and/or ameoliorating a KIT-associated disorder or disease (e.g., cancer, for example, GIST, melanoma, or lung cancer).

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with one or more antihistamines (e.g., H1- and/or H2-antihistamine treatment(s)) for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently or concomitantly with one or more antihistamines (e.g., H1- and/or H2-antihistamine treatment(s)) for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with one or more antihistamines (e.g., H1- and/or H2-antihistamine treatment) for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with one or more leukotriene receptor antagonists for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently or concomitantly with one or more leukotriene receptor antagonists for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with one or more leukotriene receptor antagonists for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-TL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently or concomitantly with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with one or more immunomodulators or anti-inflammatory agents (for example, an IgE inhibitor such as an anti-IgE antibody, e.g., omalizumab (Xolair®) or ligelizumab, an IL-4R inhibitor such as an anti-IL-4R antibody, e.g., dupilumab (Dupixent®), an IL-5R inhibitor such as an anti-IL-5R antibody, e.g., benralizumab (Fasenra®), an IL-5 inhibitor such as an anti-IL-5 antibody, e.g., mepolizumab, a Siglec 8 inhibitor such as an anti-Siglec 8 antibody, e.g., lirentelimab, a TSLP or TSLPR inhibitor such as an anti-TSLP or anti-TSLPR antibody, e.g., tezepelumab (Tezspire™), a C5aR inhibitor such as an anti-C5aR antibody, e.g., avdoralimab, and/or a CD200R inhibitor such as an anti-CD200R antibody, e.g., LY3454738) for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with an IgE inhibitor such as an anti-IgE antibody, e.g., omalizumab (Xolair®) or ligelizumab, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with an IL-4R inhibitor such as an anti-IL-4R antibody, e.g., dupilumab (Dupixent®), for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with an IL-5R inhibitor such as an anti-IL-5R antibody, e.g., benralizumab (Fasenra®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with an IL-5R inhibitor such as an anti-IL-5R antibody, e.g., benralizumab (Fasenra®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with an IL-5R inhibitor such as an anti-IL-5R antibody, e.g., benralizumab (Fasenra®), for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with an IL-5 inhibitor such as an anti-IL-5 antibody, e.g., mepolizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with an IL-5 inhibitor such as an anti-IL-5 antibody, e.g., mepolizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with an IL-5 inhibitor such as an anti-IL-5 antibody, e.g., mepolizumab, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with a Siglec 8 inhibitor such as an anti-Siglec 8 antibody, e.g., lirentelimab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with a Siglec 8 inhibitor such as an anti-Siglec 8 antibody, e.g., lirentelimab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with a Siglec 8 inhibitor such as an anti-Siglec 8 antibody, e.g., lirentelimab, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with a TSLP or TSLPR inhibitor such as an anti-TSLP or anti-TSLPR antibody, e.g., tezepelumab (Tezspire^{M}), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with a TSLP or TSLPR inhibitor such as an anti-TSLP or anti-TSLPR antibody, e.g., tezepelumab (Tezspire™), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with a TSLP or TSLPR inhibitor such as an anti-TSLP or anti-TSLPR antibody, e.g., tezepelumab (Tezspire^{T}M), for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with a C5aR inhibitor such as an anti-C5aR antibody, e.g., avdoralimab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with a C5aR inhibitor such as an anti-C5aR antibody, e.g., avdoralimab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with a C5aR inhibitor such as an anti-C5aR antibody, e.g., avdoralimab, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with a CD200R inhibitor such as an anti-CD200R antibody, e.g., LY3454738, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with a CD200R inhibitor such as an anti-CD200R antibody, e.g., LY3454738, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with a CD200R inhibitor such as an anti-CD200R antibody, e.g., LY3454738, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with one or more BTK inhibitors, e.g., remibrutinib and/or rilzabrutinib, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently or concomitantly with one or more BTK inhibitors, e.g., remibrutinib and/or rilzabrutinib, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with one or more BTK inhibitors, e.g., remibrutinib and/or rilzabrutinib, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with (1) one or more antihistamines, as described above, (2) one or more leukotriene receptor antagonists, as described above, (3) one or more immunomodulators or anti-inflammatory agents, as described above, and/or (4) one or more BTK inhibitors, as described above, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with (1) one or more antihistamines, as described above, and (2) one or more immunomodulators or anti-inflammatory agents, as described above, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with (1) one or more antihistamines, as described above, and (2) one or more immunomodulators or anti-inflammatory agents, as described above, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with (1) one or more antihistamines, as described above, and (2) one or more immunomodulators or anti-inflammatory agents, as described above, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with (1) one or more antihistamines, as described above, (2) an IgE inhibitor such as an anti-IgE antibody, e.g., omalizumab (Xolair®) or ligelizumab, and (3) an IL-4R inhibitor such as an anti-IL-4R antibody, e.g., dupilumab (Dupixent®), for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with (1) one or more antihistamines, as described above, and (2) an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with (1) one or more antihistamines, as described above, and (2) an IgE inhibitor such as an anti-IgE antibody, e.g., omalizumab (Xolair®) or ligelizumab, for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with (1) one or more antihistamines, as described above, and (2) an IgE inhibitor such as an anti-IgE antibody, e.g., omalizumab (Xolair®) or ligelizumab, for the disorder.

In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis in combination with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis simultaneously, concurrently, or concomitantly with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder. In one embodiment, an anti-KIT antibody, antigen binding fragment thereof, or conjugate described herein is administered to a subject having a mast cell related disorder such as urticaria (e.g., chronic inducible urticaria) or an eosinophil related disorder such as eosinophilic esophagitis sequentially with (1) an IgE inhibitor such as an anti-IgE antibody, e.g., omalizumab (Xolair®) or ligelizumab, and (2) an IL-4R inhibitor such as an anti-IL-4R antibody, e.g., dupilumab (Dupixent®), for the disorder.

In another specific embodiment, presented herein are combination therapies for the treatment of a KIT-associated disorder or disease (e.g., mast cell related disorder such as urticaria (e.g., chronic inducible urticaria), eosinophil related disorder such as eosinophilic esophagitis, cancer, inflammatory condition, fibrosis) which involve the administration of an amount of an anti-KIT antibody described herein (e.g., a humanized anti-KIT antibody), or an antigen-binding fragment thereof (e.g., KIT-binding fragment thereof), or an antibody conjugate thereof in combination with an amount of another therapy (e.g., chemotherapeutic agent, tyrosine kinase inhibitor, or histone deacetylase inhibitor) to a subject in need thereof. In a specific embodiment, the combination therapies result in a synergistic effect. In certain embodiments, the combination therapies result in an additive effect.

In a specific embodiment, presented herein are combination therapies for the treatment of cancer which involve the administration of an amount of an anti-KIT antibody described herein in combination with an amount of another therapy (e.g., surgery, radiation, stem cell transplantation, or chemotherapy) to a subject in need thereof. In a specific embodiment, the combination therapies result in a synergistic effect. In another specific embodiment, the combination therapies result in an additive effect.

In a specific embodiment, presented herein are combination therapies for the treatment of an inflammatory condition which involve the administration of an amount of an anti-KIT antibody described herein in combination with an amount of another therapy (e.g., anti-inflammatory therapy, for example, steroid therapy) to a subject in need thereof. In a specific embodiment, the combination therapies result in a synergistic effect. In another specific embodiment, the combination therapies result in an additive effect.

Non-limiting examples of another therapy for use in combination with antibodies described herein include, another anti-KIT antibody that immunospecifically binds to a different epitope of KIT, one or more other antibodies (e.g., anti-HER2 antibody, anti-EGFR antibody, anti-VEGF antibody), anti-inflammatory therapy, chemotherapy (e.g., microtubule disassembly blocker, antimetabolite, topisomerase inhibitor, and DNA crosslinker or damaging agent), radiation, surgery, PGP inhibitors (e.g., cyclosporine A, Verapamil), HSP-90 inhibitors (e.g., 17-AAG, STA-9090), proteosome inhibitors (e.g., Bortezomib), and tyrosine kinase inhibitors (e.g., imatinib mesylate (GLEEVEC®), sunitinib (SUTENT® or SU11248), gefitinib (IRESSA™) erlotinib (TARCEVA®), sorafenib (NEXAVAR®), pazopanib (VOTRIENT™), axitinib, bosutinib, cediranib (RECENTIN®), SPRYCEL® (dasatinib), lapatinib (TYKERB®), lestaurtinib, neratinib, nilotinib (TASIGNA®), semaxanib, toceranib (PALLADIA™), vandetanib (ZACTIMA™), and vatalanib). In a specific embodiment, another therapy for use in combination with antibodies described herein is imatinib mesylate.

Other non-limiting examples of another therapy for use in combination with antibodies described herein (e.g., a humanized anti-KIT antibody), or an antigen-binding fragment thereof (e.g., KIT-binding fragment thereof), or an antibody conjugate thereof include a histone deacetylase inhibitor, such as vorinostat or suberoylanilide hydroxamic acid (SAHA) or a compound having the chemical formula (I), (II), or (III) as set forth below. In a specific embodiment, provided herein is a method for treating cancer (e.g., GIST or lung cancer) comprising (i) administering an antibody described herein (e.g., a humanized anti-KIT antibody), or an antigen-binding fragment thereof (e.g., KIT-binding fragment thereof), or an antibody conjugate thereof; and (ii) a histone deacetylase inhibitor, for example, vorinostat or suberoylanilide hydroxamic acid (SAHA) or a compound having the chemical formula (I), (II), or (III) as set forth below.

In one embodiment, provided herein for use in the methods described herein in combination with anti-KIT antibodies are compounds of Formula (I)

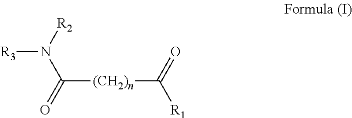

or a pharmaceutically acceptable salt, hydrate, or solvate thereof, wherein
R₁ is hydroxylamino;
each of R₂ and R₃ are independently the same as or different from each other, substituted or unsubstituted, branched or unbranched, and are hydrogen, hydroxyl, alkyl, alkenyl, cycloalkyl, aryl, alkyloxy, aryloxy, arylalkyloxy or pyridine; or R₂ and R₃ are bonded together to form a piperidine; and
n is an integer from 5 to 7.

In one embodiment, R₂ is hydrogen atom and R₃ is substituted or unsubstituted phenyl. In a certain embodiment, R₃ is phenyl substituted with methyl, cyano, nitro, trifluoromethyl, amino, aminocarbonyl, methylcyano, chloro, fluoro, bromo, iodo, 2,3-difluoro, 2,4-difluoro, 2,5-difluoro, 3,4-difluoro, 3,5-difluoro, 2,6-difluoro, 1,2,3-trifluoro, 2,3,6-trifluoro, 2,4,6-trifluoro, 3,4,5-trifluoro, 2,3,5,6-tetrafluoro, 2,3,4,5,6-pentafluoro, azido, hexyl, t-butyl, phenyl, carboxyl, hydroxyl, methoxy, phenyloxy, benzyloxy, phenylaminooxy, phenylaminocarbonyl, methoxycarbonyl, methylaminocarbonyl, dimethylamino, dimethylaminocarbonyl, or hydroxylaminocarbonyl. In another embodiment, R₃ is unsubstituted phenyl. In a further embodiment, n is 6.

In one embodiment, provided herein for use in the methods described herein in combination with anti-KIT antibodies are compounds of Formula (II)

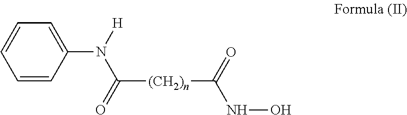

or a pharmaceutically acceptable salt, or solvate thereof, wherein n is an integer from 5 to 8. In one embodiment n is 6.

In one embodiment, provided herein for use in the methods described herein in combination with anti-KIT antibodies is a compound of Formula (III) (SAHA)

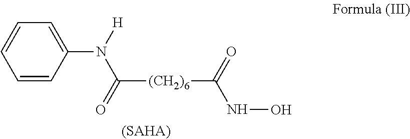

or a pharmaceutically acceptable salt, hydrate or solvate thereof.

Compounds of Formulae I-III can be synthesized according to the methods described in U.S. Reissued Patent No. RE38,506 and U.S. Pat. No. 6,087,367, each of which is herewith incorporated by reference in its entirety.

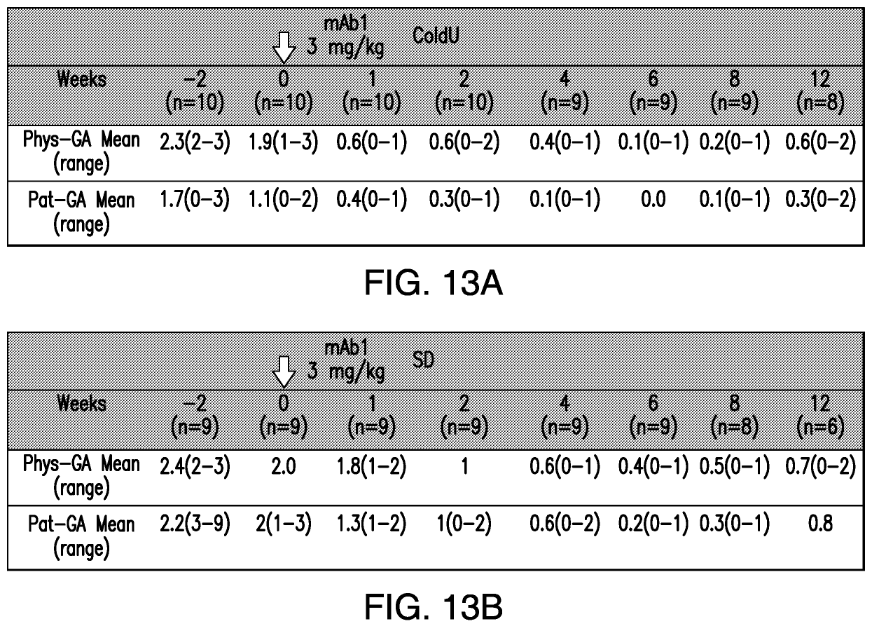

In one embodiment, provided herein for use in the methods described herein in combination with anti-KIT antibodies is a Form I polymorph of SAHA characterized by an X-ray diffraction pattern substantially similar to that set forth in FIG. 13A of U.S. Pat. No. 7,456,219, which is herewith incorporated by reference in its entirety. In one embodiment the Form I polymorph of SAHA is characterized by an X-ray diffraction pattern including characteristic peaks at about 9.0, 9.4, 17.5, 19.4, 20.0, 24.0, 24.4, 24.8, 25.0, 28.0, and 43.3 degrees 2θ, as measured with a Siemens D500 Automated Powder Diffractometer (range: 4-40 degrees 2θ; source: Cu; λ=1.54 Angstrom, 50 kV, 40 mA).

In a certain embodiment, the Form I polymorph of SAHA is characterized by a Differential Scanning Calorimetry (DSC) thermogram having a single maximum value at about 164.4±2.0° C., as measured by a Perkins Elmer DSC 6 Instrument at a heating rate of 10° C./min from 50° C. to at least 30° C. above the observed melting temperature.

The Form I polymorph of SAHA can be synthesized according to the methods described in U.S. Pat. No. 7,456,219.

In one embodiment, provided herein is a crystalline composition comprising Lysine and SAHA characterized by an X-ray diffraction pattern substantially similar to that set forth in FIG. 1 of International Patent Application Publication No. WO2008/042146, which is herewith incorporated by reference in its entirety. In another embodiment, the crystalline composition is characterized by an X-ray diffraction pattern including characteristic peaks at about 6.8, 20.1 and 23.2 degrees 2θ, as measured with a PANanalytical X'Pert Pro X-ray powder diffractometer (range: 2-40 degrees 2θ; source: Cu Kα1 and Kₐ2). In another embodiment, the crystalline composition is characterized by an X-ray diffraction pattern including characteristic peaks at about 6.8, 12.6, 18.7, 20.1 23.2, and 24.0 degrees 2θ, as measured with a PANanalytical X'Pert Pro X-ray powder diffractometer (range: 2-40 degrees 2θ; source: Cu Kα1 and Kₐ2). In another embodiment, the crystalline composition is characterized by an X-ray diffraction pattern including characteristic peaks at about 6.8, 12.0, 12.6, 16.4, 18.7, 20.1 23.2, 24.0, 29.3 degrees 2θ, as measured with a PANanalytical X'Pert Pro X-ray powder diffractometer (range: 2-40 degrees 2θ; source: Cu Kα1 and Kₐ2).

In a certain embodiment, the crystalline composition comprising Lysine and SAHA is characterized by a Differential Scanning Calorimetry (DSC) thermogram, wherein the endotherm of the crytalline composition exhibits an extrapolated onset temperature of approximately 182° C., as measured by a TA Instruments Q1000 differential scanning calorimeter at a heating rate of 10° C./min from room temperature to 300° C.

The crystalline composition comprising Lysine and SAHA can be synthesized according to the methods described in International Patent Application Publication No. WO2008/042146.

In certain embodiments, combination therapies described herein result in synergy or a synergistic effect. In a specific embodiment, a synergistic effect of a combination therapy permits the use of lower dosages (e.g., sub-optimal doses) of an anti-KIT antibody described herein and/or an additional therapy and/or less frequent administration of an anti-KIT antibody described herein or an additional therapy to a subject. In certain embodiments, the ability to utilize lower dosages of an anti-KIT antibody and/or of an additional therapy and/or to administer an anti-KIT antibody or said additional therapy less frequently reduces the toxicity associated with the administration of an anti-KIT antibody or of said additional therapy, respectively, to a subject without reducing the efficacy of an anti-KIT antibody or of said additional therapy, respectively, in the treatment of a KIT-associated disorder or disease. In some embodiments, a synergistic effect results in improved efficacy of an anti-KIT antibody described herein and/or of said additional therapies in treating a KIT-associated disorder or disease. In some embodiments, a synergistic effect of a combination of an anti-KIT antibody described herein and one or more additional therapies avoids or reduces adverse or unwanted side effects associated with the use of any single therapy.

Provided herein are methods for inhibiting KIT activity in a cell expressing KIT comprising contacting the cell with an effective amount of an antibody described herein (e.g., a humanized anti-KIT antibody), or an antigen-binding fragment thereof (e.g., KIT-binding fragment thereof), or an antibody conjugate thereof. In a specific embodiment, the methods inhibit KIT activity by at least about 10%, at least about 20%, at least about 30%, at least about 40%, or at least about 50% in the cell expressing KIT. Also provided herein are methods for inducing or enhancing apoptosis in a cell expressing KIT comprising contacting the cell with an effective amount of an antibody described herein. Also provided herein are methods for inducing or enhancing cell differentiation in a cell expressing KIT comprising contacting the cell with an effective amount of an antibody described herein.

KIT activity and, for example, the effect of an antibody on KIT activity can routinely be assessed using, e.g., cell-based assays such as those described herein.

Non-limiting examples of KIT activity which can be inhibited by the methods provided herein can include any activity of KIT known or described in the art, e.g., KIT receptor dimerization, KIT receptor phosphorylation (tyrosine phosphorylation), signaling downstream of the KIT receptor (e.g., Stat, AKT, MAPK, or Ras signaling), KIT ligand (e.g., SCF) induced transcriptional regulation (e.g., SCF-induced transcriptional activation of c-Myc), induction or enhancement of cell proliferation, or cell survival.

In certain embodiments, a method for inhibiting (e.g., partially inhibiting) KIT activity in a cell expressing KIT comprises contacting the cell with an effective amount of an antibody described herein (e.g., a humanized anti-KIT antibody), or an antigen-binding fragment thereof (e.g., KIT-binding fragment thereof), or an antibody conjugate thereof, sufficient to inhibit or antagonize KIT activity by at least about 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein and/or known to one of skill in the art (e.g., ELISA). In certain embodiments, a method for inhibiting (e.g., partially inhibiting) KIT activity in a cell expressing KIT comprises contacting the cell with an effective amount of an antibody described herein (e.g., a humanized anti-KIT antibody), or an antigen-binding fragment thereof (e.g., KIT-binding fragment thereof), or an antibody conjugate thereof, sufficient to inhibit or antagonize KIT activity by at least about 25%, 35%, 45%, 50%, 55%, or 65%, as assessed by methods described herein and/or known to one of skill in the art (e.g., ELISA). Non-limiting examples of KIT activity can include KIT receptor phosphorylation, KIT receptor signaling, KIT ligand (e.g., SCF) mediated cell proliferation, KIT ligand (e.g., SCF) mediated cell survival, and transcriptional activation of a KIT target gene (e.g., c-Myc).

In a particular embodiment, a method for inhibiting KIT activity in a cell expressing KIT comprises contacting the cell with an effective amount of an antibody described herein (e.g., a humanized anti-KIT antibody), or an antigen-binding fragment thereof (e.g., KIT-binding fragment thereof), or an antibody conjugate thereof, sufficient to inhibit (e.g., partially inhibit) or antagonize downstream KIT signaling, for example, signaling of a member of the Src family kinases, PI3-kinases, or Ras-MAPK.

In another particular embodiment, a method for inhibiting (e.g., partially inhibiting) one or more KIT activities in a cell expressing KIT, comprises contacting the cell with an effective amount of an antibody described herein sufficient to inhibit or antagonize downstream KIT signaling such as phosphorylation of MAPK, phosphorylation of AKT, or phosphorylation of Statl, Stat3, or Stat5.

In certain embodiments, a method for inhibiting (e.g., partially inhibiting) KIT activity in a cell expressing KIT comprises contacting the cell with an effective amount of an antibody described herein sufficient to inhibit or to reduce phosphorylation of AKT (e.g., KIT ligand (e.g., SCF) induced phosphorylation of AKT) by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art, e.g., Western blot or ELISA assay or immunoblotting assay. In certain embodiments, a method for inhibiting e.g., partially inhibiting) KIT activity in a cell expressing KIT comprises contacting the cell with an effective amount of an antibody described herein sufficient to inhibit or to reduce phosphorylation of AKT (e.g., KIT ligand (e.g., SCF) induced phosphorylation of AKT) by at least about 25%, 35%, 45%, 55%, or 65%, as assessed by methods described herein or known to one of skill in the art, e.g., Western blot or ELISA assay or immunoblotting assay.

In certain aspects, a method for inhibiting (e.g., partially inhibiting) KIT activity in a cell (e.g., cancer cell) expressing KIT comprises contacting the cell with an effective amount of an antibody described herein sufficient to inhibit proliferation of the cell. Cell proliferation assays are described in the art and can be readily carried out by one of skill in the art. For example, cell proliferation can be assayed by measuring Bromodeoxyuridine (BrdU) incorporation (see, e.g., Hoshino et al., 1986, Int. J. Cancer 38, 369; Campana et al., 1988, J. Immunol. Meth. 107:79) or (3H) thymidine incorporation (see, e.g., Blechman et al., Cell, 1995, 80:103-113; Chen, J., 1996, Oncogene 13:1395-403; Jeoung, J., 1995, J. Biol. Chem. 270:18367 73), by direct cell count at various time intervals (e.g., 12-hour or 24-hour intervals), or by detecting changes in transcription, translation or activity of known genes such as proto-oncogenes (e.g., fos, myc) or cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription.

In specific embodiments, a method for inhibiting (e.g., partially inhibiting) KIT activity in cells (e.g., cancer cells) expressing KIT comprises contacting the cells with an effective amount of an antibody described herein sufficient to inhibit cell proliferation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., BrdU incorporation assay). In specific embodiments, a method for inhibiting (e.g., partially inhibiting) KIT activity in cells expressing KIT comprises contacting the cells with an effective amount of an antibody described herein sufficient to inhibit cell proliferation by at least about 25%, 35%, 45%, 55%, or 65%, as assessed by methods described herein or known to one of skill in the art (e.g., BrdU incorporation assay). In specific embodiments, a method for an inhibiting or antagonizing KIT activity in cells expressing KIT comprises contacting the cells with an effective amount of an antibody described herein sufficient to inhibit cell proliferation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., BrdU incorporation assay).

In certain aspects, a method provided herein for inhibiting KIT activity in a cell (e.g., cancer cell) expressing KIT comprises contacting the cell with an effective amount of an antibody described herein sufficient to reduce or to inhibit survival of the cell. Cell survival assays are described in the art and can be readily carried out by one of skill in the art. For example, cell viability can be assessed by using trypan-blue staining or other cell death or viability markers known in the art. In a specific embodiment, the level of cellular ATP is measured to determined cell viability. In specific embodiments, cell viability is measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes can include enlargement, granularity, cells with ragged edges, a filmy appearance, rounding, detachment from the surface of the well, or other changes. These changes are given a designation of T (100% toxic), PVH (partially toxic-very heavy-80%), PH (partially toxic-heavy-60%), P (partially toxic-40%), Ps (partially toxic-slight-20%), or 0 (no toxicity-0%), conforming to the degree of cytotoxicity seen. A 50% cell inhibitory (cytotoxic) concentration (IC₅₀) is determined by regression analysis of these data.

In specific embodiments, a method provided herein for inhibiting (e.g., partially inhibiting) KIT activity in cells expressing KIT comprises contacting the cells with an effective amount of an antibody described herein sufficient to reduce or to inhibit survival of the cells by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., trypan blue exclusion assay). In specific embodiments, a method provided herein for inhibiting (e.g., partially inhibiting) KIT activity in cells expressing KIT comprises contacting the cells with an effective amount of an antibody described herein sufficient to reduce or to inhibit survival of the cells by at least about 25%, 35%, 45%, 55%, or 65%, as assessed by methods described herein or known to one of skill in the art (e.g., trypan blue exclusion assay). In specific embodiments, a method provided herein for inhibiting KIT activity in cells expressing KIT comprises contacting the cells with an effective amount of an antibody described herein sufficient to reduce or to inhibit survival of the cells by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., trypan blue assay).

In a specific embodiment, a method provided herein for inhibiting (e.g., partially inhibiting) KIT activity in cells expressing KIT comprises contacting the cells with an effective amount of an antibody described herein sufficient to induce apoptosis (i.e., programmed cell death). Methods for detecting apoptosis are described in the art and can be readily carried out by one of skill in the art. For example, flow cytometry can be used to detect activated caspase 3, an apoptosis-mediating enzyme, in cells undergoing apoptosis, or Western blotting can be used to detect cleavage of poly(ADP-ribose) polymerase (PARP (see, e.g., Smolich et al., Blood, 2001, 97:1413-1421). Cleavage of PARP is an indicator of apoptosis. In specific embodiments, a method provided herein for an inhibiting or antagonizing KIT activity in cells expressing KIT comprises contacting the cells with an effective amount of an antibody described herein sufficient to induce or enhance apoptosis by at least about 5%, 10%, 15%, 20%, 25%, 30%, 35%. 40%, 45%, 50%, 55%, 60%, 65%, 70%, 75%, 80%, 85%, 90%, 95%, 98%, or 99% as assessed by methods described herein or known to one of skill in the art (e.g., flow cytometry to detect activated caspases 3). In specific embodiments, a method provided herein for an inhibiting or antagonizing KIT activity in cells expressing KIT comprises contacting the cells with an effective amount of an antibody described herein sufficient to induce or enhance apoptosis by at least about 25%, 35%, 45%, 55%, or 65%, as assessed by methods described herein or known to one of skill in the art (e.g., flow cytometry to detect activated caspases 3). In specific embodiments, antibodies a method provided herein for inhibiting KIT activity in cells expressing KIT comprises contacting the cells with an effective amount of an antibody described herein sufficient to induce or enhance apoptosis by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., flow cytometry to detect activated caspase 3).

In a specific embodiment, a method provided herein for inhibiting (e.g., partially inhibiting) KIT activity in a cell expressing KIT comprises contacting the cells with an effective amount of an antibody described herein sufficient to induce differentiation. Methods for detecting differentiation are described in the art and can be readily carried out by one of skill in the art. For example, flow cytometry can be used to detect expression of one or more differentiation markers, or the lack of expression of one or more undifferentiated markers, in a cell contacted with an antibody described herein. Similarly, Western blotting can also be used to detect differentiation markers. Suitable differentiation markers and undifferentiated markers have been described and are one of skill in the art.

In specific embodiments, a method provided herein for inhibiting (e.g., partially inhibiting) KIT activity in cells expressing KIT comprises contacting the cells with an effective amount of an antibody described herein sufficient to induce differentiation by at least about 5%, 10%, 15%, 20%, 25%, 30%, 35%, 40%, 45%, 50%, 55%, 60%, 65%, 70%, 75%, 80%, 85%, 90%, 95%, 98%, or 99% as assessed by methods described herein or known to one of skill in the art (e.g., flow cytometry). In specific embodiments, a method provided herein for inhibiting (e.g., partially inhibiting) KIT activity in cells expressing KIT comprises contacting the cells with an effective amount of an antibody described herein sufficient to induce differentiation by at least about 25%, 35%, 45%, 55%, or 65%, as assessed by methods described herein or known to one of skill in the art (e.g., flow cytometry). In specific embodiments, a method provided herein for inhibiting KIT activity in cells expressing KIT comprises contacting the cells with an effective amount of an antibody described herein sufficient to induce differentiation by at least about 1 fold, 1.2 fold, 1.3 fold, 1.4 fold, 1.5 fold, 2 fold, 2.5 fold, 3 fold, 3.5 fold, 4 fold, 4.5 fold, 5 fold, 6 fold, 7 fold, 8 fold, 9 fold, 10 fold, 15 fold, 20 fold, 30 fold, 40 fold, 50 fold, 60 fold, 70 fold, 80 fold, 90 fold, or 100 fold as assessed by methods described herein or known to one of skill in the art (e.g., flow cytometry).

Non-limiting examples of cells which can be differentiated by the methods described herein include stem cells (e.g., embryonic stem cells, hematopoietic stem cells) and progenitor cells. Exemplary hematopoietic stem cell markers include CD38, CD34, CD59, CD133, Sca-1, and ABCG2. Non-limiting examples of neural stem cell markers include Nestin, PSA-NCAM, p75 Neurotrophin R, and Vimentin. Other non-limiting examples of stem cell markers include, Oct4, Sox2, Klf4, LIN28, Nanog, SSEA-3, SSEA-4, Notch, and Wnt.

### 5.6 Compositions

Provided herein are compositions, such as pharmaceutical compositions, comprising one or more anti-KIT antibodies (e.g., humanized antibodies) or antigen-binding fragments thereof described herein, or conjugates thereof, described herein. In particular aspects, compositions described herein can be for in vitro, in vivo, or ex vivo uses. In specific embodiments, provided herein is a pharmaceutical composition comprising an anti-KIT antibody (e.g., a humanized antibody) or an antigen-binding fragment thereof described herein or a conjugate thereof described herein and a pharmaceutically acceptable carrier or excipient.

As used herein, the term “pharmaceutically acceptable” means being approved by a regulatory agency of the Federal or a state government, or listed in the U.S. Pharmacopeia, European Pharmacopeia or other generally recognized Pharmacopeia for use in animals, and more particularly in humans.

Therapeutic formulations containing one or more antibodies (e.g., humanized antibodies) provided herein can be prepared for storage by mixing the antibody having the desired degree of purity with optional physiologically acceptable carriers, excipients or stabilizers (Remington's Pharmaceutical Sciences (1990) Mack Publishing Co., Easton, PA; Remington: The Science and Practice of Pharmacy, 21st ed. (2006) Lippincott Williams & Wilkins, Baltimore, MD), in the form of lyophilized formulations or aqueous solutions. Acceptable carriers, excipients, or stabilizers are nontoxic to recipients at the dosages and concentrations employed, and include buffers such as phosphate, citrate, and other organic acids; and/or non-ionic surfactants such as TWEEN™, PLURONICS™ or polyethylene glycol (PEG).

Formulations, such as those described herein, can also contain more than one active compounds (for example, molecules, e.g., antibody or antibodies described herein) as necessary for the particular indication being treated. In certain embodiments, formulations comprise an antibody provided herein and one or more active compounds with complementary activities that do not adversely affect each other. Such molecules are suitably present in combination in amounts that are effective for the purpose intended.

The formulations to be used for in vivo administration can be sterile. This is readily accomplished by filtration through, e.g., sterile filtration membranes.

In specific aspects, the pharmaceutical compositions provided herein contain therapeutically effective amounts of one or more of the anti-KIT antibodies (e.g., humanized antibodies) provided herein, and optionally one or more additional prophylactic of therapeutic agents, in a pharmaceutically acceptable carrier. Such pharmaceutical compositions are useful in the prevention, protection against, treatment, management or amelioration of a KIT-associated disorder, eosinophil related disorder or mast cell related disorcer, or one or more of the symptoms thereof.

Pharmaceutical carriers suitable for administration of the antibodies provided herein include any such carriers known to those skilled in the art to be suitable for the particular mode of administration.

In addition, the antibodies described herein can be formulated as the sole pharmaceutically active ingredient in the composition or can be combined with other active ingredients (such as one or more other prophylactic or therapeutic agents).

Compositions can contain one or more anti-KIT antibodies provided herein. In one embodiment, the antibodies are formulated into suitable pharmaceutical preparations, such as solutions, suspensions, powders, or elixirs, in sterile solutions or suspensions for parenteral administration. In one embodiment, the antibodies are formulated into suitable pharmaceutical preparations, such as solutions, suspensions, tablets, dispersible tablets, pills, capsules, powders, sustained release formulations or elixirs, for oral administration, as well as transdermal patch preparation and dry powder inhalers.

In such compositions, one or more antibodies provided herein (or conjugates thereof) is (are) mixed with a suitable pharmaceutical carrier. Concentrations of an antibody or antibodies in the compositions can, for example, be effective for delivery of an amount, upon administration, that treats, prevents, protects against or manages a KIT-associated disorder, eosinophil related disorder or mast cell related disorder, or one or more symptoms thereof.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

In certain aspects, an antibody (e.g., a humanized antibody) provided herein (or an antibody-drug conjugate thereof) is included in the pharmaceutically acceptable carrier in an effective amount sufficient to exert a therapeutically useful effect in the absence of, or with minimal or negligible, undesirable side effects on the patient treated. A therapeutically effective concentration can be determined empirically by testing the compounds in in vitro and in vivo systems using routine methods and then extrapolated therefrom for dosages for humans.

The concentration of antibody in the pharmaceutical composition will depend on, e.g., the physicochemical characteristics of the antibody, the dosage schedule, and amount administered as well as other factors known to those of skill in the art. In certain aspects, the concentration of antibody-drug conjugate in the pharmaceutical composition will depend on, e.g., the physicochemical characteristics of the antibody and/or the drug, the dosage schedule, and amount administered as well as other factors known to those of skill in the art.

In one embodiment, a therapeutically effective dosage produces a serum concentration of antibody of from about 0.1 ng/ml to about 50-100 μg/ml. The pharmaceutical compositions, in another embodiment, provide a dosage of from about 0.001 mg to about 2000 mg of antibody per kilogram of body weight for administration over a period of time, e.g., every day, every week, every 2 weeks, or every 3, 4 or 8 weeks. Pharmaceutical dosage unit forms can be prepared to provide from about 0.01 mg to about 2000 mg, and in one embodiment from about 10 mg to about 500 mg of the antibody and/or a combination of other optional essential ingredients per dosage unit form.

In a particular embodiment, an antibody-drug conjugate described herein is administered at an effective dosage of about 1 to 100 mg of antibody-drug conjugate per kilogram of body weight for administration over a period of time, e.g., every day, every week, every 2 weeks, or every 3 weeks.

An anti-KIT antibody described herein can be administered at once, or can be divided into a number of smaller doses to be administered at intervals of time. It is understood that the precise dosage and duration of treatment is a function of the disease being treated and can be determined empirically using known testing protocols or by extrapolation from in vivo or in vitro test data. It is to be noted that concentrations and dosage values can also vary with the severity of the condition to be alleviated. It is to be further understood that for any particular subject, specific dosage regimens can be adjusted over time according to the individual need and the professional judgment of the person administering or supervising the administration of the compositions, and that the concentration ranges set forth herein are exemplary only and are not intended to limit the scope or practice of the claimed compositions.

Upon mixing or addition of an antibody, the resulting mixture can be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and can be empirically determined.

Pharmaceutical compositions described herein are provided for administration to humans and animals, such as mammals (e.g., cat or dog), in unit dosage forms, such as sterile parenteral (e.g., intravenous) solutions or suspensions containing suitable quantities of the compounds or pharmaceutically acceptable derivatives thereof. Pharmaceutical compositions are also provided for administration to humans and animals, such as mammals (e.g., cat or dog), in unit dosage form, such as tablets, capsules, pills, powders, granules, and oral solutions or suspensions, and oil-water emulsions containing suitable quantities of the compounds or pharmaceutically acceptable derivatives thereof. The antibody is, in one embodiment, formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the antibody sufficient to produce the desired therapeutic effect, in association with the required pharmaceutical carrier, vehicle or diluent. Examples of unit-dose forms include ampoules and syringes and individually packaged tablets or capsules. Unit-dose forms can be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

In certain embodiments, one or more anti-KIT antibodies described herein are in a liquid pharmaceutical formulation. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered can also contain minor amounts of nontoxic auxiliary substances such as wetting agents, emulsifying agents, solubilizing agents, and pH buffering agents and the like.

Actual methods of preparing such dosage forms are known, or will be apparent, to those skilled in this art; for example, see, e.g., Remington's Pharmaceutical Sciences (1990) Mack Publishing Co., Easton, PA; Remington: The Science and Practice of Pharmacy, 21st ed. (2006) Lippincott Williams & Wilkins, Baltimore, MD.

Dosage forms or compositions containing antibody in the range of 0.005% to 100% with the balance made up from non-toxic carrier can be prepared. Methods for preparation of these compositions are known to those skilled in the art.

Parenteral administration, in one embodiment, is characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents. Other routes of administration may include, epidural administration, enteric administration, intracerebral administration, nasal administration, intraarterial administration, intracardiac administration, intraosseous infusion, intrathecal administration, and intraperitoneal administration.

Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Pharmaceutical carriers also include ethyl alcohol, polyethylene glycol and propylene glycol for water miscible vehicles; and sodium hydroxide, hydrochloric acid, citric acid or lactic acid for pH adjustment.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

An anti-KIT antibody described herein can be suspended in micronized or other suitable form. The form of the resulting mixture depends upon a number of factors, including the intended mode of administration and the solubility of the compound in the selected carrier or vehicle. The effective concentration is sufficient for ameliorating the symptoms of the condition and can be empirically determined.

In other embodiments, the pharmaceutical formulations are lyophilized powders, which can be reconstituted for administration as solutions, emulsions and other mixtures. They can also be reconstituted and formulated as solids or gels.

The lyophilized powder is prepared by dissolving an antibody provided herein, in a suitable solvent. In some embodiments, the lyophilized powder is sterile. The solvent can contain an excipient which improves the stability or other pharmacological component of the powder or reconstituted solution, prepared from the powder. Excipients that can be used include, but are not limited to, dextrose, sorbital, fructose, corn syrup, xylitol, glycerin, glucose, sucrose or other suitable agent. The solvent can also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

Antibodies described herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

The antibodies and other compositions provided herein can also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874. In some embodiments, the anti-KIT antibodies described herein are targeted (or otherwise administered) to the bone marrow. In some embodiments, anti-KIT antibodies described herein are targeted (or otherwise administered) to the gastrointestinal tract. In some embodiments, anti-KIT antibodies described herein are targeted (or otherwise administered) to the brain. In specific embodiments, an anti-KIT antibody described herein is capable of crossing the blood-brain barrier.

In specific embodiments, anti-KIT antibodies described herein are targeted (or otherwise administered) to an ocular tissue or organ. In particular aspects, a composition comprising anti-KIT antibodies described herein can be targeted to an ocular tissue or organ as eye drops or gels. In particular aspects, a composition comprising anti-KIT antibodies described herein can be targeted to the ear.

Provided herein is a pharmaceutical pack or kit comprising one or more containers filled with one or more of the ingredients of the pharmaceutical compositions described herein, such as one or more antibodies provided herein. Optionally associated with such container(s) can be a notice in the form prescribed by a governmental agency regulating the manufacture, use or sale of pharmaceuticals or biological products, which notice reflects approval by the agency of manufacture, use or sale for human administration.

### 5.7 Diagnostic Methods

Labeled or otherwise detectable antibodies, which immunospecifically bind to a KIT antigen can be used for diagnostic purposes to detect, diagnose, or monitor a KIT-associated disease.

Provided herein are methods for detecting KIT expression in samples obtained from patients with a KIT-associated disorder or disease. In a particular embodiment, a method for detecting KIT expression in a sample obtained from a patient comprises contacting the sample with an anti-KIT antibody described herein and detecting the expression level of KIT in the samples, for example, by correlating the binding of anti-KIT antibody to KIT with KIT expression levels. Methods for detection are known to one of skill in the art.

In certain aspects, provided herein are methods for diagnosing a patient with a KIT-associated disorder or disease. In a certain aspect, a method for diagnosing a subject with a KIT-associated disorder or disease comprises contacting cells or a sample obtained from the subject with an anti-KIT antibody described herein (or an antigen-binding fragment thereof) and detecting the expression level of KIT in the cells or the sample. In certain embodiments, a method for diagnosing a patient with a KIT-associated disorder or disease is an in vitro method. In particular embodiments, a method for diagnosing a patient with a KIT-associated disorder or disease is an ex vivo method. In certain embodiments, the method further comprises, after diagnosis, administering to the patient an antibody or antigen binding fragment described herein.

In certain aspects, provided herein are methods for the detection of a KIT-associated disease comprising: (a) assaying the expression of a KIT antigen in cells or a tissue sample of a subject using one or more antibodies described herein; and (b) comparing the level of the KIT antigen with a control level, e.g., levels in normal tissue samples (e.g., from a patient not having a KIT-associated disease, or from the same patient before disease onset), whereby an increase in the assayed level of KIT antigen compared to the control level of the KIT antigen is indicative of a KIT-associated disease.

Methods for detection are known to one of skill in the art. For example, the anti-KIT antibody can be conjugated to a detectable molecule (e.g., as described in section 5.1.1), and the detectable molecule can be visualized using standard techniques (e.g., microscopy). Antibodies described herein can be used to assay KIT antigen levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as ELISA and the radioimmunoassay (RIA). Suitable antibody assay labels are known in the art and include enzyme labels, such as, glucose oxidase; radioisotopes, such as iodine (¹²5I, ¹²¹I), carbon (C), sulfur (³⁵S), tritium (3H), indium (¹²¹In), and technetium (⁹⁹Tc); luminescent labels, such as luminol; and fluorescent labels, such as fluorescein and rhodamine, and biotin. In specific embodiments, diagnostic methods described herein involve using naked or unlabeled antibodies not conjugated to a detectable marker, and the naked or unlabeled antibodies are detected indirectly, e.g., by using a secondary antibody, which can be labeled.

In certain embodiments, high expression of KIT in a sample relative to a normal control sample (e.g., sample obtained from a healthy patient not suffering from a KIT-associated disorder or disease) indicates that the patient is suffering from a KIT-associated disorder or disease.

A method for diagnosing a patient with a KIT-associated disorder or disease, such as cancer, in a sample obtained from a patient comprises contacting the sample with an anti-KIT antibody described herein and detecting the expression level of KIT in the sample. In certain embodiments, high expression of KIT in a sample relative to a normal control sample (e.g., sample obtained from a healthy patient not suffering from a KIT-associated disorder or disease) indicates that the patient is suffering from a KIT-associated disorder or disease.

In certain embodiments, a sample can be a tumor sample derived from, or comprising tumor cells from, a patient's tumor. Examples of tumor samples herein include, but are not limited to, tumor biopsies, circulating tumor cells, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples. In certain embodiments, a sample is a fixed tumor sample which has been histologically preserved using a fixative. In some embodiments, a sample is a formalin-fixed tumor sample which has been preserved using formaldehyde as the fixative. In certain embodiments, a sample is an embedded tumor sample which is surrounded by a firm and generally hard medium such as paraffin, wax, celloidin, or a resin. Embedding makes possible the cutting of thin sections for microscopic examination or for generation of tissue microarrays (TMAs). In particular embodiments, a sample is a paraffin-embedded tumor sample which is surrounded by a purified mixture of solid hydrocarbons derived from petroleum. In certain embodiments, a sample is a frozen tumor sample which is, or has been, frozen. In a specific embodiment, a sample, for example, a paraffin-embedded sample or frozen sample, is sectioned.

In certain aspects, a cancer or biological sample which displays KIT expression, amplification, or activation is one which, in a diagnostic test, expresses (including overexpresses) a KIT receptor, has amplified KIT gene, and/or otherwise demonstrates activation or phosphorylation of a KIT receptor.

Also provided herein is the detection and diagnosis of a KIT-associated disease in a human. In one embodiment, diagnosis comprises: a) administering (for example, parenterally, subcutaneously, or intraperitoneally) to a subject an effective amount of a labeled antibody described herein; b) waiting for a time interval following the administering for permitting the labeled antibody to preferentially concentrate at sites in the subject where the KIT antigen is expressed (and for unbound labeled molecule to be cleared to background level); c) determining background level; and d) detecting the labeled antibody in the subject, such that detection of labeled antibody above the background level indicates that the subject has a KIT-mediated disease. Background level can be determined by various methods including, comparing the amount of labeled molecule detected to a standard value previously determined for a particular system.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ⁹⁹Tc. The labeled antibody will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982)).

Depending on several variables, including the type of label used and the mode of administration, the time interval following the administration for permitting the labeled antibody to preferentially concentrate at sites in the subject and for unbound labeled antibody to be cleared to background level is 6 to 48 hours or 6 to 24 hours or 6 to 12 hours. In another embodiment the time interval following administration is 5 to 20 days or 5 to 10 days.

In one embodiment, monitoring of a KIT-mediated disease is carried out by repeating the method for diagnosing the a KIT-mediated disease, for example, one month after initial diagnosis, six months after initial diagnosis, one year after initial diagnosis, etc.

Presence of the labeled molecule can be detected in the subject using methods known in the art for in vivo scanning. These methods depend upon the type of label used. Skilled artisans will be able to determine the appropriate method for detecting a particular label. Methods and devices that can be used in the diagnostic methods of the invention include, but are not limited to, computed tomography (CT), whole body scan such as position emission tomography (PET), magnetic resonance imaging (MRI), and sonography.

In a specific embodiment, the molecule is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

### 6. EXAMPLES

The examples herein are offered by way of illustration, and not by way of limitation.

### 6.1 Example 1

Nucleic acid molecules encoding the Fc mutated H (heavy chain) and L (light chain) amino acid sequences of an anti-KIT antibody (SEQ ID NO: 23 and SEQ ID NO: 24 respectively, see Table 6 below) were cloned directly into an expression vector. Constructs were confirmed by sequencing and the vector was transfected into CHO cells. Stable transfections to establish cell lines expressing the antibody were carried out and were subsequently drug-selected. The best expressing lines were selected based on an above-background IgG titre in the supernatant, and expanded in the presence of drug selection with screening for IgG expression using an Octet® QKe system at every stage.

TABLE 6
DNA Sequences encoding heavy and light chain sequences of an anti-KIT
antibody.

| | |
|---|---|
| Heavy | atggagtggtcctgggtgttcctgttctttctgtccgtgaccacaggcgtgcacagccaggtgcagctggtgcagtc |
| Chain | tggagctgaggtgaagaagccaggagcttctgtgaagctgtcctgcaaggccagcggctacaccttcacagacta |
| Sequence | ctatatcaactgggtgagacaggctcctggcaagggcctggagtggatcgctcgcatctatccaggctctggcaa |
| (DNA): | cacctactataatgagaagtttaagggccgggccaccctgacagctgataagagcacctctacagcttacatgcag |
| | ctgtccagcctgagatccgaggacaccgccgtgtacttctgcgctcgcggcgtgtactattttgattattggggcca |
| | gggcaccacagtgaccgtgtcttccgctagcacaaagggcccttccgtgtttccactggctcccagctctaagtcc |
| | accagcggaggaacagccgctctgggctgtctggtgaaggactatttcccagagcccgtgaccgtgagctggaa |
| | ctctggcgccctgaccagcggagtgcatacatttcctgctgtgctgcagtccagcggcctgtactctctgtcttccgt |
| | ggtgaccgtgccaagctcttccctgggcacccagacatatatctgcaacgtgaatcacaagccatccaatacaaag |
| | gtggacaagaaggtggagcccaagagctgtgataagacccatacatgccccccttgtcctgctccagaggctca |
| | gggaggaccatccgtgttcctgtttccacccaagcctaaggacaccctgtacatcacaagggagccagaggtgac |
| | ctgcgtggtggtggacgtgagccacgaggatcccgaggtgaagttcaactggtacgtggatggcgtggaggtgc |
| | ataatgccaagacaaagccaagggaggagcagtacaatagcacctatcgggtggtgtctgtgctgacagtgctgc |
| | accaggactggctgaacggcaaggagtacaagtgccaggtgtctaataaggccctgcccgctcctatcgagaag |
| | accatctccaaggccaagggccagcctagggagccacaggtgtacacactgcctccaagccgggacgagctga |
| | ccaagaaccaggtgtctctgacatgtctggtgaagggcttctatccctctgatatcgctgtggagtgggagtccaat |
| | ggccagcctgagaacaattacaagaccacaccccctgtgctggactccgatggcagcttctttctgtattccaagct |
| | gaccgtggataagagcaggtggcagcagggcaacgtgttttcttgttccgtgatgcatgaggctctgcacaatcatt |
| | acacacagaagagcctgtctctgtcccctggc (SEQ ID NO: 23). |
| Light | atgtccgtgccaacccaggtgctgggactgctgctgctgtggctgaccgacgccaggtgcgatatcgtgatgaca |
| Chain | cagtccccttccagcctgtctgcttccgtgggcgacagagtgaccatcacctgtaaggccagccagaacgtgcgc |
| Sequence | accaatgtggcttggtaccagcagaagccaggcaaggcccccaaggctctgatctatagcgcctcttacaggtat |
| (DNA) | agcggagtgcctgaccggttcaccggatccggaagcggaacagacttcaccctgacaatctcttccctgcagcct |
| | gaggacttcgctgattacttttgccagcagtacaactcttatccaaggaccttcggcggcggcacaaaggtggaga |
| | tcaagcggaccgtggccgctccaagcgtgttcatctttcccccttctgacgagcagctgaagtctggcacagcctc |
| | cgtggtgtgcctgctgaacaacttctaccccagagaggccaaggtgcagtggaaggtggataacgctctgcagtc |
| | tggcaattcccaggagagcgtgaccgagcaggactctaaggattccacatatagcctgagctctaccctgacact |
| | gtctaaggccgattacgagaagcacaaggtgtatgcttgcgaggtgacccatcagggcctgtccagcccagtgac |
| | aaagtccttcaatcgcggcgagtgt (SEQ ID NO: 24). |

The resulting antibody after removal of the leader sequences was designated antibody mAb 1. The heavy and light chain amino acid sequences of the antibody (after removal of the leader sequences) are set forth in Table 7 below.

TABLE 7
Full-length heavy and light chain amino acid sequences of mAb1.

| | |
|---|---|
| mAb1 | QVQLVQSGAEVKKPGASVKLSCKASGYTFTDYYINWVRQAPGKGLEWI |
| full-length | ARIYPGSGNTYYNEKFKGRATLTADKSTSTAYMQLSSLRSEDTAVYFCA |
| heavy | RGVYYFDYWGQGTTVTVSSASTKGPSVFPLAPSSKSTSGGTAALGCLVK |
| chain | DYFPEPVTVSWNSGALTSGVHTFPAVLQSSGLYSLSSVVTVPSSSLGTQ |
| amino | TYICNVNHKPSNTKVDKKVEPKSCDKTHTCPPCPAPEAQGGPSVFLFPP |
| acid | KPKDTLYITREPEVTCVVVDVSHEDPEVKFNWYVDGVEVHNAKTKPRE |
| sequence | EQYNSTYRVVSVLTVLHQDWLNGKEYKCQVSNKALPAPIEKTISKAKG |
| (including | QPREPQVYTLPPSRDELTKNQVSLTCLVKGFYPSDIAVEWESNGQPEN |
| Fc): | NYKTTPPVLDSDGSFFLYSKLTVDKSRWQQGNVFSCSVMHEALHNHYT |
| | QKSLSLSPG (SEQ ID NO: 21) |
| mAb1 | DIVMTQSPSSLSASVGDRVTITCKASQNVRTNVAWYQQKPGKAPKALIY |
| full-length | SASYRYSGVPDRFTGSGSGTDFTLTISSLQPEDFADYFCQQYNSYPRTFG |
| light chain | GGTKVEIKRTVAAPSVFIFPPSDEQLKSGTASVVCLLNNFYPREAKVQW |
| amino | KVDNALQSGNSQESVTEQDSKDSTYSLSSTLTLSKADYEKHKVYACEV |
| acid | THQGLSSPVTKSFNRGEC (SEQ ID NO: 22) |
| sequence: | |

The Fc domain of the heavy chain of this antibody comprises non-naturally occurring amino acids 234A, 235Q, 322Q, 252Y, 254T and 256E as numbered by the EU index as set forth in Kabat. These six non-naturally occurring amino acids are shown in bold underlined text in the sequence above.

mAb 1 was determined not to induce significant degranulation of FcgRI-expressing human mast cells (as shown by 0% release of beta-hexosaminidase from human mast cells in culture) compared to a corresponding antibody with a wild-type (unmutated) IgG1 Fc domain (“mAbc”). Release of beta-hexosaminidase from human mast cells in culture (in presence of IFN gamma) was reduced by more than 50% with mAb 1 compared to mAbc. Additionally, mAb 1 did not show significant Fc receptor-dependent KIT agonist activity (as determined by KIT phosphorylation) compared to mAbc, even when cross-linked on THP-1 cells. Fc receptor-dependent KIT agonist activity (as determined by KIT phosphorylation with Fc receptors crosslinked) was reduced by more than 50% with mAb1 compared to mAbc.

### 6.2 Example 2

Healthy volunteers were given a single infusion of mAb1 at 0.3, 1, 3, or 9 mg/kg or placebo. Total plasma tryptase levels were measured using an Immunocap® assay that detects both the alpha and beta forms of tryptase. For each cohort, tryptase values were normalized to 100% for pre-treatment values and to 0% for the lower limit of quantitation of the assay (1 ng/mL). Mean values and standard error of the mean were plotted by dose.

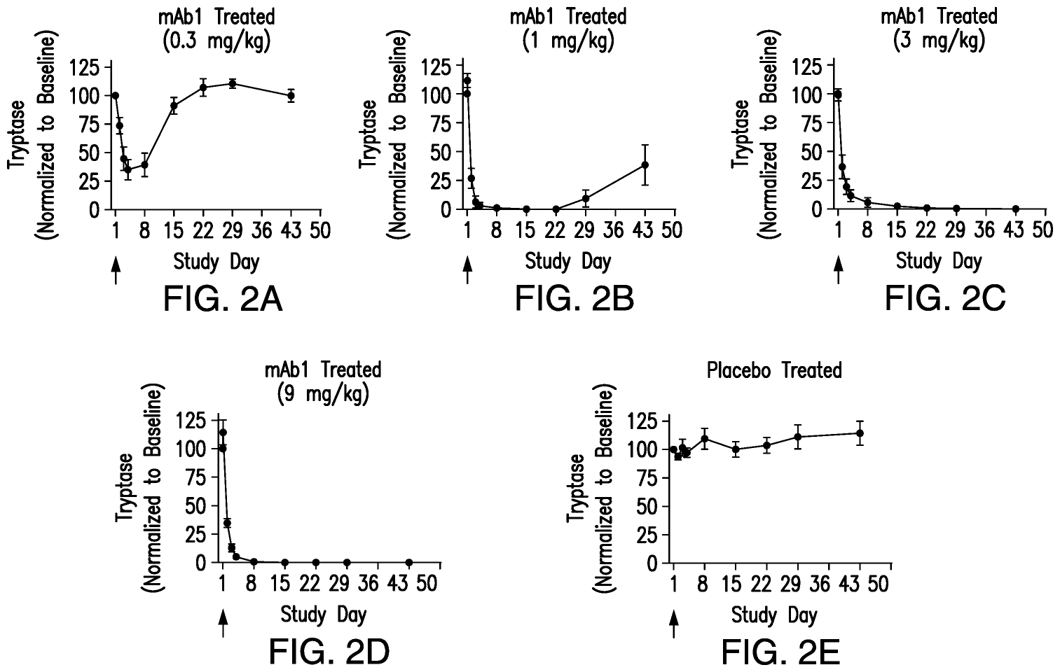

Results are shown in FIGS. 2A-2E, which indicates that mAb1 suppressed plasma tryptase in a dose-dependent manner.

### 6.3 Example 3

A single dose of mAb1 resulted in a prolonged decrease in tryptase values below the level of assay quantitation (1 ng/mL). Values below detection level were plotted arbitrarily as 0.5 ng/mL. Absolute plasma tryptase values were shown.

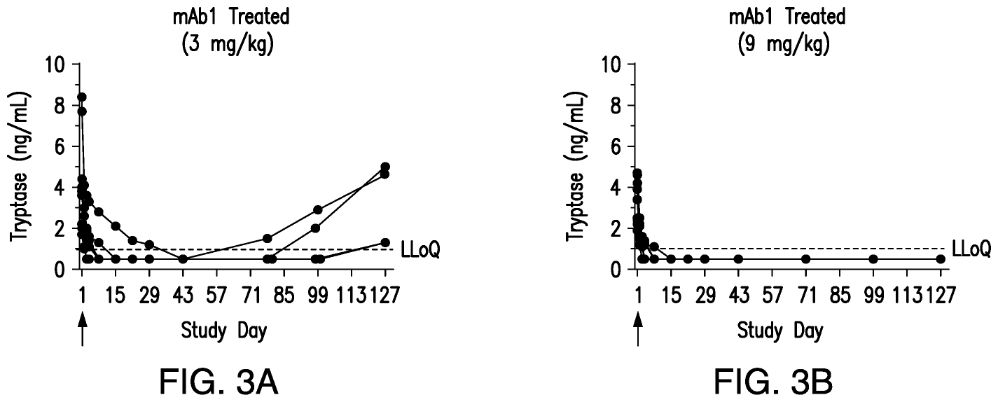

Results are shown in FIGS. 3A and 3B, which indicates that a single dose of mAb1 provided durable tryptase suppression at both 3 mg/kg and 9 mg/kg.

### 6.4 Example 4

Healthy volunteers were given a single infusion of mAb1 at 0.3, 1, 3, or 9 mg/kg or placebo. Plasma levels of stem cell factor (SCF), the only ligand for the c-KIT/CD117 receptor, were measured with an assay developed in house using a Meso Scale Diagnostics (MSD) platform. SCF plasma levels increased in a dose-dependent manner, which was consistent with allosteric blockade of SCF to the KIT receptor. Mean values and standard error of the mean were plotted by dose.

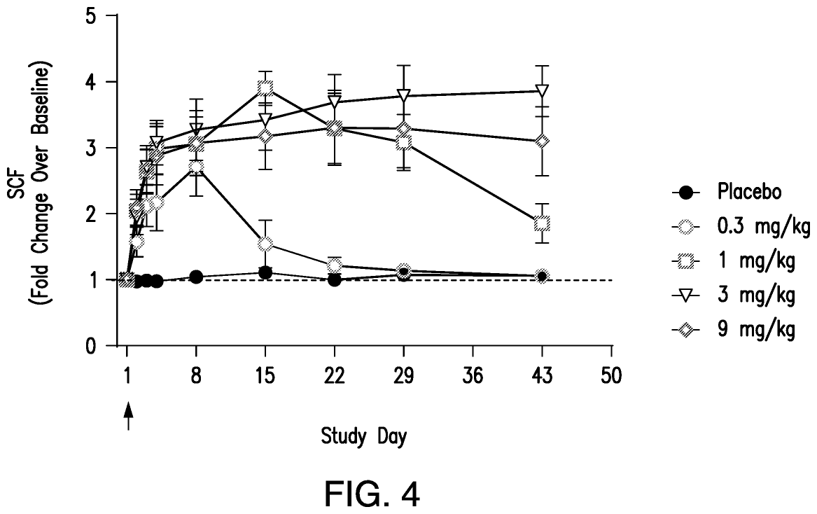

Results are shown in FIG. 4, which indicates that mAb1 induced a dose-dependent increase in plasma SCF levels.

### 6.5 Example 5

M-07e cells were serum-starved, then pre-treated with i) mAb1, ii) a corresponding antibody with the same variable region sequences but an unmutated (wild type) human IgG1 sequence (“mAbc”), iii) an isotype control antibody or iv) a small molecule kinase inhibitor that targets KIT (imatinib), and then stimulated with Stem Cell Factor (SCF). Phosphorylation was assessed by Western blotting.

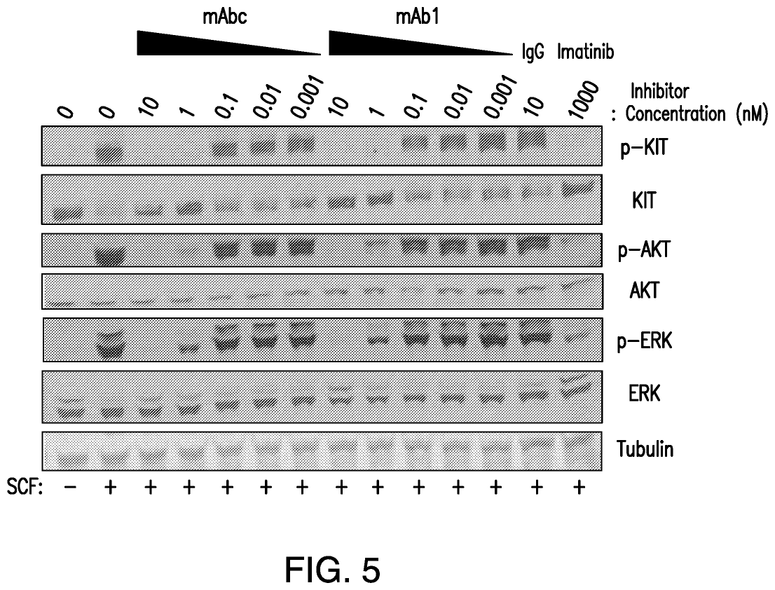

Results from one experiment are shown in FIG. 5. Data shown are representative of 3 independent experiments. IgG=human IgG1 isotype control antibody; p-KIT=tyrosine phosphorylated KIT; p-AKT=phosphorylated AKT; p-ERK=phosphorylated ERK1/2. Collectively, these results indicate that mAb1 was a more potent inhibitor of SCF-induced activation of wild-type KIT and downstream intracellular signaling pathways than the small molecule KIT inhibitor tested.

### 6.6 Example 6

The effects of mAb1 and imatinib on SCF-dependent M-07e cell proliferation were also characterized.

M-07e cells were serum-starved, then pre-treated with i) mAb1, ii) a corresponding antibody with the same variable region sequences but an unmutated (wild type) human IgG1 sequence (“mAbc”) or iii) imatinib, and then stimulated with SCF. Cells were incubated for 6 days at 37° C.

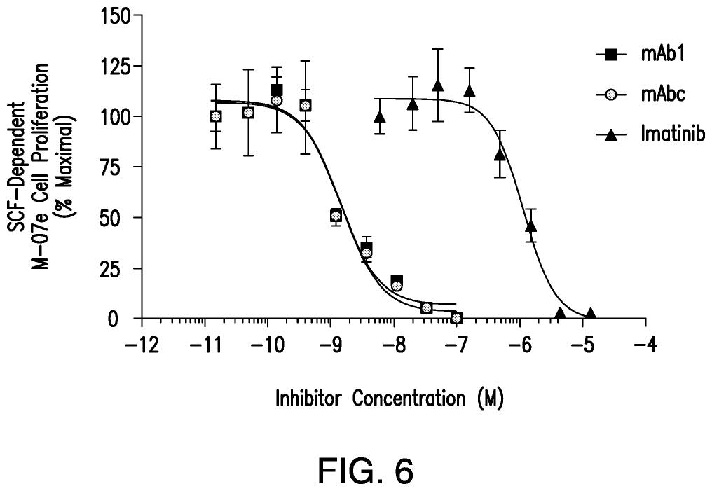

Results are shown in FIG. 6. Data are representative of 3 independent experiments and are presented as means of technical triplicates and standard errors of the means.

Both mAb1 and mAbc showed similar dose-dependent inhibition of M-07e cell proliferation, having mean IC50 values of 1.11±0.15 nM and 1.12±0.22 nM (±SEM; n=5;

FIG. 6), respectively. By comparison, imatinib inhibited SCF-dependent M-07e cell proliferation less potently, with a mean IC50 of 228±39 nM (±SEM) from 3 independent experiments. The antagonist activity of mAb1 was comparable to mAbc, indicating that the mutations introduced into the Fc region of mAb1 did not affect its ability to inhibit KIT.

### 6.7 Example 7

The binding affinities of mAb1 for recombinant human Fc-gamma receptors (FcTRs) and human neonatal Fc Receptor (FcRn) were characterized and equilibrium KD values were generated.

Binding to recombinant human Fc receptors was measured by bio-layer interferometry using an Octet® QKe instrument. Histidine-tagged Fc receptors were captured on anti-Penta-His biosensors and then exposed to serial dilutions of mAbc or mAb1 for 2-3 minutes, followed by a dissociation step ranging from 5-10 minutes. Curve fitting was performed using the instrument's analysis software. Binding to FcRn was performed with association and dissociation steps at pH 6.0 as well as pH 7.2.

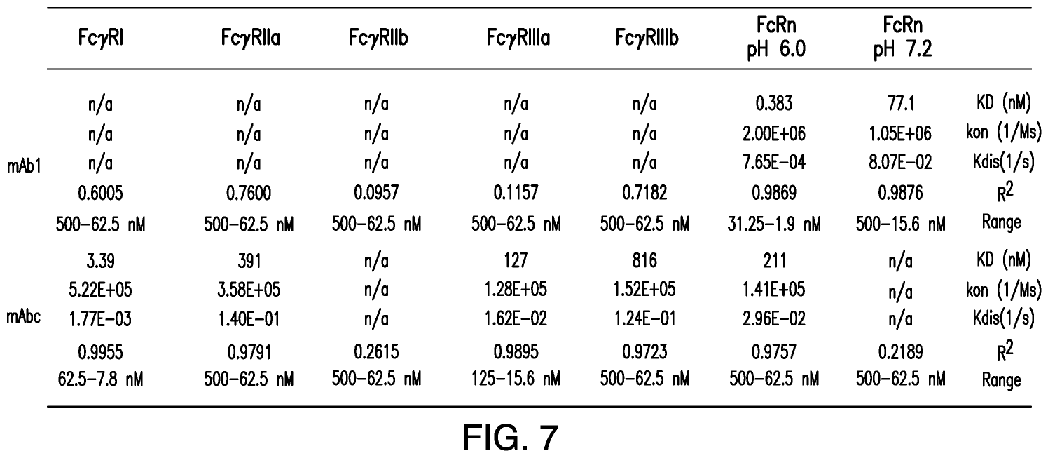

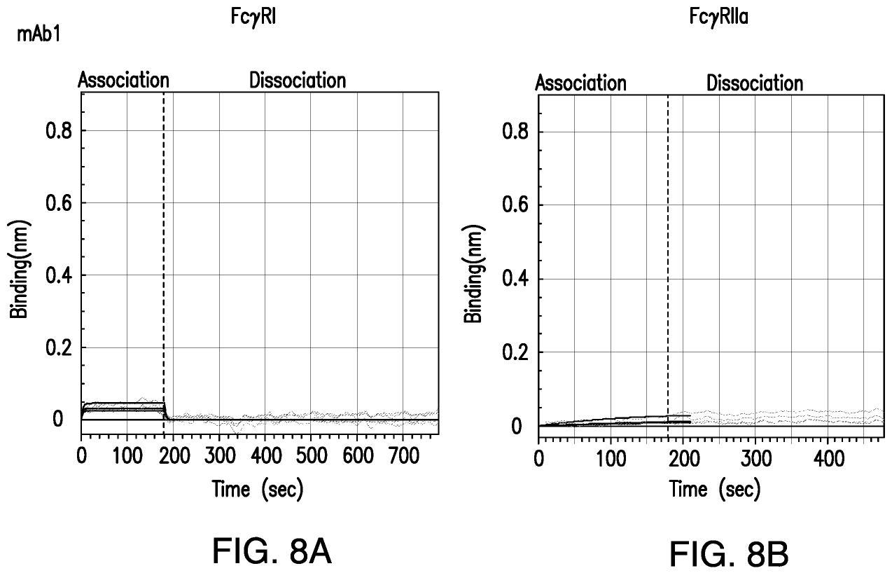

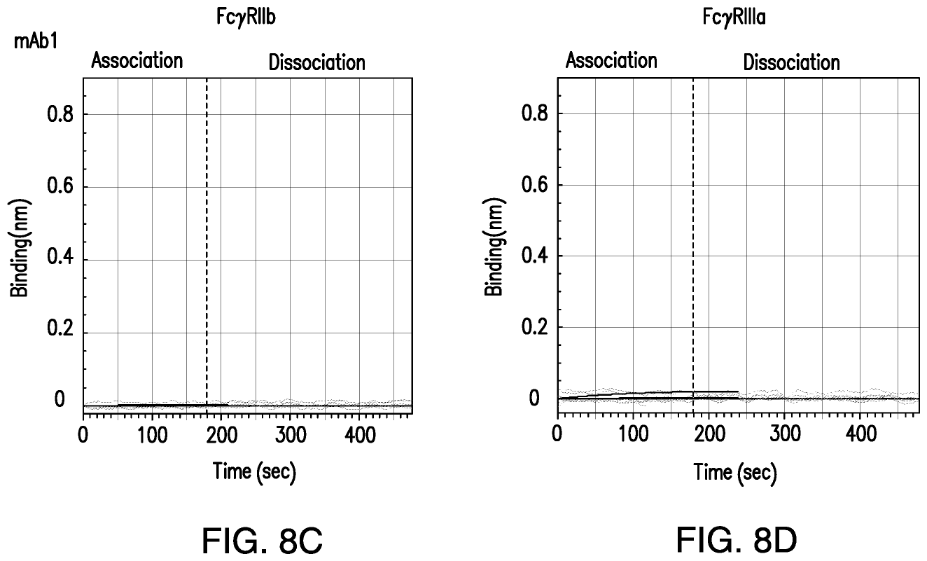

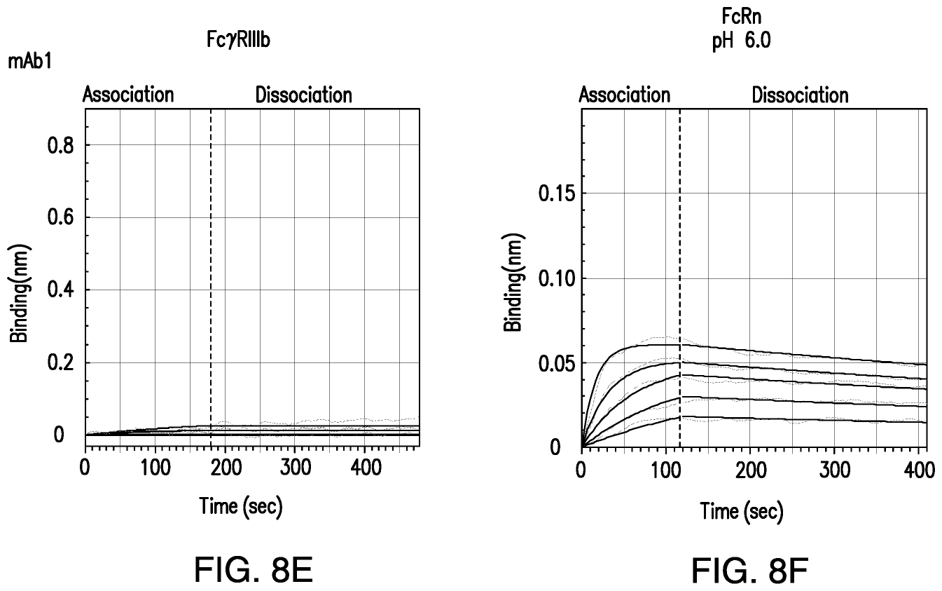

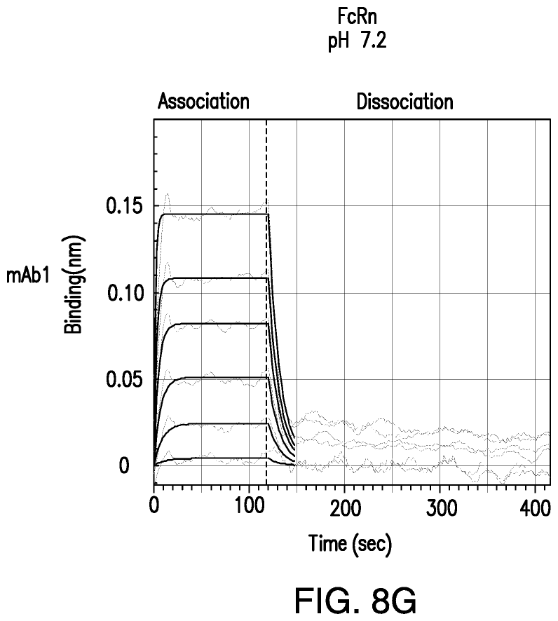

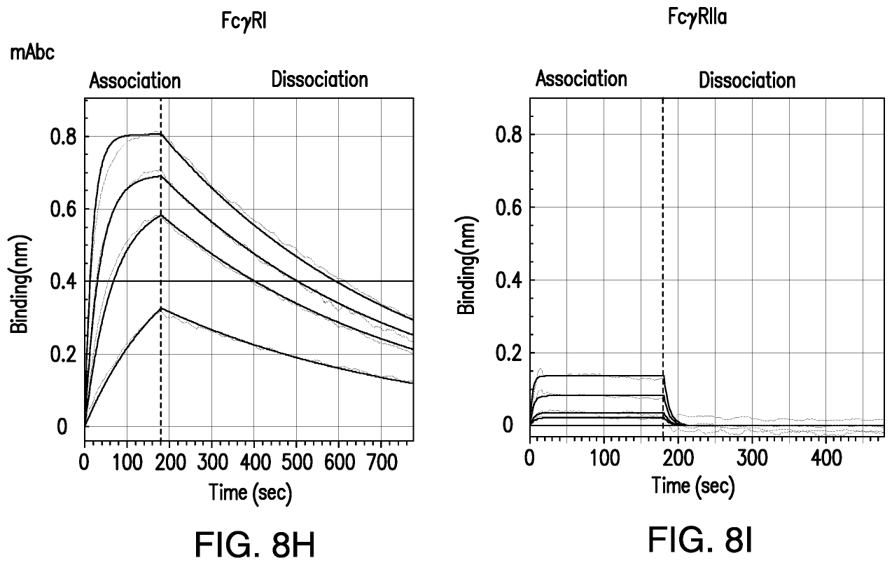

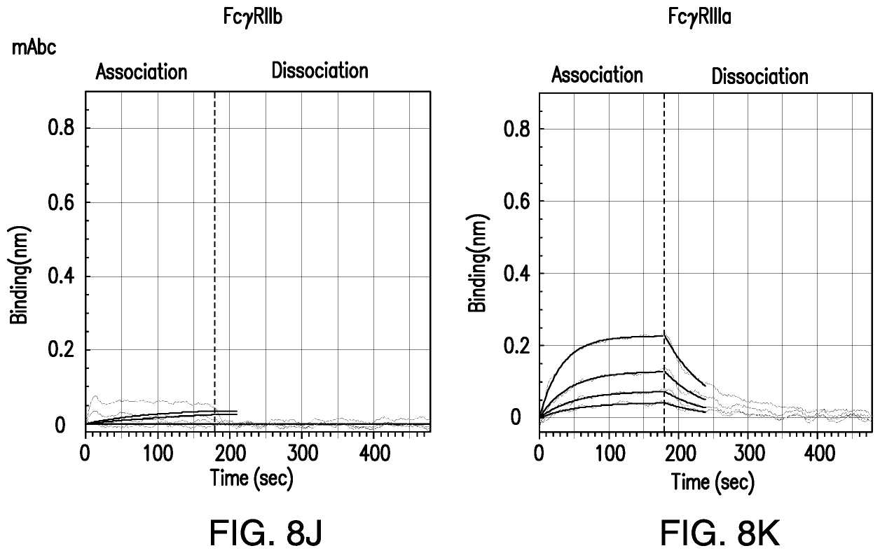

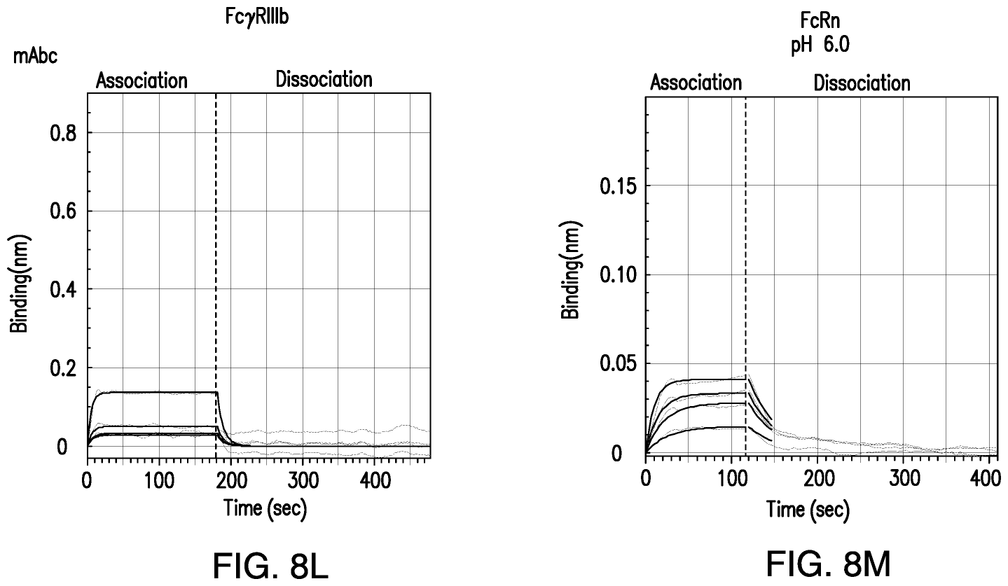

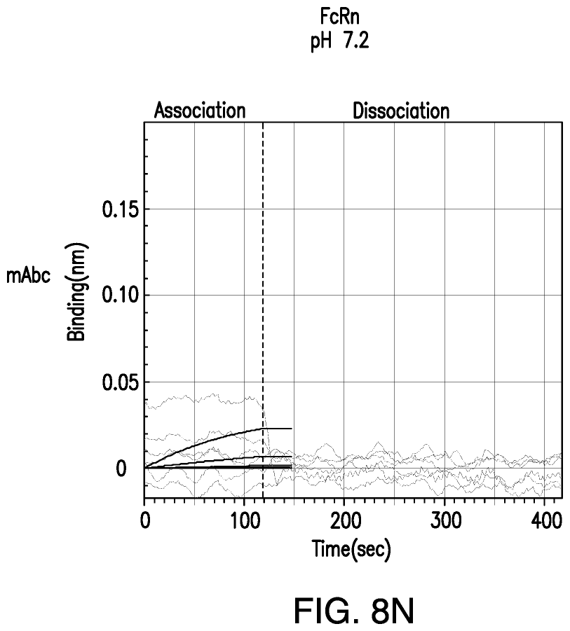

Results are shown in FIGS. 7 and 8A-8N, which show that mAbc bound with high affinity to FcγRI (KD=3.39 nM), with intermediate affinity to FcγRIIIa (KD=127 nM) and weak affinity to FcγRIIa (KD=391 nM) and FcγRIIIb (KD=816 nM). No binding to FcγRIIb was observed. In contrast, no binding of mAb1 (concentration=500 nM) to any recombinant human FcTRs was detected.

Binding to the human neonatal Fc Receptor (FcRn) was tested at physiological pH 7.2 as well as at pH 6.0 to simulate endocytic conditions. At pH 7.2, binding of mAbc to FcRn was not observed whereas mAb1 bound with intermediate affinity (KD=77.1 nM). At pH 6.0, mAbc bound to FcRn with intermediate affinity (KD=211 nM). However, mAb1 bound to FcRn at pH 6.0 with significantly higher affinity and showed very slow dissociation (KD=0.38 nM).

These data indicate that the Fc mutations in mAb1 abolished FcTR interactions while enhancing interactions with FcRn in vitro.

### 6.8 Example 8

Antibody-dependent cellular cytotoxicity (ADCC) activity of mAb1 was evaluated using a commercially available ADCC Reporter Bioassay Kit (Promega Corporation, Madison, WI). The ADCC Reporter Bioassay used engineered Jurkat cells that stably expressed the FcγRIIIa receptor V158 (high affinity) variant, and an nuclear factor of activated T cells (NFAT) response element driving expression of firefly luciferase as effector cells. Binding of FcγRIIIa receptors on the surface of effector cells to the Fc effector portion of antibodies bound to antigen on target cells would result in crosslinking and activation of FcγRIIIa signaling, which would induce expression of the NFAT reporter gene. To assess the ability of mAb1 and mAbc to induce ADCC, KIT-expressing M-07e cells, transfected CHO cells (CHO-WT KIT), and human small cell lung cancer H526 cells were used as target cells. In addition, untransfected CHO cells, which did not express KIT, were used as control target cells. ADCC was considered to have been activated if a dose-dependent increase in the resulting reporter gene signal was observed.

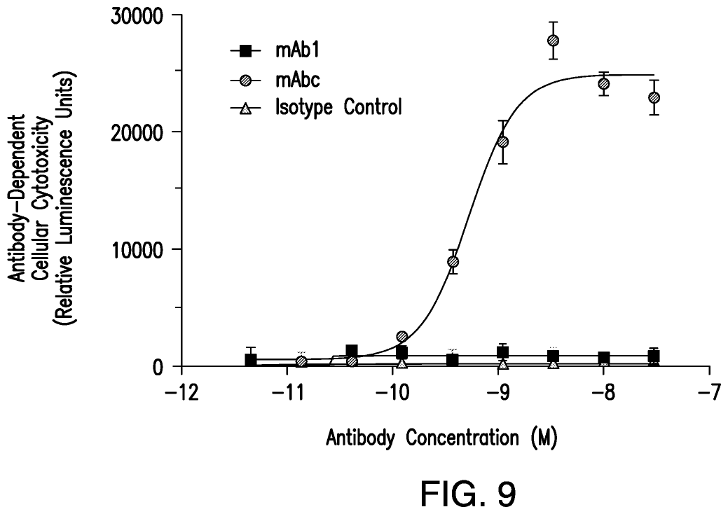

As shown in FIG. 9, mAbc triggered an ADCC response against KIT-expressing M-07e target cells, whereas mAb1 or the isotype control did not. Similar results were observed with CHO-WT KIT and H526 cells. Together, these data demonstrate that, unlike mAbc, mAb1 did not trigger ADCC responses against the KIT-expressing target cells examined.

### 6.9 Example 9

Fresh whole blood was incubated overnight with either 40 nM huIgG1 isotype control or mAb1 at 0.02, 0.2, or 40 nM, either in solution or dry coated as indicated. Phytohemagglutinin (PHA) (10 μg/mL) and lipopolysaccharides (LPS) (10 μg/mL) were used as positive controls. Plasma samples were harvested and stored frozen at −80° C. Cytokine levels were determined by utilizing Multiplexing Laser Bead Technology performed by Eve Technologies (Calgary, Alberta, Canada).

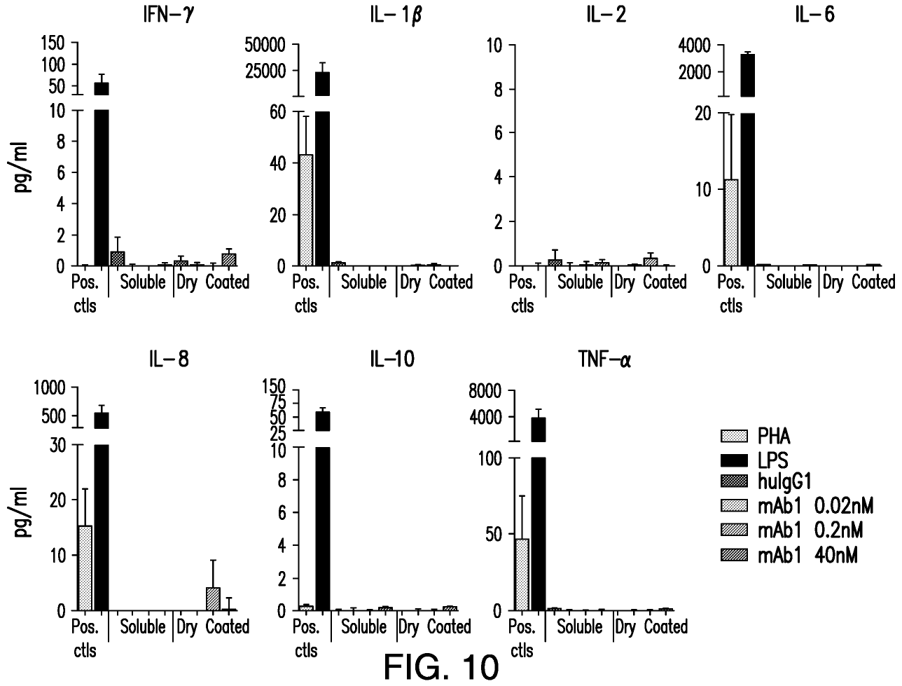

Results are shown in FIG. 10 in which cytokine concentrations for individual donors represent average of duplicate samples. Mean cytokine concentrations for all donors (n=6) were plotted with error bars representing SEM. The results show that overall very little specific cytokine induction was observed with mAb1 relative to the human IgG1 isotype control.

### 6.10 Example 10

A human patient presents and is diagnosed with a mast cell related disorder. The patient is administered mAb1 intravenously and is monitored before, during, and after the treatment for response by clinical assessments.

### 6.11 Example 11—a Study of mAb1 in Patients with Chronic Spontaneous Urticaria

Described in this example is a protocol for a randomized, double-blind, placebo-controlled, Phase 1 multiple ascending dose study to assess the safety, pharmacokinetics, and pharmacodynamics of mAb1 as add-on therapy in patients with chronic spontaneous urticaria. The study type is interventional (clinical trial). The allocation of patients is randomized. The intervention model is parallel assignment. Masking type is double-blind (participant and investigator).

The purpose of the study is to explore the safety, pharmacodynamics, and pharmacokinetics of ascending doses of mAb1 in patients with chronic spontaneous urticaria who remain symptomatic despite treatment with antihistamines. There is a screening period of up to 2 weeks, a 12-week double-blind treatment period and a 12-week follow-up period after treatment. Patients receive multiple doses of mAb1 or placebo as add-on therapy to their antihistamine. Adult patients aged 18 years to 75 years of all sexes are eligible for the study. Healthy volunteers are not accepted. It is estimated that this study will enroll 40 patients. For one arm of the study, patients receive mAb1 administered intravenously every 4-8 weeks; for the other arm of the study, patients receive normal saline administered intravenously every 4-8 weeks.

Primary Outcome Measures:
1. Safety as assessed by the incidence and severity of adverse events (time frame: from Day 1 (first dose) to Day 169 (last follow-up visit)). Safety of multiple, ascending doses of mAb1 as determined by drug related adverse events.

Secondary Outcome Measures:
1. Pharmacokinetic evaluation (time frame: from Day 1 (before first dose) to Day 169 (last follow-up visit)). mAb1 serum concentrations are measured at specified visits.
2. Pharmacodynamic evaluation (time frame: from Day 1 (first dose) to Day 169 (last follow-up visit)). The change from baseline for Urticaria Activity Score (UAS7) in patients who have received mAb1 vs. placebo.
3. Pharmacodynamic evaluation (time frame: from Day 1 (before first dose) to Day 169 (last follow-up visit)). The effect of mAb1 on tryptase and stem cell factor levels.
4. Pharmacodynamic evaluation (time frame: from Day 1 (first dose) to Day 169 (last follow-up visit)). The change from baseline for Hives Severity Score (HSS7) in patients who have received mAb1 vs. placebo.
5. Pharmacodynamic evaluation (time frame: from Day 1 (first dose) to Day 169 (last follow-up visit)). The change from baseline for Itch Severity Score (ISS7) in patients who have received mAb1 vs. placebo.
6. Safety evaluation (time frame: from Day 1 (before dosing) to Day 169 (last follow-up visit)). Assessment of immunogenicity by measuring the development of anti-mAb1 antibodies.

Key Inclusion Criteria:
1. Males and females, 18-75 years old.
2. Diagnosis of chronic spontaneous urticaria (CSU) despite the use of H1-antihistamines alone or in combination with H2-antihistamines and/or leukotriene receptor antagonists, as defined by:
a. Diagnosis of CSU for >/=6 months.
b. The presence of itch and hives for >/=6 consecutive weeks at any time prior to Visit 1 despite current use of H1-antihistamines.
c. UAS7 of >/=16 and HSS7 of >/=8 during the 7 days before treatment.
d. In-clinic UAS>/=4 on one of the screening visit days.
e. Use of H1-antihistamines alone or in combination with H2-antihistamines and/or leukotriene receptor antagonists for at least 3 days immediately prior to study entry and throughout the study.
3. Other than CSU, have no other significant medical conditions that would cause additional risk or interfere with study procedures.
4. Normal blood counts and liver function tests.
5. Both males and females of child-bearing potential must agree to use highly effective contraceptives during the study and for 150 days afterwards after treatment.
6. Willing and able to complete a daily symptom electronic diary for the duration of the study and adhere to the study visit schedule.

Key Exclusion Criteria:
1. Women who are pregnant or nursing.
2. Clearly defined cause for chronic urticaria.
3. Known HIV, hepatitis B or hepatitis C infection.
4. Vaccination with a live vaccine within 4 weeks prior to study drug administration (subjects must agree to avoid vaccination during the study). Inactivated vaccines are allowed such as seasonal influenza for injection.
5. History of anaphylaxis.

There are additional criteria that the treating doctor reviews with a candidate to confirm eligibility for the study.

### 6.12 Example 12—A Single Dose Study of the Safety, Pharmacokinetics and Pharmacodynamics of mAb1 in Patients With Cold Contact Urticaria, Symptomatic Dermographism, or Cholinergic Urticaria

Described herein is a protocol for an open label, Phase 1 single dose study to assess the safety, pharmacokinetics, and pharmacodynamics of mAb1 as add-on therapy in patients with cold contact urticaria, symptomatic dermographism, or cholinergic urticaria. The study type is interventional (clinical trial). The intervention mode is single group assignment.

This study is an open label Phase 1 study evaluating the safety, pharmacokinetics, and pharmacodynamics of a single dose of mAb1 in patients with cold contact urticaria, symptomatic dermographism, or cholinergic urticaria who remain symptomatic despite treatment with antihistamines. It is estimated that ten patients with cold contact urticaria, ten patients with symptomatic dermographism, and ten patients with cholinergic urticaria will be enrolled in three separate cohorts for a total of 30 patients. Prospective patients are screened with tests in clinic as well as daily at home diaries for 2 weeks prior to enrollment. A single dose of mAb1 is administered intravenously on Day 1. Post-treatment, patients are followed for 12 weeks. Adult patients aged 18 years to 75 years of all sexes are eligible for the study. Healthy volunteers are not accepted.

Primary Outcome Measures:
1. Safety as assessed by the incidence and severity of adverse events (time frame: from Day 1 through week 12). Safety of a single dose of mAb1 as determined by adverse events.

Secondary Outcome Measures:
1. For patients with cold contact urticaria, change in critical temperature thresholds (CTT) (time frame: from Day 1 to Day 85). The change from baseline in critical temperature thresholds over time as determined by provocation testing using the TempTest®.
2. For patients with symptomatic dermographism, change in provocation thresholds (time frame: from Day 1 to Day 85). The change from baseline in provocation thresholds over time as determined by provocation testing using the FricTest®.
3. For patients with cholinergic urticaria, changes in baseline Urticaria Activity Score Provocation (UASprovo) (time frame: From Day 1 to Day 85). Changes from baseline and percentage of responders as measured by UASprovo.
4. Changes from baseline in Urticaria Control Test (UCT) (time frame: from Day 1 to Day 85). Changes from baseline and percentage of responders for the UCT and modified UCT.
5. Blood biomarkers (time frame: from Day 1 to Day 85). Pre-treatment and post-treatment blood samples are collected and analyzed for changes in Stem Cell Factor.
6. Blood biomarkers (time frame: from Day 1 to Day 85). Pre-treatment and post-treatment blood samples are collected and analyzed for changes in tryptase.
7. Pharmacokinetic evaluation (time frame: from Day 1 to Day 85). mAb1 concentrations are measured.
8. Immunogenicity evaluation (time frame: from Day 1 to Day 85). Patients are monitored for the development of anti-drug antibodies.

Key Inclusion Criteria:
1. Diagnosis of cold contact urticaria, symptomatic dermographism, or cholinergic urticaria which does not respond to antihistamines. Diagnosis for ≥3 months; symptoms of both hive (wheal) and itch/burning/painful sensation despite concurrent use of anti-histamines. During screening, in clinic, for cold contact urticaria, patients must have a positive cold stimulation test; for symptomatic dermographism, patients must have a positive FricTest®; and for cholinergic urticaria, patients must have a positive pulse-controlled ergometry (PCE) provocation test. Subject is on stable dose of antihistamines.
2. Other than a diagnosis of cold contact urticaria, symptomatic dermographism, or cholinergic urticaria, no other conditions which would introduce additional risk factors or would interfere with the study procedures, as determined by the investigator, based on a medical evaluation.
3. Female and male patients must use highly effective contraception from the time of the screening visit and for at least 150 days after receipt of study treatment.
4. Willing and able to comply with all study requirements and procedures including completion of a daily medication diary and questionnaires.

Key Exclusion Criteria:
1. A clearly defined diagnosis of hives or angioedema other than chronic urticaria.
2. Receipt of prior biologic therapy (e.g., omalizumab, dupilumab, ligelizumab) within past 3 months.
3. Treatment with immunosuppressives (e.g., systemic corticosteroids, cyclosporine, methotrexate, dapsone, cyclophosphamide, tacrolimus and mycophenolate mofetil, hydroxychloroquine or others) within 4 weeks or 5 half-lives.
4. Active COVID-19 infection.
5. HIV, hepatitis B or hepatitis C infection.

There are additional criteria that the treating doctor reviews with a candidate to confirm eligibility for the study.

### 6.13 Example 13—mAb1 Reduced Disease Activity and Tryptase Levels in Patients with Chronic Inducible Urticaria

### 6.13.1 Study Background and Summary

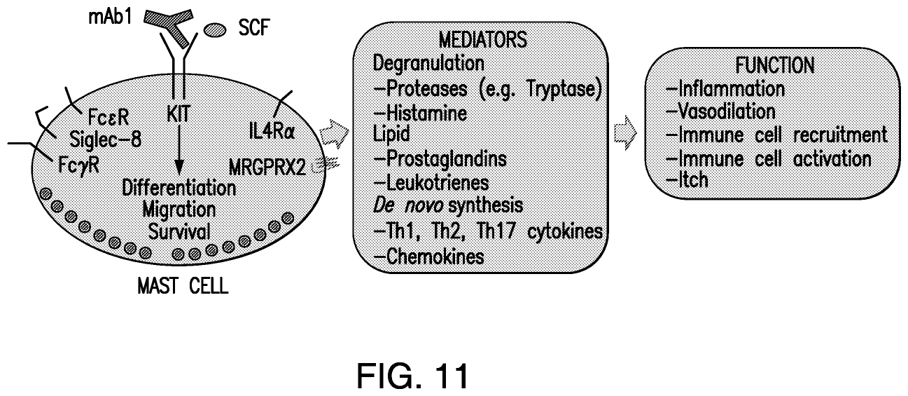

Chronic inducible urticaria (CIndU) is characterized by mast cell (MC)-driven wheals in response to triggers such as cold in cold urticaria (ColdU) or scratching of the skin in symptomatic dermographism (SD). These diseases, which are often severe and debilitating, can significantly impact patients' lives. MCs require activation of their KIT receptors by stem cell factor for survival, proliferation, and differentiation. MC burden is correlated with circulating tryptase, a protease secreted specifically by MCs. mAb1 is a monoclonal anti-KIT antibody that was engineered to selectively inhibit stem cell factor (SCF)-dependent KIT activation (see FIG. 11). mAb1 demonstrated a profound dose related reduction of circulating tryptase and was overall well-tolerated in healthy volunteers. In this study, patients with CIndU benefited from treatment with mAb1.

### 6.13.2 Study Design and Methods

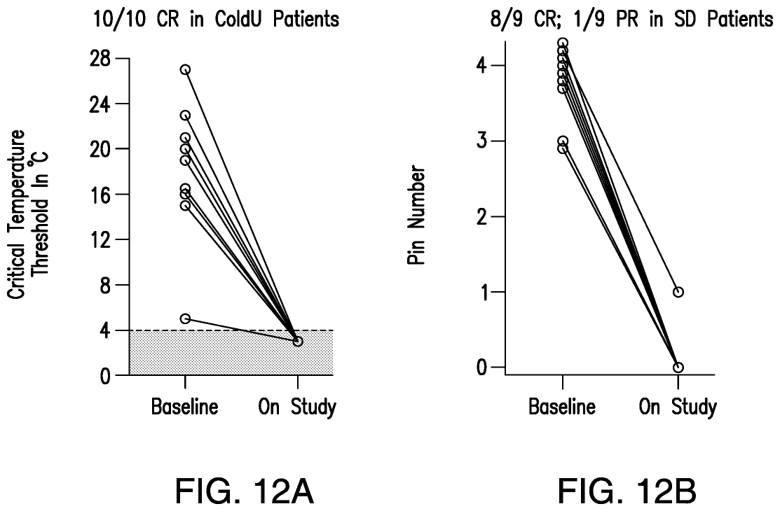

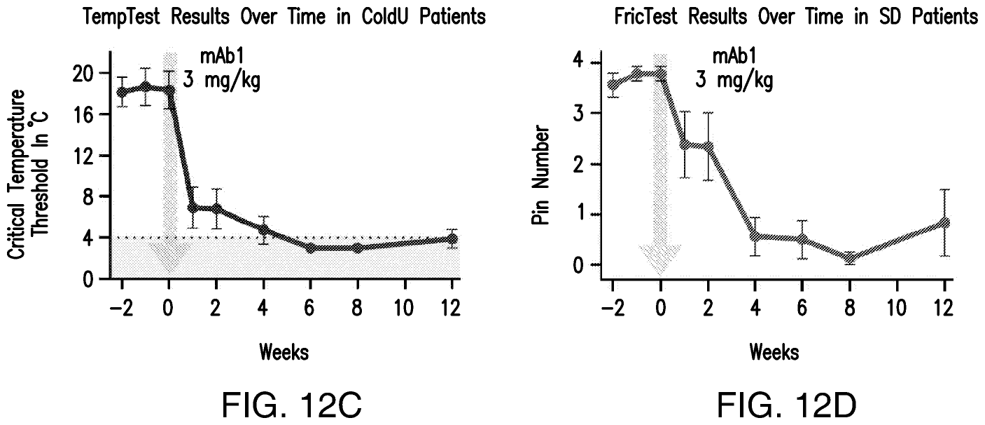

In this ongoing open-label, Phase 1b trial, patients with ColdU and SD refractory to antihistamine treatment received a single IV infusion of mAb1 at 3 mg/kg (as add-on treatment to H1-antihistamines) with a 12-week follow-up. Patients' symptoms were induced via provocation testing that resembled real life triggering situations. Primary objective was to evaluate safety/tolerability of mAb1 (adverse events and clinical lab tests). Secondary and exploratory objectives included pharmacokinetic and pharmacodynamic assessments, including changes from baseline provocation thresholds, measurement of tryptase and stem cell factor levels, clinical activity outcomes (impact on urticaria symptoms, disease control, clinical response), quality of life assessments and measurement of tissue mast cells through skin biopsies. Secondary objectives included evaluating the effect of mAb1 on clinical activity and serum tryptase. Activity endpoints included provocation test (TempTest®/ColdU; FricTest®/SD), physician's global assessment (Phys-GA), and patient's global assessment (Pat-GA) of disease severity. Mean±standard error (SE) are shown in FIGS. 12C-12D, FIGS. 14A-14C, FIGS. 15A-15D and FIGS. 16A-16D for provocation tests, biomarkers and hematology, respectively. Skin MC numbers assessed using non-lesional skin biopsies were enumerated by tryptase staining.

The study was amended to add a cohort of patients with cholinergic urticaria.

The study was performed essentially according to the clinical trial protocol described in Example 12.

### 6.13.3 Study Status

Twenty patients received study drug (i.e., mAb1) by a single intravenous infusion at 3 mg/kg and were included in the safety analysis. Eleven had ColdU and 9 patients had SD. Patients had high disease activity as assessed by provocation threshold testing. In patients with ColdU, baseline critical temperature thresholds were 18.9° C./66° F. (range: 5-27° C./41-80.6° F.). In patients with SD, baseline FricTest® thresholds were 3.8 (range: 3-4) of 4 pins.

Nineteen patients received full dose and were included in the activity analysis. Fourteen of 19 patients completed the 12-week observation period; 5 were ongoing.

### 6.13.4 Demographics and Baseline Disease Characteristics

See Table 8 below for characterization of the 20 patients. All of the patients had prior antihistamine treatment.

TABLE 8
Demographics and baseline disease characteristics.

| | | ColdU (N = 11) | | SD (N = 9) | | All (N = 20) | |
|---|---|---|---|---|---|---|---|
| Age median (range) years | | 43 | (27-65) | 41 | (27-56) | 42 | (27-65) |
| Gender Female, n (%) | | 6 | (54.5%) | 4 | (44.4%) | 10 | (50%) |
| Race | White, n (%) | 10 | (90.9%) | 9 | (100%) | 19 | (95%) |
| | Asian, n (%) | 1 | (9.1%) | 0 | (0%) | 1 | (5%) |
| Ethnicity | Hispanic or Latino | 1 | (9.1%) | 0 | (0%) | 1 | (5%) |
| Weight median (range) kg | | 77.0 | (61.0-93.0) | 88.2 | (57.0-122.0) | 80.0 | (57.0-122.0) |
| Disease Duration | <5 yr, n (%) | 6 | (54.5%) | 4 | (44.4%) | 10 | |
| | ≥5 yr, n (%) | 5 | (45.5%) | 5 | (55.6%) | 10 | |
| History of Angioedema | | 6 | (54.5%) | 0 | | 6 | (30%) |
| Provocation Threshold Mean (range) | | 18.9 | (5.0-27.0)° C. | 3.8 | (3-4) pins | | |
| Phys-GA ≥2, n (%) | | 10 | (91%) | 9 | (100%) | 19 | (95%) |
| Pat-GA ≥2, n (%) | | 6 | (55%) | 8 | (89%) | 14 | (70%) |
| Prior Medication | H1 Antihistamine | 11 | (100%) | 9 | (100%) | 20 | (100%) |
| | Biologics (omalizumab) | 1 | (9%) | 2 | (22%) | 3 | (15%) |

| | | | | | | |
|---|---|---|---|---|---|---|
| Tryptase median (range) ng/ml | 3.6 | (2.1-5.5) | 4.7 | (1.6-8.6) | 4.1 | (1.6-8.6) |

### 6.13.5 Study Results

As shown in FIGS. 12A-12D, a single dose of mAb1 (3 mg/kg) resulted in a rapid, profound, and durable response in patients with CIndU refractory to antihistamines. Complete response (CR) was achieved in 95% (18/19) patients (100% (10/10) in ColdU (FIG. 12A) and 89% (8/9) in SD (FIG. 12B) patients). Complete response was observed in all 3 patients (1 ColdU patient and 2 SD patients) with prior Xolair® (omalizumab) experience, including two who were Xolair® refractory. Rapid onset of response after dosing and sustained durability were observed. Most patients with ColdU and SD experienced a complete response by week 1 and by week 4, respectively. The CR was sustained for a median duration of 77 days in ColdU and 57 days in SD patients who completed the 12 week follow-up period (i.e., 8 ColdU patients and 6 SD patients) (FIGS. 12C and 12D). One of the 19 patients (an SD patient) experienced a partial response (PR). CR=negative provocation test at ≤4° C. (for ColdU) or 0 pins (for SD). PR=improvement by 4° C. (for ColdU) or ≤2 pins (for SD).

Improved disease activity as assessed by Phys-GA and Pat-GA was consistent with the complete response as measured by the provocation test (FIGS. 13A and 13B, for ColdU and SD patients, respectively).

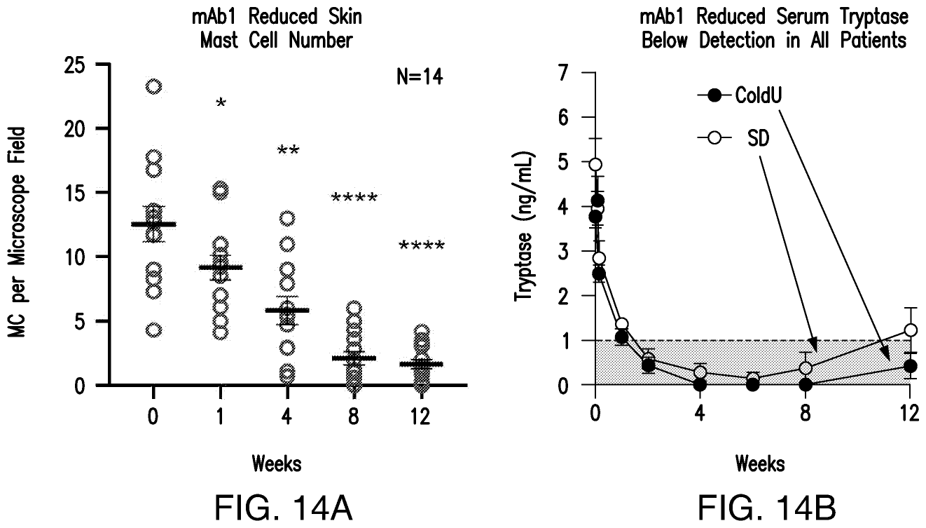

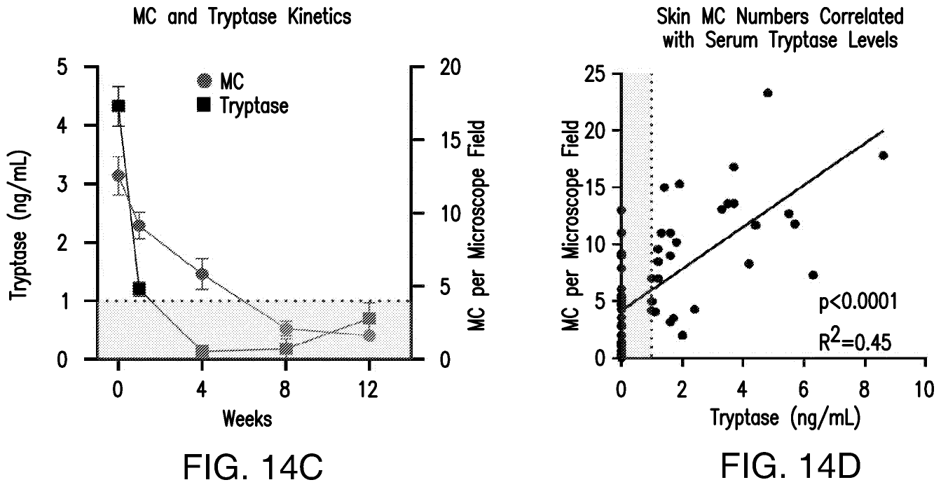

As shown in FIGS. 14A and 14B, a single 3 mg/kg mAb1 dose resulted in a rapid, marked, and durable depletion of skin MCs (87% depletion, see FIG. 14A) and suppression of serum tryptase (FIG. 14B) as measured through biopsy. The MC and tryptase kinetics exhibited similar trends over time (FIG. 14C). In addition, skin MC numbers positively correlated with serum tryptase levels (FIG. 14D).

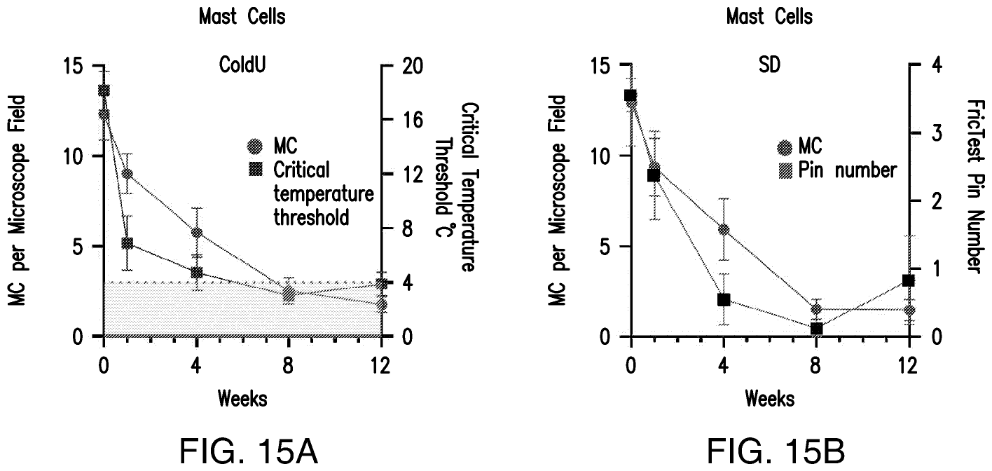

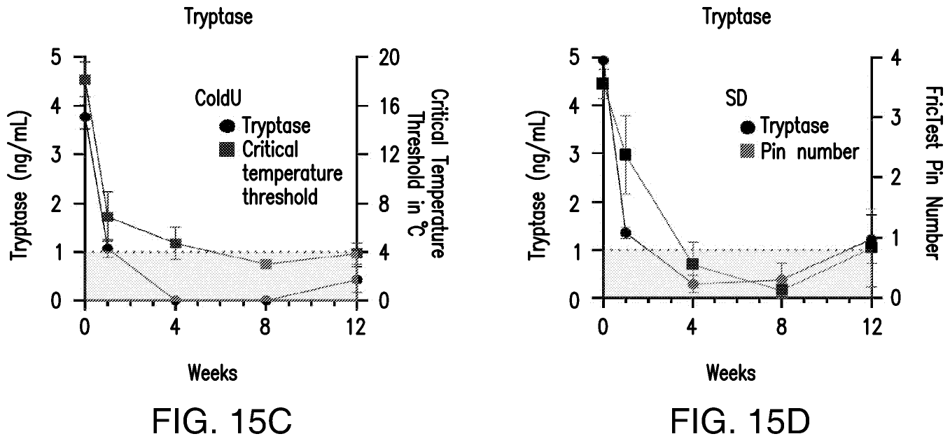

The kinetics of skin MC and serum tryptase depletion mirrored clinical activity. In particular, the kinetics of skin MC and serum tryptase depletion mirrored decreases in provocation thresholds (FIGS. 15A-15D). The data confirmed that serum tryptase level is a robust pharmacodynamic biomarker for assessing MC burden and clinical activity in patients with CIndU and potentially in other diseases with mast cell driven involvement.

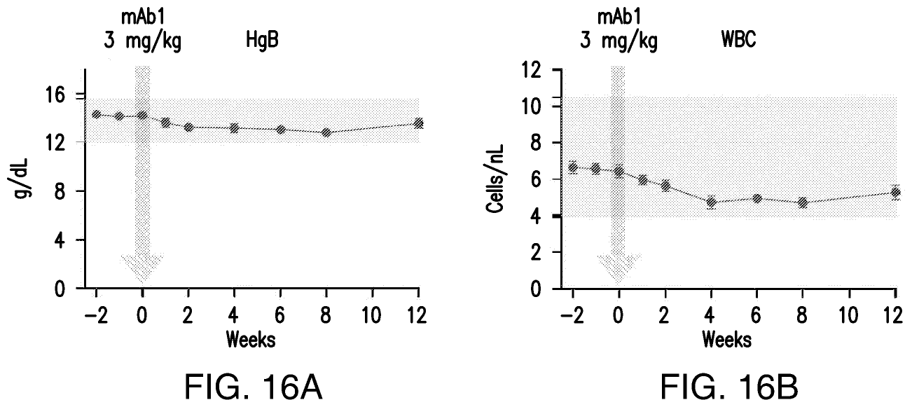

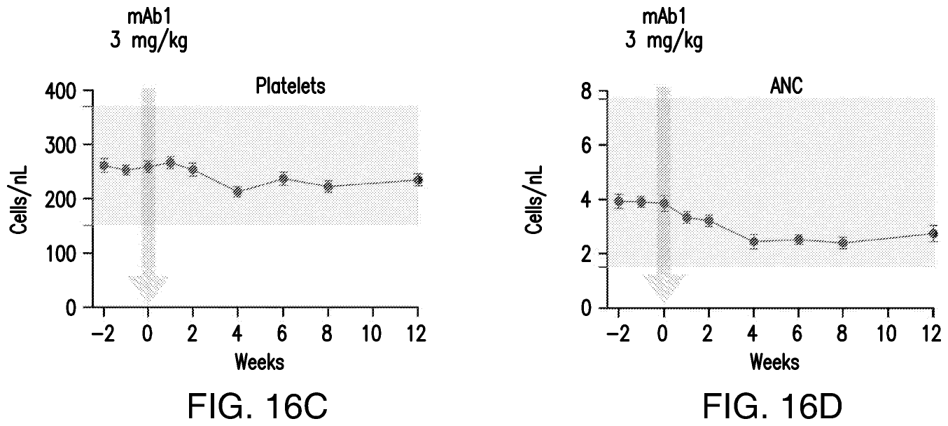

In addition, mAb1 demonstrated favorable safety and tolerability. mAb1 was generally well tolerated in patients with CIndU. The most common adverse events were hair color changes (14/20 (70%)), infusion reactions (9/20 (45%)), and taste disorders (8/20 (40%)). Hair color changes (generally small areas of hair color lightening) and taste disorders (generally partial changes of ability to taste salt) were consistent with inhibiting KIT signaling in other cell types and were expected to be fully reversible. Most adverse events were mild. Hair color changes improved upon longer observation period. Infusion reactions, generally manifested as hives and itching, resolved spontaneously. A single severe infusion reaction of brief loss of consciousness occurred in a patient with a history of fainting and was not attributed to MC activation as measured by serum tryptase monitoring. The patient rapidly recovered. Taste disorders were selective and transient. Hematology parameters generally remained within the normal ranges (see FIGS. 16A-16D). Mild, transient, and asymptomatic decreases in hemoglobin and white blood cell (WBC) parameters were noted (see FIGS. 16A-16D). However, there was no evidence of clinically significant decreases in hematology parameters. This was an important finding for a KIT inhibitor.

Together, the data show that mAb1 demonstrated unprecedented MC depletion with a favorable safety profile, providing a significant potential as a therapy for CIndU for quick, lasting, and meaningful relief, indicating its potential to impact other diseases with mast cell involvement, and opening opportunities for the evaluation of MC involvement across many diseases.

### 6.14 Example 14—mAb1 Demonstrated Rapid and Sustained Clinical Response and Improved Quality of Life in Patients with Chronic Inducible Urticaria

6.14.1 Introduction and objectives:

Chronic inducible urticaria (CIndU) is a group of mast cell (MC)-driven diseases characterized by itchy wheals due to known triggers, such as cold in cold urticaria (ColdU). MC surface receptor KIT is required for MC activation and survival. mAb1 is a monoclonal anti-KIT antibody that has demonstrated profound suppression of circulating tryptase, a marker of MC number, in a previous study in healthy adults.

### 6.14.2 Materials & Methods:

This example describes interim results from an open-label, phase 1 trial of mAb1 (NCT04548869) in adults with CIndU: ColdU or symptomatic dermographism (SD) refractory to antihistamines. Patients received a single intravenous (IV) infusion of mAb1 at 3 mg/kg and were followed for 12 weeks. Safety results are described herein for 19 patients (ColdU: n=10, SD: n=9) receiving study drug; clinical effect results are described herein for 18 patients receiving full dose. Disease activity was assessed by critical temperature threshold (CTT) per TempTest® for ColdU and critical friction threshold (CFT) per FricTest® for SD; a complete response was defined as negative provocation test. Study assessments also included urticaria control test (UCT) and dermatology life quality index (DLQI).

### 6.14.3 Results:

As of the last assessment (Week 2-12), 17/18 patients achieved complete response to provocation testing. In ColdU patients, mean CTT was 18.6° C. (range: 5-27° C.) at Baseline; with a median post-treatment follow-up of 10 weeks, all 9 patients achieved complete response as of the last assessment (Week 4-12) with 6/9 patients achieving complete response by Week 2. In SD patients, mean CFT was 3.8 (range: 3-4) at baseline; with a median post-treatment follow-up of 7 weeks, 8/9 patients achieved complete response as of the last assessment (Week 2-12) with 3/9 patients achieving complete response by Week 2. All 7 (6 ColdU and 1 SD) patients who completed the Week 12 assessments maintained complete response. Reduction in provocation threshold was accompanied by durable serum tryptase suppression: from Baseline 4.4±1.6 ng/ml to near or below the limit of detection at Week 2 in most patients and maintained through Week 8. Mean UCT score improved from 6 (range: 0-13) at baseline to 13 (10-16) at Week 4, 14 patients achieved UCT≥12 (well-controlled), of which 5 achieved UCT=16 (complete control) by Week 4. DLQI scores at baseline and Week 4 were available for 15 patients. Mean DLQI score improved from 11 (range: 2-21) at baseline to 2 (range: 0-8) at Week 4, 8 patients achieved a score of 0 or 1 (no disease impact on quality of life) by Week 4. The most common adverse events were hair color changes in 11/19, infusion reactions in 9/19, and taste disorder in 8/19, most events were mild.

### 6.14.4 Conclusions:

A single 3 mg/kg dose of mAb1 demonstrated a rapid and sustained clinical response and tryptase suppression, improved quality of life, and was well-tolerated overall. These data support KIT-driven MC suppression as a potential therapeutic approach for the management of CIndU.

### 6.15 Example 15—mAb1 Demonstrated Rapid and Sustained Clinical Response and Improved Quality of Life in Patients with Chronic Inducible Urticaria

### 6.15.1 Study Background and Summary

Chronic inducible urticaria (CIndU) is a mast cell (MC)-driven disease characterized by itch and wheals triggered by cold in cold urticaria (ColdU), or skin scratching in symptomatic dermographism (SD). The activation of KIT receptors by stem cell factor is essential for survival, proliferation, and differentiation of MCs. mAb1 is a monoclonal anti-KIT antibody that was engineered to selectively inhibit stem cell factor (SCF)-dependent KIT activation (see FIG. 11). In healthy volunteers, mAb1 induced a profound dose-dependent reduction in circulating tryptase, a biomarker of MC burden, and was overall well-tolerated. As described in Example 13, a single dose of mAb1 (3 mg/kg) was generally well-tolerated and resulted in rapid and durable complete responses (negative provocation test) in antihistamine-refractory CIndU (ColdU and SD) patients. This Example 15 describes additional data obtained from the same study. The data presented in this Example 15 show the effect of mAb1 on urticaria control and quality of life (QoL) of these patients.

### 6.15.2 Study Design and Methods

In this ongoing open-label, Phase 1b trial, patients with ColdU and SD refractory to antihistamine treatment received a single IV infusion of mAb1 at 3 mg/kg with a 12-week follow-up. Primary objective was to evaluate safety/tolerability of mAb1 (adverse events and clinical lab tests). Secondary objectives included evaluating the effect of mAb1 on clinical effect and serum tryptase. Clinical effect assessments included provocation test (TempTest®/ColdU; FricTest®/SD), urticarial control test (UCT) and dermatology life quality index (DLQI).

The study was performed essentially according to the clinical trial protocol described in Example 12.

### 6.15.3 Study Status

Twenty-one patients received study drug (i.e., mAb1) by a single intravenous infusion at 3 mg/kg and were included in the safety analysis. Eleven had ColdU and 10 patients had SD.

Twenty patients received a full dose of study drug and were included in UCT, DLQI, and provocation test data.

Twenty ofTwenty-one patients completed the 12-week observation period; 1 was ongoing.

### 6.15.4 Demographics and Baseline Characteristics

See Table 9 below for characterization of the 21 patients. All of the patients had prior antihistamine treatment.

TABLE 9
Demographics and baseline characteristics.

| | | ColdU (N = 11) | | SD (N = 10) | | All (N = 21) | |
|---|---|---|---|---|---|---|---|
| Age median (range) years | | 43 | (27-65) | 39 | (25-56) | 41 | (25-65) |
| Gender Female, n (%) | | 6 | (54.5%) | 4 | (40.0%) | 10 | (47.6%) |
| Race | White, n (%) | 10 | (90.9%) | 10 | (100%) | 20 | (95.2%) |
| | Asian, n (%) | 1 | (9.1%) | 0 | (0%) | 1 | (4.8%) |
| Ethnicity | Hispanic or Latino | 1 | (9.1%) | 0 | (0%) | 1 | (4.8%) |
| Weight median (range) kg | | 77.0 | (61.0-93.0) | 85.7 | (57.0-122.0) | 81.5 | (57.0-122.0) |
| Disease Duration | <5 yr, n (%) | 5 | (45.5%) | 4 | (40%) | 9 | (42.9%) |
| | ≥5 yr, n (%) | 6 | (54.5%) | 6 | (60%) | 12 | (57.1%) |
| History of Angioedema | | 6 | (54.5%) | 0 | | 6 | (28.6%) |
| Provocation Threshold Mean (range) | | 18.9 | (5-27)° C. | 3.5 | (2-4) Pins | | |
| UCT Mean (range) | | 7 | (2-13) | 4.9 | (0-10) | 6.0 | (0-13) |
| DLQI Mean (range) | | 10.8 | (3-17) | 11.4 | (2-21) | 11.1 | (2-21) |
| Prior Medication | H1 Antihistamines | 11 | (100%) | 10 | (100%) | 21 | (100%) |
| | Biologics (omalizumab) | 1 | (9%) | 2 | (20%) | 3 | (14.3%) |

| | | | | | | |
|---|---|---|---|---|---|---|
| Tryptase median (range) ng/ml | 3.8 | (2.4-5.5) | 4.6 | (1.3-8.6) | 4.2 | (1.3-8.6) |

### 6.15.5 Study Results

In patients with CIndU refractory to antihistamines, a single dose of mAb1 (3 mg/kg) resulted in rapid, profound, and durable responses in 100% of patients with 95% achieving complete response, as described in Example 13.

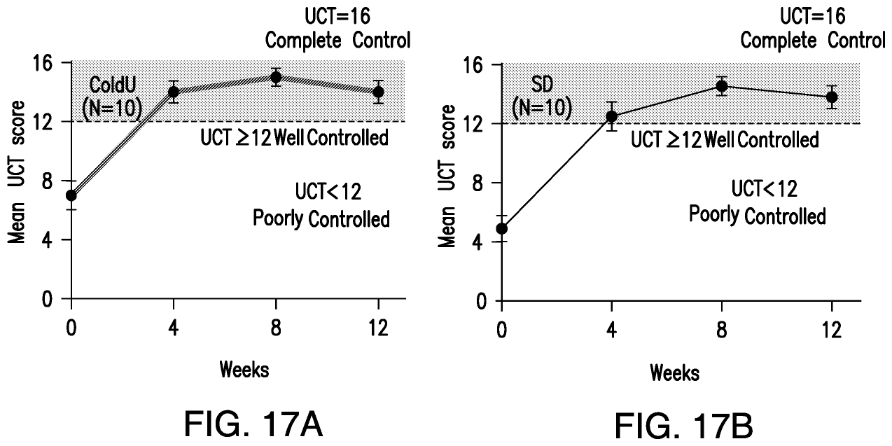

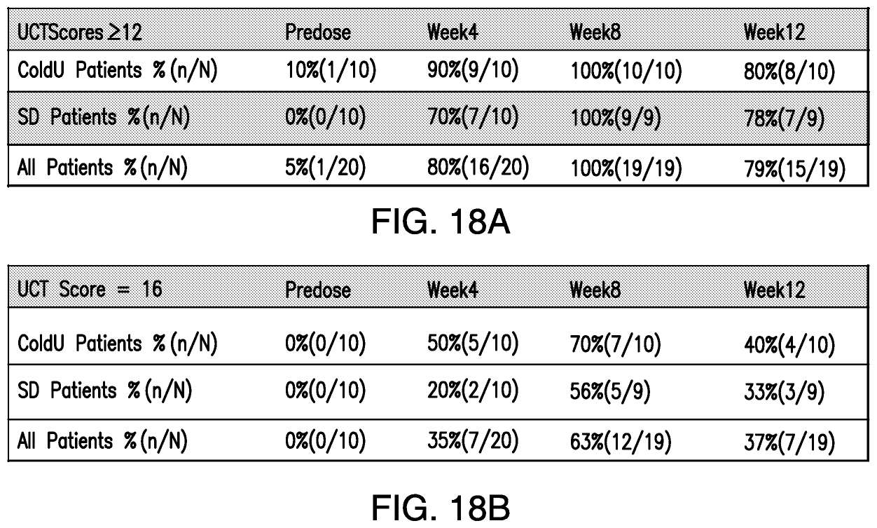

This noteworthy response to provocation testing was also accompanied by markedly improved urticarial control and quality of life. A single 3 mg/kg dose of mAb1 resulted in rapid and sustained improvement in urticaria control in ColdU and SD patients (FIGS. 17A, 17B, 18A and 18B). Rapid improvement in the UCT score was noted within 4 weeks and sustained to Week 12 (FIGS. 17A, 17B, 18A and 18B). 80% and 100% patients achieved “well controlled” status (UCT≥12) by Week 4 and 8, respectively (FIG. 18A). 63% of patients achieved “complete control” status (UCT=16) by Week 8 (FIG. 18B).

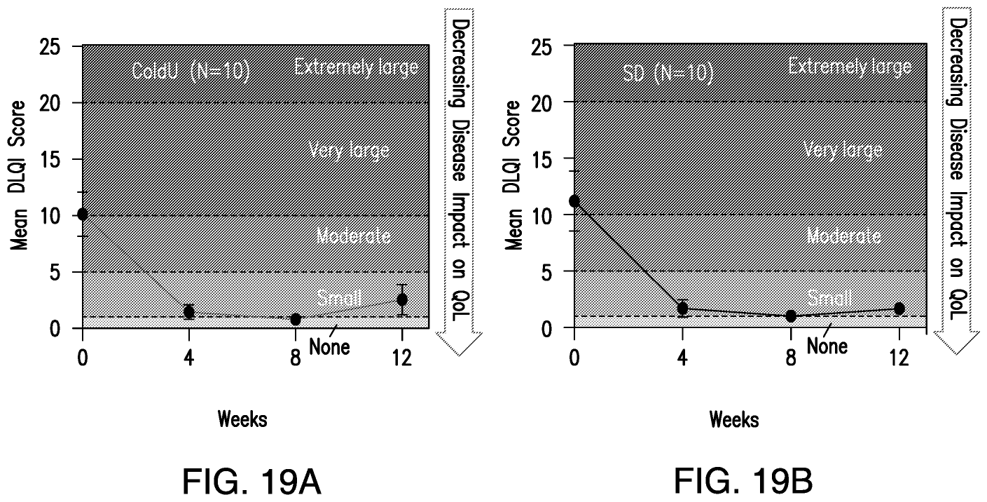

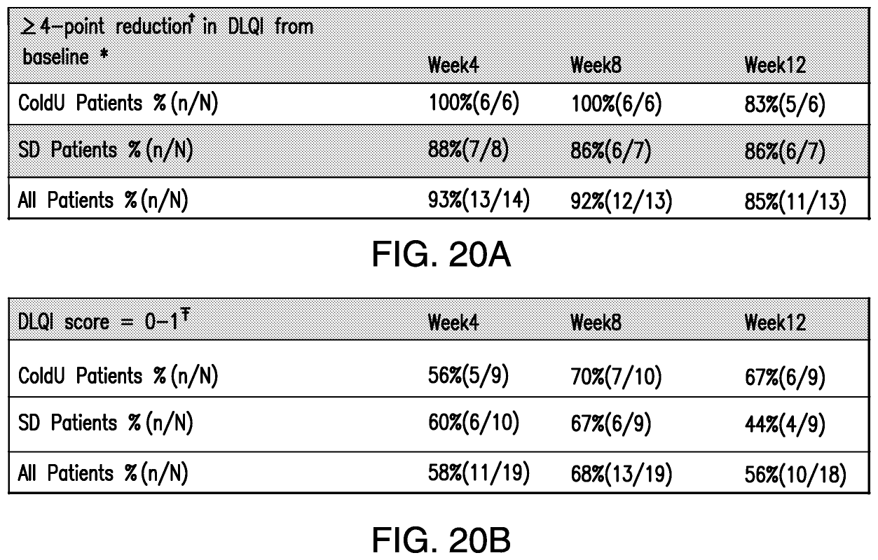

mAb1 also greatly reduced disease impact on the quality of life of patients with ColdU and SD (FIGS. 19A and 19B). 93% and 92% patients achieved≥4-point reduction (minimal clinically important difference (MCID)) in DLQI by Week 4 and 8, respectively (FIG. 20A). 58% and 68% patients achieved DLQI score of 0-1 (no impact of disease on quality of life) by Week 4 and 8, respectively (FIG. 20B).

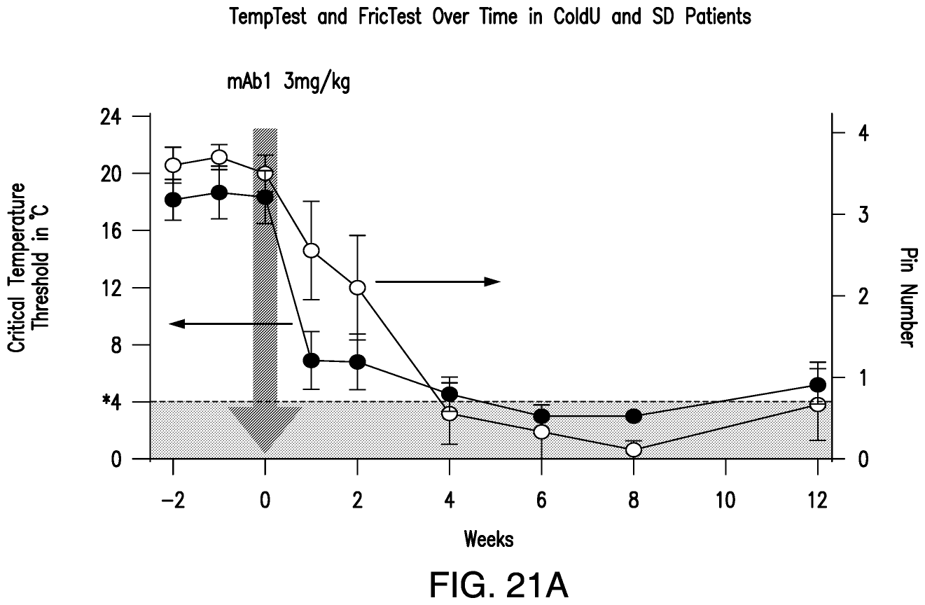

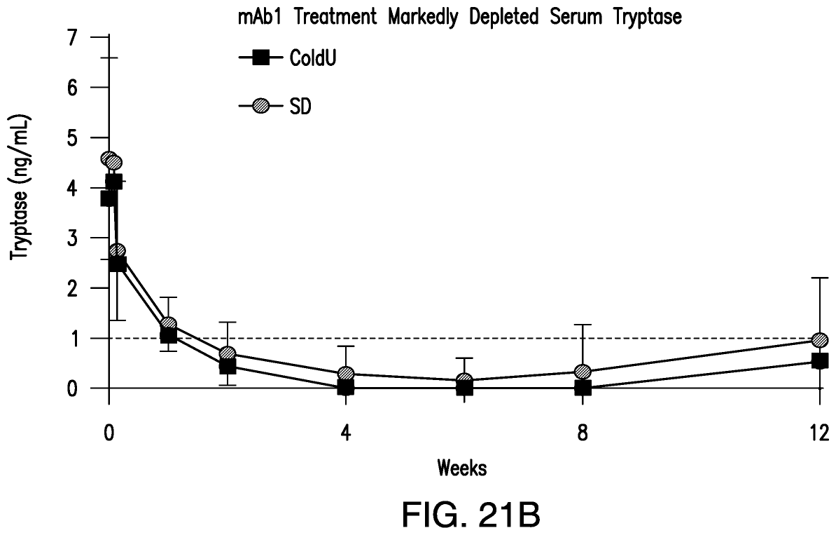

In addition, as described above, rapid and durable improvement in provocation tests was achieved with a 95% complete response (FIG. 21A). Rapid, durable and profound tryptase reduction was also achieved (FIG. 21). The rapid and durable improvement in provocation response mirrored the reduction in tryptase (FIGS. 21A and 21).

mAb1 was generally well tolerated in patients with CIndU (ColdU and SD). The most common adverse events were hair color changes (15/21 (71%)), infusion reactions (9/21 (43%)), and taste disorders (8/21 (38%)). Hair color changes and taste disorders were consistent with inhibiting KIT signaling in other cell types and were expected to be fully reversible. Most adverse events were mild. Hair color changes improved upon longer observation period. Infusion reactions were mostly mild, generally manifested as hives and itching, and resolved spontaneously. A single severe infusion reaction occurred that was not attributed to MC activation. Taste disorders were selective and transient. Hematology parameters generally remained within the normal ranges. Mild, transient, and asymptomatic decreases in hemoglobin and white blood cell (WBC) parameters were noted. However, there was no evidence of clinically significant decreases in hematology parameters.

Together, the data show that mAb1 is generally safe and well tolerated, and that it has significant potential as a therapy for CIndU, and other mast cell-related diseases.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1. An antibody which immunospecifically binds to human KIT, comprising:
(i) a light chain variable region (“VL”) comprising VL CDR1, VL CDR2, and VL CDR3 having the amino acid sequences of SEQ ID NO: 2, SEQ ID NO: 3, and SEQ ID NO: 4, respectively;
(ii) a heavy chain variable region (“VH”) comprising VH CDR1, VH CDR2, and VH CDR3 having the amino acid sequences of SEQ ID NO: 5, SEQ ID NO: 6, and SEQ ID NO: 7, respectively; and
(iii) a modified human IgG1 Fc region or domain which comprises non-naturally occurring amino acids 234A, 235Q and 322Q as numbered by the EU index as set forth in Kabat.

2. The antibody of claim 1, wherein the modified human IgG1 Fc region or domain further comprises non-naturally occurring amino acids 252Y, 254T and 256E as numbered by the EU index as set forth in Kabat.

3. The antibody of claim 1 or 2, wherein:
(i) the VL comprises the amino acid sequence:
(SEQ ID NO: 17)
DIVMTQSPSX_{K1}LSASVGDRVTITCKASQNVR
TNVAWYQQKPGKAPKX_{K2}LIYSASYRYSGVPD
RFX_{K3}GSGSGTDFTLTISSLQX_{K4}EDFAXKSY
X_{K6}CQQYNSYPRTFGGGTKVEIK,
wherein X_{K1} is an amino acid with an aromatic or aliphatic hydroxyl side chain, X_{K2} is an amino acid with an aliphatic or aliphatic hydroxyl side chain, X_{K3} is an amino acid with an aliphatic hydroxyl side chain, X_{K4} is an amino acid with an aliphatic hydroxyl side chain or is P, X_{K5} is an amino acid with a charged or acidic side chain and X_{K6} is an amino acid with an aromatic side chain; and
(ii) the VH comprises the amino acid sequence:
(SEQ ID NO: 18)
QVQLVQSGAEX_{H1}KKPGASVKX_{H2}SCKASGYT
FTDYYINWVX_{H3}QAPGKGLEWIARIYPGSGNT
YYNEKFKGRX_{H4}TXHSTAX_{H6}KSTSTAYMX_{H7}
LSSLRSEDX_{H8}AVYFCARGVYYFDYWGQGTTV
TVSS,
wherein X_{H1} to X_{H8} is any amino acid.
wherein X_{H1} is an amino acid with an aliphatic side chain, X_{H2} is an amino acid with an aliphatic side chain, X_{H3} is an amino acid with a polar or basic side chain, X_{H4} is an amino acid with an aliphatic side chain, X_{H15} is an amino acid with an aliphatic side chain, X_{H6} is an amino acid with an acidic side chain, X_{H7} is an amino acid with an acidic or amide derivative side chain, and X_{K8} is an amino acid with an aliphatic hydroxyl side chain.

4. The antibody of claim 3, wherein X_{K1} is the amino acid F or S, X_{K2} is the amino acid A or S, X_{K3} is the amino acid T or S, X_{K4} is the amino acid S or P, X_{K5} is the amino acid D or T, X_{K6} is the amino acid F or Y, X_{H1} is the amino acid L or V, X_{H2} is the amino acid L or V, X_{H3} is the amino acid K or R, X_{H4} is the amino acid V or A, X_{H5} is the amino acid L or I, X_{H6} is the amino acid E or D, X_{H7} is the amino acid Q or E, and X_{H8} is the amino acid S or T.

5. The antibody of any one of claims 1 to 4, wherein:
i) the VL comprises the amino acid sequence of SEQ ID NO: 13, 14, 15 or 16, and
ii) the VH comprises the amino acid sequence of SEQ ID NO: 8, 9, 10, 11 or 12.

6. The antibody of any one of claims 1 to 5, comprising:
(i) a VL comprising an amino acid sequence of SEQ ID NO: 14;
(ii) a VH comprising an amino acid sequence of SEQ ID NO: 10; and
(iii) a modified human IgG1 Fc region or domain comprising non-naturally occurring amino acids 234A, 235Q and 322Q as numbered by the EU index as set forth in Kabat.

7. The antibody of any one of claims 1 to 5, comprising:
(i) a VL comprising an amino acid sequence of SEQ ID NO: 14;
(ii) a VH comprising an amino acid sequence of SEQ ID NO: 10; and
(iii) a modified human IgG1 Fc region or domain comprising non-naturally occurring amino acids 234A, 235Q, 322Q, 252Y, 254T and 256E as numbered by the EU index as set forth in Kabat.

8. The antibody of any one of claims 1 to 5, which comprises a heavy chain comprising the amino acid sequence:
(SEQ ID NO: 21)
QVQLVQSGAEVKKPGASVKLSCKASGYTFTDYYINWVRQA
PGKGLEWIARIYPGSGNTYYNEKFKGRATLTADKSTSTAY
MQLSSLRSEDTAVYFCARGVYYFDYWGQGTTVTVSSASTK
GPSVFPLAPSSKSTSGGTAALGCLVKDYFPEPVTVSWNSG
ALTSGVHTFPAVLQSSGLYSLSSVVTVPSSSLGTQTYICN
VNHKPSNTKVDKKVEPKSCDKTHTCPPCPAPEAQGGPSVF
LFPPKPKDTLYITREPEVTCVVVDVSHEDPEVKFNWYVDG
VEVHNAKTKPREEQYNSTYRVVSVLTVLHQDWLNGKEYKC
QVSNKALPAPIEKTISKAKGQPREPQVYTLPPSRDELTKN
QVSLTCLVKGFYPSDIAVEWESNGQPENNYKTTPPVLDSD
GSFFLYSKLTVDKSRWQQGNVFSCSVMHEALHNHYTQKSL
SLSPG.

9. The antibody of any one of claims 1 to 5, which comprises a light chain comprising the amino acid sequence:
(SEQ ID NO: 22)
DIVMTQSPSSLSASVGDRVTITCKASQNVRTNVAWYQQKP
GKAPKALIYSASYRYSGVPDRFTGSGSGTDFTLTISSLOP
EDFADYFCQQYNSYPRTFGGGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSENRGEC.

10. The antibody of any one of claims 1 to 5, which comprises a heavy chain comprising the amino acid sequence:
(SEQ ID NO: 21)
QVQLVQSGAEVKKPGASVKLSCKASGYTFTDYYINWVRQA
PGKGLEWIARIYPGSGNTYYNEKFKGRATLTADKSTSTAY
MQLSSLRSEDTAVYFCARGVYYFDYWGQGTTVTVSSASTK
GPSVFPLAPSSKSTSGGTAALGCLVKDYFPEPVTVSWNSG
ALTSGVHTFPAVLQSSGLYSLSSVVTVPSSSLGTQTYICN
VNHKPSNTKVDKKVEPKSCDKTHTCPPCPAPEAQGGPSVF
LFPPKPKDTLYITREPEVTCVVVDVSHEDPEVKFNWYVDG
VEVHNAKTKPREEQYNSTYRVVSVLTVLHQDWLNGKEYKC
QVSNKALPAPIEKTISKAKGQPREPQVYTLPPSRDELTKN
QVSLTCLVKGFYPSDIAVEWESNGQPENNYKTTPPVLDSD
GSFFLYSKLTVDKSRWQQGNVFSCSVMHEALHNHYTQKSL
SLSPG;
and a light chain comprising the amino acid sequence:
(SEQ ID NO: 22)
DIVMTQSPSSLSASVGDRVTITCKASQNVRTNVAWYQQKP
GKAPKALIYSASYRYSGVPDRFTGSGSGTDFTLTISSLQP
EDFADYFCQQYNSYPRTFGGGTKVEIKRTVAAPSVFIFPP
SDEQLKSGTASVVCLLNNFYPREAKVQWKVDNALQSGNSQ
ESVTEQDSKDSTYSLSSTLTLSKADYEKHKVYACEVTHQG
LSSPVTKSFNRGEC.

11. A conjugate comprising the antibody of any one of claims 1 to 10 linked to an agent.

12. A pharmaceutical composition comprising the antibody of any one of claims 1 to 10, or the conjugate of claim 11, and a pharmaceutically acceptable carrier.

13. A polynucleotide or combination of polynucleotides comprising nucleotide sequences encoding the antibody of any one of claims 1 to 10, or the VH and VL of said antibody.

14. A vector or combination of vectors comprising the polynucleotide or combination of polynucleotides of claim 13.

15. A host cell comprising the vector or combination of vectors of claim 14 or the polynucleotide or combination of polynucleotides of claim 13.

16. A kit comprising the antibody of any one of claims 1 to 10, the conjugate of claim 11, or the pharmaceutical composition of claim 12.

17. A method for protecting against, treating or managing a KIT-associated disorder, comprising administering to a subject in need thereof a therapeutically effective amount of the antibody of any one of claims 1 to 10, the conjugate of claim 11, or the pharmaceutical composition of claim 12.

18. The method of claim 17, wherein the KIT-associated disorder is a mast cell related disorder, an eosinophil related disorder, a cancer, asthma, an inflammatory condition, rheumatoid arthritis, an allergic inflammation, inflammatory bowel disease, a gastrointestinal disorder, or fibrosis.

19. The method of claim 18, wherein the KIT-associated disorder is a mast cell related disorder.

20. The method of claim 19, wherein the mast cell related disorder is chronic urticaria.

21. The method of claim 20, wherein the chronic urticaria is chronic inducible urticaria.

22. The method of claim 21, wherein the chronic inducible urticaria is cold urticaria.

23. The method of claim 21, wherein the chronic inducible urticaria is symptomatic dermographism.

24. The method of claim 21, wherein the chronic inducible urticaria is cholinergic urticaria.

25. The method of claim 20, wherein the chronic urticaria is chronic spontaneous urticaria.

26. The method of claim 18, wherein the KIT-associated disorder is an eosinophil related disorder.

27. The method of any one of claims 17 to 26, further comprising administering a second therapeutic agent to the subject.

28. The method of claim 27, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.

29. A method for inhibiting KIT activity in a cell expressing KIT, comprising contacting the cell with an effective amount of the antibody of any one of claims 1 to 10, the conjugate of claim 11, or the pharmaceutical composition of claim 12.

30. The method of claim 29, wherein the method inhibits KIT activity by at least about 10% in the cell expressing KIT.

31. An in vitro method for diagnosing a subject with a KIT-associated disorder, wherein the method comprises contacting cells or a sample obtained from the subject with the antibody of any one of claims 1 to 10 and detecting the expression level of KIT in the cells or the sample.

32. A method of making an antibody, wherein said method comprises culturing, and/or expressing the antibody using, the host cell of claim 15.

33. The method of claim 32, further comprising purifying the antibody obtained from said host cell.

34. A method for protecting against, treating or managing chronic urticaria in a subject, comprising administering to a subject in need thereof a therapeutically effective amount of (1) an antibody which immunospecifically binds to human KIT or an antigen binding fragment thereof, (2) a conjugate comprising the antibody or antigen binding fragment thereof linked to an agent, or (3) a pharmaceutical composition comprising the antibody or antigen binding fragment thereof or the conjugate, and a pharmaceutically acceptable carrier.

35. The method of claim 34, wherein the human KIT comprises the amino acid sequence of SEQ ID NO: 1.

36. The method of claim 34 or 35, wherein the antibody specifically binds to a D4 or D5 region of human KIT.

37. The method of any one of claims 34 to 36, wherein the antibody comprises a modified Fe region or domain.

38. The method of any one of claims 34 to 37, wherein the antibody comprises a modified human Fc region or domain.

39. The method of any one of claims 34 to 38, wherein the antibody has reduced Fc receptor binding activity.

40. The method of claim 39, wherein the antibody has reduced FcTR binding activity.

41. The method of any one of claims 33 to 40, wherein the antibody does not induce significant degranulation of FegRI-expressing human mast cells.

42. The method of any one of claims 33 to 41, wherein the antibody does not show significant Fec receptor-dependent KIT agonist activity.

43. The method of any one of claims 33 to 42, wherein the antibody comprises:
(A) (i) a VL comprising VL CDR1, VL CDR2, and VL CDR3 having the amino acid sequences of SEQ ID NO: 2, SEQ ID NO: 3, and SEQ ID NO: 4, respectively; and
(ii) a VH comprising VH CDR1, VH CDR2, and VH CDR3 having the amino acid sequences of SEQ ID NO: 5, SEQ ID NO: 6, and SEQ ID NO: 7, respectively;
(B) (i) a VL comprising VL CDR1, VL CDR2, and VL CDR3 having the amino acid sequences of SEQ ID NO: 2, SEQ ID NO: 3, and SEQ ID NO: 4, respectively; and
(ii) a VH comprising VH CDR1, VH CDR2, and VH CDR3 having the amino acid sequences of SEQ ID NO: 25, SEQ ID NO: 26, and SEQ ID NO: 27, respectively;
(C) (i) a VL comprising VL CDR1, VL CDR2, and VL CDR3 having the amino acid sequences of SEQ ID NO: 28, SEQ ID NO: 29, and SEQ ID NO: 30, respectively; and
(ii) a VI comprising VH CDR1, VH CDR2, and VH CDR3 having the amino acid sequences of SEQ ID NO: 25, SEQ ID NO 31, and SEQ ID NO: 32, respectively;
(D) (i) a VL comprising VL CDR1, VL CDR2, and VL CDR3 having the amino acid sequences of SEQ ID NO: 2, SEQ ID NO: 3, and SEQ ID NO: 4, respectively; and
(ii) a VH comprising VH CDR1, VH CDR2, and VH CDR3 having the amino acid sequences of SEQ ID NO: 33, SEQ ID NO: 34, and SEQ ID NO: 27, respectively; or
(E) (i) a VL comprising VL CDR1, VL CDR2, and VL CDR3 having the amino acid sequences of SEQ ID NO: 35, SEQ ID NO: 36, and SEQ ID NO: 37, respectively; and
(ii) a VH comprising VH CDR1, VH CDR2, and VH CDR3 having the amino acid sequences of SEQ ID NO: 38, SEQ ID NO 39, and SEQ ID NO: 40, respectively.

44. The method of claim 43, wherein the chronic urticaria is chronic inducible urticaria.

45. The method of claim 44, wherein the chronic inducible urticaria is cold urticaria.

46. The method of claim 44, wherein the chronic inducible urticaria is symptomatic dermographism.

47. The method of claim 44, wherein the chronic inducible urticaria is cholinergic urticaria.

48. The method of claim 43, wherein the chronic urticaria is chronic spontaneous urticaria.

49. The method of any one of claims 33 to 48, further comprising administering a second therapeutic agent to the subject.

50. The method of claim 49, wherein said second therapeutic agent is a chemotherapeutic agent, a histone deacetylase inhibitor, an antibody, a cytokine, a tyrosine kinase inhibitor, an antihistamine, a leukotriene receptor antagonist, an immunomodulator, or an anti-inflammatory agent.
